(19) United States
(12) Patent Application Publication
(10) Pub. No.: US 2025/0109139 A1
(43) Pub. Date: Apr. 3, 2025

(54) TRICYCLIC COMPOUND, AND PREPARATION METHOD THEREFOR AND USE THEREOF

(71) Applicants: SHANGHAITECH UNIVERSITY, Shanghai (CN); CENTER FOR EXCELLENCE IN MOLECULAR CELL SCIENCE, CHINESE ACADEMY OF SCIENCES, Shanghai (CN)

(72) Inventors: Jianjun CHENG, Shanghai (CN); Sheng WANG, Shanghai (CN); Ruiquan LIU, Shanghai (CN); Jianzhong QI, Shanghai (CN); Luyu FAN, Shanghai (CN); Jing YU, Shanghai (CN)

(21) Appl. No.: 18/729,910

(22) PCT Filed: Jan. 19, 2023
(86) PCT No.: PCT/CN2023/073216

§ 371 Date: Jul. 18, 2024

(30) Foreign Application Priority Data
Jan. 29, 2022 (CN) 202210112894.8

(51) Int. Cl. C07D 493/04; A61K 31/4355; A61K 31/437; A61K 31/4375; A61K 31/4985; A61K 31/53; A61K 31/538; A61K 31/541; A61K 31/55; C07D 487/04; C07D 519/00

(52) U.S. Cl. CPC C07D 493/04; A61K 31/4355; A61K 31/437; A61K 31/4375; A61K 31/4985; A61K 31/53; A61K 31/538; A61K 31/541; A61K 31/55; C07D 487/04; C07D 519/00

(57) ABSTRACT

Disclosed are a tricyclic compound, and a preparation method therefor and the use thereof. The tricyclic compound has a structure as shown in formula I, and can be used for treating various mental diseases and neurodegenerative diseases such as schizophrenia, depression and Parkinson's disease.

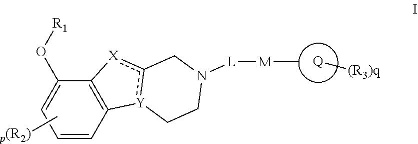

The present application claims the right of the priority of Chinese patent application 202210112894.8 filed on Jan. 29, 2022. The contents of the above Chinese patent application are incorporated herein by reference in their entireties.

### TECHNICAL FIELD

The present disclosure relates to a tricyclic compound, a preparation method therefor, and a use thereof.

### BACKGROUND

Monoamine G protein-coupled receptors (GPCRs), such as dopamine receptors and 5-hydroxytryptamine (5-HT) receptors, are associated with various mental diseases and neurodegenerative diseases, such as schizophrenia, depression, and Parkinson's disease. Accordingly, most antipsychotics and neurodegenerative disease-modifying drugs exert their efficacy by modulating the function of monoamine GPCRs such as dopamine receptors and 5-hydroxytryptamine receptors.

Dopamine receptors have five subtypes (D₁₋₅), where D₁ and D₅ belong to the D₁-like class of receptors, which are mainly coupled with Gₛ proteins, and increase intracellular cyclic adenosine monophosphate (cAMP) levels after activation; D₂, D₃, and D₄ belong to the D₂-like class of receptors, which are mainly coupled with Gi proteins, and decrease intracellular cAMP levels after activation. Abnormalities in the dopaminergic signaling pathway are associated with various diseases, such as schizophrenia and Parkinson's disease. Small molecule antagonists or partial agonists targeting dopamine D₂ receptors are effective antipsychotics, such as Haloperidol, Olanzapine, Risperidone, Aripiprazole, and Cariprazine, all of which act at dopamine D₂ receptors as their primary targets. Dopamine D₃ receptors, which belong to the same subfamily as D₂, are also important targets for many antipsychotics (e.g., Cariprazine). Highly selective D₃ receptor antagonists or partial agonists also have potential for the treatment of drug addiction.

Of the five subtypes of dopamine receptors, D₂ receptors have been the most widely and intensively studied. Dopamine D₂ receptor ligands can be divided into various forms such as agonists, antagonists, or partial agonists depending on their intrinsic activity. Clinically used drugs such as Pramipexole, Ropinirole, and Rotigotine are dopamine D₂ receptor agonists, which exert a therapeutic effect by activating dopamine D₂ receptors, and are used for the treatment of diseases such as Parkinson's disease and restless legs syndrome. Atypical antipsychotics such as Haloperidol, Olanzapine, and Risperidone are dopamine D₂ receptor antagonists, which exert their efficacy by antagonizing D₂ receptors. The latest generation of antipsychotics, namely Aripiprazole, Brexpiprazole, and Cariprazine, are dopamine D₂ receptor partial agonists, which can exert a stabilizing effect on dopaminergic signaling, and are also known as dopamine stabilizers.

It is worth noting that most of the currently known dopamine D₂ antagonists and partial agonists have the disadvantage of poor selectivity. For drugs targeting dopamine D₂ receptors, the effect of the compound on other targets can affect the efficacy and toxic side effects of the drug. For example, for dopamine D₂ receptor antagonists, antagonism of 5-HT_{2A} receptors by the drug can improve the extrapyramidal side effects of the compound, in which case increased antagonism of 5-HT_{2A} receptors is beneficial. In fact, 5-HT_{2A} is another important target of antipsychotics, and is a primary target of multi-target “atypical” antipsychotics. The relative affinity of antipsychotics for 5-HT_{2A} receptors and dopamine D₂ receptors is an important basis for distinguishing between “typical” and “atypical” drugs. However, effects on many other targets, such as antagonism of histamine H₁, antagonism of 5-HT₂c receptors, and off-target effects of drugs on cholinergic and adrenergic receptors, are responsible for many toxic side effects of most of the current antipsychotics, such as weight gain and diabetes induction. The off-target inhibitory effect of some drugs on cholinergic activity is also a possible cause of further deterioration of cognitive function in patients.

Recent studies have demonstrated that 5-HT_{2A} receptor antagonism is not necessary for the antipsychotic activity of dopamine D₂ receptor partial agonists, and that D₂ receptor partial agonists with reduced 5-HT_{2A} receptor antagonism have better efficacy in improving negative symptoms and cognitive function of schizophrenia (Chen et al., Nat Neurosci 2022, 25, 39-49).

Serotonin 5-HT_{1A} receptor is another target that is closely related to the treatment of mental diseases such as schizophrenia. 5-HT_{1A} receptor agonists have shown promising clinical applications in the treatment of depression and anxiety, and improvement of negative symptoms and cognitive function in patients with schizophrenia. For dopamine D₂ receptor antagonists, the agonistic or partial agonistic effects of the drug on 5-HT_{1A} receptors can also improve the extrapyramidal side effects of the drug. Atypical antipsychotics such as Aripiprazole, Brexpiprazole, and Cariprazine all have partial agonistic effects on 5-HT_{1A} receptors. Tandospirone, which is used for the treatment of generalized anxiety disorder; Gepirone, which has antidepressant and anxiolytic effects; Buspirone, which is used for the treatment of symptoms associated with anxiety; and Ipsapirone, which has antidepressant and anxiolytic effects, all have pharmacological effects of 5-HT_{1A} receptor agonists. Flibanserin, a 5-HT_{1A} receptor agonist, was approved by the U.S. FDA in 2015 for the treatment of low libido in women. 5-HT_{1A} receptor agonists or partial agonists are an important direction for the development of new drugs such as novel antipsychotics, antidepressants, and anxiolytics.

Therefore, the development of small molecule compounds with new functional characteristics and selectivity for monoamine GPCRs such as dopamine D₂ receptors and 5-HT_{1A} receptors has the potential to obtain novel antipsychotics to enhance the efficacy and to reduce the related toxic side effects.

### Content of the Present Invention

In order to overcome the above problems in the prior art, the present disclosure provides a tricyclic compound, a preparation method therefor, and a use thereof. The compound of the present disclosure can be used for the treatment of various mental diseases and neurodegenerative diseases such as schizophrenia, depression, and Parkinson's disease.

The present disclosure provides a compound of formula I, or a pharmaceutically acceptable salt, isotope derivative, enantiomer, diastereomer, tautomer, or solvate thereof,

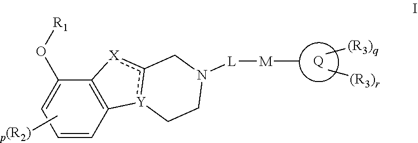

wherein
X is N, O, NRₐ, or CR_{b};
Y is C or N;
[symbol] is a double bond or a single bond;
Rₐ and R_{b} are each independently H or C₁-C₆ alkyl;
L is —(CR_{c}R_{d})ₘ— or —(CR_{c}R_{d})ₙ₁—CH═CH—(CR_{c}R_{d})ₙ₂—;
R_{c} and R_{d} are each independently H or C₁-C₆ alkyl;
m, n1, and n2 are each independently 1, 2, 3, 4, 5, or 6;
M is absent, —O—, or —NH—C(O)—;
ring Q is a saturated or partially unsaturated 3- to 8-membered carbocyclic ring, a saturated or partially unsaturated 3- to 8-membered heterocyclic ring, a 6- to 10-membered aromatic ring, a 5- to 10-membered heteroaromatic ring, or an 8- to 11-membered fused bicyclic ring; one of the rings in the 8- to 11-membered fused bicyclic ring is a saturated or partially unsaturated 5- to 7-membered carbocyclic ring or a saturated or partially unsaturated 5- to 7-membered heterocyclic ring, and the other ring is benzene ring or a 5- to 6-membered heteroaromatic ring;
R₁ is C₁-C₆ alkyl or halo-C₁-C₆ alkyl;
each R₂ is independently F, Cl, Br, I, hydroxyl, C₁-C₆ alkyl, C₁-C₆ alkoxy, halo-C₁-C₆ alkyl, or halo-C₁-C₆ alkoxy;
each R₃ is independently F, Cl, Br, I, hydroxyl, C₁-C₆ alkyl, C₁-C₆ alkoxy, halo-C₁-C₆ alkyl, halo-C₁-C₆ alkoxy, —NH—C(O)Rₑ, or —NH—S(O)₂Rₑ;
or, two R₃ together with the atom to which they are attached form a 3- to 8-membered cycloalkyl ring;
Rₑ is C₁-C₆ alkyl, —NH₂, —NHR_{g}, —NR_{f}R_{g}, or 5- to 6-membered heteroaryl;
R_{f} and R_{g} are each independently C₁-C₆ alkyl;
R₄ is oxo (═O);
p, q, and r are each independently 0, 1, 2, 3, or 4;
the number of heteroatoms in the heterocyclic ring, heteroaromatic ring, and heteroaryl is each independently 1, 2, or 3, and the heteroatoms are each independently N, O, or S.

In some embodiments, Rₐ and R_{b} are H.

In some embodiments, R_{c} and R_{d} are H.

In some embodiments, m is 2, 3, or 4.

In some embodiments, m is 1.

In some embodiments, n1 and n2 are 1.

In some embodiments, L is —(CH₂)₂—, —(CH₂)₃—, —(CH₂)₄—, or —(CH₂)—CH═CH—(CH₂)—.

In some embodiments, L is —(CH₂)—.

In some embodiments, in M, —NH—C(O)— is attached to L through an N atom.

In some embodiments, each R₁ is independently C₁-C₆ alkyl, such as methyl, ethyl, or isopropyl.

In some embodiments, each R₃ is independently F, Cl, Br, I, hydroxyl, C₁-C₆ alkyl, —NH—C(O)Rₑ, or —NH—S(O)₂Rₑ; preferably, each R₃ is independently C₁-C₆ alkyl or —NH—C(O)Rₑ.

In some embodiments, p is 0.

In some embodiments, ring Q is attached to M through a C atom.

In some embodiments, in ring Q, the 3- to 8-membered carbocyclic ring is a 4-membered, 5-membered, 6-membered, or 7-membered carbocyclic ring.

In some embodiments, in ring Q, the 3- to 8-membered heterocyclic ring is a 6-membered heterocyclic ring.

In some embodiments, in ring Q, the heteroatom in the 3- to 8-membered heterocyclic ring is N or O, such as an N atom.

In some embodiments, in ring Q, the 6- to 10-membered aromatic ring is benzene ring.

In some embodiments, in ring Q, the number of heteroatoms in the 5- to 10-membered heteroaromatic ring is 1 or 2.

In some embodiments, in ring Q, one of the rings in the 8- to 11-membered fused bicyclic ring is a saturated or partially unsaturated 5- to 7-membered heterocyclic ring, and the other ring is benzene ring or a 5- to 6-membered heteroaromatic ring, wherein the number of heteroatoms in the 5- to 7-membered heterocyclic ring and the 5- to 6-membered heteroaromatic ring is each independently 1 or 2, and the heteroatom is N.

In some embodiments, ring Q is

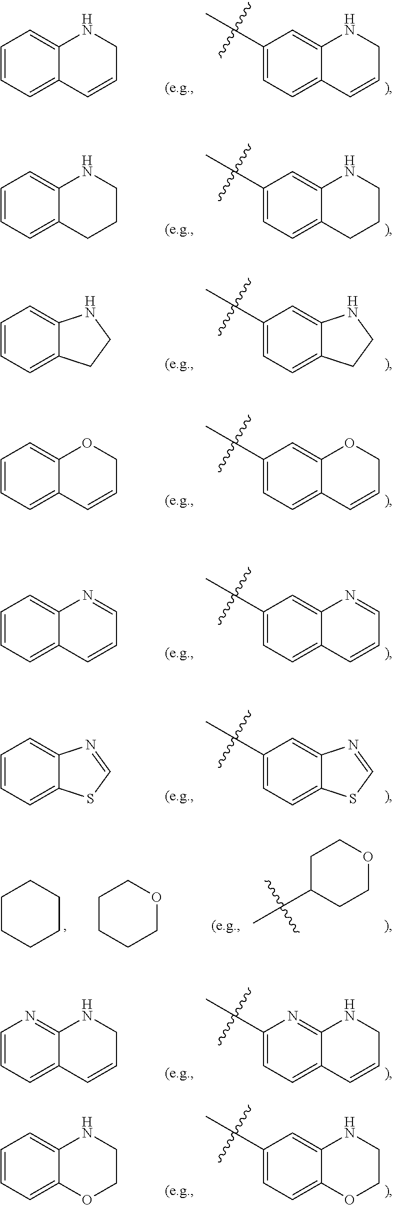

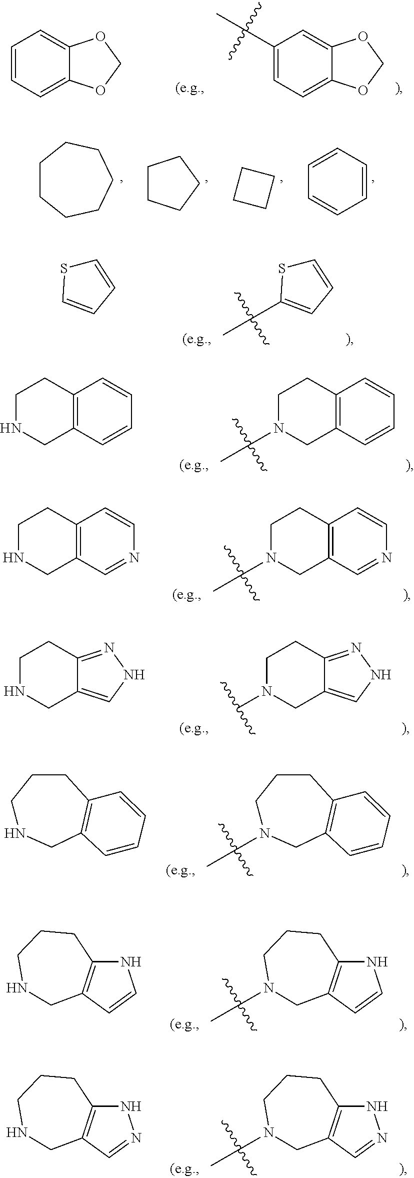

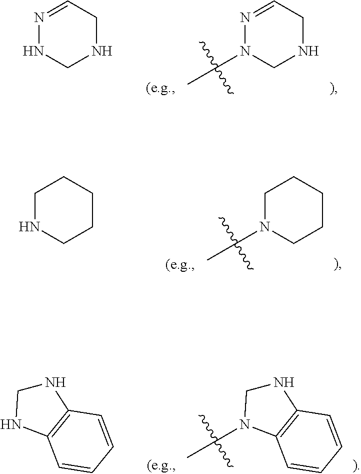 [SMILES: C1=NNCNC1 ; *N1CNCC=N1 ; C1CCNCC1 ; *N1CCCCC1 ; c1ccc2c(c1)NCN2 ; *N1CNc2ccccc21]

In some embodiments,

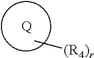

is

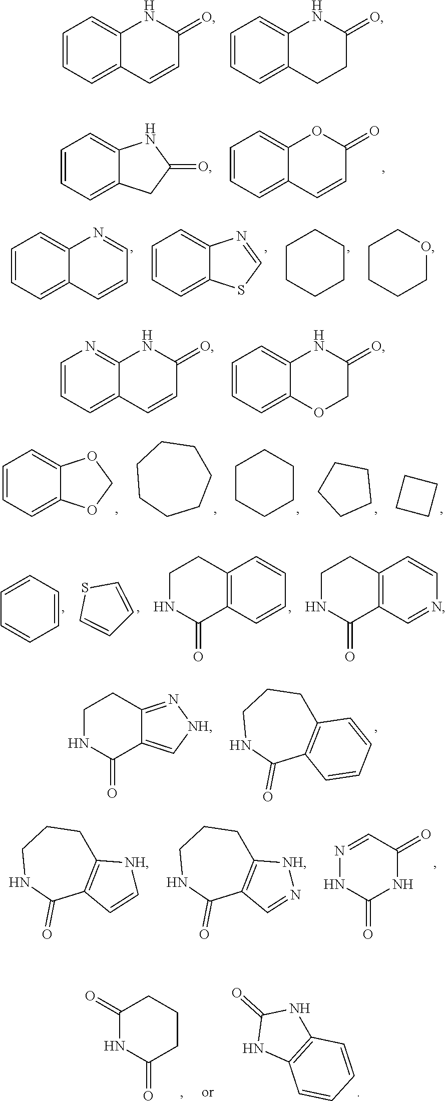

In some embodiments,

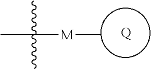

is

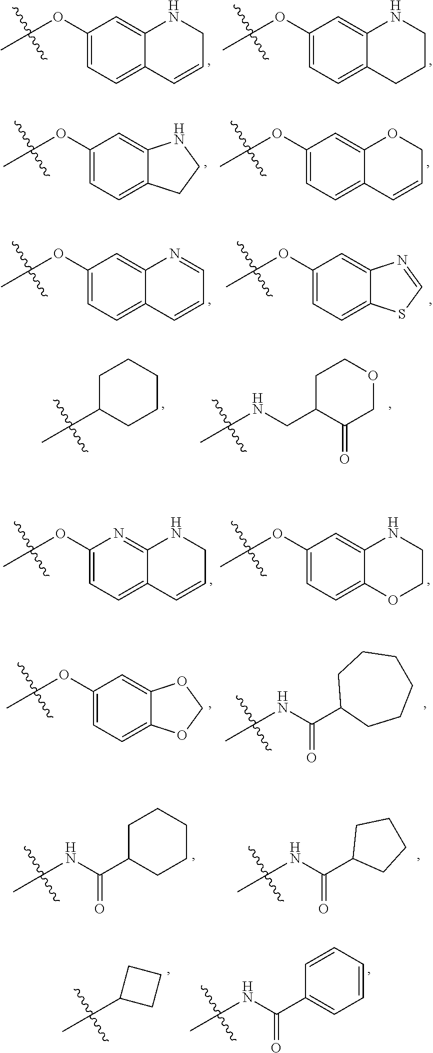

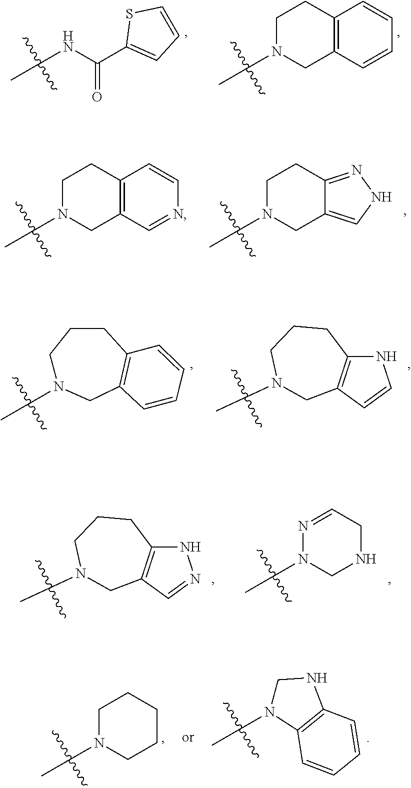

In some embodiments, q is 0, 1, or 2.

In some embodiments, r is 0, 1, or 2.

In some embodiments,

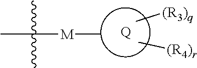

is

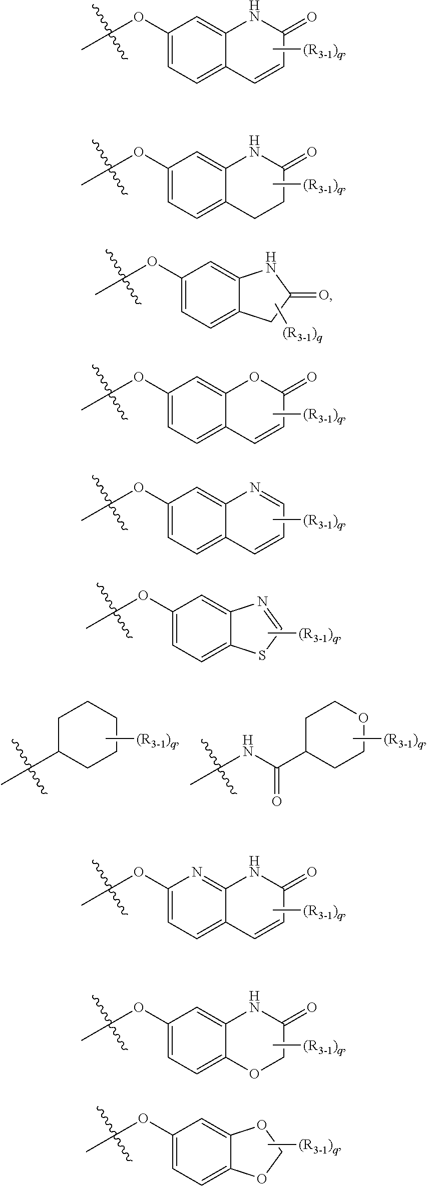

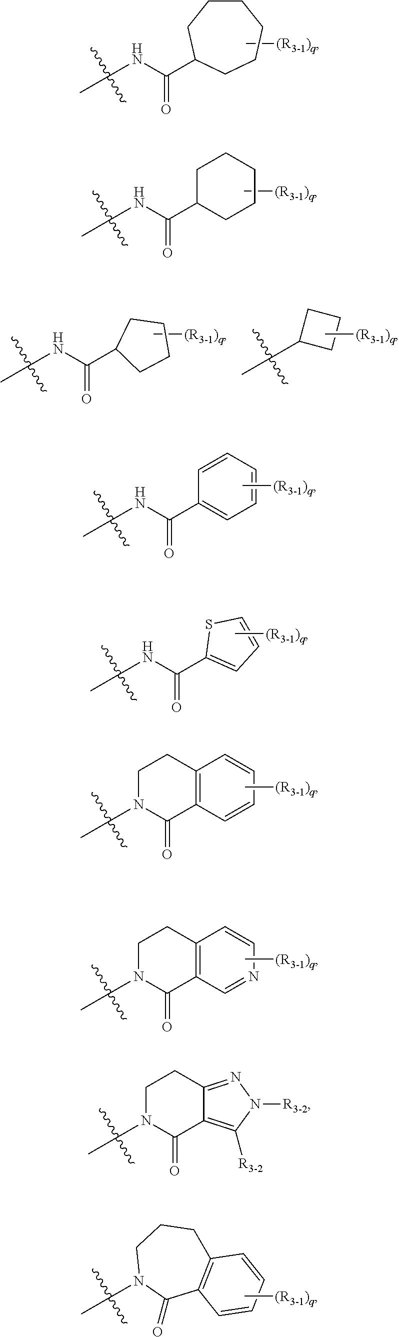

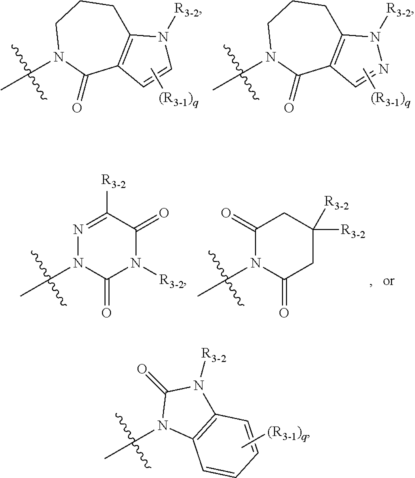

wherein R₃₋₁ is R₃, R₃₋₂ is H or R₃, and q and R₃ are as defined in any one of the embodiments of the present disclosure.

In some embodiments, in

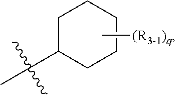

each R₃₋₁ is independently —NH—C(O)Rₑ or —NH—S(O)₂Rₑ.

In some embodiments,

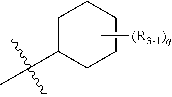

is

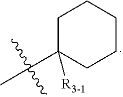

In some embodiments, in

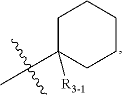

R₃₋₁ is hydroxyl.

In some embodiments,

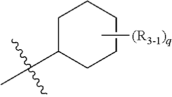

is

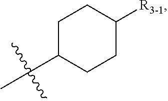

such as

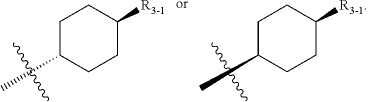

In some embodiments, in

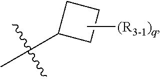

each R₃₋₁ is independently —NH—C(O)Rₑ or —NH—S(O)₂R.

In some embodiments, in

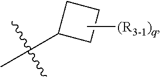

each R₃₋₁ is independently —NH—C(O)Rₑ or —NH—S(O)₂Rₑ.

In some embodiments, each R₃-1 is independently F, Cl, Br, I, hydroxyl, C₁-C₆ alkyl, or C₁-C₆ alkoxy.

In some embodiments,

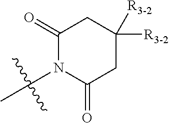

is

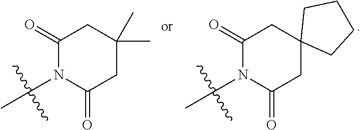 [SMILES: *N1C(=O)CC(C)(C)CC1=O ; *N1C(=O)CC2(CCCC2)CC1=O]

In some embodiments,

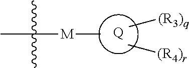

is

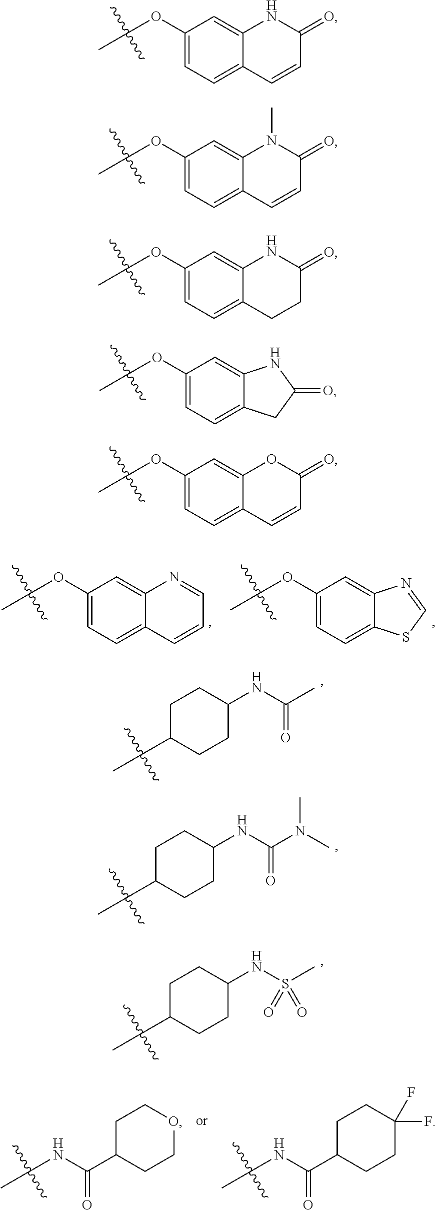

In some embodiments,

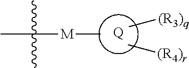

is

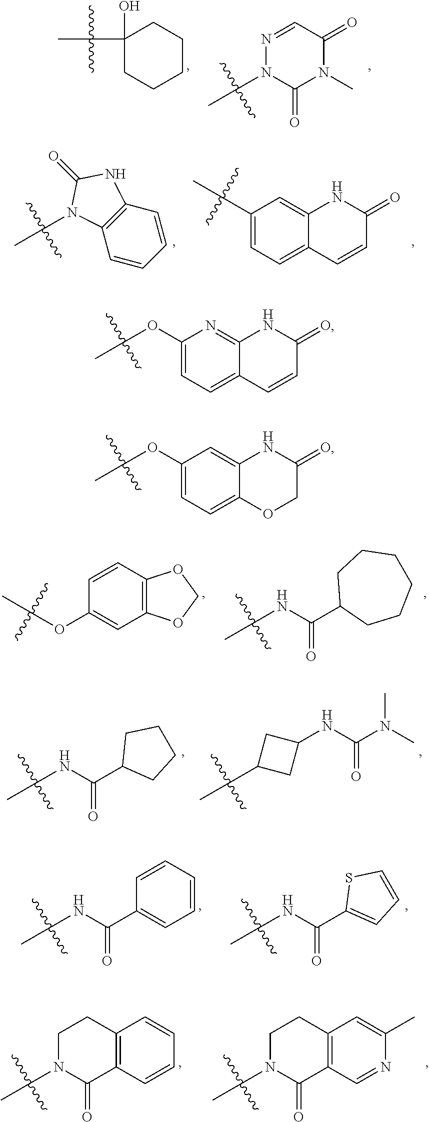

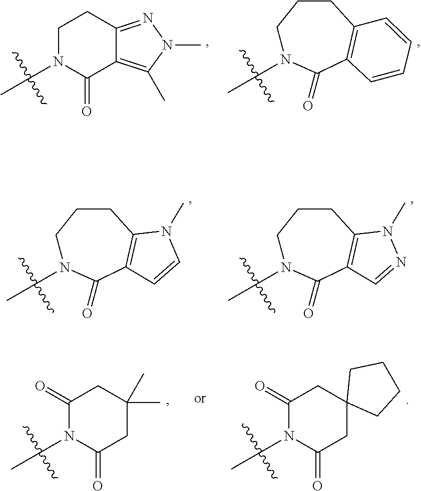 [SMILES: *N1CCc2nn(C)c(C)c2C1=O ; *N1CCCc2ccccc2C1=O ; *N1CCCc2c(ccn2C)C1=O ; *N1CCCc2c(cnn2C)C1=O ; *N1C(=O)CC(C)(C)CC1=O ; *N1C(=O)CC2(CCCC2)CC1=O]

In some embodiments,

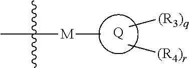

is

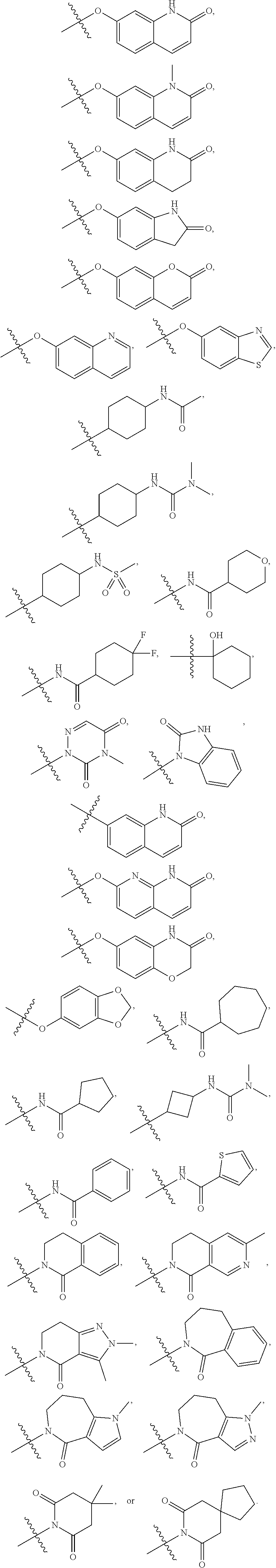

In some embodiments,

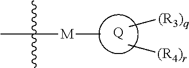

is any one of the following schemes:

scheme (1):

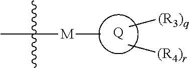

is

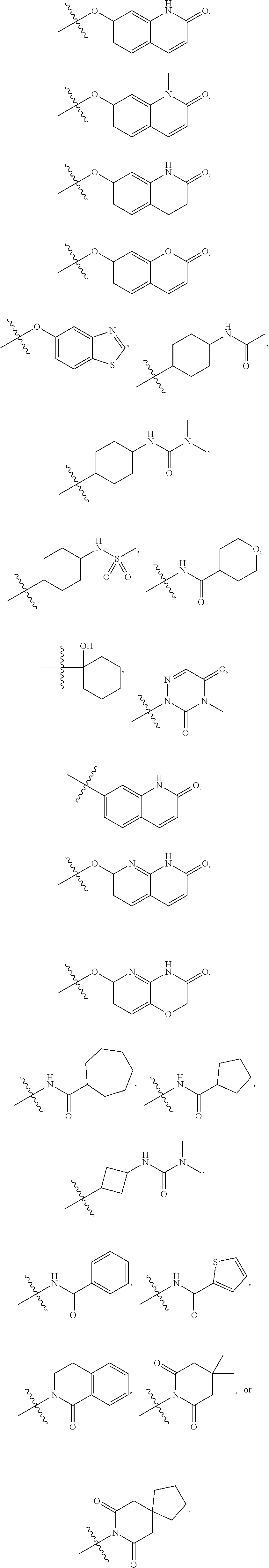

scheme (2):

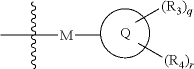

is

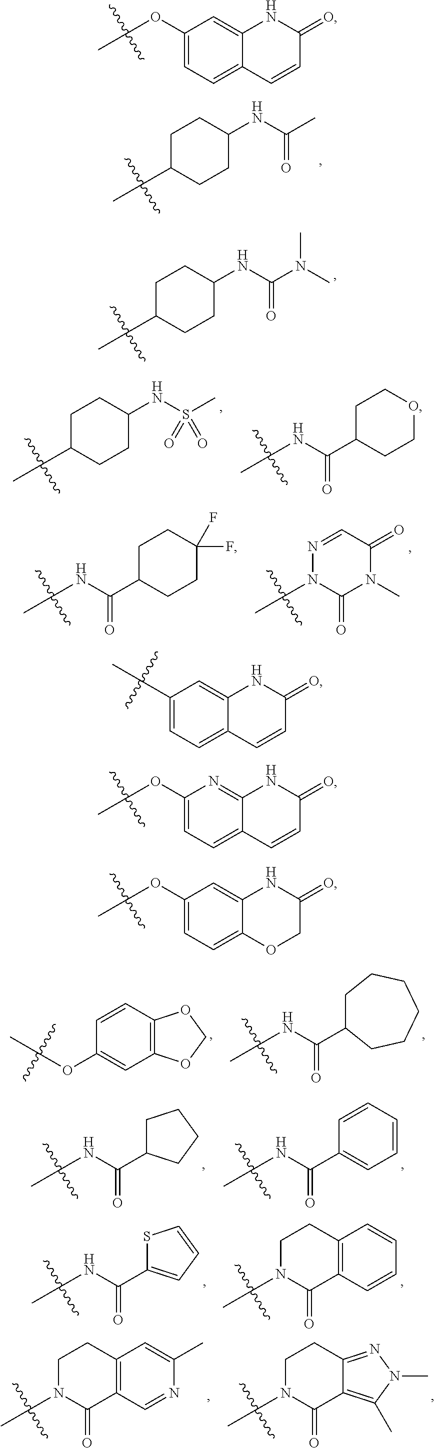

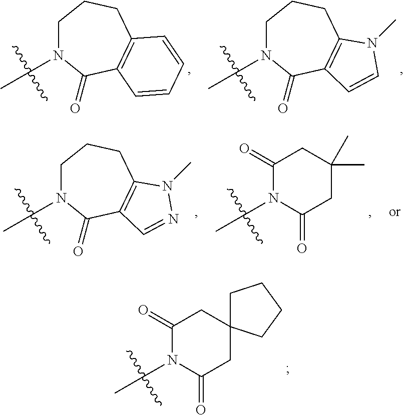

scheme (3):

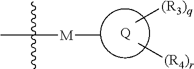

is

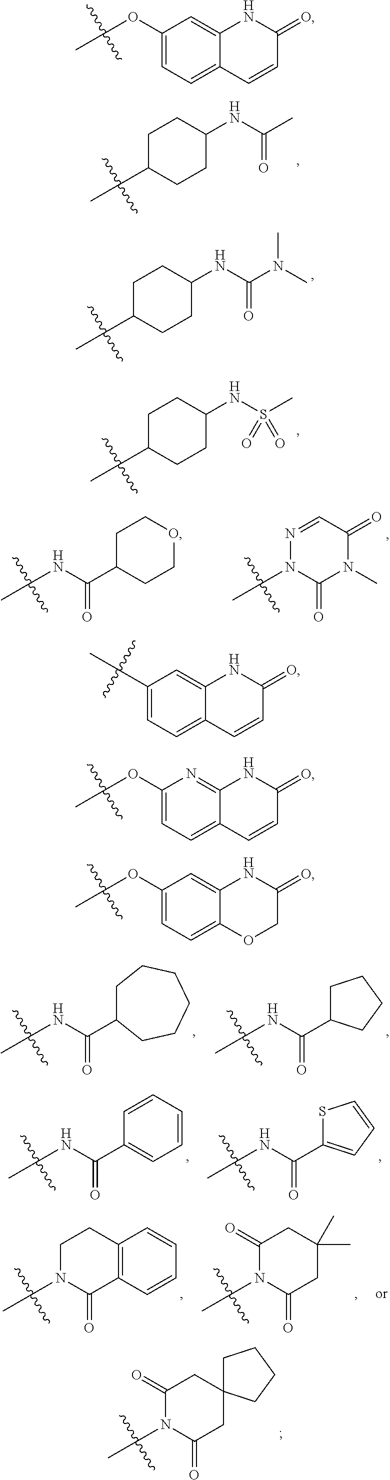

scheme (4):

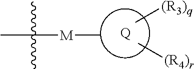

is

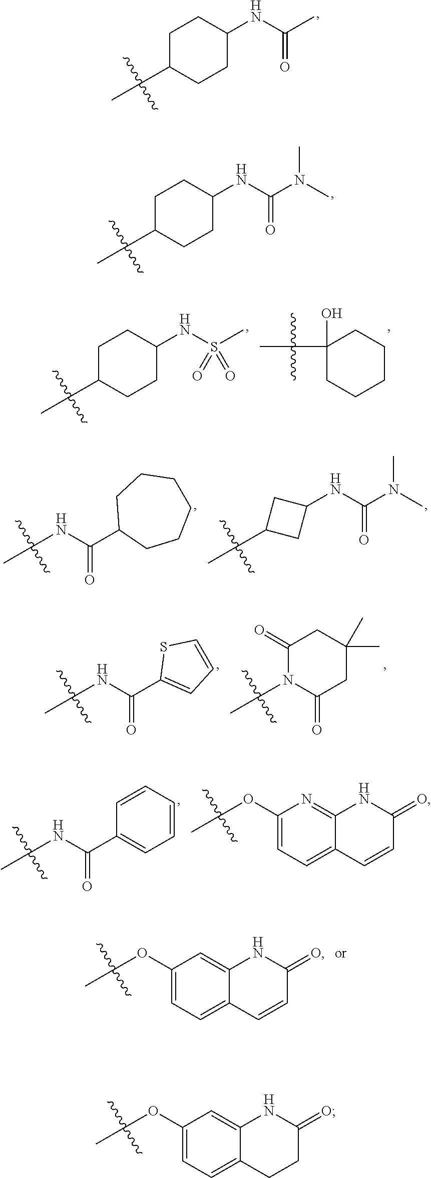

scheme (5):

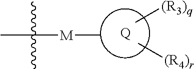

is

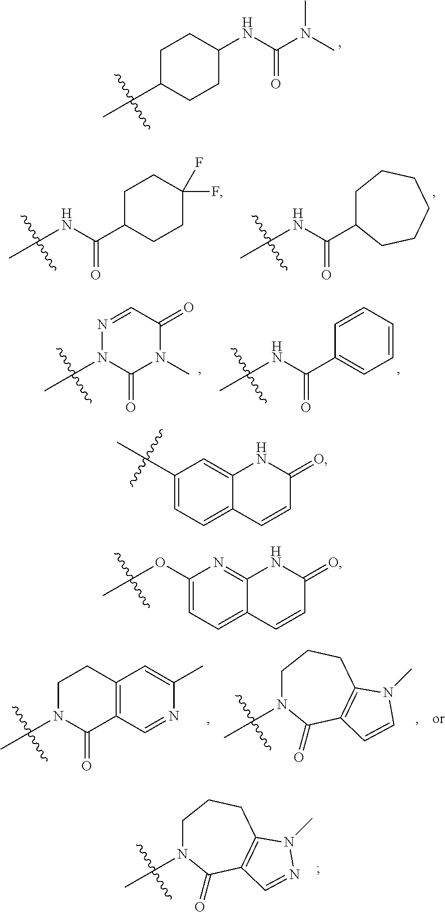

scheme (6):

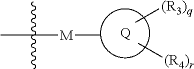

is

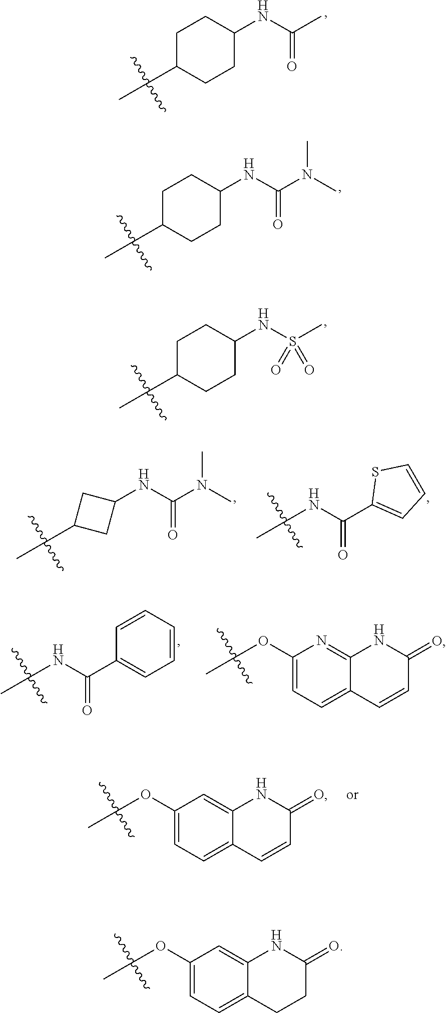

In some embodiments, L is —(CR_{c}R_{d})—, and M is absent.

In some embodiments, L is —(CR_{c}R_{d})₂—, and M is absent or —NH—C(O)—.

In some embodiments, L is —(CR_{c}R_{d})₃—, and M is —O—.

In some embodiments, L is —(CR_{c}R_{d})₃—, and M is absent.

In some embodiments, L is —(CR_{c}R_{d})₄—, and M is —O—.

In some embodiments, L is —(CR_{c}R_{d})₄—, and M is absent.

In some embodiments, L is —(CR_{c}R_{d})—CH═CH—(CR_{c}R_{d})—, and M is —O—.

In some embodiments, in ring Q, the 3- to 8-membered carbocyclic ring is a 5- to 8-membered carbocyclic ring, preferably a 5- to 7-membered carbocyclic ring, such as a 6- to 7-membered carbocyclic ring.

In some embodiments, in ring Q, the 3- to 8-membered heterocyclic ring is a nitrogen-containing 6-membered heterocyclic ring, such as piperidine;
In some embodiments, in ring Q, the 5- to 10-membered heteroaromatic ring is

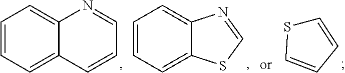

In some embodiments, in ring Q, the 8- to 11-membered fused bicyclic ring is a 8- to 10-membered fused bicyclic ring.

In some embodiments, in ring Q, one of the rings in the 8- to 11-membered fused bicyclic ring is a saturated or partially unsaturated 5- to 7-membered heterocyclic ring, wherein the 5- to 7-membered heterocyclic ring is a 5- to 6-membered heterocyclic ring, such as a 6-membered heterocyclic ring.

In some embodiments, in ring Q, one of the rings in the 8- to 11-membered fused bicyclic ring is a saturated or partially unsaturated 5- to 7-membered heterocyclic ring, wherein the 5- to 7-membered heterocyclic ring contains at most one nitrogen atom or at most one oxygen atom.

In some embodiments, L is —(CR_{c}R_{d})₂—, and in ring Q, the saturated or partially unsaturated 5- to 7-membered heterocyclic ring in the 8- to 11-membered fused bicyclic ring contains at most one N.

In some embodiments, L is —(CR_{c}R_{d})₄—, M is —O—, and the benzene ring or the 5- to 6-membered heteroaromatic ring in the 8- to 11-membered fused bicyclic ring is a 5- to 6-membered aromatic heterocyclic ring.

In some embodiments, ring Q is a saturated 3- to 8-membered carbocyclic ring, a saturated or partially unsaturated 3- to 8-membered heterocyclic ring, a 6- to 10-membered aromatic ring, a 5- to 10-membered heteroaromatic ring, or an 8- to 11-membered fused bicyclic ring; one of the rings in the 8- to 11-membered fused bicyclic ring is a saturated or partially unsaturated 5- to 7-membered carbocyclic ring or a saturated or partially unsaturated 5- to 7-membered heterocyclic ring, and the other ring is benzene ring or a 5- to 6-membered heteroaromatic ring.

In some embodiments, ring Q is a saturated 3- to 8-membered carbocyclic ring, a saturated or partially unsaturated 3- to 8-membered heterocyclic ring, phenyl, a 5- to 10-membered heteroaromatic ring, or an 8- to 10-membered fused bicyclic ring; one of the rings in the 8- to 10-membered fused bicyclic ring is a saturated or partially unsaturated 5- to 7-membered carbocyclic ring or a saturated or partially unsaturated 5- to 6-membered heterocyclic ring, and the other ring is benzene ring or a 5- to 6-membered heteroaromatic ring.

In some embodiments, the compound of formula I has any one of the following structures:

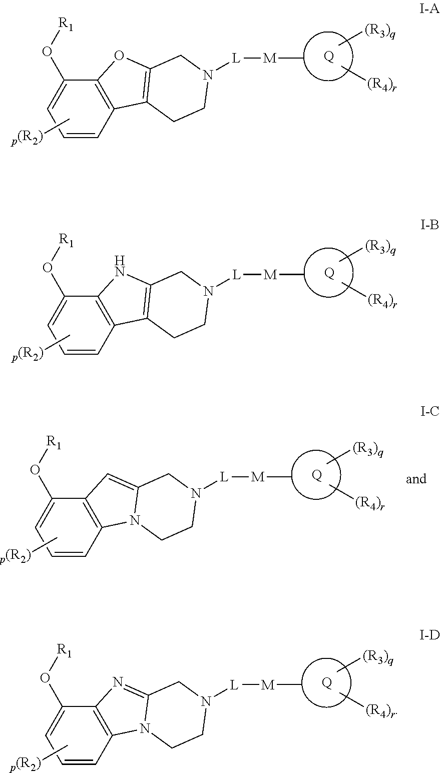

wherein each variable is as defined in any one of the embodiments of the present disclosure.

In some embodiments, the compound is any one of the following schemes:
scheme (1):
X is N, O, NRₐ, or CR_{b};
Y is C or N;
L is —(CR_{c}R_{d})ₘ— or —(CR_{c}R_{d})ₙ₁—CH═CH—(CR_{c}R_{d})ₙ₂—;
R_{c} and R_{d} are each independently H;
m is 1, 2, 3, or 4;
n1 and n2 are 1;
P is 0;
r is 0, 1, or 2;
q is 0, 1, or 2;
M is absent, —O—, or —NH—C(O)—;
ring Q is a saturated 3- to 8-membered carbocyclic ring, a saturated or partially unsaturated 3- to 8-membered heterocyclic ring, a 6- to 10-membered aromatic ring, a 5- to 10-membered heteroaromatic ring, or an 8- to 11-membered fused bicyclic ring; one of the rings in the 8- to 11-membered fused bicyclic ring is a saturated or partially unsaturated 5- to 7-membered carbocyclic ring or a saturated or partially unsaturated 5- to 7-membered heterocyclic ring, and the other ring is benzene ring or a 5- to 6-membered heteroaromatic ring;
R₁ is C₁-C₆ alkyl;
each R₃ is independently F, Cl, Br, I, hydroxyl, C₁-C₆ alkyl, —NH—C(O)Rₑ, or —NH—S(O)₂Rₑ; preferably, each R₃ is independently hydroxyl, C₁-C₆ alkyl, —NH—C(O)Rₑ, or —NH—S(O)₂Rₑ; or, two R₃ together with the atom to which they are attached form a 3- to 8-membered cycloalkyl ring;
Rₑ is C₁-C₆ alkyl, —NH₂, —NHR_{g}, —NR_{f}R_{g}, or 5- to 6-membered heteroaryl;
R_{f} and R_{g} are each independently C₁-C₆ alkyl;
R₄ is oxo (═O);
the number of heteroatoms in the heterocyclic ring, heteroaromatic ring, and heteroaryl is each independently 1, 2, or 3, and the heteroatoms are each independently N, O, or S.

scheme (2):
L is —(CR_{c}R_{d})ₘ— or —(CR_{c}R_{d})ₙ₁—CH═CH—(CR_{c}R_{d})ₙ₂—;
R_{c} and R_{d} are each independently H;
m is 1, 2, 3, or 4;
n1 and n2 are 1;
P is 0;
r is 0, 1, or 2;
q is 0, 1, or 2;
M is absent, —O—, or —NH—C(O)—;
ring Q is a saturated 3- to 8-membered carbocyclic ring, a saturated or partially unsaturated 3- to 8-membered heterocyclic ring, phenyl, a 5- to 10-membered heteroaromatic ring, or an 8- to 10-membered fused bicyclic ring; one of the rings in the 8- to 10-membered fused bicyclic ring is a saturated or partially unsaturated 5- to 7-membered carbocyclic ring or a saturated or partially unsaturated 5- to 6-membered heterocyclic ring, and the other ring is benzene ring or a 5- to 6-membered heteroaromatic ring; in the saturated or partially unsaturated 5- to 6-membered heterocyclic ring in the 8- to 10-membered fused bicyclic ring, the 5- to 6-membered heterocyclic ring contains one oxygen atom, one nitrogen atom, two nitrogen atoms, or one oxygen atom and one nitrogen atom;
R₁ is C₁-C₆ alkyl;
R₃ is hydroxyl, C₁-C₆ alkyl, —NH—C(O)Rₑ, or —NH—S(O)₂Rₑ; or, two R₃ together with the atom to which they are attached form a 3- to 8-membered cycloalkyl ring;
Rₑ is C₁-C₆ alkyl, —NH₂, —NHR_{g}, —NR_{f}R_{g}, or 5- to 6-membered heteroaryl;
R_{f} and R_{g} are each independently C₁-C₆ alkyl;
R₄ is oxo (═O);
the number of heteroatoms in the heterocyclic ring, heteroaromatic ring, and heteroaryl is each independently 1, 2, or 3, and the heteroatoms are each independently N, O, or S;
scheme (3):
X is N or 0;
Y is C;
P is 0;
r is 0, 1, or 2;
q is 0, 1, or 2;
L is —(CR_{c}R_{d})ₘ—; R_{c} and R_{d} are each independently H; m is 2, 3, or 4;
when m is 2, M is absent or —NH—C(O)—, and ring Q is a saturated 5- to 8-membered carbocyclic ring, a saturated or partially unsaturated 3- to 8-membered heterocyclic ring, phenyl, a 5- to 10-membered heteroaromatic ring, or an 8- to 11-membered fused bicyclic ring; one of the rings in the 8- to 11-membered fused bicyclic ring is a saturated or partially unsaturated 5- to 7-membered carbocyclic ring or a saturated or partially unsaturated 5- to 7-membered heterocyclic ring, and the other ring is benzene ring or a 5- to 6-membered heteroaromatic ring;
when m is 3, M is absent; ring Q is a partially unsaturated 3- to 8-membered heterocyclic ring or an 8- to 11-membered fused bicyclic ring; one of the rings in the 8- to 11-membered fused bicyclic ring is a saturated or partially unsaturated 5- to 7-membered heterocyclic ring, and the other ring is benzene ring;
when m is 4, M is —O—, and ring Q is an 8- to 11-membered fused bicyclic ring; one of the rings in the 8- to 11-membered fused bicyclic ring is a saturated or partially unsaturated 5- to 7-membered heterocyclic ring, and the other ring is a 5- to 6-membered heteroaromatic ring;
or, when m is 4, M is absent;
R₁ is C₁-C₆ alkyl;
each R₃ is independently F, Cl, Br, I, C₁-C₆ alkyl, or —NH—C(O)Rₑ; preferably, each R₃ is independently F, Cl, Br, I, C₁-C₆ alkyl, or —NH—C(O)Rₑ; or, two R₃ together with the atom to which they are attached form a 3- to 8-membered cycloalkyl ring;
Rₑ is C₁-C₆ alkyl, —NH₂, —NHR_{g}, —NR_{f}R_{g}, or 5- to 6-membered heteroaryl;
R_{f} and R_{g} are each independently C₁-C₆ alkyl;
R₄ is oxo (═O);
the number of heteroatoms in the heterocyclic ring, heteroaromatic ring, and heteroaryl is each independently 1, 2, or 3, and the heteroatoms are each independently N, O, or S;
in the saturated or partially unsaturated 5- to 7-membered heterocyclic ring in the 8- to 11-membered fused bicyclic ring, the 5- to 7-membered heterocyclic ring contains one oxygen atom, one nitrogen atom, two oxygen atoms, or one oxygen atom and one nitrogen atom;
scheme (4):
X is N or 0;
Y is C;
P is 0;
r is 0, 1, or 2;
q is 0, 1, or 2;
L is —(CR_{c}R_{d})ₘ—; R_{c} and R_{d} are each independently H; m is 2, 3, or 4;
when m is 2, M is absent or —NH—C(O)—, and ring Q is a saturated 5- to 8-membered carbocyclic ring, a saturated or partially unsaturated 3- to 8-membered heterocyclic ring, phenyl, a 5- to 10-membered heteroaromatic ring, or an 8- to 10-membered fused bicyclic ring; one of the rings in the 8- to 10-membered fused bicyclic ring is a saturated or partially unsaturated 5- to 7-membered carbocyclic ring or a saturated or partially unsaturated 5- to 6-membered heterocyclic ring, and the other ring is benzene ring or a 5- to 6-membered heteroaromatic ring;
when m is 3, M is absent, and ring Q is a partially unsaturated 3- to 8-membered heterocyclic ring or an 8- to 10-membered fused bicyclic ring; one of the rings in the 8- to 11-membered fused bicyclic ring is a saturated or partially unsaturated 5- to 6-membered heterocyclic ring, and the other ring is benzene ring;
when m is 4, M is —O—, and ring Q is an 8- to 10-membered fused bicyclic ring; one of the rings in the 8- to 10-membered fused bicyclic ring is a saturated or partially unsaturated 5- to 6-membered heterocyclic ring, and the other ring is a 5- to 6-membered heteroaromatic ring;
or, when m is 4, M is absent; ring Q is a saturated or partially unsaturated 3- to 8-membered heterocyclic ring or an 8- to 10-membered fused bicyclic ring; one of the rings in the 8- to 6-membered fused bicyclic ring is a saturated or partially unsaturated 5- to 6-membered heterocyclic ring, and the other ring is benzene ring;
R₁ is C₁-C₆ alkyl;
each R₃ is independently C₁-C₆ alkyl or —NH—C(O)Rₑ; or, two R₃ together with the atom to which they are attached form a 3- to 8-membered cycloalkyl ring;
Rₑ is C₁-C₆ alkyl, —NH₂, —NHR_{g}, —NR_{f}R_{g}, or 5- to 6-membered heteroaryl;
R_{f} and R_{g} are each independently C₁-C₆ alkyl;
R₄ is oxo (═O);
the number of heteroatoms in the heterocyclic ring, heteroaromatic ring, and heteroaryl is each independently 1, 2, or 3, and the heteroatoms are each independently N, O, or S;
in the saturated or partially unsaturated 5- to 6-membered heterocyclic ring in the 8 to 10-membered fused bicyclic ring, the 5- to 6-membered heterocyclic ring contains one oxygen atom, one nitrogen atom, or one oxygen atom and one nitrogen atom;
scheme (5):
X is O; Y is C; P is 0;
L is —(CR_{c}R_{d})ₘ— or —(CR_{c}R_{d})ₙ₁₁—CH═CH—(CR_{c}R_{d})ₙ₂—; R_{c} and R_{d} are each independently H; m is 1, 2, 3, or 4;
when L is —(CR_{c}R_{d})ₙ₁l-CH═CH—(CR_{c}R_{d})ₙ₂—, n1 and n2 are 1, M is —O—, and ring Q is

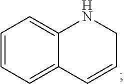

when L is —(CR_{c}R_{d})ₘ— and m is 1, then M is absent, ring Q is a saturated 6-membered carbocyclic ring, and R₃ is hydroxyl;
when L is —(CR_{c}R_{d})ₘ— and m is 2, then M is absent or —NH—C(O)—, ring Q is a saturated 3- to 8-membered carbocyclic ring, piperidine, thiophene, or phenyl, each R₃ is independently C₁-C₆ alkyl or —NH—C(O)Rₑ, and Rₑ is C₁-C₆ alkyl;
when L is —(CR_{c}R_{d})ₘ— and m is 3, then M is —O—, and ring Q is

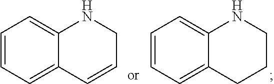

when L is —(CR_{c}R_{d})ₘ— and m is 4, then M is —O— or absent, and ring Q is piperidine,

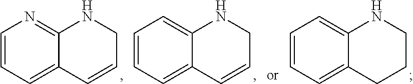

when ring Q is piperidine, each R₃ is independently C₁-C₆ alkyl;
when ring Q is

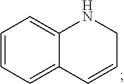

q is 0;
R₁ is C₁-C₆ alkyl;
scheme (6):
X is N; Y is C; P is 0;
L is —(CR_{c}R_{d})ₘ— or —(CR_{c}R_{d})ₙ₁l-CH═CH—(CR_{c}R_{d})ₙ₂—; R_{c} and R_{d} are each independently H; m is 2 or 3;
when L is —(CR_{c}R_{d})ₘ— and m is 2, then M is absent, ring Q is a saturated 5- to 7-membered carbocyclic ring, each R₃ is independently —NH—C(O)Rₑ, and Rₑ is C₁-C₆ alkyl;
when L is —(CR_{c}R_{d})ₘ— and m is 3, then M is —O—, ring Q is

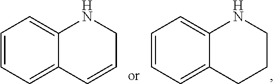

and q is 0;
R₁ is methyl;
scheme (7):
X is C; Y is N; P is 0;
L is —(CR_{c}R_{d})ₘ— or —(CR_{c}R_{d})ₙ₁—CH═CH—(CR_{c}R_{d})ₙ₂—; R_{c} and R_{d} are each independently H; m is 3 or 4;
when L is —(CR_{c}R_{d})₁l-CH═CH—(CR_{c}R_{d})ₙ₂—, n1 and n2 are 1, M is —O—, and ring Q is

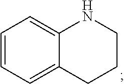

when L is —(CR_{c}R_{d})ₘ— and m is 3, then M is —O—, and ring Q is

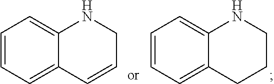

when L is —(CR_{c}R_{d})ₘ— and m is 4, then M is —O—, and ring Q is;

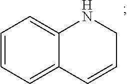

scheme (8):
X is O; Y is C; P is 0;
L is —(CR_{c}R_{d})ₘ—; R_{c} and R_{d} are each independently H; m is 2 or 4;
when L is —(CR_{c}R_{d})ₘ— and m is 2, then M is absent or —NH—C(O)—, and ring Q is a saturated 6- to 7-membered carbocyclic ring, phenyl, or an 8- to 11-membered fused bicyclic ring; one of the rings in the 8- to 11-membered fused bicyclic ring is a saturated 5- to 7-membered heterocyclic ring, and the other ring is a 5- to 6-membered heteroaromatic ring; the 5- to 6-membered heteroaromatic ring contains one or two nitrogen atoms;
when L is —(CR_{c}R_{d})ₘ— and m is 4, then M is absent or —O—;
when L is —(CR_{c}R_{d})ₘ—, m is 4, and M is absent, then ring Q is a partially unsaturated 6-membered heterocyclic ring or

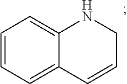

when ring Q is a partially unsaturated 6-membered heterocyclic ring, the heteroatom in the partially unsaturated 6-membered heterocyclic ring is a nitrogen atom, and the number of heteroatoms is each independently 1, 2, or 3;
when L is —(CR_{c}R_{d})ₘ—, m is 4, and M is —O—, then ring Q is a partially unsaturated 6-membered heterocyclic ring or

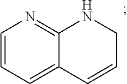

each R₃ is independently F, C₁-C₆ alkyl, or —NH—C(O)Rₑ, and Rₑ is C₁-C₆ alkyl;
scheme (9):
X is O; Y is C; P is 0;
L is —(CR_{c}R_{d})ₘ— or —(CR_{c}R_{d})ₙ₁—CH═CH—(CR_{c}R_{d})ₙ₂—; R_{c} and R_{d} are each independently H; m is 2 or 4;
when L is —(CR_{c}R_{d})ₙ₁—CH═CH—(CR_{c}R_{d})ₙ₂—, n1 and n2 are 1, M is —O—, and ring Q is

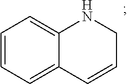

when L is —(CR_{c}R_{d})ₘ— and m is 2, then M is absent or —NH—C(O)—, ring Q is a saturated 4- to 6-membered carbocyclic ring, thiophene, or phenyl, each R₃ is independently C₁-C₆ alkyl or —NH—C(O)Rₑ, and Rₑ is C₁-C₆ alkyl;
when L is —(CR_{c}R_{d})ₘ— and m is 4, then M is —O— or absent, and ring Q is piperidine,

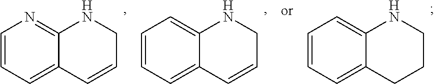

q is 0;
R₁ is C₁-C₆ alkyl;
scheme (10):
X is N; Y is C; P is 0;
L is —(CR_{c}R_{d})ₘ—; R_{c} and R_{d} are each independently H; m is 2 or 3;
when L is —(CR_{c}R_{d})ₘ— and m is 2, then M is absent, ring Q is a saturated 6-membered carbocyclic ring, each R₃ is independently —NH—C(O)Rₑ, and Rₑ is C₁-C₆ alkyl;
when L is —(CR_{c}R_{d})ₘ— and m is 3, then M is —O—, ring Q is

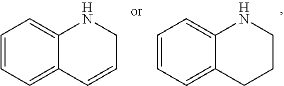

and q is 0;
R₁ is methyl;
scheme (11):
X is C; Y is N; P is 0;
L is —(CR_{c}R_{d})ₘ— or —(CR_{c}R_{d})ₙ₁—CH═CH—(CR_{c}R_{d})ₙ₂—; R_{c} and R_{d} are each independently H; m is 3 or 4;
when L is —(CR_{c}R_{d})ₙ₁—CH═CH—(CR_{c}R_{d})ₙ₂—, n1 and n2 are 1, M is —O—, and ring Q is

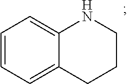

when L is —(CR_{c}R_{d})ₘ— and m is 3 then M is

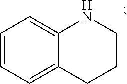

when L is —(CR_{c}R_{d})ₘ— and m is 4, then M is —O—, and ring Q is

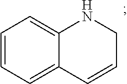

In some embodiments, the compound of formula I has any one of the following structures:

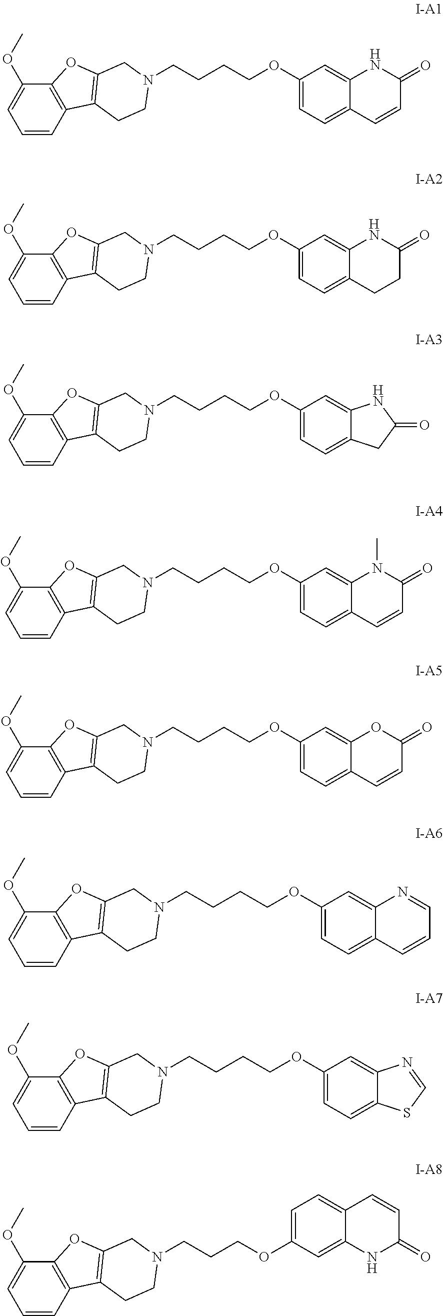

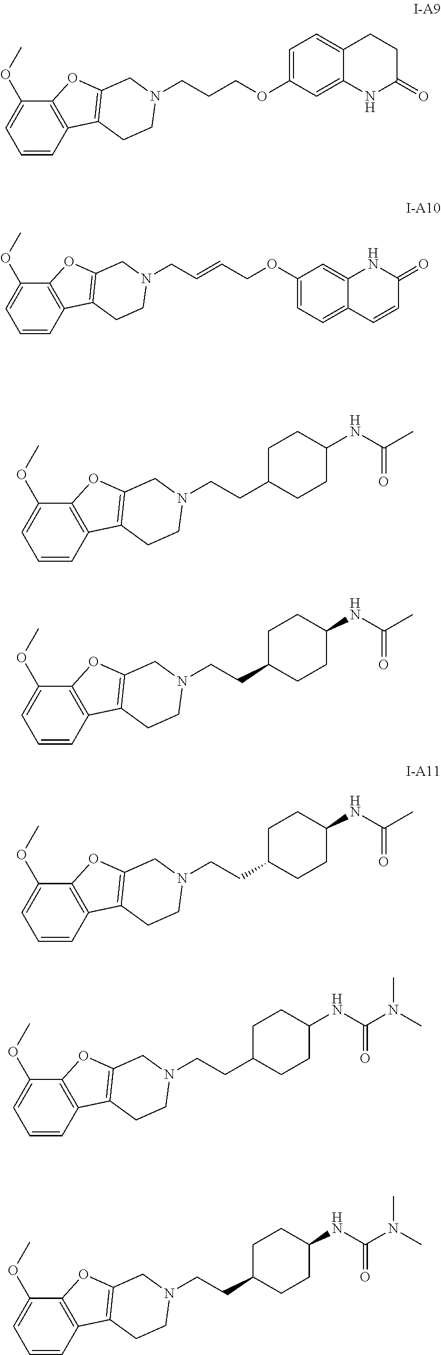

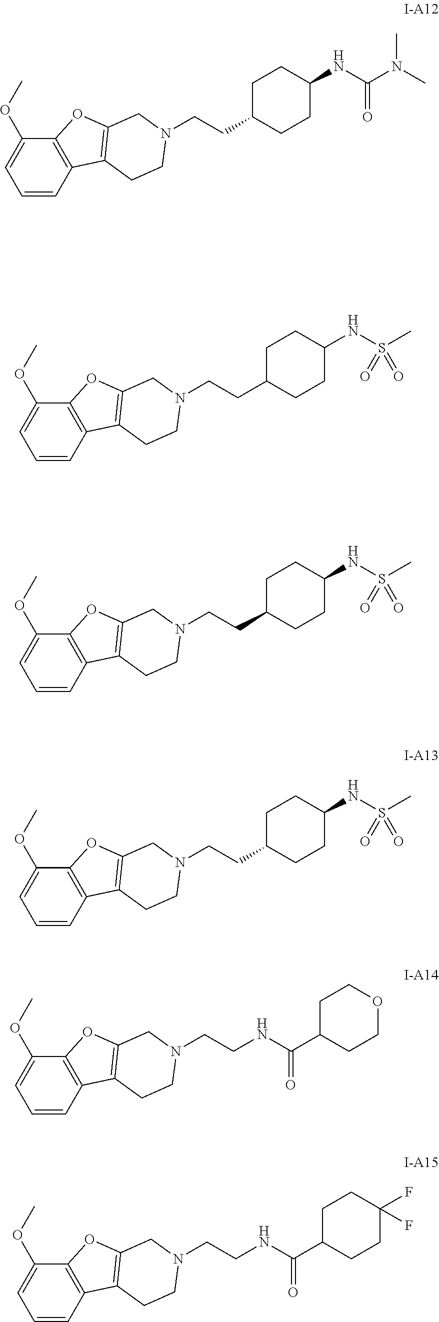 [SMILES: COc1cccc2c3c(oc12)CN(CC[C@H]1CC[C@H](NC(=O)N(C)C)CC1)CC3 ; COc1cccc2c3c(oc12)CN(CCC1CCC(NS(C)(=O)=O)CC1)CC3 ; COc1cccc2c3c(oc12)CN(CC[C@H]1CC[C@@H](NS(C)(=O)=O)CC1)CC3 ; COc1cccc2c3c(oc12)CN(CC[C@H]1CC[C@H](NS(C)(=O)=O)CC1)CC3 ; COc1cccc2c3c(oc12)CN(CCNC(=O)C1CCOCC1)CC3 ; COc1cccc2c3c(oc12)CN(CCNC(=O)C1CCC(F)(F)CC1)CC3]

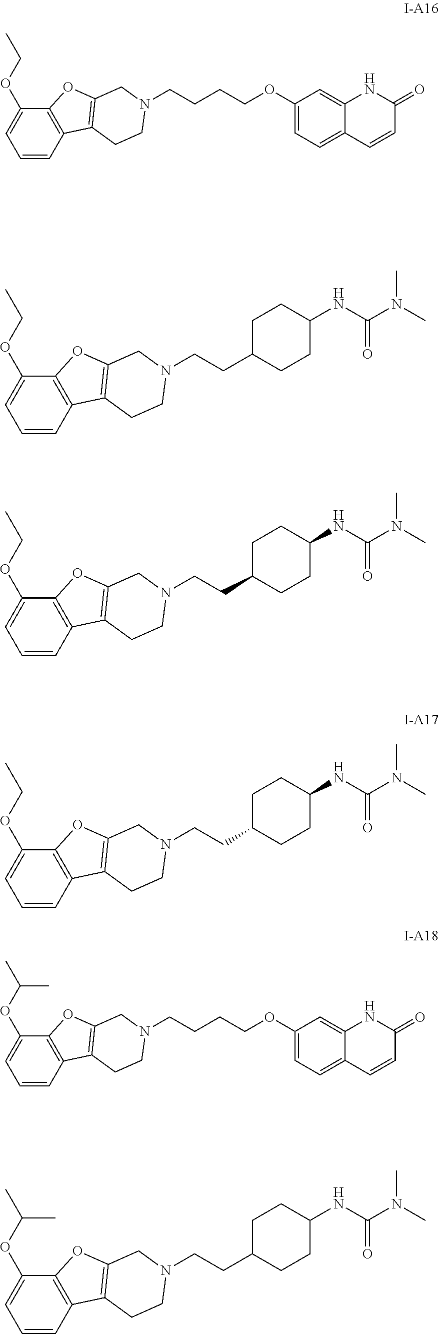 [SMILES: CCOc1cccc2c3c(oc12)CN(CCCCOc1ccc2ccc(=O)[nH]c2c1)CC3 ; CCOc1cccc2c3c(oc12)CN(CCC1CCC(NC(=O)N(C)C)CC1)CC3 ; CCOc1cccc2c3c(oc12)CN(CC[C@H]1CC[C@@H](NC(=O)N(C)C)CC1)CC3 ; CCOc1cccc2c3c(oc12)CN(CC[C@H]1CC[C@H](NC(=O)N(C)C)CC1)CC3 ; CC(C)Oc1cccc2c3c(oc12)CN(CCCCOc1ccc2ccc(=O)[nH]c2c1)CC3 ; CC(C)Oc1cccc2c3c(oc12)CN(CCC1CCC(NC(=O)N(C)C)CC1)CC3]

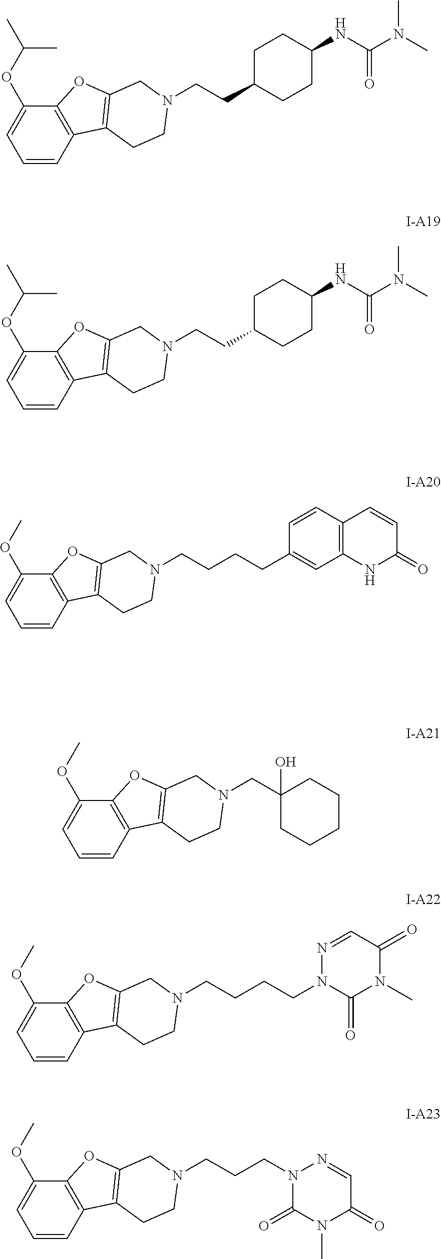 [SMILES: CC(C)Oc1cccc2c3c(oc12)CN(CC[C@H]1CC[C@@H](NC(=O)N(C)C)CC1)CC3 ; CC(C)Oc1cccc2c3c(oc12)CN(CC[C@H]1CC[C@H](NC(=O)N(C)C)CC1)CC3 ; COc1cccc2c3c(oc12)CN(CCCCc1ccc2ccc(=O)[nH]c2c1)CC3 ; COc1cccc2c3c(oc12)CN(CC1(O)CCCCC1)CC3 ; COc1cccc2c3c(oc12)CN(CCCCn1ncc(=O)n(C)c1=O)CC3 ; COc1cccc2c3c(oc12)CN(CCCn1ncc(=O)n(C)c1=O)CC3]

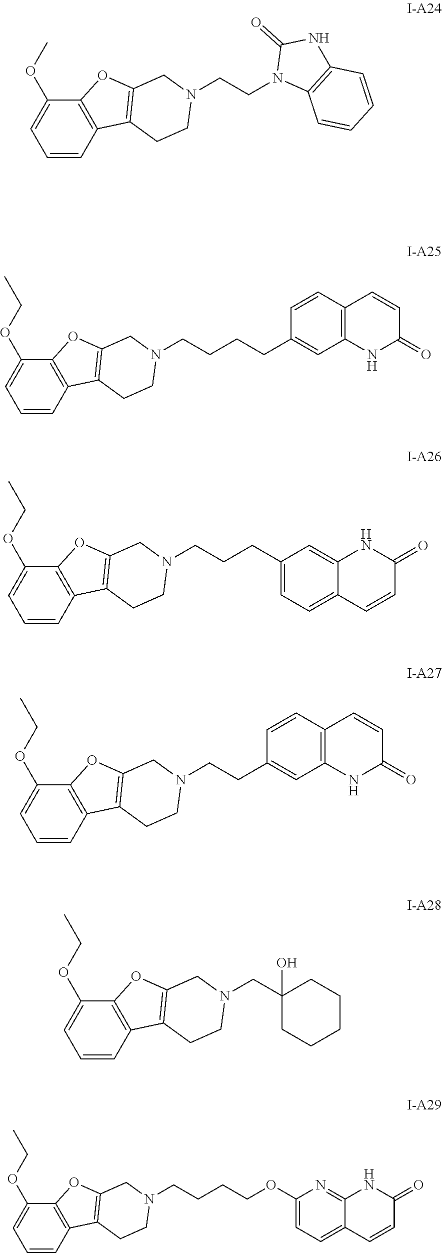 [SMILES: COc1cccc2c3c(oc12)CN(CCn1c(=O)[nH]c2ccccc21)CC3 ; CCOc1cccc2c3c(oc12)CN(CCCCc1ccc2ccc(=O)[nH]c2c1)CC3 ; CCOc1cccc2c3c(oc12)CN(CCCc1ccc2ccc(=O)[nH]c2c1)CC3 ; CCOc1cccc2c3c(oc12)CN(CCc1ccc2ccc(=O)[nH]c2c1)CC3 ; CCOc1cccc2c3c(oc12)CN(CC1(O)CCCCC1)CC3 ; CCOc1cccc2c3c(oc12)CN(CCCCOc1ccc2ccc(=O)[nH]c2n1)CC3]

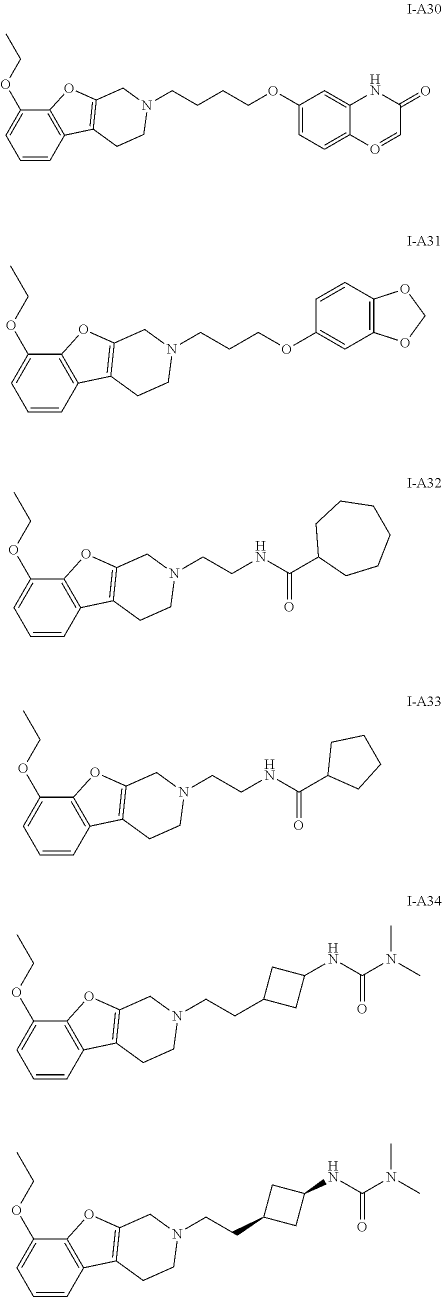

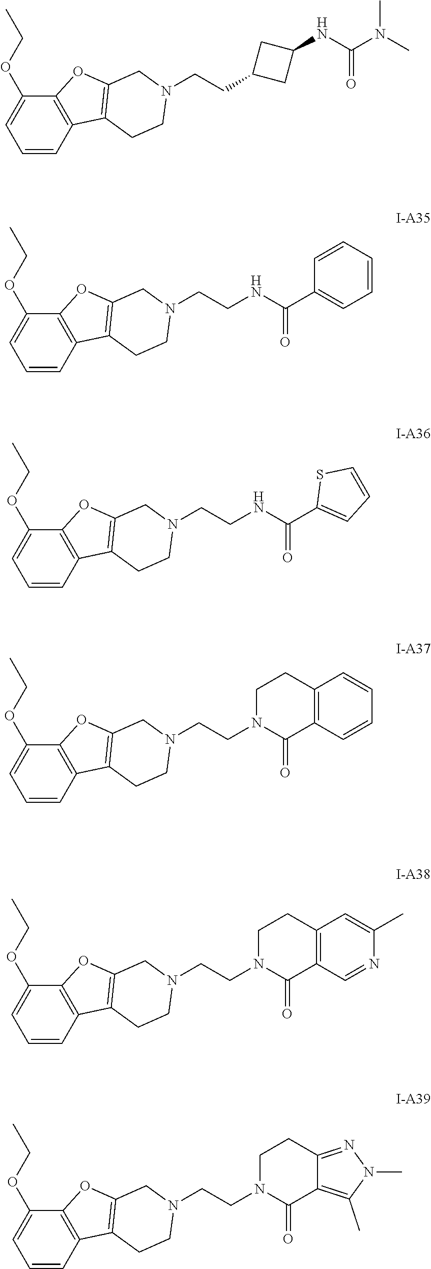 [SMILES: CCOc1cccc2c3c(oc12)CN(CC[C@H]1C[C@H](NC(=O)N(C)C)C1)CC3 ; CCOc1cccc2c3c(oc12)CN(CCNC(=O)c1ccccc1)CC3 ; CCOc1cccc2c3c(oc12)CN(CCNC(=O)c1cccs1)CC3 ; CCOc1cccc2c3c(oc12)CN(CCN1CCc2ccccc2C1=O)CC3 ; CCOc1cccc2c3c(oc12)CN(CCN1CCc2cc(C)ncc2C1=O)CC3 ; CCOc1cccc2c3c(oc12)CN(CCN1CCc2nn(C)c(C)c2C1=O)CC3]

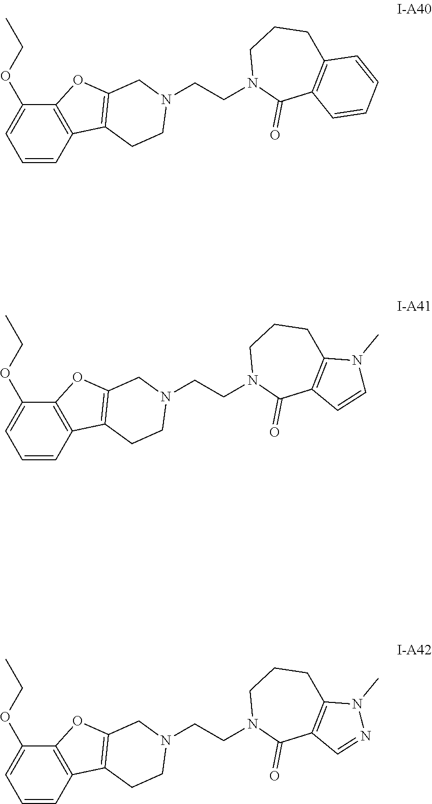 [SMILES: CCOc1cccc2c3c(oc12)CN(CCN1CCCc2ccccc2C1=O)CC3 ; CCOc1cccc2c3c(oc12)CN(CCN1CCCc2c(ccn2C)C1=O)CC3 ; CCOc1cccc2c3c(oc12)CN(CCN1CCCc2c(cnn2C)C1=O)CC3]

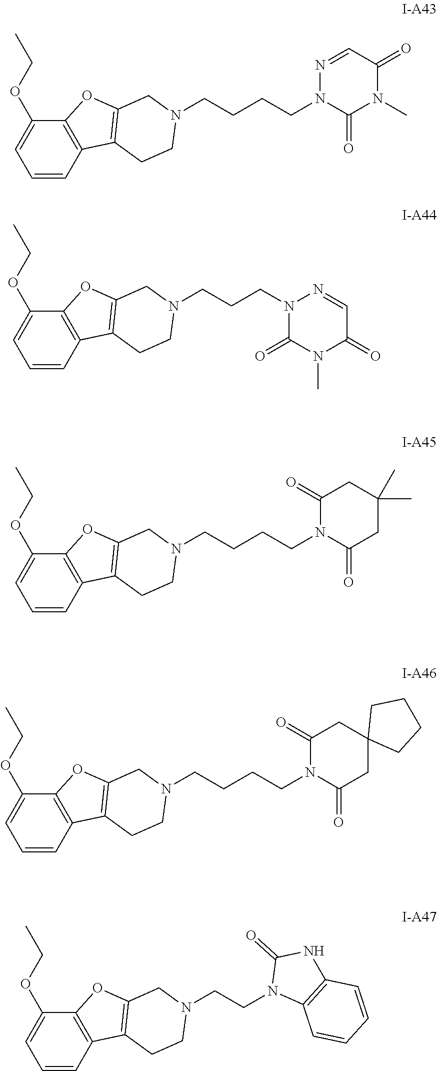 [SMILES: CCOc1cccc2c3c(oc12)CN(CCCCn1ncc(=O)n(C)c1=O)CC3 ; CCOc1cccc2c3c(oc12)CN(CCCn1ncc(=O)n(C)c1=O)CC3 ; CCOc1cccc2c3c(oc12)CN(CCCCN1C(=O)CC(C)(C)CC1=O)CC3 ; CCOc1cccc2c3c(oc12)CN(CCCCN1C(=O)CC2(CCCC2)CC1=O)CC3 ; CCOc1cccc2c3c(oc12)CN(CCn1c(=O)[nH]c2ccccc21)CC3]

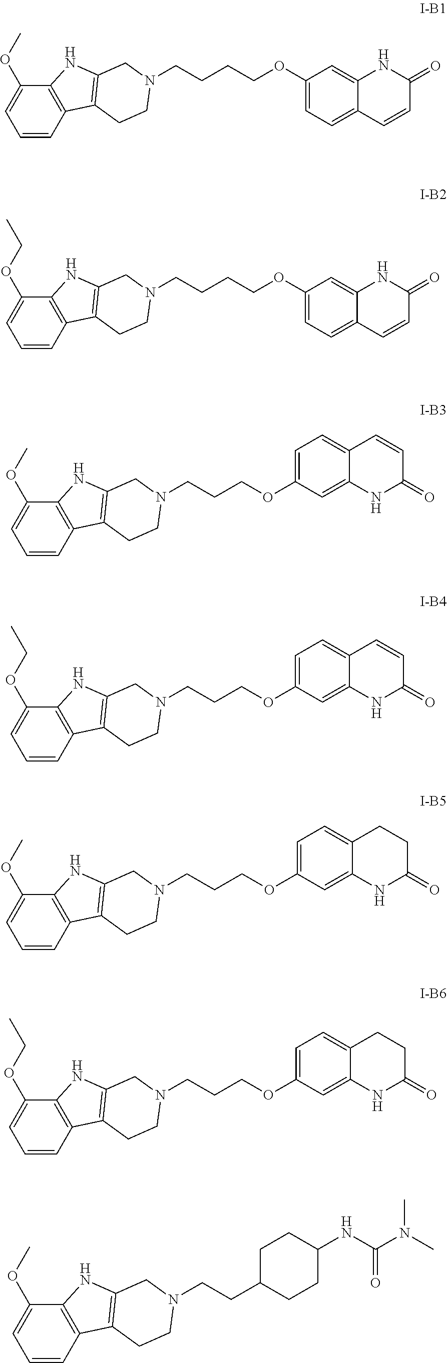

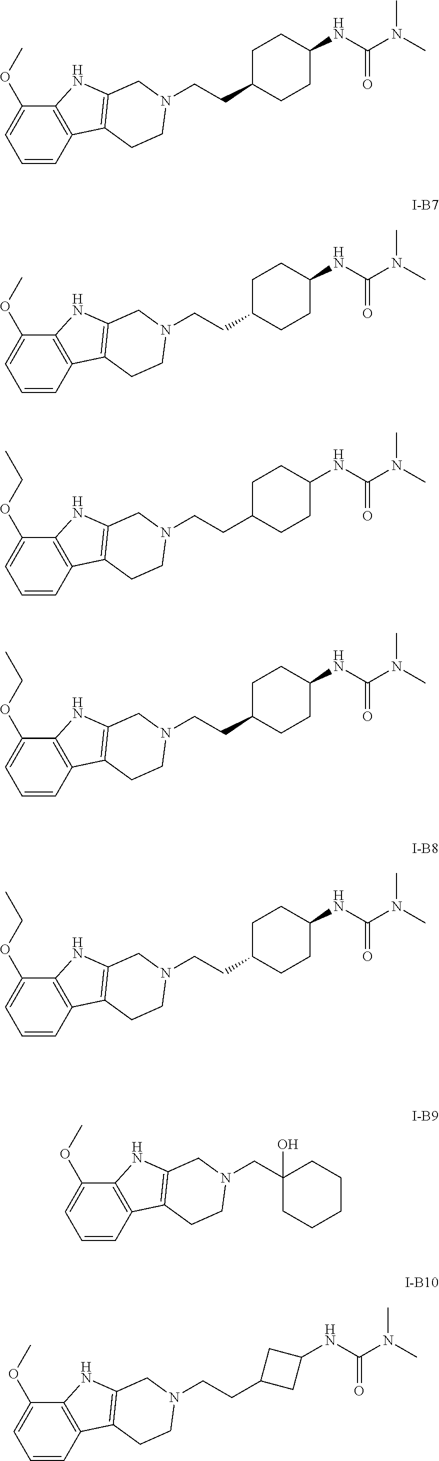

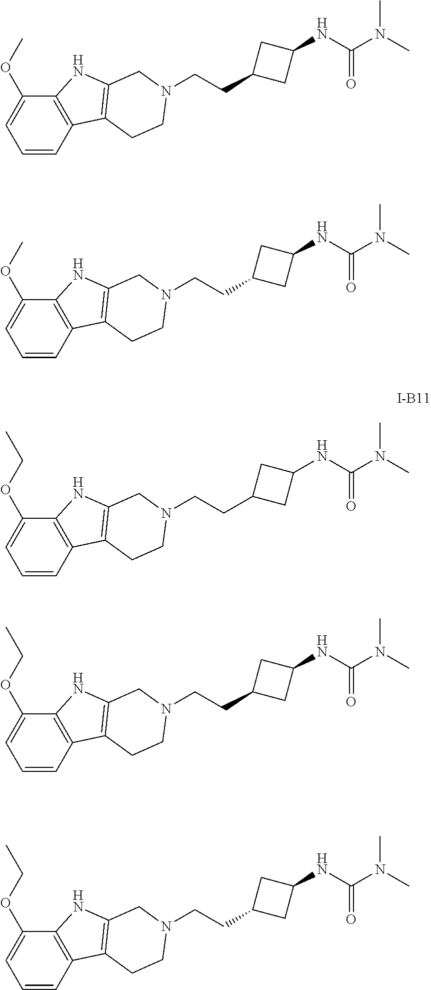 [SMILES: COc1cccc2c3c([nH]c12)CN(CC[C@H]1C[C@@H](NC(=O)N(C)C)C1)CC3 ; COc1cccc2c3c([nH]c12)CN(CC[C@H]1C[C@H](NC(=O)N(C)C)C1)CC3 ; CCOc1cccc2c3c([nH]c12)CN(CCC1CC(NC(=O)N(C)C)C1)CC3 ; CCOc1cccc2c3c([nH]c12)CN(CC[C@H]1C[C@@H](NC(=O)N(C)C)C1)CC3 ; CCOc1cccc2c3c([nH]c12)CN(CC[C@H]1C[C@H](NC(=O)N(C)C)C1)CC3]

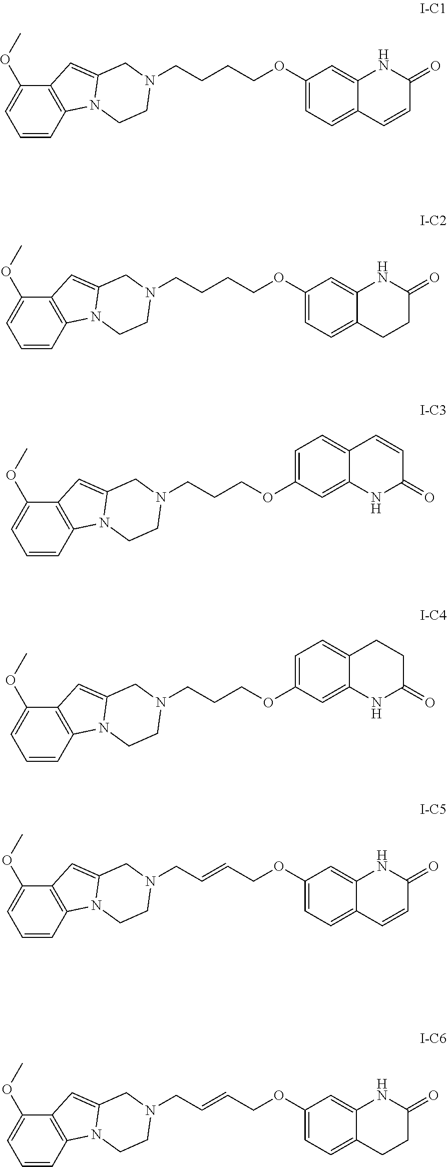 [SMILES: COc1cccc2c1cc1n2CCN(CCCCOc2ccc3ccc(=O)[nH]c3c2)C1 ; COc1cccc2c1cc1n2CCN(CCCCOc2ccc3c(c2)NC(=O)CC3)C1 ; COc1cccc2c1cc1n2CCN(CCCOc2ccc3ccc(=O)[nH]c3c2)C1 ; COc1cccc2c1cc1n2CCN(CCCOc2ccc3c(c2)NC(=O)CC3)C1 ; COc1cccc2c1cc1n2CCN(C/C=C/COc2ccc3ccc(=O)[nH]c3c2)C1 ; COc1cccc2c1cc1n2CCN(C/C=C/COc2ccc3c(c2)NC(=O)CC3)C1]

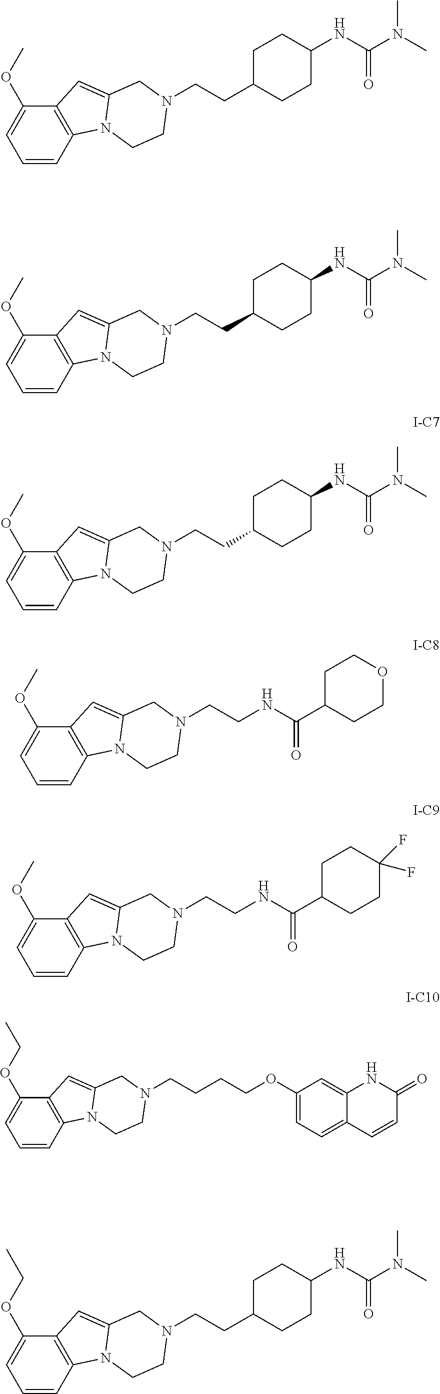

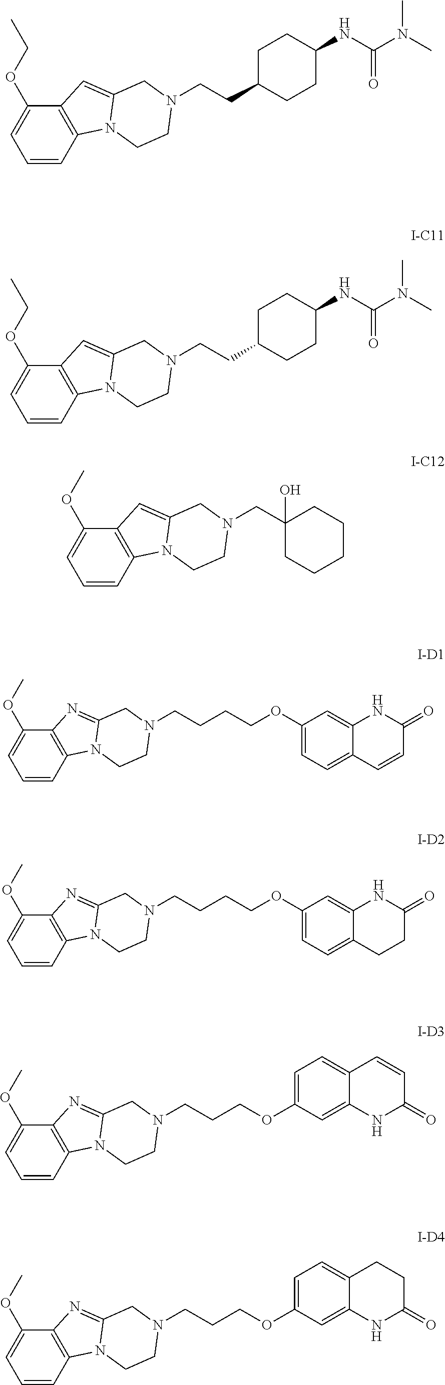

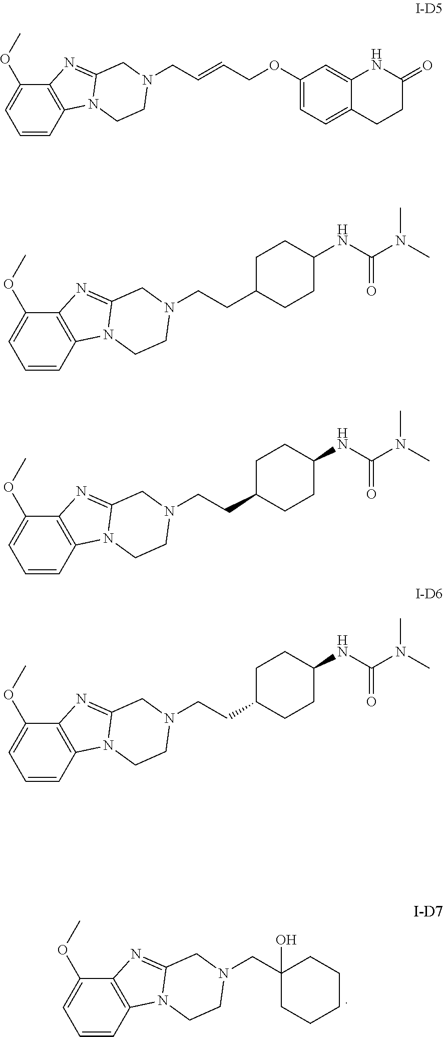 [SMILES: COc1cccc2c1nc1n2CCN(C/C=C/COc2ccc3c(c2)NC(=O)CC3)C1 ; COc1cccc2c1nc1n2CCN(CCC2CCC(NC(=O)N(C)C)CC2)C1 ; COc1cccc2c1nc1n2CCN(CC[C@H]2CC[C@@H](NC(=O)N(C)C)CC2)C1 ; COc1cccc2c1nc1n2CCN(CC[C@H]2CC[C@H](NC(=O)N(C)C)CC2)C1 ; COc1cccc2c1nc1n2CCN(CC2(O)CCCCC2)C1]

The present disclosure also provides a preparation method for the compound of formula I, comprising the step of: conducting a coupling reaction as follows between a compound of formula II and a compound of formula III (e.g., under the action of DIPEA) in a solvent (e.g., a mixed solvent of N,N-dimethylformamide and water) to obtain the compound of formula I;

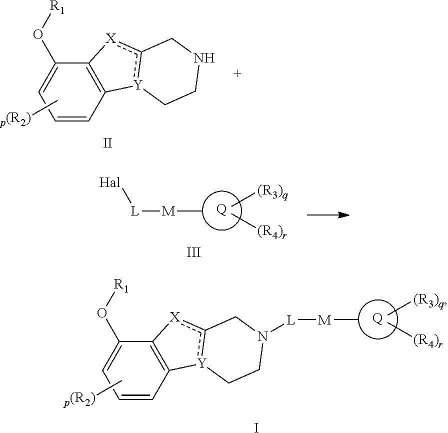

wherein Hal is halogen (e.g., Br); other variables are as defined in any one of the embodiments of the present disclosure.

The present disclosure also provides a pharmaceutical composition comprising the compound of formula I, or the pharmaceutically acceptable salt, isotope derivative, enantiomer, diastereomer, tautomer, or solvate thereof, and a pharmaceutically acceptable excipient.

The present disclosure also provides a use of the pharmaceutical composition in the manufacture of a medicament for the treatment of a mental disease or neurodegenerative disease.

The present disclosure also provides a use of the compound of formula I, or the pharmaceutically acceptable salt, isotope derivative, enantiomer, diastereomer, tautomer, or solvate thereof in the manufacture of a medicament for the treatment of a mental disease or neurodegenerative disease.

In some embodiments, the mental disease or neurodegenerative disease is schizophrenia, depression, or Parkinson's disease.

### DEFINITION OF TERMS

Unless otherwise specified, the following terms and phrases when used herein have the following meanings. A specific term or phrase should not be considered indefinite or unclear in the absence of a particular definition, but should be understood in the ordinary sense. When a trading name appears herein, it is intended to refer to its corresponding commodity or active ingredient thereof.

In the present disclosure, the term “substituted” or “substituent” means that a hydrogen atom in a group is replaced by a specified group. When the position of substitution is not specified, the substitution may be at any position, but is allowed only when it results in a stable or chemically viable chemical. For example, the structure

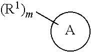

indicates that the hydrogen atoms on ring A are substituted by m R₁. In addition, when ring A is a fused bicyclic ring, either of the bicyclic rings can be substituted. For example, if not otherwise stated, the structure

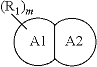

indicates that the hydrogen atoms on ring A1 and/or ring A2 are substituted by m R₁.

When any variable (such as R) occurs in the constitution or structure of the compound more than once, the definition of the variable at each occurrence is independent. Thus, for example, if a group is substituted by 0 to 2 R, the group is optionally substituted by up to two R, wherein the definition of R at each occurrence is independent. Moreover, a combination of the substituent and/or the variant thereof is allowed only when the combination results in a stable compound.

When the number of a linking group is absent, it means that the linking group is a single bond. For example, when L in A-L-Z is absent, the structure formed is A-Z.

In the present disclosure, the term “alkyl” refers to a linear or branched, saturated, monovalent hydrocarbon group. C₁-C₆ alkyl refers to an alkyl group having 1 to 6 carbon atoms, which is preferably C₁-C₄ alkyl, such as methyl, ethyl, n-propyl, isopropyl, n-butyl, isobutyl, sec-butyl, or tert-butyl.

In the present disclosure, the term “haloalkyl” refers to a group in which one or more (e.g., 2, 3, 4, 5, or 6) hydrogen atoms in an alkyl group are substituted by halogens, wherein each of the halogens is independently F, Cl, Br, or I. Halo-C₁-C₆ alkyl refers to C₁-C₆ alkyl substituted by one or more halogens, wherein the C₁-C₆ alkyl is as defined above.

In the present disclosure, the term “alkoxy” refers to —O-alkyl, wherein the alkyl is as defined above. C₁-C₆ alkoxy refers to —O—(C₁-C₆ alkyl), wherein the C₁-C₆ alkyl is as defined above.

In the present disclosure, the term “carbocyclic ring” refers to a saturated, partially unsaturated, or aromatic monocyclic or polycyclic (e.g., fused, spiro, or bridged) cyclic group consisting of carbon atoms. In a saturated carbocyclic ring, each carbon atom on the ring is saturated. Examples of the saturated carbocyclic ring include, but are not limited to,

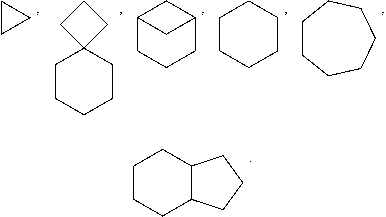

In an aromatic carbocyclic ring, each ring is aromatic. Examples of the aromatic carbocyclic ring include, but are not limited to,

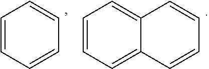

In a partially unsaturated carbocyclic ring, at least one carbon atom on the ring is saturated and at least one carbon atom is unsaturated. Examples of the partially unsaturated carbocyclic ring include, but are not limited to,

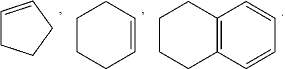

The 3- to 8-membered carbocyclic ring may specifically be a 3-membered, 4-membered, 5-membered, 6-membered, 7-membered, or 8-membered carbocyclic ring.

In the present disclosure, the term “heterocyclic ring” refers to a saturated, partially unsaturated, or aromatic monocyclic or polycyclic (e.g., fused, spiro, or bridged) cyclic group consisting of carbon atoms and at least one heteroatom, wherein the heteroatom is independently selected from N, O, and S. In a saturated heterocyclic ring, both the carbon atoms and heteroatoms on the ring are saturated. Examples of the saturated heterocyclic ring include, but are not limited to,

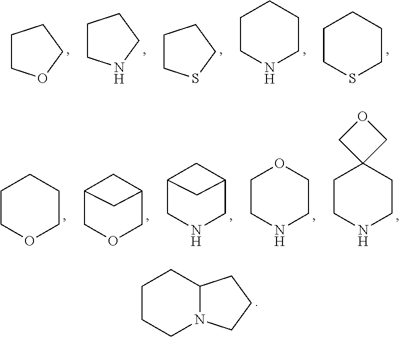

In an aromatic heterocyclic ring, each ring is aromatic. Examples of the aromatic heterocyclic ring include, but are not limited to,

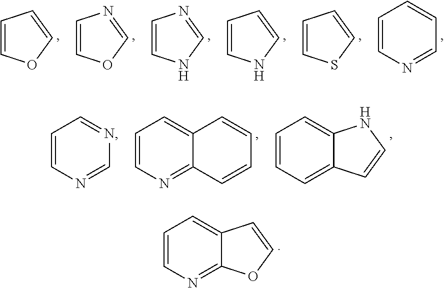

In a partially unsaturated heterocyclic ring, at least one atom on the ring is saturated and at least one atom is unsaturated. Examples of the partially unsaturated heterocyclic ring include, but are not limited to,

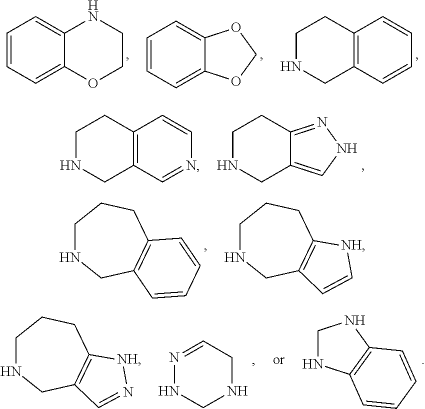

The 3- to 8-membered heterocyclic ring may specifically be a 3-membered, 4-membered, 5-membered, 6-membered, 7-membered, or 8-membered heterocyclic ring. The 5- to 7-membered heterocyclic ring may specifically be a 5-membered, 6-membered, or 7-membered heterocyclic ring.

In the present disclosure, the term “aromatic ring” refers to an aromatic carbocyclic ring, wherein each of the rings is aromatic. The 6- to 10-membered aromatic ring may specifically be benzene ring or a naphthalene ring.

In the present disclosure, the term “heteroaromatic ring” refers to an aromatic heterocyclic ring, wherein each of the rings is aromatic. Examples of the heteroaromatic ring include, but are not limited to,

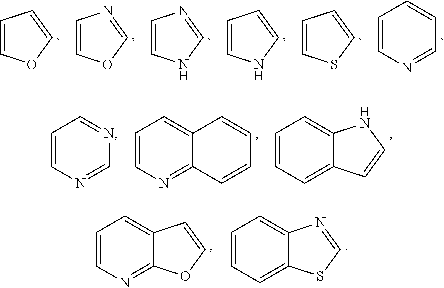

The 5- to 10-membered heteroaromatic ring may specifically be a 5-membered, 6-membered, 7-membered, 8-membered, 9-membered, or 10-membered heteroaromatic ring.

In the present disclosure, the term “fused bicyclic ring” refers to a fused ring consisting of two monocyclic rings, wherein the attachment site to the other structure may be located on either monocyclic ring. The 8- to 11-membered fused bicyclic ring may specifically be a 8-membered, 9-membered, 10-membered, or 11-membered fused bicyclic ring.

In the present disclosure, the term “cycloalkyl” refers to a monocyclic or polycyclic (e.g., fused, spiro, or bridged) monovalent hydrocarbon group in which each carbon atom is saturated. The 3- to 8-membered cycloalkyl may specifically be 3-membered, 4-membered, 5-membered, 6-membered, 7-membered, or 8-membered cycloalkyl, including cyclopropyl, cyclobutyl, cyclopentyl, cyclohexyl, etc. Specific examples of cycloalkyl include, but are not limited to, cyclopropyl, cyclobutyl, cyclopentyl, cyclohexyl,

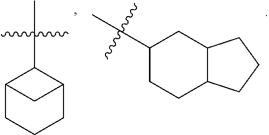 [SMILES: *C1C2CCCC1C2 ; *C1CCC2CCCC2C1]

The compounds of the present disclosure and their structures are also meant to include all isomeric forms (including stereoisomers and tautomers, wherein stereoisomers are for example enantiomers, diastereomers, geometric isomers (such as cis-trans isomers), and conformational isomers). They can be defined as (R)-/(S)-, (D)-/(L)-, or (R,R)-/(R,S)-/(S,S)- in terms of absolute stereochemistry for amino acids. The present disclosure includes all such possible isomers, as well as their racemic, enantiomerically enriched, and optically pure forms. Optically active (+) and (−), (R)- and (S)-, and (R,R)-/(R,S)-/(S,S)- or (D)- and (L)-isomers can be synthesized using chiral starting materials, prepared by chiral resolution, or resolved using conventional techniques such as, but not limited to, high-performance liquid chromatography (HPLC) using chiral columns. When the compound described herein contains an olefinic double bond or other center of geometric asymmetry, the compound is intended to include both E and Z geometric isomers unless otherwise specified. The bond “ [symbol] ” does not specify a configuration in the chemical structure, that is, if there is configurational isomerism in the chemical structure, the bond “ [symbol] ” may be “ [symbol] ” or “ [symbol] ”, or contain both “ [symbol] ” and “ [symbol] ” configurations. Similarly, all tautomeric forms are also intended to be included.

In the present disclosure, the term “tautomer” refers to a proton shift from one atom of a molecule to another position in the same molecule. The present disclosure includes tautomers of any one of the compounds.

In the present disclosure, the term “isotope derivative” refers to a compound that differs in structure only by having one or more isotopically enriched atoms. For example, compounds having the structure of the present disclosure, except for having “deuterium” or “tritium” in place of hydrogen, or ¹⁸F-fluorine labeling (¹⁸F isotope) in place of fluorine, or ¹¹C—, ¹³C—, or ¹⁴C-enriched carbon (¹¹C—, ¹³C—, or ¹⁴C-carbon labeling; ¹¹C—, ¹³C—, or ¹⁴C-isotope) in place of a carbon atom, are within the scope of the present disclosure. Such compounds can be used as analytical tools or probes in, for example, bioassays, or can be used as tracers for in vivo diagnostic imaging of diseases, or as tracers for pharmacodynamic, pharmacokinetic, or receptor studies. In the present disclosure, the isotope derivative is, for example, a deuterated derivative.

In the present disclosure, the term “pharmaceutically acceptable” refers to a substance (e.g., a carrier or diluent) that does not affect the biological activity or properties of the compounds of the present disclosure and is relatively non-toxic, that is, the substance can be administered to an individual without causing an adverse biological reaction or interacting in an undesirable manner with any component contained in the composition.

In the present disclosure, the term “pharmaceutically acceptable salt” refers to a salt formed by a suitable non-toxic organic acid, inorganic acid, organic base, or inorganic base with a compound, which retains the biological activity of the compound. The organic acid may be a variety of organic acids conventional in the art that are capable of forming salts, preferably one or more of methanesulfonic acid, p-toluenesulfonic acid, maleic acid, fumaric acid, citric acid, tartaric acid, malic acid, lactic acid, formic acid, acetic acid, propionic acid, trifluoroacetic acid, oxalic acid, succinic acid, benzoic acid, isethionic acid, naphthalenesulfonic acid, and salicylic acid. The inorganic acid may be a variety of inorganic acids conventional in the art that are capable of forming salts, preferably one or more of hydrochloric acid, sulfuric acid, and phosphoric acid. The organic base may be a variety of organic bases conventional in the art that are capable of forming salts, preferably one or more of pyridines, imidazoles, pyrazines, indoles, purines, tertiary amines, and anilines. The tertiary amine organic base is preferably triethylamine and/or N,N-diisopropylethylamine. The aniline organic base is preferably N,N-dimethylaniline. The pyridine organic base is preferably one or more of pyridine, methylpyridine, 4-dimethylaminopyridine, and 2-methyl-5-ethylpyridine. The inorganic base may be a variety of inorganic bases conventional in the art that are capable of forming salts, preferably one or more of alkali metal hydrides, alkali metal hydroxides, alkali metal alkoxides, potassium carbonate, sodium carbonate, lithium carbonate, cesium carbonate, potassium bicarbonate, and sodium bicarbonate. The alkali metal hydride is preferably sodium hydride and/or potassium hydride. The alkali metal hydroxide is preferably one or more of sodium hydroxide, potassium hydroxide, and lithium hydroxide. The alkali metal alkoxide is preferably one or more of sodium methoxide, sodium ethoxide, potassium tert-butoxide, and sodium tert-butoxide.

In the present disclosure, the term “solvate” refers to a substance formed by a compound or a salt thereof with a suitable solvent. The solvent is preferably water or an organic solvent.

In the present disclosure, the term “patient” includes any animal, preferably a mammal, more preferably a human.

On the basis of common knowledge in the art, the above preferred conditions can be arbitrarily combined to obtain the preferred examples of the present disclosure.

The reagents and raw materials used in the present disclosure are commercially available.

The positive and progressive effect of the present disclosure is that the compounds of the present disclosure have excellent efficacy against various mental diseases and neurodegenerative diseases such as schizophrenia, depression, and Parkinson's disease, and can thus be used as effective drugs for the treatment of the diseases.

### DETAILED DESCRIPTION OF THE PREFERRED EMBODIMENT

The present disclosure is further described below by way of examples, but is not limited to the scope of the described examples. In the following examples, experimental methods without specified conditions are selected according to conventional methods and conditions, or according to the product instructions.

In the following examples, “reacted overnight” means 12 to 18 hours.

### Example 1: Preparation of compound 7-(4-(8-methoxy-3,4-dihydrobenzofuro[2,3-c]pyridin-2(1H)-yl)butoxy)quinolin-2(1H)-one (I-A1)

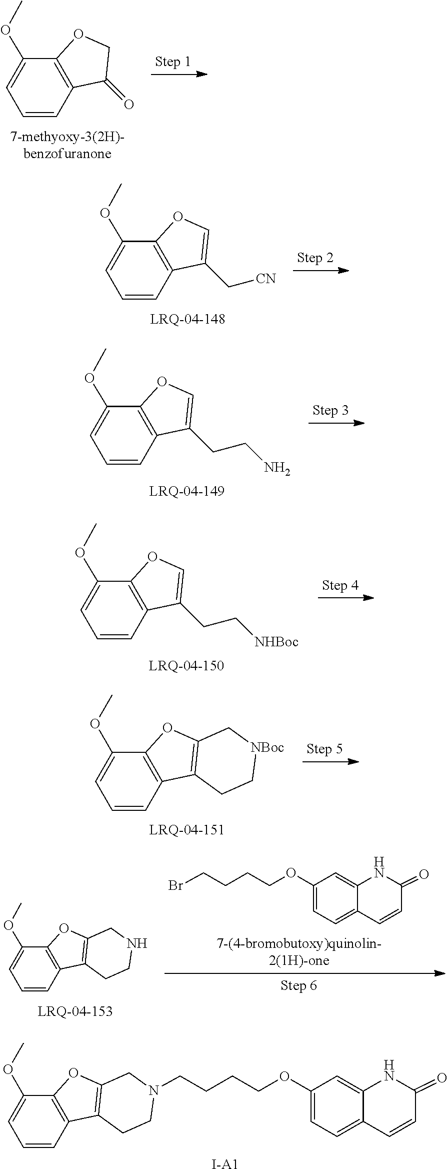

Step 1: LRQ-04-148 was synthesized with reference to patent WO2006064355A2. ¹H NMR (800 MHz, CDCl₃) δ 7.66 (s, 1H), 7.23 (t, J=7.9 Hz, 1H), 7.18-7.15 (m, 1H), 6.89-6.85 (m, 1H), 4.02 (s, 3H), 3.75 (d, J=1.2 Hz, 2H). HHRMS (ESI) calculated for C₁₁H₁₀NO₂⁺ [M+H]⁺: 188.0706, found: 188.0702.

Step 2: LRQ-04-149 was synthesized with reference to patent WO2006064355A2. ¹H NMR (800 MHz, CDCl₃) δ 7.64 (s, 1H), 7.25-7.20 (m, 1H), 7.17 (t, J=7.8 Hz, 1H), 6.84-6.79 (m, 1H), 3.99 (s, 3H), 3.30 (t, J=7.1 Hz, 2H), 3.21-3.18 (m, 2H). HRMS (ESI) calculated for C₁₁H₁₄NO₂⁺ [M+H]⁺: 192.1019, found: 192.1019.

Step 3: LRQ-04-149 (500 mg, 2.62 mmol) and Et₃N (796 mg, 7.86 mmol) were dissolved in dichloromethane (10 mL), then (Boc)₂O (714 mg, 3.27 mmol) was added thereto, and the reaction mixture was reacted at room temperature overnight. After the reaction was completed, the reaction mixture was diluted with saturated sodium bicarbonate aqueous solution (10 mL), extracted with dichloromethane (20 mL*3), washed with saturated brine, dried over anhydrous sodium sulfate, and the solvent was removed by evaporation under reduced pressure to obtain LRQ-04-150 as a white solid, which was directly used in the next reaction step without purification. HRMS (ESI) calculated for C₁₆H₂₂NO₄⁺ [M+H]⁺: 292.1543, found: 292.1549.

Step 4: LRQ-04-150 and paraformaldehyde (157 mg, 5.24 mmol) were dissolved in toluene (10 mL), then p-toluenesulfonic acid (25 mg, 0.13 mmol) was added thereto, and the reaction mixture was refluxed overnight. After the reaction was completed, the reaction mixture was diluted with saturated sodium bicarbonate aqueous solution (10 mL), extracted with ethyl acetate (20 mL*3), washed with saturated brine, dried over anhydrous sodium sulfate, and the solvent was removed by evaporation under reduced pressure to obtain LRQ-04-151 as a white solid, which was directly used in the next reaction step without purification. HRMS (ESI) calculated for C₁₇H₂₂NO₄⁺ [M+H]⁺: 304.1543, found: 304.1545.

Step 5: LRQ-04-151 was dissolved in a 4 M solution of HCl in dioxane (8 mL), and the reaction mixture was reacted at room temperature overnight. After the reaction was completed, the solvent was removed by evaporation under reduced pressure. The residue was dissolved in diethyl ether (8 mL), stirred at room temperature for 15 minutes, and filtered under reduced pressure. The filter residue was rinsed with diethyl ether and dried under reduced pressure to obtain LRQ-04-153 (202 mg, three-step reaction yield: 38%) as a white solid. ¹H NMR (800 MHz, MeOH-d₄) δ 7.22 (t, J=7.9 Hz, 1H), 7.16-7.11 (m, 1H), 6.97-6.93 (m, 1H), 4.45 (s, 2H), 3.97 (s, 3H), 3.61 (t, J=6.0 Hz, 2H), 3.07-3.01 (m, 2H). ¹³C NMR (201 MHz, MeOH-d₄) δ 146.92, 145.61, 145.53, 129.59, 125.35, 112.87, 112.35, 108.77, 56.60, 43.13, 41.95, 19.03. HRMS (ESI) calculated for C₁₂H₁₄NO₂⁺ [M+H]⁺: 204.1019, found: 204.1016.

Step 6: LRQ-04-153 (100 mg, 0.49 mmol) was dissolved in DMF (8 mL), then DIPEA (189 mg, 1.47 mmol) and 7-(4-bromobutoxy)quinolin-2(1H)-one (218 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a reddish brown solid (119 mg, yield: 58%). ¹H NMR (800 MHz, MeOH-d₄) δ 7.79-7.71 (m, 1H), 7.45 (d, J=8.6 Hz, 1H), 7.14-7.07 (m, 1H), 7.01 (d, J=7.5 Hz, 1H), 6.85-6.72 (m, 3H), 6.46-6.39 (m, 1H), 4.07 (t, J=5.5 Hz, 2H), 3.96 (s, 3H), 3.71 (s, 2H), 2.96-2.83 (m, 2H), 2.78-2.66 (m, 4H), 1.94-1.76 (m, 4H). ¹³C NMR (201 MHz, MeOH-d₄) δ 165.02, 161.97, 150.89, 145.68, 144.26, 141.87, 140.60, 129.93, 129.73, 123.88, 117.73, 114.93, 113.27, 112.28, 111.75, 106.83, 99.26, 68.39, 57.53, 56.36, 50.83, 50.39, 27.48, 24.04, 20.90. HRMS (ESI) calculated for C₂₅H₂₇N₂O₄⁺ [M+H]⁺: 419.1965, found: 419.1971. HPLC: 99.54% (λ=254 nm, t_{R}=11.18 min).

### Example 2: Preparation of compound 7-(4-(8-methoxy-3,4-dihydrobenzofuro[2,3-c]pyridin-2(1H)-yl)butoxy)-3,4-dihydroquinolin-2(1H)-one (I-A2)

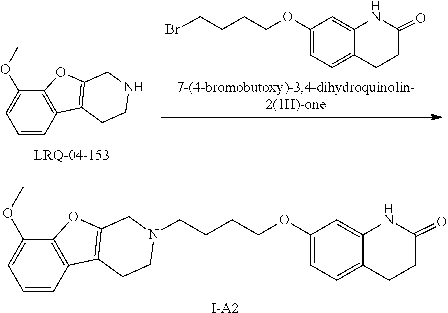

LRQ-04-153 (100 mg, 0.49 mmol) was dissolved in DMF (8 mL), then DIPEA (189 mg, 1.47 mmol) and 7-(4-bromobutoxy)-3,4-dihydroquinolin-2(1H)-one (220 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a white solid (113 mg, yield: 55%). ¹H NMR (800 MHz, CDCl₃) δ 8.77 (s, 1H), 7.13 (t, J=7.8 Hz, 1H), 7.05-6.97 (m, 2H), 6.76 (d, J=7.9 Hz, 1H), 6.53-6.48 (m, 1H), 6.39-6.34 (m, 1H), 3.99 (s, 3H), 3.95 (t, J=6.1 Hz, 2H), 3.72 (s, 2H), 2.90-2.83 (m, 4H), 2.73 (t, J=5.4 Hz, 2H), 2.71-2.66 (m, 2H), 2.62-2.58 (m, 2H), 1.87-1.80 (m, 2H), 1.80-1.74 (m, 2H). ¹³C NMR (201 MHz, CDCl₃) δ 172.24, 158.70, 151.21, 145.25, 143.72, 138.28, 129.74, 128.66, 123.30, 115.78, 111.86, 111.30, 108.86, 106.03, 102.36, 67.89, 57.04, 56.10, 50.43, 50.11, 31.16, 27.17, 24.64, 23.93, 20.87. HRMS (ESI) calculated for C₂₅H₂₉N₂O₄⁺ [M+H]⁺: 421.2122, found: 421.2123. HPLC: 97.33% (λ=254 nm, t_{R}=11.25 min).

### Example 3: Preparation of compound 6-(4-(8-methoxy-3,4-dihydrobenzofuro[2,3-c]pyridin-2(1H)-yl)butoxy)indolin-2-one (I-A3)

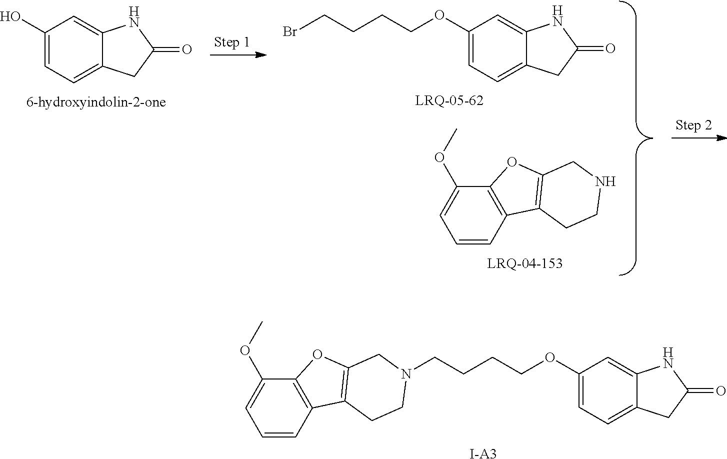

Step 1: 6-Hydroxyindolin-2-one (2.17 g, 14.55 mmol) was dissolved in DMF (40 mL), then K₂CO₃ (2.1 g, 14.55 mmol) and 1,4-dibromobutane (9.42 g, 43.65 mmol) were sequentially added thereto, and the reaction mixture was reacted at room temperature overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:4) to obtain LRQ-05-62 (0.25 g, yield: 6%) as a reddish brown solid. ¹H NMR (800 MHz, CDCl₃) δ 8.47 (s, 1H), 7.11-7.07 (m, 1H), 6.55-6.50 (m, 1H), 6.47 (d, J=1.6 Hz, 1H), 3.97 (t, J=6.0 Hz, 2H), 3.52-3.45 (m, 4H), 2.09-2.04 (m, 2H), 1.96-1.90 (m, 2H). ¹³C NMR (201 MHz, CDCl₃) δ 178.61, 159.29, 143.59, 125.28, 117.21, 107.92, 97.74, 67.30, 35.78, 33.52, 29.57, 27.98. HRMS (ESI) calculated for C₁₂H₁₅BrNO₂⁺ [M+H]⁺: 284.0281, found: 284.0289.

Step 2: LRQ-04-153 (100 mg, 0.49 mmol) was dissolved in DMF (8 mL), then DIPEA (189 mg, 1.47 mmol) and LRQ-05-62 (209 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (46 mg, yield: 23%). ¹H NMR (800 MHz, CDCl₃) δ 7.72 (s, 1H), 7.14 (t, J=7.8 Hz, 1H), 7.07 (d, J=8.2 Hz, 1H), 7.04 (d, J=7.7 Hz, 1H), 6.78 (d, J=7.9 Hz, 1H), 6.54-6.50 (m, 1H), 6.43 (d, J=2.0 Hz, 1H), 4.03-3.95 (m, 5H), 3.76 (s, 2H), 3.46-3.40 (m, 2H), 2.96-2.90 (m, 2H), 2.80-2.70 (m, 4H), 1.88-1.84 (m, 2H), 1.83-1.80 (m, 2H). ¹³C NMR (201 MHz, CDCl₃) δ 175.12, 159.33, 148.50, 145.32, 143.85, 143.56, 129.93, 125.24, 123.49, 117.14, 111.87, 111.37, 107.81, 106.23, 97.75, 67.96, 57.42, 56.16, 50.42, 49.87, 35.69, 29.83, 27.11, 20.50. HRMS (ESI) calculated for C₂₄H₂₇N₂O₄⁺ [M+H]⁺: 407.1965, found: 407.1966. HPLC: 96.65% (λ=254 nm, t_{R}=11.28 min).

### Example 4: Preparation of compound 7-(4-(8-methoxy-3,4-dihydrobenzofuro[2,3-c]pyridin-2(1H)-yl)butoxy)-1-methylquinolin-2(1H)-one (I-A4)

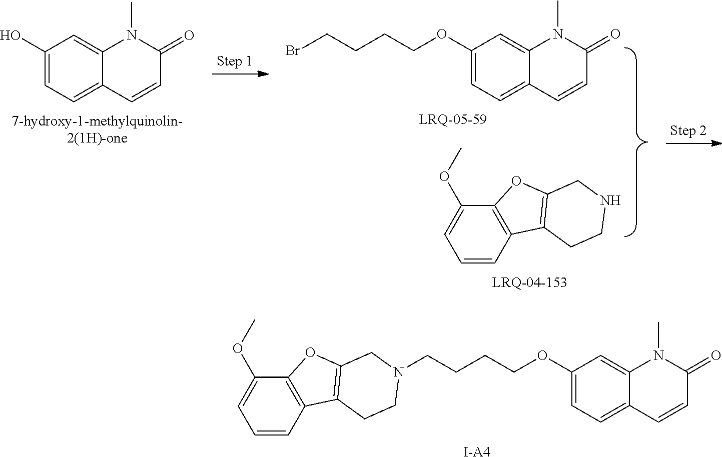

Step 1: 7-Hydroxy-1-methylquinolin-2(1H)-one (2.55 g, 14.55 mmol) was dissolved in DMF (40 mL), then K₂CO₃ (2.1 g, 14.55 mmol) and 1,4-dibromobutane (9.42 g, 43.65 mmol) were sequentially added thereto, and the reaction mixture was reacted at room temperature overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:4) to obtain LRQ-05-59 (2.16 g, yield: 48%) as a yellow solid. ¹H NMR (800 MHz, CDCl₃) δ 7.59 (d, J=9.4 Hz, 1H), 7.45 (d, J=8.5 Hz, 1H), 6.82-6.79 (m, 1H), 6.79-6.76 (m, 1H), 6.55 (d, J=9.4 Hz, 1H), 4.13-4.06 (m, 2H), 3.68 (s, 3H), 3.54-3.24 (m, 2H), 2.13-2.04 (m, 2H), 2.03-1.94 (m, 2H). ¹³C NMR (201 MHz, CDCl₃) δ 162.89, 161.21, 141.85, 138.85, 130.23, 118.69, 115.11, 110.00, 99.42, 67.21, 33.39, 30.19, 29.63, 27.94. HRMS (ESI) calculated for C₁₄H₁₇BrNO₂⁺ [M+H]⁺: 310.0437, found: 310.0441.

Step 2: LRQ-04-153 (100 mg, 0.49 mmol) was dissolved in DMF (8 mL), then DIPEA (189 mg, 1.47 mmol) and LRQ-05-59 (228 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a reddish brown solid (108 mg, yield: 51%). ¹H NMR (800 MHz, CDCl₃) δ 7.56 (d, J=9.4 Hz, 1H), 7.42 (d, J=8.6 Hz, 1H), 7.12 (t, J=7.8 Hz, 1H), 7.02 (d, J=7.7 Hz, 1H), 6.82-6.78 (m, 1H, 1H), 6.77-6.74 (m, 2H), 6.52 (d, J=9.3 Hz, 1H), 4.11 (t, J=6.2 Hz, 2H), 3.98 (s, 3H), 3.72 (s, 2H), 3.65 (s, 3H), 2.89 (t, J=5.4 Hz, 2H), 2.76-2.68 (m, 4H), 1.94-1.87 (m, 2H), 1.85-1.79 (m, 2H). ¹³C NMR (201 MHz, CDCl₃) δ 162.87, 161.33, 151.12, 145.25, 143.71, 141.76, 138.83, 130.14, 129.68, 123.34, 118.44, 114.95, 111.85, 111.27, 110.05, 106.02, 99.36, 68.10, 56.97, 56.09, 50.51, 50.06, 29.55, 27.19, 23.89, 20.88. HRMS (ESI) calculated for C₂₆H₂₉N₂O₄⁺ [M+H]⁺: 433.2122, found: 433.2125. HPLC: 97.65% (λ=254 nm, t_{R}=12.43 min).

### Example 5: Preparation of compound 7-(4-(8-methoxy-3,4-dihydrobenzofuro[2,3-c]pyridin-2(1H)-yl)butoxy)-2H-chromen-2-one (I-A5)

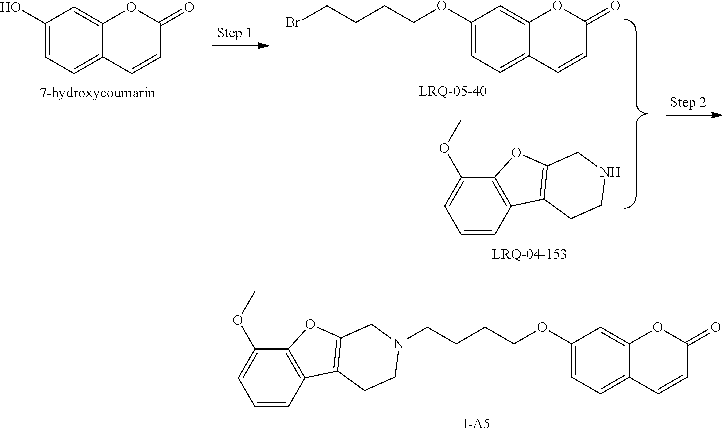

Step 1: 7-Hydroxycoumarin (2.36 g, 14.55 mmol) was dissolved in DMF (40 mL), then K₂CO₃ (2.1 g, 14.55 mmol) and 1,4-dibromobutane (9.42 g, 43.65 mmol) were sequentially added thereto, and the reaction mixture was reacted at room temperature overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:4) to obtain LRQ-05-40 (2.37 g, yield: 55%) as a white solid. ¹H NMR (800 MHz, CDCl₃) δ 7.63 (d, J=9.4 Hz, 1H), 7.38-7.34 (m, 1H), 6.84-6.81 (m, 1H), 6.79 (d, J=2.3 Hz, 1H), 6.24 (d, J=9.4 Hz, 1H), 4.05 (t, J=6.1 Hz, 2H), 3.49 (t, J=6.6 Hz, 2H), 2.11-2.05 (m, 2H), 2.02-1.96 (m, 2H). ¹³C NMR (201 MHz, CDCl₃) δ 162.20, 161.30, 156.02, 143.50, 128.90, 113.28, 112.99, 112.71, 101.50, 67.66, 33.31, 29.44, 27.79. HRMS (ESI) calculated for C₁₃H₁₄BrO₃⁺ [M+H]⁺: 297.0121, found: 297.0429.

Step 2: LRQ-04-153 (100 mg, 0.49 mmol) was dissolved in DMF (8 mL), then DIPEA (189 mg, 1.47 mmol) and LRQ-05-40 (219 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a white solid (117 mg, yield: 57%). ¹H NMR (800 MHz, CDCl₃) δ 7.60 (d, J=9.4 Hz, 1H), 7.33 (d, J=8.6 Hz, 1H), 7.13 (t, J=7.8 Hz, 1H), 7.04-7.00 (m, 1H), 6.84-6.80 (m, 1H), 6.79 (d, J=2.1 Hz, 1H), 6.76 (d, J=7.9 Hz, 1H), 6.24-6.19 (m, 1H), 4.06 (t, J=6.2 Hz, 2H), 3.99 (s, 3H), 3.72 (s, 2H), 2.89 (t, J=5.3 Hz, 2H), 2.77-2.67 (m, 4H), 1.93-1.85 (m, 2H), 1.84-1.73 (m, 2H). ¹³C NMR (201 MHz, CDCl₃) δ 162.36, 161.37, 155.99, 151.13, 145.28, 143.75, 143.53, 129.73, 128.83, 123.34, 113.07, 113.05, 112.56, 111.88, 111.33, 106.06, 101.45, 68.36, 56.95, 56.13, 50.51, 50.12, 26.98, 23.87, 20.89. HRMS (ESI) calculated for C₂₅H₂₆NO₅⁺ [M+H]⁺: 420.1805, found: 420.1807. HPLC: 96.57% (λ=254 nm, t_{R}=11.40 min).

### Example 6: Preparation of compound 8-methoxy-2-(4-(quinolin-7-yloxy)butyl)-1,2,3,4-tetrahydrobenzofuro[2,3-c]pyridine (I-A6)

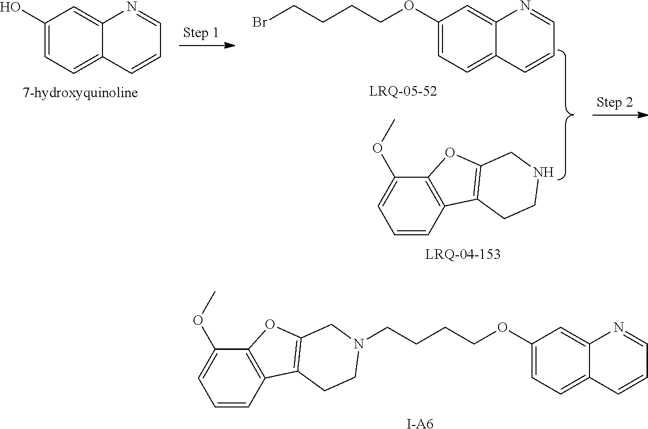

Step 1: 7-Hydroxyquinoline (2.11 g, 14.55 mmol) was dissolved in DMF (40 mL), then K₂CO₃ (2.1 g, 14.55 mmol) and 1,4-dibromobutane (9.42 g, 43.65 mmol) were sequentially added thereto, and the reaction mixture was reacted at room temperature overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:4) to obtain LRQ-05-52 (2.52 g, yield: 62%) as a yellow solid. ¹H NMR (800 MHz, CDCl₃) δ 8.82 (d, J=2.8 Hz, 1H), 8.07 (d, J=8.0 Hz, 1H), 7.74-7.66 (m, 1H), 7.40 (d, J=1.9 Hz, 1H), 7.27-7.25 (m, 1H), 7.23-7.16 (m, 1H), 4.15 (t, J=6.1 Hz, 2H), 3.50 (t, J=6.6 Hz, 2H), 2.16-2.06 (m, 2H), 2.06-1.98 (m, 2H). ¹³C NMR (201 MHz, CDCl₃) δ 160.04, 150.57, 149.90, 135.92, 128.96, 123.68, 120.15, 119.11, 107.95, 67.23, 33.47, 29.65, 27.88. HRMS (ESI) calculated for C₁₃H₁₅BrNO⁺ [M+H]⁺: 280.0332, found: 280.0339.

Step 2: LRQ-04-153 (100 mg, 0.49 mmol) was dissolved in DMF (8 mL), then DIPEA (189 mg, 1.47 mmol) and LRQ-05-52 (206 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (110 mg, yield: 56%). ¹H NMR (800 MHz, CDCl₃) δ 8.81-8.75 (m, 1H), 8.06-7.99 (m, 1H), 7.66 (d, J=8.9 Hz, 1H), 7.39 (d, J=2.2 Hz, 1H), 7.24-7.20 (m, 1H), 7.20-7.15 (m, 1H), 7.11 (t, J=7.8 Hz, 1H), 7.01 (d, J=7.6 Hz, 1H), 6.76-6.71 (m, 1H), 4.14 (t, J=6.3 Hz, 2H), 3.97 (s, 3H), 3.70 (s, 2H), 2.85 (t, J=5.6 Hz, 2H), 2.71 (t, J=5.5 Hz, 2H), 2.70-2.67 (m, 2H), 1.95-1.88 (m, 2H), 1.85-1.78 (m, 2H). ¹³C NMR (201 MHz, CDCl₃) δ 160.11, 151.31, 150.45, 149.88, 145.20, 143.67, 135.79, 129.74, 128.83, 123.53, 123.21, 120.14, 118.93, 111.83, 111.26, 107.84, 105.96, 67.88, 57.07, 56.05, 50.41, 50.15, 26.98, 24.03, 20.90. HRMS (ESI) calculated for C₂₅H₂₇N₂O₃⁺ [M+H]⁺: 403.2016, found: 403.2011. HPLC: 96.75% (λ=254 nm, t_{R}=10.06 min).

### Example 7: Preparation of compound 2-(4-(benzo[d]thiazol-5-yloxy)butyl)-8-methoxy-1,2,3,4-tetrahydrobenzofuro[2,3-c]pyridine (I-A7)

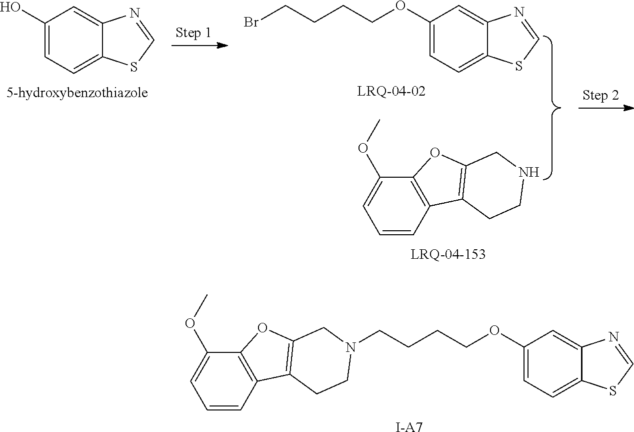

Step 1: 5-Hydroxybenzothiazole (2.20 g, 14.55 mmol) was dissolved in DMF (40 mL), then K₂CO₃ (2.1 g, 14.55 mmol) and 1,4-dibromobutane (9.42 g, 43.65 mmol) were sequentially added thereto, and the reaction mixture was reacted at room temperature overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:4) to obtain LRQ-04-02 (2.96 g, yield: 72%) as a colorless oily liquid. ¹H NMR (800 MHz, CDCl₃) δ 8.97 (s, 1H), 7.81-7.78 (m, 1H), 7.61-7.57 (m, 1H), 7.10-7.06 (m, 1H), 4.09 (t, J=6.1 Hz, 2H), 3.50 (t, J=6.7 Hz, 2H), 2.14-2.06 (m, 2H), 2.03-1.97 (m, 2H). ¹³C NMR (201 MHz, CDCl₃) δ 158.37, 155.11, 154.72, 125.74, 122.19, 116.52, 106.55, 67.44, 33.52, 29.64, 27.98. HRMS (ESI) calculated for C₁₁H₁₃BrNOS⁺ [M+H]⁺: 285.9896, found: 285.9897.

Step 2: LRQ-04-153 (100 mg, 0.49 mmol) was dissolved in DMF (8 mL), then DIPEA (189 mg, 1.47 mmol) and LRQ-04-02 (210 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a reddish brown solid (92 mg, yield: 46%). ¹H NMR (800 MHz, CDCl₃) δ 8.96 (s, 1H), 7.80-7.76 (m, 1H), 7.59 (d, J=2.3 Hz, 1H), 7.13 (t, J=7.8 Hz, 1H), 7.11-7.06 (m, 1H), 7.03 (d, J=7.7 Hz, 1H), 6.77 (d, J=7.9 Hz, 1H), 4.10 (t, J=6.2 Hz, 2H), 4.00 (s, 3H), 3.78 (s, 2H), 2.97-2.90 (m, 2H), 2.80-2.72 (m, 4H), 1.95-1.89 (m, 2H), 1.89-1.82 (m, 2H). ¹³C NMR (201 MHz, CDCl₃) δ 158.48, 155.04, 154.74, 145.31, 143.83, 129.67, 125.61, 123.38, 122.14, 116.57, 111.88, 111.35, 111.30, 106.56, 106.16, 68.12, 56.92, 56.15, 50.40, 50.03, 27.12, 23.91, 20.68. HRMS (ESI) calculated for C₂₃H₂₅N₂O₃S⁺ [M+H]⁺: 409.1580, found: 409.1587. HPLC: 95.00% (λ=254 nm, t_{R}=11.37 min).

### Example 8: Preparation of compound 7-(3-(8-methoxy-3,4-dihydrobenzofuro[2,3-c]pyridin-2(1H)-yl)propoxy)quinolin-2(1H)-one (I-A8)

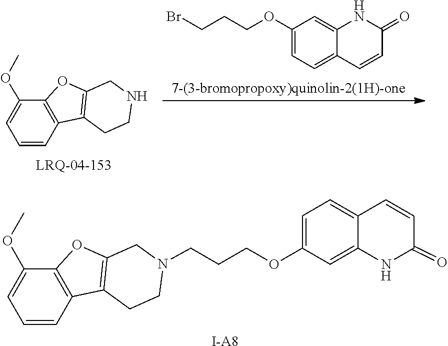

LRQ-04-153 (100 mg, 0.49 mmol) was dissolved in DMF (8 mL), then DIPEA (189 mg, 1.47 mmol) and 7-(3-bromopropoxy)quinolin-2(1H)-one (208 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (48 mg, yield: 24%). ¹H NMR (800 MHz, CDCl₃) δ 11.84 (s, 1H), 7.70 (d, J=9.4 Hz, 1H), 7.43 (d, J=9.3 Hz, 1H), 7.13 (t, J=7.8 Hz, 1H), 7.04 (d, J=7.7 Hz, 1H), 6.84-6.80 (m, 2H), 6.77 (d, J=7.9 Hz, 1H), 6.52 (d, J=9.4 Hz, 1H), 4.17 (t, J=6.1 Hz, 2H), 4.00 (s, 3H), 3.74 (s, 2H), 2.90 (t, J=5.5 Hz, 2H), 2.83 (t, J=7.2 Hz, 2H), 2.78-2.73 (m, 2H), 2.14-2.08 (m, 2H). ¹³C NMR (201 MHz, CDCl₃) δ 164.85, 161.44, 145.32, 143.78, 140.93, 140.41, 139.38, 129.85, 129.21, 123.32, 118.18, 114.35, 112.56, 111.97, 111.36, 106.05, 99.25, 66.58, 56.17, 54.06, 50.62, 50.35, 27.38, 21.05. HRMS (ESI) calculated for C₂₄H₂₅N₂O₄⁺ [M+H]⁺: 405.1809, found: 405.1811. HPLC: 97.28% (λ=254 nm, t_{R}=11.56 min).

### Example 9: Preparation of compound 7-(3-(8-methoxy-3,4-dihydrobenzofuro[2,3-c]pyridin-2(1H)-yl)propoxy)-3,4-dihydroquinolin-2(1H)-one (I-A9)

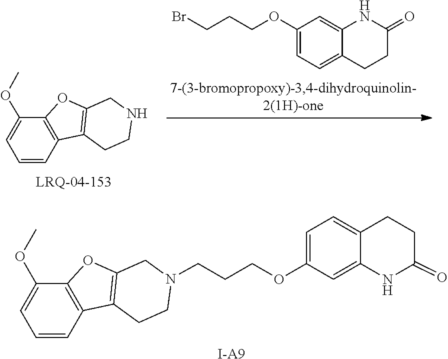

LRQ-04-153 (100 mg, 0.49 mmol) was dissolved in DMF (8 mL), then DIPEA (189 mg, 1.47 mmol) and 7-(3-bromopropoxy)-3,4-dihydroquinolin-2(1H)-one (209 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (46 mg, yield: 23%). ¹H NMR (800 MHz, CDCl₃) δ 8.08 (s, 1H), 7.14 (t, J=7.8 Hz, 1H), 7.05-7.02 (m, 2H), 6.77 (d, J=7.9 Hz, 1H), 6.55-6.51 (m, 1H), 6.35-6.32 (m, 1H), 4.03 (t, J=6.2 Hz, 2H), 4.00 (s, 3H), 3.72 (s, 2H), 2.91-2.86 (m, 5H), 2.80 (t, J=7.2 Hz, 2H), 2.73 (t, J=5.3 Hz, 2H), 2.63-2.59 (m, 2H), 2.11-1.98 (m, 2H). ¹³C NMR (201 MHz, CDCl₃) δ 171.83, 158.73, 151.45, 145.31, 143.76, 138.23, 129.83, 128.80, 123.33, 115.95, 111.94, 111.34, 108.81, 106.03, 102.38, 66.36, 56.15, 54.08, 50.59, 50.31, 31.23, 27.46, 24.73, 21.04. HRMS (ESI) calculated for C₂₄H₂₇N₂O₄⁺ [M+H]⁺: 407.1965, found: 407.1958. HPLC: 96.06% (λ=254 nm, t_{R}=11.55 min).

### Example 10: Preparation of compound (E)-7-((4-(8-methoxy-3,4-dihydrobenzofuro[2,3-c]pyridin-2(1H)-yl)but-2-en-1-yl)oxy)quinolin-2(1H)-one (I-A10)

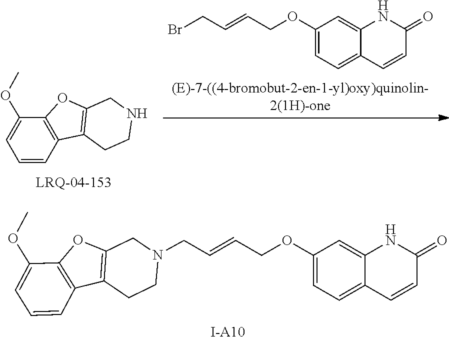

LRQ-04-153 (100 mg, 0.49 mmol) was dissolved in DMF (8 mL), then DIPEA (189 mg, 1.47 mmol) and (E)-7-((4-bromobut-2-en-1-yl)oxy)quinolin-2(1H)-one (217 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (150 mg, yield: 74%). ¹H NMR (800 MHz, CDCl₃) δ 12.27 (s, 1H), 7.70 (d, J=9.4 Hz, 1H), 7.43 (d, J=8.6 Hz, 1H), 7.12 (t, J=7.8 Hz, 1H), 7.01 (d, J=7.7 Hz, 1H), 6.85 (s, 1H), 6.84-6.79 (m, 1H), 6.75 (d, J=7.9 Hz, 1H), 6.55 (d, J=9.4 Hz, 1H), 6.08-6.01 (m, 1H), 5.98-5.91 (m, 1H), 4.71-4.61 (m, 2H), 3.99 (s, 3H), 3.72 (s, 2H), 3.33 (d, J=6.4 Hz, 2H), 2.91-2.84 (m, 2H), 2.72 (t, J=5.2 Hz, 2H). ¹³C NMR (201 MHz, CDCl₃) δ 165.02, 160.92, 151.36, 145.30, 143.76, 140.86, 140.45, 131.59, 129.82, 129.20, 128.14, 123.29, 118.29, 114.45, 112.89, 111.88, 111.33, 106.05, 99.44, 68.51, 59.02, 56.15, 50.17, 50.02, 20.95. HRMS (ESI) calculated for C₂₅H₂₅N₂O₄⁺ [M+H]⁺: 417.1809, found: 417.1811. HPLC: 99.16% (λ254 nm, t_{R}=11.76 min).

### Example 11: Preparation of compound N-(trans-4-(2-(8-methoxy-3,4-dihydrobenzofuro[2,3-c]pyridin-2(1H)-yl)ethyl)cyclohexyl)acetamide (I-A11)

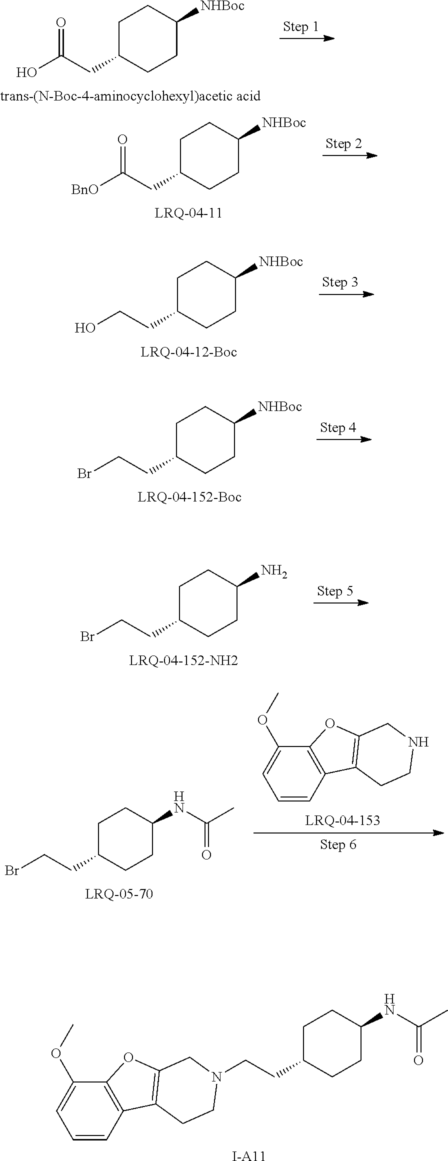

Step 1: NaOH (62 mg, 1.56 mmol) was dissolved in H₂O (10 mL), then K₂CO₃ (440 mg, 3.16 mmol), trans-(N-Boc-4-aminocyclohexyl)acetic acid (200 mg, 0.78 mmol), and benzyl bromide (540 mg, 3.16 mmol) were sequentially added thereto, and the reaction mixture was refluxed for 3 hours. After the reaction was completed, the reaction mixture was cooled to room temperature, extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:2) to obtain LRQ-04-11 (220 mg, yield: 83%) as a white solid. ¹H NMR (800 MHz, CDCl₃) δ 7.38-7.30 (m, 5H), 5.10 (s, 2H), 4.37 (s, 1H), 3.42-3.29 (m, 1H), 2.24 (d, J=6.8 Hz, 2H), 2.03-1.92 (m, 2H), 1.82-1.70 (m, 3H), 1.43 (s, 9H), 1.16-1.00 (m, 4H). ¹³C NMR (201 MHz, CDCl₃) δ 172.79, 155.33, 136.15, 128.68 (2C), 128.33, 128.30 (2C), 79.22, 66.25, 49.58, 41.52, 34.18 (2C), 33.25, 31.71 (2C), 28.56 (3C). HRMS (ESI) calculated for C₂₀H₂₉NO₄Na⁺ [M+H]⁺: 370.1989, found: 370.1988.

Step 2: LRQ-04-11 (200 mg, 0.63 mmol) was dissolved in THE (10 mL). The reaction mixture was replaced with nitrogen three times, then cooled to −10° C., and DIBAL-H (1.9 mL, 1.0 M in tetrahydrofuran) was added thereto. The reaction mixture was then warmed to 0° C. and reacted for 6 hours. After the reaction was completed, the reaction mixture was added with saturated potassium sodium tartrate aqueous solution (5 mL), then stirred at room temperature for 1 hour, extracted with ethyl acetate (20 mL*3), washed with saturated brine, dried over anhydrous sodium sulfate, and the solvent was removed by evaporation under reduced pressure to obtain LRQ-04-12-Boc as a white solid, which was directly used in the next reaction step without purification. HRMS (ESI) calculated for C₁₃H₂₆NO₃⁺ [M+H]⁺: 244.1907, found: 244.1910.

Step 3: LRQ-04-12-Boc was dissolved in DCM (10 mL), and the reaction mixture was cooled to 0° C. Carbon tetrabromide (500 mg, 1.41 mmol) and triphenylphosphine (500 mg, 1.88 mmol) were sequentially added thereto, and the reaction mixture was reacted at room temperature overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-04-152-Boc (113 mg, yield: 59%) as a white solid. ¹H NMR (800 MHz, CDCl₃) δ 3.45-3.39 (m, 2H), 3.39-3.33 (m, 1H), 2.04-1.95 (m, 2H), 1.81-1.72 (m, 4H), 1.50-1.37 (m, 10H), 1.14-1.06 (m, 2H), 1.06-0.97 (m, 2H). ¹³C NMR (201 MHz, CDCl₃) δ 155.37, 79.22, 49.88, 39.68, 35.38, 33.33 (2C), 31.87, 31.30 (2C), 28.58 (3C). HHRMS (ESI) calculated for C₁₃H₂₅BrNO₂⁺ [M+H]⁺: 306.1063, found: 306.1066.

Step 4: LRQ-04-152-Boc (113 mg, 0.38 mmol) was dissolved in DCM (10 mL), and the reaction mixture was cooled to 0° C. Trifluoroacetic acid (1 mL) was added thereto, and the reaction mixture was reacted for 2 hours. After the reaction was completed, the solvent was removed by evaporation under reduced pressure to obtain LRQ-04-12-NH2 as a colorless oily liquid, which was directly used in the next reaction step without purification. HRMS (ESI) calculated for C₈H₁₇BrN⁺ [M+H]⁺: 206.0539, found: 206.0541.

Step 5: LRQ-04-12-NH2 was dissolved in DCM (10 mL), then triethylamine (115 mg, 1.14 mmol) and acetyl chloride (33 mg, 0.42 mmol) were sequentially added thereto, and the reaction mixture was reacted at room temperature overnight. After the reaction was completed, the reaction mixture was diluted with saturated sodium bicarbonate aqueous solution (10 mL), extracted with dichloromethane (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-05-70 (43 mg, yield: 46%) as a yellow solid. ¹H NMR (800 MHz, CDCl₃) δ 5.48-5.27 (m, 1H), 3.74-3.64 (m, 1H), 3.42 (t, J=7.0 Hz, 2H), 2.01-1.96 (m, 2H), 1.94 (s, 3H), 1.80-1.73 (m, 4H), 1.48-1.41 (m, 1H), 1.14-1.08 (m, 2H), 1.08-1.01 (m, 2H). ¹³C NMR (201 MHz, CDCl₃) δ 169.34, 48.63, 39.59, 35.29, 32.97 (2C), 31.86, 31.17 (2C), 23.70. HRMS (ESI) calculated for C₁₀H₁₉BrNO⁺ [M+H]⁺: 248.0645, found: 248.0638.

Step 6: LRQ-04-153 (24 mg, 0.12 mmol) was dissolved in DMF (8 mL), then DIPEA (45 mg, 0.35 mmol) and LRQ-05-70 (42 mg, 0.17 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (20 mg, yield: 44%). ¹H NMR (800 MHz, CDCl₃) δ 7.12 (t, J=7.8 Hz, 1H), 7.02 (d, J=7.7 Hz, 1H), 6.79-6.71 (m, 1H), 5.37 (d, J=8.0 Hz, 1H), 3.98 (s, 3H), 3.74-3.62 (m, 3H), 2.83 (t, J=5.5 Hz, 2H), 2.71 (t, J=5.4 Hz, 2H), 2.65-2.58 (m, 2H), 2.01-1.94 (m, 2H), 1.93 (s, 3H), 1.81-1.74 (m, 2H), 1.53-1.46 (m, 2H), 1.31-1.26 (m, 1H), 1.11-1.04 (m, 4H). ¹³C NMR (201 MHz, CDCl₃) δ 169.35, 151.31, 145.26, 143.72, 129.78, 123.29, 111.86, 111.30, 106.02, 56.12, 55.31, 50.46, 50.23, 48.73, 35.26, 34.36, 33.15 (2C), 31.95 (2C), 23.67, 20.89. HHRMS (ESI) calculated for C₂₂H₃₁N₂O₃⁺ [M+H]⁺: 371.2329, found: 371.2333. HPLC: 96.63% (λ=254 nm, t_{R}=10.56 min).

### Example 12: Preparation of compound 3-(trans-4-(2-(8-methoxy-3,4-dihydrobenzofuro[2,3-c]pyridin-2(1H)-yl)ethyl)cyclohexyl)-1,1-dimethylurea (I-A12)

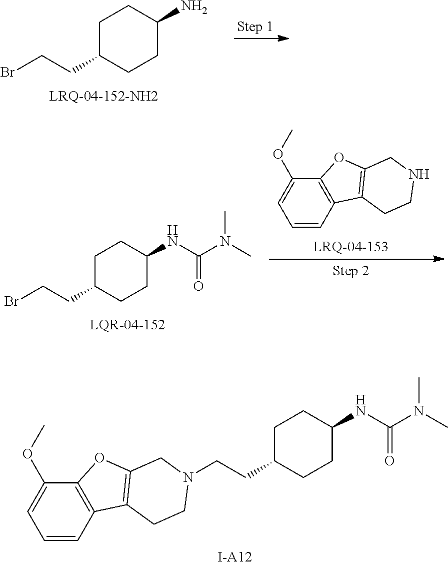

Step 1: LRQ-04-12-NH2 was dissolved in DCM (10 mL), then triethylamine (115 mg, 1.14 mmol) and dimethylcarbamoyl chloride (45 mg, 0.42 mmol) were sequentially added thereto, and the reaction mixture was reacted at room temperature overnight. After the reaction was completed, the reaction mixture was diluted with saturated sodium bicarbonate aqueous solution (10 mL), extracted with dichloromethane (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-04-152 (42 mg, yield: 40%) as a yellow solid. ¹H NMR (800 MHz, CDCl₃) δ 4.14 (s, 1H), 3.61-3.53 (m, 1H), 3.43 (t, J=7.0 Hz, 2H), 2.87 (s, 6H), 2.06-1.97 (m, 2H), 1.80-1.71 (m, 4H), 1.48-1.39 (m, 1H), 1.14-0.99 (m, 4H). ¹³C NMR (201 MHz, CDCl₃) δ 157.92, 49.84, 39.67, 36.27 (2C), 35.46, 33.88 (2C), 32.01, 31.39 (2C). HRMS (ESI) calculated for C₁₁H₂₂BrN₂O⁺ [M+H]⁺: 277.0910, found: 277.0911.

Step 2: LRQ-04-153 (24 mg, 0.12 mmol) was dissolved in DMF (8 mL), then DIPEA (45 mg, 0.35 mmol) and LRQ-04-152 (47 mg, 0.17 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (17 mg, yield: 35%). ¹H NMR (800 MHz, CDCl₃) δ 7.11 (t, J=7.8 Hz, 1H), 7.02 (d, J=7.7 Hz, 1H), 6.77-6.73 (m, 1H), 4.13 (d, J=7.5 Hz, 1H), 3.98 (s, 3H), 3.67 (s, 2H), 3.60-3.52 (m, 1H), 2.86 (s, 6H), 2.84 (t, J=5.5 Hz, 2H), 2.74-2.67 (m, 2H), 2.66-2.58 (m, 2H), 2.02-1.98 (m, 2H), 1.80-1.76 (m, 2H), 1.54-1.46 (m, 2H), 1.30-1.25 (m, 1H), 1.11-1.04 (m, 4H). ¹³C NMR (201 MHz, CDCl₃) δ 157.94, 151.31, 145.26, 143.73, 129.79, 123.27, 111.86, 111.30, 106.01, 56.12, 55.41, 50.45, 50.24, 49.93, 36.24 (2C), 35.45, 34.39, 34.07 (2C), 32.17 (2C), 20.89. HRMS (ESI) calculated for C₂₃H₃₄N₃O₃⁺ [M+H]⁺: 400.2595, found: 400.2608. HPLC: 98.43% (Q=254 nm, t_{R}=10.65 min).

### Example 13: Preparation of compound N-(trans-4-(2-(8-methoxy-3,4-dihydrobenzofuro[2,3-c]pyridin-2(1H)-yl)ethyl)cyclohexyl)methanesulfonamide (I-A13)

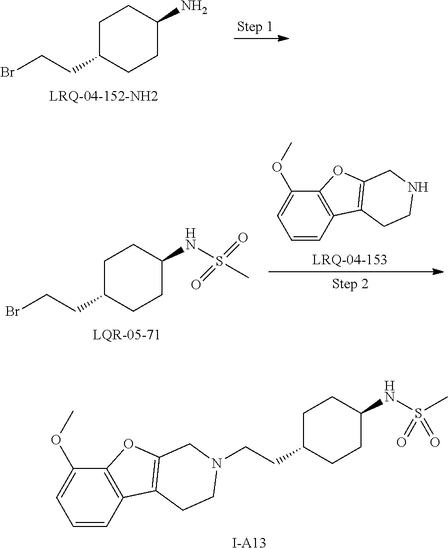

Step 1: LRQ-04-12-NH2 was dissolved in DCM (10 mL), then triethylamine (115 mg, 1.14 mmol) and methanesulfonyl chloride (48 mg, 0.42 mmol) were sequentially added thereto, and the reaction mixture was reacted at room temperature overnight. After the reaction was completed, the reaction mixture was diluted with saturated sodium bicarbonate aqueous solution (10 mL), extracted with dichloromethane (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-05-71 (53 mg, yield: 49%) as a white solid. ¹H NMR (800 MHz, CDCl₃) δ 4.33-4.22 (m, 1H), 3.42 (t, J=7.0 Hz, 2H), 3.30-3.20 (m, 1H), 2.97 (s, 3H), 2.11-2.01 (m, 2H), 1.85-1.79 (m, 2H), 1.78-1.71 (m, 2H), 1.52-1.40 (m, 1H), 1.34-1.19 (m, 2H), 1.11-0.96 (m, 2H). ¹³C NMR (201 MHz, CDCl₃) δ 53.26, 42.37, 39.42, 34.97, 34.25 (2C), 31.60, 31.20 (2C). HRMS (ESI) calculated for C₉H₁₉BrNO₂S⁺ [M+H]⁺: 284.0314, found: 284.0317.

Step 2: LRQ-04-153 (24 mg, 0.12 mmol) was dissolved in DMF (8 mL), then DIPEA (45 mg, 0.35 mmol) and LRQ-05-71 (48 mg, 0.17 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (19 mg, yield: 38%). ¹H NMR (800 MHz, CDCl₃) δ 7.15 (t, J=7.8 Hz, 1H), 7.05-7.02 (m, 1H), 6.80-6.76 (m, 1H), 4.36-4.26 (m, 1H), 4.00 (s, 3H), 3.82 (s, 2H), 3.29-3.18 (m, 1H), 3.03-2.93 (m, 5H), 2.83-2.77 (m, 2H), 2.76-2.70 (m, 2H), 2.09-2.02 (m, 2H), 1.85-1.77 (m, 2H), 1.59-1.51 (m, 2H), 1.31-1.28 (m, 1H), 1.25-1.20 (m, 2H), 1.12-1.02 (m, 2H). ¹³C NMR (201 MHz, CDCl₃) δ 151.31, 145.34, 143.91, 129.40, 123.57, 111.83, 111.38, 106.37, 56.17, 54.75, 53.28, 50.29, 49.70, 42.26, 34.82, 34.35 (2C), 33.53, 31.91 (2C), 20.20. HRMS (ESI) calculated for C₂₁H₃₁N₂O₄S⁺ [M+H]⁺: 407.1999, found: 407.1996. HPLC: 99.02% (λ=254 nm, t_{R}=10.85 min).

### Example 14: Preparation of compound N-(2-(8-methoxy-3,4-dihydrobenzofuro[2,3-c]pyridin-2(1H)-yl)ethyl)tetrahydro-2H-pyran-4-carboxamide (I-A14)

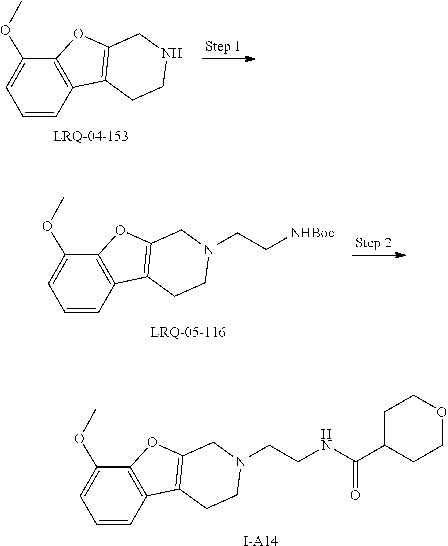

Step 1: LRQ-04-153 (340 mg, 1.67 mmol) and DIPEA (1.3 g, 10.02 mmol) were dissolved in DMF (10 mL), then N-Boc-2-bromoethylamine (561 mg, 2.51 mmol) was added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-05-116 (200 mg, yield: 35%) as a yellow solid. ¹H NMR (800 MHz, CDCl₃) δ 7.14 (t, J=7.8 Hz, 1H), 7.04 (d, J=7.7 Hz, 1H), 6.77 (d, J=7.9 Hz, 1H), 4.00 (s, 3H), 3.70 (s, 2H), 3.37-3.27 (m, 2H), 2.90-2.84 (m, 2H), 2.78-2.69 (m, 4H), 1.44 (s, 9H). HRMS (ESI) calculated for C₁₉H₂₇N₂O₄⁺ [M+H]⁺: 347.1965, found: 347.1963.

Step 2: LRQ-05-116 (70 mg, 0.20 mmol) was dissolved in DCM (10 mL), and the reaction mixture was cooled to 0° C. Trifluoroacetic acid (1 mL) was added thereto, and the reaction mixture was reacted for 2 hours. After the reaction was completed, the solvent was removed by evaporation under reduced pressure to obtain a colorless oily liquid, which was then dissolved in dichloromethane (10 mL). DIPEA (155 mg, 1.20 mmol) and tetrahydro-2H-pyran-4-carbonyl chloride (44 mg, 0.30 mmol) were added thereto, and the reaction mixture was reacted at room temperature overnight. After the reaction was completed, the reaction mixture was diluted with saturated sodium bicarbonate aqueous solution (10 mL), extracted with dichloromethane (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain a white solid (48 mg, yield: 77%). ¹H NMR (800 MHz, CDCl₃) δ 7.15 (t, J=7.8 Hz, 1H), 7.06-7.02 (m, 1H), 6.81-6.74 (m, 1H), 6.18-6.09 (m, 1H), 4.07-3.92 (m, 5H), 3.70 (s, 2H), 3.48-3.42 (m, 2H), 3.42-3.34 (m, 2H), 2.87 (t, J=5.6 Hz, 2H), 2.76 (t, J=5.9 Hz, 2H), 2.73 (t, J=5.3 Hz, 2H), 2.36-2.29 (m, 1H), 1.83-1.76 (m, 2H), 1.75-1.70 (m, 2H). ¹³C NMR (201 MHz, CDCl₃) δ 174.47, 150.99, 145.36, 143.75, 129.65, 123.49, 111.97, 111.31, 106.19, 67.41 (2C), 56.17, 55.46, 50.15, 50.04, 42.30, 36.44, 29.43 (2C), 20.97. HRMS (ESI) calculated for C₂₀H₂₇N₂O₄⁺ [M+H]⁺: 359.1965, found: 359.1962. HPLC: 98.69% (λ=254 nm, t_{R}=11.69 min).

### Example 15: Preparation of compound 4,4-difluoro-N-(2-(8-methoxy-3,4-dihydrobenzofuro[2,3-c]pyridin-2(1H)-yl)ethyl)cyclohexane-1-carboxamide (I-A15)

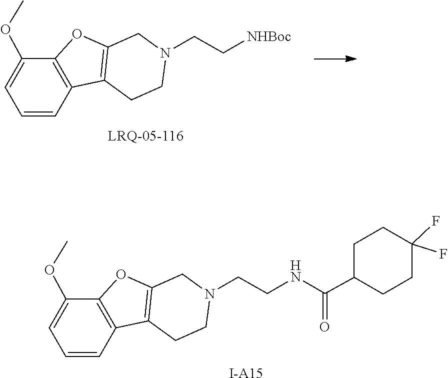

LRQ-05-116 (70 mg, 0.20 mmol) was dissolved in DCM (10 mL), and the reaction mixture was cooled to 0° C. Trifluoroacetic acid (1 mL) was added thereto, and the reaction mixture was reacted for 2 hours. After the reaction was completed, the solvent was removed by evaporation under reduced pressure to obtain a colorless oily liquid, which was then dissolved in dichloromethane (10 mL). DIPEA (155 mg, 1.20 mmol) and 4,4-difluoro-cyclohexanecarbonyl chloride (68 mg, 0.30 mmol) were added thereto, and the reaction mixture was reacted at room temperature overnight. After the reaction was completed, the reaction mixture was diluted with saturated sodium bicarbonate aqueous solution (10 mL), extracted with dichloromethane (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain a white solid (43 mg, yield: 63%). ¹H NMR (800 MHz, CDCl₃) δ 7.15 (t, J=7.8 Hz, 1H), 7.06-7.01 (m, 1H), 6.79 (d, J=7.9 Hz, 1H), 6.17-6.07 (m, 1H), 4.00 (s, 3H), 3.70 (s, 2H), 3.47-3.39 (m, 2H), 2.87 (t, J=5.6 Hz, 2H), 2.76 (t, J=5.9 Hz, 2H), 2.73 (t, J=5.5 Hz, 2H), 2.21-2.11 (m, 3H), 1.94-1.87 (m, 2H), 1.86-1.79 (m, 2H), 1.76-1.65 (m, 2H). ¹³C NMR (201 MHz, CDCl₃) δ 174.23, 150.98, 145.37, 143.76, 129.64, 123.51, 122.79 (t, J=449.2 Hz), 111.97, 111.32, 106.20, 56.17, 55.44, 50.14, 50.06, 42.88, 36.47, 32.98 (t, J=25.2 Hz) (2C), 26.07, 26.02, 20.97. HRMS (ESI) calculated for C₂₁H₂₇F₂N₂O₃⁺ [M+H]⁺: 393.1984, found: 393.1979. HPLC: 99.04% (λ254 nm, t_{R}=11.88 min).

### Example 16: Preparation of compound 7-(4-(8-ethoxy-3,4-dihydrobenzofuro[2,3-c]pyridin-2(1H)-yl)butoxy)quinolin-2(1H)-one (I-A16)

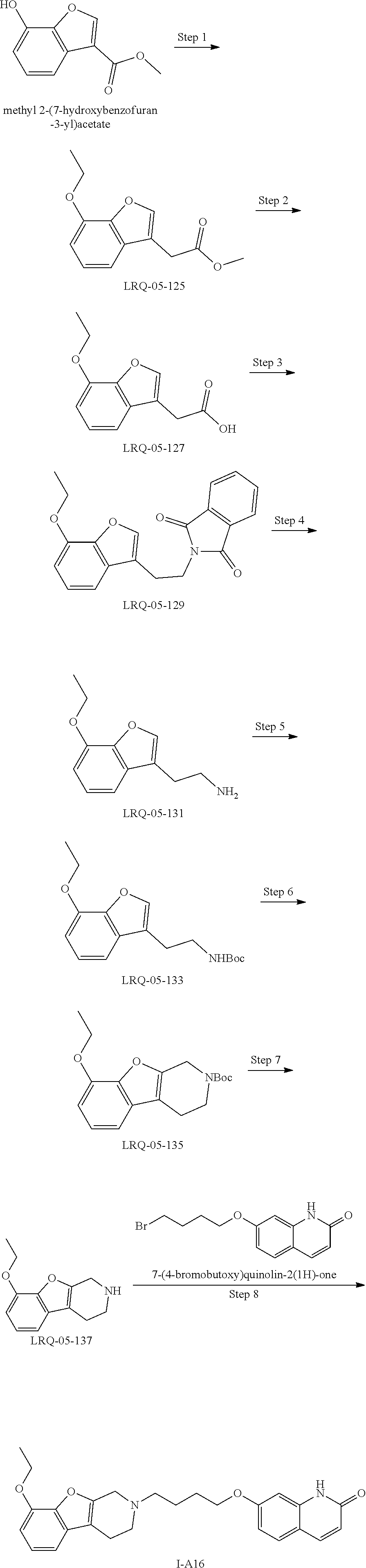

Step 1: Methyl 2-(7-hydroxybenzofuran-3-yl)acetate (2.0 g, 9.70 mmol) and K₂CO₃ (1.3 g, 9.70 mmol) were dissolved in DMF (10 mL), then iodoethane (4.54 g, 29.10 mmol) was added thereto, and the reaction mixture was stirred and reacted at room temperature overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:10) to obtain LRQ-05-125 (2.2 g, yield: 97%) as a colorless oily liquid. ¹H NMR (800 MHz, CDCl₃) δ 7.63 (s, 1H), 7.19-7.10 (m, 2H), 6.83-6.79 (m, 1H), 4.25 (q, J=7.0 Hz, 2H), 3.72 (s, 3H), 3.70 (s, 2H), 1.51 (t, J=7.0 Hz, 3H). HRMS (ESI) calculated for C₁₃H₁₅O₄⁺ [M+H]⁺: 235.0965, found: 235.0964.

Step 2: LRQ-05-125 (2.2 g, 9.40 mmol) was dissolved in THE (10 mL). The reaction mixture was replaced with nitrogen three times, then cooled to −10° C., and DIBAL-H (28.2 mL, 1.0 M in tetrahydrofuran) was added thereto. The reaction mixture was then warmed to 0° C. and reacted for 6 hours. After the reaction was completed, the reaction mixture was added with saturated potassium sodium tartrate aqueous solution (80 mL), then stirred at room temperature for 1 hour, extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:4) to obtain LRQ-05-127 (1.5 g, yield: 77%) as a light yellow oily liquid. ¹H NMR (800 MHz, CDCl₃) δ 7.51 (s, 1H), 7.17-7.12 (m, 2H), 6.83-6.79 (m, 1H), 4.25 (q, J=7.0 Hz, 2H), 3.95-3.88 (m, 2H), 2.96-2.88 (m, 2H), 1.51 (t, J=7.0 Hz, 3H). HRMS (ESI) calculated for C₁₂H₁₅O₃⁺ [M+H]⁺: 207.1016, found: 207.1013.

Step 3: LRQ-05-127 (1.5 g, 7.28 mmol), triphenylphosphine (2.86 g, 10.92 mmol), and phthalimide (1.61 g, 10.92 mmol) were dissolved in tetrahydrofuran (50 mL). The reaction mixture was then cooled to 0° C., and DEAD (1.90 g, 10.92 mmol) was added thereto. The reaction mixture was then warmed to room temperature and reacted overnight. After the reaction was completed, the reaction mixture was diluted with water (10 mL), extracted with ethyl acetate (50 mL*3), washed with saturated brine, dried over anhydrous sodium sulfate, and the solvent was removed by evaporation under reduced pressure to obtain LRQ-05-129 as a light yellow oily liquid, which was directly used in the next reaction step without purification. HRMS (ESI) calculated for C₂₀H₁₈NO₄⁺ [M+H]⁺: 336.1230, found: 336.1224.

Step 4: LRQ-05-129 was dissolved in a mixture of methanol (80 mL) and dichloromethane (20 mL), then hydrazine hydrate (911 mg, 18.2 mmol) was added thereto, and the reaction mixture was refluxed overnight. After the reaction was completed, the solvent was removed by evaporation under reduced pressure to obtain LRQ-05-131 as a light yellow oily liquid, which was directly used in the next reaction step without purification. HRMS (ESI) calculated for C₁₂H₁₆NO₂⁺ [M+H]⁺: 206.1176, found: 206.1182.

Step 5: LRQ-05-131 and Et₃N (2.21 g, 21.84 mmol) were dissolved in dichloromethane (10 mL), then (Boc)₂O (1.99 g, 9.10 mmol) was added thereto, and the reaction mixture was reacted at room temperature overnight. After the reaction was completed, the reaction mixture was diluted with saturated sodium bicarbonate aqueous solution (10 mL), extracted with dichloromethane (20 mL*3), washed with saturated brine, dried over anhydrous sodium sulfate, and the solvent was removed by evaporation under reduced pressure to obtain LRQ-05-133 as a light yellow oily liquid, which was directly used in the next reaction step without purification. HRMS (ESI) calculated for C₁₇H₂₄NO₄⁺ [M+H]⁺: 306.1700, found: 306.1703.

Step 6: LRQ-05-133 and paraformaldehyde (437 mg, 14.56 mmol) were dissolved in toluene (20 mL), then p-toluenesulfonic acid (88 mg, 0.51 mmol) was added thereto, and the reaction mixture was refluxed overnight. After the reaction was completed, the reaction mixture was diluted with saturated sodium bicarbonate aqueous solution (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:10) to obtain LRQ-05-135 (750 mg, four-step reaction yield: 33%) as a light yellow oily liquid. ¹H NMR (800 MHz, CDCl₃) δ 7.13 (t, J=7.8 Hz, 1H), 7.03 (d, J=6.9 Hz, 1H), 6.80-6.76 (m, 1H), 4.62 (s, 2H), 4.30-4.20 (m, 2H), 3.81-3.66 (m, 2H), 2.78-2.65 (m, 2H), 1.49 (s, 9H), 1.28 (t, J=6.0 Hz, 3H). HRMS (ESI) calculated for C₁₈H₂₄NO₄⁺ [M+H]⁺: 318.1700, found: 318.1701.

Step 7: LRQ-05-135 (750 mg, 2.36 mmol) was dissolved in a 4 M solution of HCl in dioxane (10 mL), and the reaction mixture was reacted at room temperature overnight. After the reaction was completed, the solvent was removed by evaporation under reduced pressure. The residue was dissolved in diethyl ether (8 mL), stirred at room temperature for 15 minutes, and then filtered under reduced pressure. The filter residue was rinsed with diethyl ether and dried under reduced pressure to obtain LRQ-05-137 (436 mg, yield: 85%) as a white solid. ¹H NMR (800 MHz, CDCl₃) δ 7.12 (t, J=7.8 Hz, 1H), 7.03 (d, J=7.7 Hz, 1H), 6.77 (d, J=7.9 Hz, 1H), 4.26 (q, J=7.0 Hz, 2H), 4.04 (s, 2H), 3.17 (t, J=5.5 Hz, 2H), 2.78-2.65 (m, 2H), 1.51 (t, J=7.0 Hz, 3H). HRMS (ESI) calculated for C₁₃H₁₆NO₂⁺ [M+H]⁺: 218.1176, found: 218.1182.

Step 8: LRQ-05-137 (50 mg, 0.23 mmol) was dissolved in DMF (8 mL), then DIPEA (178 mg, 1.38 mmol) and 7-(4-bromobutoxy)quinolin-2(1H)-one (103 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain an orange-yellow solid (47 mg, yield: 47%). ¹H NMR (800 MHz, CDCl₃) δ 7.74-7.68 (m, 1H), 7.46-7.40 (m, 1H), 7.11 (t, J=7.8 Hz, 1H), 7.02 (d, J=7.7 Hz, 1H), 6.83-6.78 (m, 2H), 6.76 (d, J=7.9 Hz, 1H), 6.53 (d, J=9.4 Hz, 1H), 4.25 (q, J=7.0 Hz, 2H), 4.11 (t, J=6.2 Hz, 2H), 3.74 (s, 2H), 2.94-2.86 (m, 2H), 2.79-2.67 (m, 4H), 1.93-1.88 (m, 2H), 1.86-1.79 (m, 2H), 1.51 (t, J=7.0 Hz, 3H). ¹³C NMR (201 MHz, CDCl₃) δ 164.86, 161.49, 150.69, 145.73, 144.59, 140.92, 140.44, 131.36, 129.19, 123.29, 118.13, 114.29, 112.69, 111.89, 111.20, 107.21, 99.08, 68.19, 64.56, 50.52, 50.27, 49.71, 32.94, 29.84, 27.15, 15.09. HRMS (ESI) calculated for C₂₆H₂₉N₂O₄⁺ [M+H]⁺: 433.2122, found: 433.2124. HPLC: 97.89% (Q=254 nm, t_{R}=12.44 min).

### Example 17: Preparation of compound 3-(trans-4-(2-(8-ethoxy-3,4-dihydrobenzofuro[2,3-c]pyridin-2(1H)-yl)ethyl)cyclohexyl)-1,1-dimethylurea (I-A17)

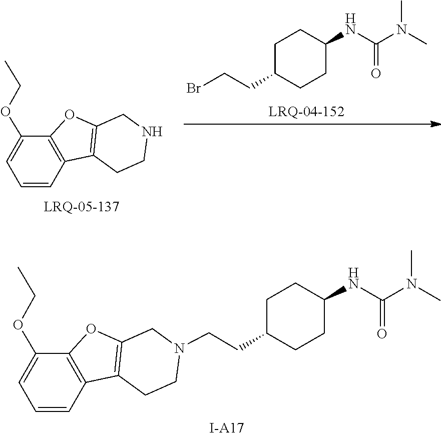

LRQ-05-137 (50 mg, 0.23 mmol) was dissolved in DMF (8 mL), then DIPEA (178 mg, 1.38 mmol) and LRQ-04-152 (94 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid I (11 mg, yield: 12%). ¹H NMR (800 MHz, CDCl₃) δ 7.10 (t, J=7.8 Hz, 1H), 7.01 (d, J=7.7 Hz, 1H), 6.77-6.73 (m, 1H), 4.24 (q, J=7.0 Hz, 2H), 3.69 (s, 2H), 3.61-3.53 (m, 1H), 2.87 (s, 6H), 2.86-2.82 (m, 2H), 2.75-2.70 (m, 2H), 2.65-2.61 (m, 2H), 2.02-2.00 (m, 2H), 1.81-1.76 (m, 2H), 1.53-1.48 (m, 5H), 1.31-1.28 (m, 1H), 1.10-1.05 (m, 4H). HRMS (ESI) calculated for C₂₄H₃₆N₃O₃⁺ [M+H]⁺: 414.2751, found: 414.2748. HPLC: 97.97% (λ=254 nm, t_{R}=12.47 min).

### Example 18: Preparation of compound 7-(4-(8-isopropoxy-3,4-dihydrobenzofuro[2,3-c]pyridin-2(1H)-yl)butoxy)quinolin-2(1H)-one (I-A18)

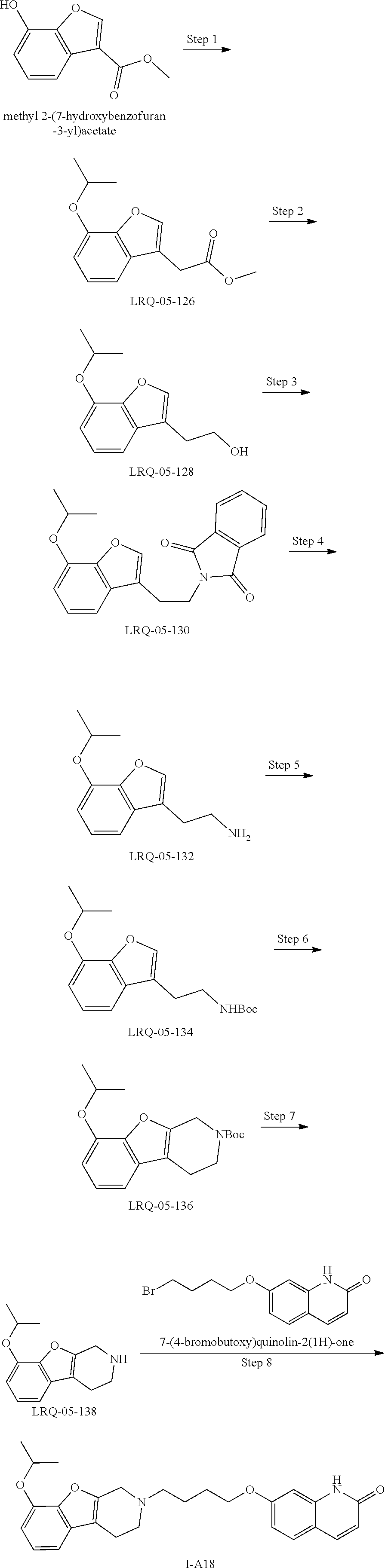

Step 1: Methyl 2-(7-hydroxybenzofuran-3-yl)acetate (2.0 g, 9.70 mmol) and K₂CO₃ (1.3 g, 9.70 mmol) were dissolved in DMF (10 mL), then 2-bromopropane (3.58 g, 29.10 mmol) was added thereto, and the reaction mixture was stirred and reacted at room temperature overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:10) to obtain LRQ-05-126 (2.3 g, yield: 98%) as a colorless oily liquid. ¹H NMR (800 MHz, CDCl₃) δ 7.62 (s, 1H), 7.17-7.11 (m, 2H), 6.85-6.81 (m, 1H), 4.83-4.74 (m, 1H), 3.73 (s, 3H), 3.69 (s, 2H), 1.42 (d, J=6.1 Hz, 6H). HRMS (ESI) calculated for C₁₄H₁₇O₄⁺ [M+H]⁺: 249.1121, found: 249.1115.

Step 2: LRQ-05-126 (2.3 g, 9.50 mmol) was dissolved in THE (10 mL). The reaction mixture was replaced with nitrogen three times, then cooled to −10° C., and DIBAL-H (28.5 mL, 1.0 M in tetrahydrofuran) was added thereto. The reaction mixture was then warmed to 0° C. and reacted for 6 hours. After the reaction was completed, the reaction mixture was added with saturated potassium sodium tartrate aqueous solution (80 mL), then stirred at room temperature for 1 hour, extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:4) to obtain LRQ-05-128 (1.6 g, yield: 77%) as a white solid. ¹H NMR (800 MHz, CDCl₃) δ 7.51 (s, 1H), 7.14 (d, J=4.4 Hz, 2H), 6.85-6.81 (m, 1H), 4.83-4.75 (m, 1H), 3.94-3.89 (m, 2H), 2.96-2.90 (m, 2H), 1.43 (d, J=6.1 Hz, 6H). HRMS (ESI) calculated for C₁₃H₁₇O₃⁺ [M+H]⁺: 221.1172, found: 221.1171.

Step 3: LRQ-05-128 (1.6 g, 7.32 mmol), triphenylphosphine (2.88 g, 10.97 mmol), and phthalimide (1.61 g, 10.97 mmol) were dissolved in tetrahydrofuran (50 mL). The reaction mixture was then cooled to 0° C., and DEAD (1.91 g, 10.97 mmol) was added thereto. The reaction mixture was then warmed to room temperature and reacted overnight. After the reaction was completed, the reaction mixture was diluted with water (10 mL), extracted with ethyl acetate (50 mL*3), washed with saturated brine, dried over anhydrous sodium sulfate, and the solvent was removed by evaporation under reduced pressure to obtain LRQ-05-130 as a white solid, which was directly used in the next reaction step without purification. HRMS (ESI) calculated for C₂₁H₂₀NO₄⁺ [M+H]⁺: 350.1387, found: 350.1388.

Step 4: LRQ-05-130 was dissolved in a mixture of methanol (80 mL) and dichloromethane (20 mL), then hydrazine hydrate (916 mg, 18.3 mmol) was added thereto, and the reaction mixture was refluxed overnight. After the reaction was completed, the solvent was removed by evaporation under reduced pressure to obtain LRQ-05-132 as an orange-yellow solid, which was directly used in the next reaction step without purification. HRMS (ESI) calculated for C₁₃H₁₈NO₂⁺ [M+H]⁺: 220.1332, found: 220.1336.

Step 5: LRQ-05-132 and Et₃N (2.22 g, 21.96 mmol) were dissolved in dichloromethane (10 mL), then (Boc)₂O (2.00 g, 9.15 mmol) was added thereto, and the reaction mixture was reacted at room temperature overnight. After the reaction was completed, the reaction mixture was diluted with saturated sodium bicarbonate aqueous solution (10 mL), extracted with dichloromethane (20 mL*3), washed with saturated brine, dried over anhydrous sodium sulfate, and the solvent was removed by evaporation under reduced pressure to obtain LRQ-05-134 as a light yellow oily liquid, which was directly used in the next reaction step without purification. HRMS (ESI) calculated for C₁₈H₂₆NO₄⁺ [M+H]⁺: 320.1856, found: 320.1858.

Step 6: LRQ-05-134 and paraformaldehyde (440 mg, 14.64 mmol) were dissolved in toluene (20 mL), then p-toluenesulfonic acid (88 mg, 0.51 mmol) was added thereto, and the reaction mixture was refluxed overnight. After the reaction was completed, the reaction mixture was diluted with saturated sodium bicarbonate aqueous solution (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:10) to obtain LRQ-05-136 (890 mg, four-step reaction yield: 37%) as a light yellow oily liquid. ¹H NMR (800 MHz, CDCl₃) δ 7.12 (t, J=7.8 Hz, 1H), 7.02 (d, J=7.4 Hz, 1H), 6.82-6.79 (m, 1H), 4.83-4.75 (m, 1H), 4.62 (s, 2H), 3.80-3.66 (m, 2H), 2.77-2.62 (m, 2H), 1.49 (s, 10H), 1.43 (d, J=6.1 Hz, 6H). HRMS (ESI) calculated for C₁₉H₂₆NO₄⁺ [M+H]⁺: 332.1856, found: 332.1849.

Step 7: LRQ-05-136 (890 mg, 2.69 mmol) was dissolved in a 4 M solution of HCl in dioxane (10 mL), and the reaction mixture was reacted at room temperature overnight. After the reaction was completed, the solvent was removed by evaporation under reduced pressure. The residue was dissolved in diethyl ether (8 mL), stirred at room temperature for 15 minutes, and then filtered under reduced pressure. The filter residue was rinsed with diethyl ether and dried under reduced pressure to obtain LRQ-05-138 (577 mg, yield: 83%) as a white solid. ¹H NMR (800 MHz, CDCl₃) δ 7.11 (t, J=7.8 Hz, 1H), 7.02 (d, J=7.6 Hz, 1H), 6.80 (d, J=7.9 Hz, 1H), 4.80-4.75 (m, 1H), 4.07 (s, 2H), 3.25-3.17 (m, 2H), 2.78-2.69 (m, 2H), 1.42 (d, J=6.1 Hz, 6H). HRMS (ESI) calculated for C₁₄H₁₈NO₂⁺ [M+H]⁺: 232.1332, found: 232.1334.

Step 8: LRQ-05-138 (50 mg, 0.22 mmol) was dissolved in DMF (8 mL), then DIPEA (171 mg, 1.32 mmol) and 7-(4-bromobutoxy)quinolin-2(1H)-one (97 mg, 0.33 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain an orange-yellow solid (42 mg, yield: 44%). ¹H NMR (800 MHz, CDCl₃) δ 7.71 (d, J=9.4 Hz, 1H), 7.43 (d, J=9.0 Hz, 1H), 7.10 (t, J=7.8 Hz, 1H), 7.01 (d, J=7.6 Hz, 1H), 6.82-6.75 (m, 3H), 6.53 (d, J=9.4 Hz, 1H), 4.82-4.73 (m, 1H), 4.15-4.05 (m, 2H), 3.75 (s, 2H), 2.96-2.85 (m, 2H), 2.78-2.67 (m, 4H), 1.93-1.86 (m, 2H), 1.86-1.78 (m, 2H), 1.42 (d, J=6.1 Hz, 6H). ¹³C NMR (201 MHz, CDCl₃) δ 164.88, 161.48, 146.94, 144.82, 143.48, 140.93, 140.45, 129.39, 129.19, 123.27, 118.13, 114.30, 112.69, 111.86, 111.26, 109.57, 99.09, 71.50, 68.19, 57.00, 50.53, 50.27, 29.85, 27.13, 23.99, 22.37 (2C). HRMS (ESI) calculated for C₂₇H₃₁N₂O₄⁺ [M+H]⁺: 447.2278, found: 447.2273. HPLC: 97.68% (Q=254 nm, t_{R}=12.45 min).

### Example 19: Preparation of compound 3-(trans-4-(2-(8-isopropoxy-3,4-dihydrobenzofuro[2,3-c]pyridin-2(1H)-yl)ethyl)cyclohexyl)-1,1-dimethylurea (I-A19)

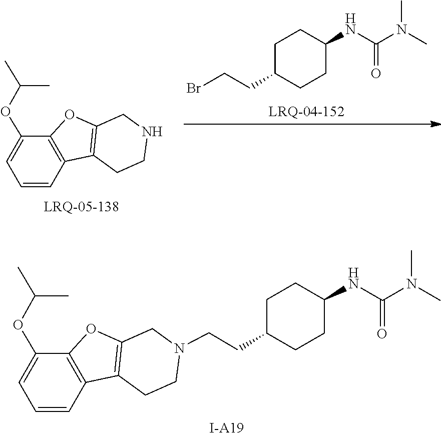

LRQ-05-138 (50 mg, 0.22 mmol) was dissolved in DMF (8 mL), then DIPEA (171 mg, 1.32 mmol) and LRQ-04-152 (89 mg, 0.33 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (22 mg, yield: 24%). ¹H NMR (800 MHz, CDCl₃) δ 7.09 (t, J=7.8 Hz, 1H), 7.01-6.98 (m, 1H), 6.79-6.74 (m, 1H), 4.83-4.72 (m, 1H), 3.68 (s, 2H), 3.62-3.51 (m, 1H), 2.86 (s, 6H), 2.85-2.81 (m, 2H), 2.74-2.68 (m, 2H), 2.66-2.58 (m, 2H), 2.02-1.99 (m, 2H), 1.81-1.76 (m, 2H), 1.53-1.47 (m, 2H), 1.40 (d, J=6.1 Hz, 6H), 1.28 (s, 1H), 1.09-1.05 (m, 4H). HHRMS (ESI) calculated for C₂₅H₃₈N₃O₃⁺ [M+H]⁺: 428.2908, found: 428.2901. HPLC: 97.43% (λ=254 nm, t_{R}=12.47 min).

### Example 20: Preparation of compound 7-(2-(8-methoxy-3,4-dihydrobenzofuro[2,3-c]pyridin-2(1H)-yl)butyl)quinolin-2(1H)-one (I-A20) (IHCH-5219)

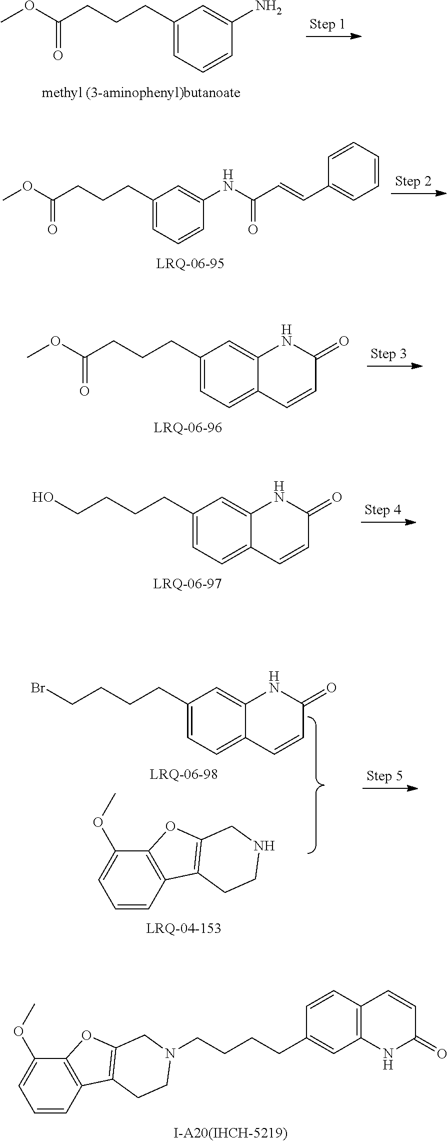

Step 1: Methyl (3-aminophenyl)butanoate (1.00 g, 5.17 mmol) was dissolved in dichloromethane (10 mL), and the reaction mixture was cooled to 0° C. Pyridine (818 mg, 10.34 mmol) and cinnamoyl chloride (1.29 g, 7.76 mmol) were then sequentially added thereto, and the reaction mixture was warmed to room temperature and reacted overnight. After the reaction was completed, the reaction mixture was diluted with saturated sodium bicarbonate aqueous solution (10 mL), extracted with ethyl acetate (50 mL*3), washed with saturated brine, dried over anhydrous sodium sulfate, and the solvent was removed by evaporation under reduced pressure to obtain LRQ-06-95 as an orange-yellow oily liquid, which was directly used in the next reaction step without purification. HHRMS (ESI) calculated for C₂₀H₂₂NO₃⁺ [M+H]⁺: 324.1594, found: 324.1597.

Step 2: LRQ-06-95 was dissolved in chlorobenzene (20 mL). The reaction mixture was replaced with nitrogen three times, then added with aluminum chloride (3.45 g, 25.85 mmol), heated to 95° C., and reacted for 3 hours. After the reaction was completed, the reaction mixture was cooled to 0° C., diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-06-96 (780 mg, two-step reaction yield: 61%) as a light yellow solid. ¹H NMR (800 MHz, CDCl₃) δ 7.81 (d, J=9.3 Hz, 1H), 7.50 (d, J=8.0 Hz, 1H), 7.22 (s, 1H), 7.10-7.07 (m, 1H), 6.69 (d, J=9.4 Hz, 1H), 3.68 (s, 3H), 2.79-2.73 (m, 2H), 2.36 (t, J=7.4 Hz, 2H), 2.03-1.99 (m, 2H). HRMS (ESI) calculated for C₁₄H₁₆NO₃⁺ [M+H]⁺: 246.1125, found: 246.1119.

Step 3: LRQ-06-96 (780 mg, 3.18 mmol) was dissolved in THE (20 mL). The reaction mixture was replaced with nitrogen three times, then cooled to −10° C., and DIBAL-H (10 mL, 1.0 M in tetrahydrofuran) was added thereto. The reaction mixture was then warmed to 0° C. and reacted for 6 hours. After the reaction was completed, the reaction mixture was added with saturated potassium sodium tartrate aqueous solution (10 mL), then stirred at room temperature for 1 hour, extracted with ethyl acetate (20 mL*3), washed with saturated brine, dried over anhydrous sodium sulfate, and the solvent was removed by evaporation under reduced pressure to obtain LRQ-06-97 as a yellow oily liquid, which was directly used in the next reaction step without purification. HRMS (ESI) calculated for C₁₃H₁₆NO₂⁺ [M+H]⁺: 218.1176, found: 218.1178.

Step 4: LRQ-06-97 was dissolved in DCM (10 mL), and the reaction mixture was cooled to 0° C. Carbon tetrabromide (1.58 g, 4.77 mmol) and triphenylphosphine (1.67 g, 6.36 mmol) were sequentially added thereto, and the reaction mixture was reacted at room temperature overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:2) to obtain LRQ-06-98 (45 mg, two-step reaction yield: 5%) as a light yellow solid. ¹H NMR (600 MHz, MeOH-d₄) δ 7.89 (d, J=9.4 Hz, 1H), 7.58 (d, J=8.1 Hz, 1H), 7.26 (s, 1H), 7.13 (d, J=7.9 Hz, 1H), 6.64 (d, J=9.4 Hz, 1H), 3.49 (t, J=6.7 Hz, 2H), 2.89 (t, J=7.9 Hz, 2H), 1.97-1.92 (m, 2H), 1.83-1.77 (m, 2H). HRMS (ESI) calculated for C₁₃H₁₅BrNO⁺ [M+H]⁺: 280.0332, found: 280.0328.

Step 5: LRQ-04-153 (21 mg, 0.10 mmol) was dissolved in DMF (8 mL), then DIPEA (84 mg, 0.65 mmol) and LRQ-06-98 (45 mg, 0.16 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a light yellow solid (13 mg, yield: 32%). ¹H NMR (800 MHz, CDCl₃) δ 12.16 (s, 1H), 7.76 (d, J=9.4 Hz, 1H), 7.51-7.41 (m, 1H), 7.22 (s, 1H), 7.12 (t, J=7.8 Hz, 1H), 7.06 (d, J=7.9 Hz, 1H), 7.02 (d, J=7.7 Hz, 1H), 6.80-6.72 (m, 1H), 6.70-6.58 (m, 1H), 3.99 (s, 3H), 3.68 (s, 2H), 2.88-2.80 (m, 2H), 2.79-2.70 (m, 4H), 2.67-2.59 (m, 2H), 1.78-1.69 (m, 2H), 1.68-1.58 (m, 2H)¹³C NMR (201 MHz, CDCl₃) δ 164.78, 151.38, 146.11, 145.27, 143.74, 141.00, 138.78, 129.81, 127.82, 123.69, 123.28, 120.44, 118.29, 115.57, 111.89, 111.33, 105.97, 57.38, 56.12, 50.58, 50.24, 36.03, 29.22, 27.10, 20.97. HRMS (ESI) calculated for C₂₅H₂₇N₂O₃⁺ [M+H]⁺: 403.2016, found: 403.2011. HPLC: 98.65% (λ=254 nm, t_{R}=11.68 min).

### Example 21: Preparation of compound 1-((8-methoxy-3,4-dihydrobenzofuro[2,3-c]pyridin-2(1H)-yl)methyl)cyclohexan-1-ol (I-A21) (IHCH-5228)

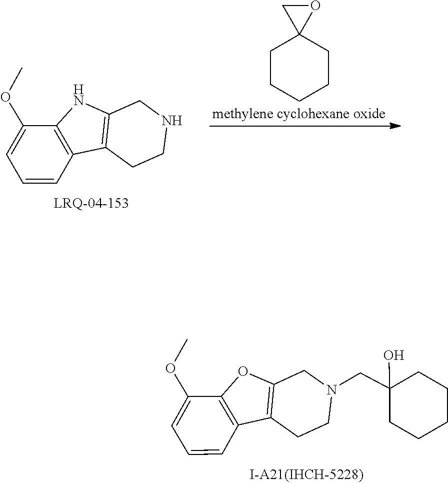 [SMILES: COc1cccc2c3c([nH]c12)CNCC3 ; C1CCC2(CC1)CO2 ; COc1cccc2c3c(oc12)CN(CC1(O)CCCCC1)CC3]

LRQ-04-153 (50 mg, 0.25 mmol) was dissolved in anhydrous ethanol (10 mL), then methylene cyclohexane oxide (138 mg, 1.25 mmol) was added thereto, and the reaction mixture was heated to 60° C. and reacted overnight. After the reaction was completed, the solvent was removed by evaporation under reduced pressure, and the residue was purified by silica gel column chromatography (methanol/dichloromethane=1:10) to obtain a white solid (32 mg, yield: 42%). ¹H NMR (800 MHz, CDCl₃) δ 7.14 (t, J=7.8 Hz, 1H), 7.07-7.02 (m, 1H), 6.77 (d, J=7.9 Hz, 1H), 4.00 (s, 3H), 3.87 (s, 2H), 3.07-2.95 (m, 2H), 2.78-2.68 (m, 2H), 2.57 (s, 2H), 1.69-1.62 (m, 2H), 1.61-1.53 (m, 3H), 1.50-1.42 (m, 2H), 1.39-1.32 (m, 2H), 1.30-1.23 (m, 1H). ¹³C NMR (201 MHz, CDCl₃) δ 151.46, 145.34, 143.65, 129.73, 123.40, 112.07, 111.31, 106.09, 70.73, 66.18, 56.15, 52.80, 52.74, 36.61 (2C), 26.00, 22.25 (2C), 21.96. HRMS (ESI) calculated for C₁₉H₂₆NO₃⁺ [M+H]⁺: 316.1907, found: 316.1908. HPLC: 97.68% (λ=254 nm, t_{R}=11.41 min).

### Example 22: Preparation of compound 2-(4-(8-methoxy-3,4-dihydrobenzofuro[2,3-c]pyridin-2(1H)-yl)butyl)-4-methyl-1,2,4-triazine-3,5(2H,4H)-dione (I-A22) (IHCH-5201)

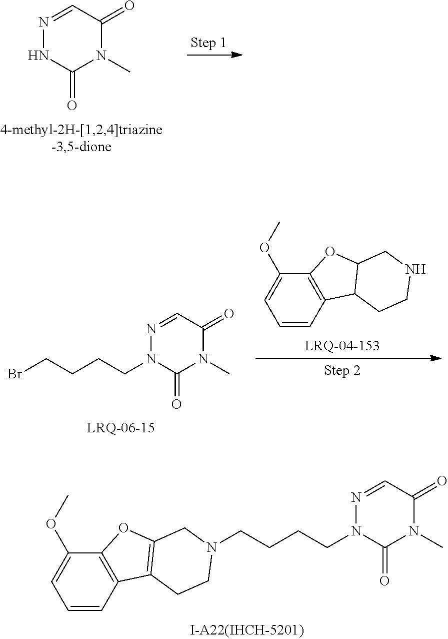

Step 1: 4-Methyl-2H-[1,2,4]triazine-3,5-dione (2.25 g, 17.70 mmol) was dissolved in DMF (20 mL), then NaH (0.85 g, 21.24 mmol) and 1,4-dibromobutane (11.46 g, 53.10 mmol) were sequentially added thereto, and the reaction mixture was reacted at room temperature overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:4) to obtain LRQ-06-15 (2.19 g, yield: 47%) as a light yellow oily liquid. ¹H NMR (800 MHz, CDCl₃) δ 7.39 (s, 1H), 4.01 (t, J=6.7 Hz, 2H), 3.43 (t, J=6.3 Hz, 2H), 3.33 (s, 3H), 1.96-1.84 (m, 4H). HRMS (ESI) calculated for C₈H₁₃BrN₃O₂⁺ [M+H]⁺: 262.0186, found: 262.0188.

Step 2: LRQ-04-153 (50 mg, 0.25 mmol) was dissolved in DMF (8 mL), then DIPEA (191 mg, 1.48 mmol) and LRQ-06-15 (97 mg, 0.37 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a light yellow solid (31 mg, yield: 33%). ¹H NMR (800 MHz, CDCl₃) δ 7.39 (s, 1H), 7.13 (t, J=7.8 Hz, 1H), 7.03 (d, J=7.7 Hz, 1H), 6.78-6.74 (m, 1H), 4.06-4.01 (m, 2H), 3.99 (s, 3H), 3.69 (s, 2H), 3.33 (s, 3H), 2.89-2.81 (m, 2H), 2.77-2.70 (m, 2H), 2.69-2.62 (m, 2H), 1.88-1.80 (m, 2H), 1.69-1.62 (m, 2H). ¹³C NMR (201 MHz, CDCl₃) δ 156.32, 151.16, 148.96, 145.29, 143.75, 133.90, 129.75, 123.33, 111.89, 111.32, 106.03, 56.84, 56.14, 51.74, 50.49, 50.19, 27.10, 26.18, 24.36, 20.93. HRMS (ESI) calculated for C₂₀H₂₅N₄O₄⁺ [M+H]⁺: 385.1870, found: 385.1865. HPLC: 98.02% (Q=254 nm, t_{R}=10.85 min).

### Example 23: Preparation of compound 2-(3-(8-methoxy-3,4-dihydrobenzofuro[2,3-c]pyridin-2(1H)-yl)propyl)-4-methyl-1,2,4-triazine-3,5(2H,4H)-dione (I-A23) (IHCH-5202)

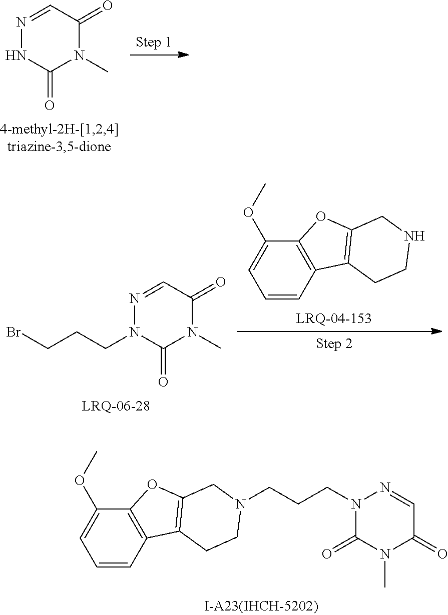

Step 1: 4-Methyl-2H-[1,2,4]triazine-3,5-dione (2.25 g, 17.70 mmol) was dissolved in DMF (20 mL), then NaH (0.85 g, 21.24 mmol) and 1,3-dibromopropane (11.25 g, 53.10 mmol) were sequentially added thereto, and the reaction mixture was reacted at room temperature overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:4) to obtain LRQ-06-28 (1.78 g, yield: 41%) as a colorless oily liquid. ¹H NMR (800 MHz, CDCl₃) δ 7.41 (s, 1H), 4.04 (t, J=6.6 Hz, 2H), 3.43 (t, J=6.2 Hz, 2H), 3.20 (s, 3H), 1.91-1.82 (m, 2H). HRMS (ESI) calculated for C₇H₁₁BrN₃O₂⁺ [M+H]⁺: 248.0029, found: 248.0031.

Step 2: LRQ-04-153 (50 mg, 0.25 mmol) was dissolved in DMF (8 mL), then DIPEA (191 mg, 1.48 mmol) and LRQ-06-28 (91 mg, 0.37 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (35 mg, yield: 38%). ¹H NMR (800 MHz, CDCl₃) δ 7.37 (s, 1H), 7.12 (t, J=7.8 Hz, 1H), 7.01 (d, J=7.7 Hz, 1H), 6.75 (d, J=7.9 Hz, 1H), 4.13-4.06 (m, 2H), 3.99 (s, 3H), 3.66 (s, 2H), 3.26 (s, 3H), 2.86-2.77 (m, 2H), 2.71-2.66 (m, 4H), 2.07-1.99 (m, 2H). ¹³C NMR (201 MHz, CDCl₃) δ 156.31, 151.20, 149.02, 145.25, 143.68, 133.79, 129.69, 123.33, 111.80, 111.30, 106.01, 56.11, 54.38, 50.48, 50.24, 50.18, 26.98, 25.87, 20.97. HRMS (ESI) calculated for C₁₉H₂₃N₄O₄⁺ [M+H]⁺: 371.1714, found: 371.1712. HPLC: 96.59% (Q=254 nm, t_{R}=10.54 min).

### Example 24: Preparation of compound 1-(2-(-methoxy-3,4-dihydrobenzofuro[2,3-c]pyridin-2(1H)-yl)ethyl)-1,3-dihydro-2H-benzo[d]imidazol-2-one (I-A24) (IHCH-5223)

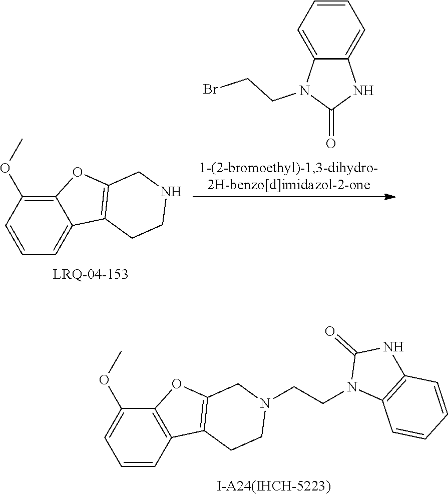 [SMILES: COc1cccc2c3c(oc12)CNCC3 ; O=c1[nH]c2ccccc2n1CCBr ; COc1cccc2c3c(oc12)CN(CCn1c(=O)[nH]c2ccccc21)CC3]

LRQ-04-153 (50 mg, 0.25 mmol) was dissolved in DMF (8 mL), then DIPEA (191 mg, 1.48 mmol) and 1-(2-bromoethyl)-1,3-dihydro-2H-benzo[d]imidazol-2-one (92 mg, 0.38 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a light yellow solid (35 mg, yield: 42%). ¹H NMR (800 MHz, CDCl₃) δ 9.72 (s, 1H), 7.13 (t, J=7.8 Hz, 1H), 7.10-7.06 (m, 2H), 7.06-7.03 (m, 2H), 7.03-7.00 (m, 1H), 6.76 (d, J=7.9 Hz, 1H), 4.12 (t, J=6.9 Hz, 2H), 3.99 (s, 3H), 3.83 (s, 2H), 3.04-2.92 (m, 4H), 2.77-2.68 (m, 2H). ¹³C NMR (201 MHz, CDCl₃) δ 155.57, 149.30, 145.29, 143.72, 130.41, 129.75, 128.07, 123.34, 121.68, 121.48, 111.96, 111.34, 109.80, 107.97, 106.07, 56.16, 54.67, 50.52, 50.36, 39.17, 20.90. HRMS (ESI) calculated for C₂₁H₂₂N₃O₃⁺ [M+H]⁺: 364.1656, found: 364.1657. HPLC: 95.01% (λ=254 nm, t_{R}=11.84 min).

### Example 25: Preparation of compound 7-(2-(8-ethoxy-3,4-dihydrobenzofuro[2,3-c]pyridin-2(1H)-yl)butyl)quinolin-2(1H)-one (I-A25) (IHCH-5225)

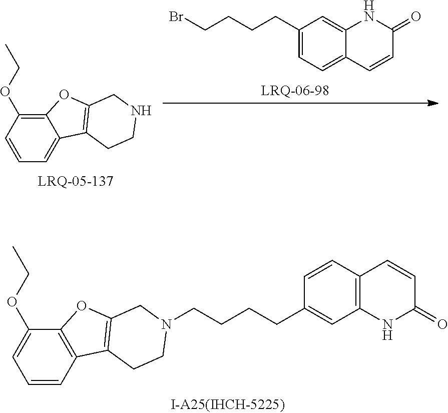 [SMILES: CCOc1cccc2c3c(oc12)CNCC3 ; O=c1ccc2ccc(CCCCBr)cc2[nH]1 ; CCOc1cccc2c3c(oc12)CN(CCCCc1ccc2ccc(=O)[nH]c2c1)CC3]

LRQ-05-137 (22 mg, 0.10 mmol) was dissolved in DMF (8 mL), then DIPEA (84 mg, 0.65 mmol) and LRQ-06-98 (45 mg, 0.16 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a reddish brown solid (10 mg, yield: 23%). ¹H NMR (800 MHz, CDCl₃) δ 12.06 (s, 1H), 7.74 (d, J=9.4 Hz, 1H), 7.48-7.42 (m, 1H), 7.26 (s, 1H), 7.09 (t, J=7.8 Hz, 1H), 7.05-7.02 (m, 1H), 7.00 (d, J=7.6 Hz, 1H), 6.77-6.71 (m, 1H), 6.62 (d, J=9.4 Hz, 1H), 4.22 (q, J=7.0 Hz, 2H), 3.78 (s, 2H), 2.98-2.89 (m, 2H), 2.79-2.67 (m, 6H), 1.75-1.67 (m, 4H), 1.48 (t, J=7.0 Hz, 3H). ¹³C NMR (201 MHz, CDCl₃) δ 164.55, 145.86, 144.55, 143.97, 142.25, 140.93, 138.77, 129.63, 127.80, 123.63, 123.36, 120.43, 118.26, 115.53, 111.81, 111.18, 107.27, 64.51, 57.04, 50.50, 49.95, 35.85, 29.00, 26.63, 20.47, 15.03. HRMS (ESI) calculated for C₂₆H₂₉N₂O₃⁺ [M+H]⁺: 417.2173, found: 417.2177. HPLC: 98.08% (λ=254 nm, t_{R}=11.69 min).

### Example 26: Preparation of compound 7-(2-(8-ethoxy-3,4-dihydrobenzofuro[2,3-c]pyridin-2(1H)-yl)propyl)quinolin-2(1H)-one (I-A26) (IHCH-5218)

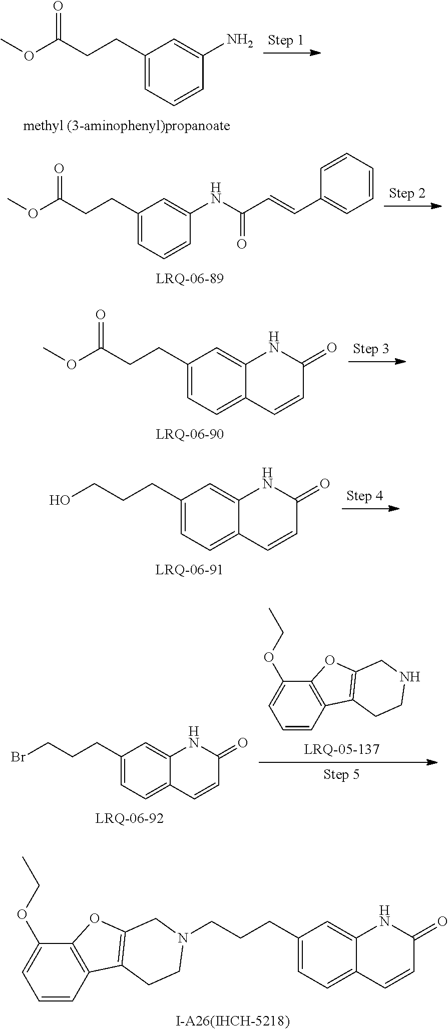

Step 1: Methyl (3-aminophenyl)propanoate (1.00 g, 5.58 mmol) was dissolved in dichloromethane (10 mL), and the reaction mixture was cooled to 0° C. Pyridine (883 mg, 11.16 mmol) and cinnamoyl chloride (1.39 g, 8.37 mmol) were then sequentially added thereto, and the reaction mixture was warmed to room temperature and reacted overnight. After the reaction was completed, the reaction mixture was diluted with saturated sodium bicarbonate aqueous solution (10 mL), extracted with ethyl acetate (50 mL*3), washed with saturated brine, dried over anhydrous sodium sulfate, and the solvent was removed by evaporation under reduced pressure to obtain LRQ-06-89 as a colorless oily liquid, which was directly used in the next reaction step without purification. HRMS (ESI) calculated for C₁₉H₂₀NO₃⁺ [M+H]⁺: 310.1438, found: 310.1438.

Step 2: LRQ-06-89 was dissolved in chlorobenzene (20 mL). The reaction mixture was replaced with nitrogen three times, then added with aluminum chloride (3.72 g, 27.90 mmol), heated to 95° C., and reacted for 3 hours. After the reaction was completed, the reaction mixture was cooled to 0° C., diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-06-90 (954 mg, two-step reaction yield: 74%) as a white solid. ¹H NMR (800 MHz, CDCl₃) δ 7.82 (d, J=9.4 Hz, 1H), 7.54 (d, J=8.0 Hz, 1H), 7.27 (s, 1H), 7.14-7.11 (m, 1H), 6.70 (d, J=9.3 Hz, 1H), 3.70 (s, 3H), 2.83-2.77 (m, 2H), 2.66 (t, J=7.4 Hz, 2H) HRMS (ESI) calculated for C₁₃H₁₄NO₃⁺ [M+H]⁺: 232.0968, found: 232.0966.

Step 3: LRQ-06-90 (520 mg, 2.25 mmol) was dissolved in THE (20 mL). The reaction mixture was replaced with nitrogen three times, then cooled to −10° C., and DIBAL-H (7 mL, 1.0 M in tetrahydrofuran) was added thereto. The reaction mixture was then warmed to 0° C. and reacted for 6 hours. After the reaction was completed, the reaction mixture was added with saturated potassium sodium tartrate aqueous solution (10 mL), then stirred at room temperature for 1 hour, extracted with ethyl acetate (20 mL*3), washed with saturated brine, dried over anhydrous sodium sulfate, and the solvent was removed by evaporation under reduced pressure to obtain LRQ-06-91 as a light yellow solid, which was directly used in the next reaction step without purification. HRMS (ESI) calculated for C₁₂H₁₄NO₂⁺ [M+H]⁺: 204.1019, found: 204.1024.

Step 4: LRQ-06-91 was dissolved in DCM (10 mL), and the reaction mixture was cooled to 0° C. Carbon tetrabromide (1.12 g, 3.38 mmol) and triphenylphosphine (1.18 g, 4.50 mmol) were sequentially added thereto, and the reaction mixture was reacted at room temperature overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:2) to obtain LRQ-06-92 (370 mg, two-step reaction yield: 62%) as a white solid. ¹H NMR (600 MHz, CDCl₃) δ 7.86 (d, J=9.3 Hz, 1H) 7.58 (d, J=8.0 Hz, 1H), 7.26 (s, 1H), 7.14 (d, J=7.9 Hz, 1H), 6.75 (d, J=9.3 Hz, 1H), 3.41 (t, J=6.5 Hz, 2H), 2.93-2.86 (m, 2H), 2.23-2.20 (m, 2H). HRMS (ESI) calculated for C₁₂H₁₃BrNO⁺ [M+H]⁺: 266.0175, found: 266.0169.

Step 5: LRQ-05-137 (40 mg, 0.18 mmol) was dissolved in DMF (8 mL), then DIPEA (140 mg, 1.08 mmol) and LRQ-06-92 (72 mg, 0.27 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a light yellow solid (22 mg, yield: 30%). ¹H NMR (800 MHz, CDCl₃) δ 12.62 (s, 1H), 7.78 (d, J=9.4 Hz, 1H), 7.48 (d, J=8.0 Hz, 1H), 7.28 (s, 1H), 7.12-7.06 (m, 2H), 7.03-7.00 (m, 1H), 6.78-6.73 (m, 1H), 6.68 (d, J=9.4 Hz, 1H), 4.24 (q, J=7.0 Hz, 2H), 3.69 (s, 2H), 2.87-2.80 (m, 2H), 2.80-2.77 (m, 2H), 2.74-2.70 (m, 2H), 2.66-2.59 (m, 2H), 2.00-1.91 (m, 2H), 1.50 (t, J=7.0 Hz, 3H). ¹³C NMR (201 MHz, CDCl₃) δ 164.99, 151.43, 145.76, 144.54, 143.88, 140.99, 138.84, 129.93, 127.81, 123.66, 123.22, 120.42, 118.33, 115.73, 111.88, 111.16, 107.10, 64.50, 56.68, 50.52, 50.28, 33.70, 29.02, 21.01, 15.06. HHRMS (ESI) calculated for C₂₅H₂₇N₂O₃⁺ [M+H]⁺: 403.2016, found: 403.2017. HPLC: 97.83% (λ=254 nm, t_{R}=11.70 min).

### Example 27: Preparation of compound 7-(2-(8-ethoxy-3,4-dihydrobenzofuro[2,3-c]pyridin-2(1H)-yl)ethyl)quinolin-2(1H)-one (I-A27) (IHCH-5217)

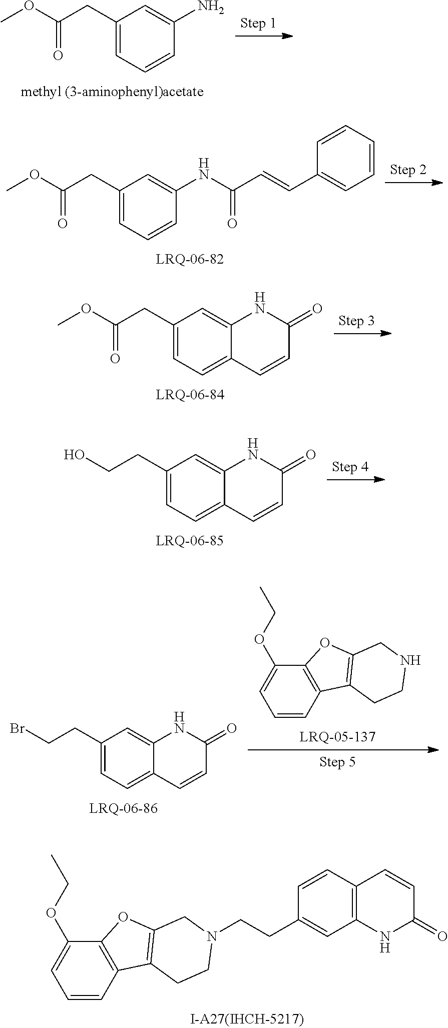

Step 1: Methyl (3-aminophenyl)acetate (1.00 g, 6.05 mmol) was dissolved in dichloromethane (10 mL), and the reaction mixture was cooled to 0° C. Pyridine (957 mg, 12.10 mmol) and cinnamoyl chloride (1.52 g, 9.08 mmol) were then sequentially added thereto, and the reaction mixture was warmed to room temperature and reacted overnight. After the reaction was completed, the reaction mixture was diluted with saturated sodium bicarbonate aqueous solution (10 mL), extracted with ethyl acetate (50 mL*3), washed with saturated brine, dried over anhydrous sodium sulfate, and the solvent was removed by evaporation under reduced pressure to obtain LRQ-06-82 as a brownish yellow oily liquid, which was directly used in the next reaction step without purification. HRMS (ESI) calculated for C₁₈H₁₈NO₃⁺ [M+H]⁺: 296.1281, found: 296.1288.

Step 2: LRQ-06-82 was dissolved in chlorobenzene (20 mL). The reaction mixture was replaced with nitrogen three times, then added with aluminum chloride (4.03 g, 30.25 mmol), heated to 95° C., and reacted for 3 hours. After the reaction was completed, the reaction mixture was cooled to 0° C., diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-06-84 (604 mg, two-step reaction yield: 46%) as a light yellow solid. ¹H NMR (800 MHz, CDCl₃) δ 7.81 (d, J=9.4 Hz, 1H), 7.54 (d, J=8.0 Hz, 1H), 7.32 (s, 1H), 7.18-7.15 (m, 1H), 6.71 (d, J=9.4 Hz, 1H), 3.74 (s, 2H), 3.71 (s, 3H). HHRMS (ESI) calculated for C₁₂H₁₂NO₃⁺ [M+H]⁺: 218.0812, found: 218.0813.

Step 3: LRQ-06-84 (560 mg, 2.57 mmol) was dissolved in THE (20 mL). The reaction mixture was replaced with nitrogen three times, then cooled to −10° C., and DIBAL-H (8 mL, 1.0 M in tetrahydrofuran) was added thereto. The reaction mixture was then warmed to 0° C. and reacted for 6 hours. After the reaction was completed, the reaction mixture was added with saturated potassium sodium tartrate aqueous solution (10 mL), then stirred at room temperature for 1 hour, extracted with ethyl acetate (20 mL*3), washed with saturated brine, dried over anhydrous sodium sulfate, and the solvent was removed by evaporation under reduced pressure to obtain LRQ-06-85 as a light yellow solid, which was directly used in the next reaction step without purification. HRMS (ESI) calculated for C₁₁H₁₂NO₂⁺ [M+H]⁺: 190.0863, found: 190.0865.

Step 4: LRQ-06-85 was dissolved in DCM (10 mL), and the reaction mixture was cooled to 0° C. Carbon tetrabromide (1.28 g, 3.86 mmol) and triphenylphosphine (1.35 g, 5.14 mmol) were sequentially added thereto, and the reaction mixture was reacted at room temperature overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-06-86 (300 mg, two-step reaction yield: 46%) as a white solid. ¹H NMR (600 MHz, CDCl₃) δ 7.88 (d, J=9.4 Hz, 1H), 7.58 (d, J=8.0 Hz, 1H), 7.26 (s, 1H), 7.16 (d, J=7.9 Hz, 1H), 6.76 (d, J=9.4 Hz, 1H), 3.63 (t, J=7.3 Hz, 2H), 3.29 (t, J=7.3 Hz, 2H). HRMS (ESI) calculated for C₁₁H₁₁BrNO⁺ [M+H]⁺: 252.0019, found: 252.0013.

Step 5: LRQ-05-137 (40 mg, 0.18 mmol) was dissolved in DMF (8 mL), then DIPEA (140 mg, 1.08 mmol) and LRQ-06-86 (68 mg, 0.27 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a light yellow solid (14 mg, yield: 20%). ¹H NMR (800 MHz, CDCl₃) δ 12.20 (s, 1H), 7.79-7.70 (m, 1H), 7.46 (d, J=7.9 Hz, 1H), 7.24 (s, 1H), 7.11 (t, J=7.7 Hz, 2H), 7.02 (d, J=7.7 Hz, 1H), 6.76 (d, J=7.9 Hz, 1H), 6.68-6.61 (m, 1H), 4.25 (q, J=7.0 Hz, 2H), 3.81 (s, 2H), 3.04-2.97 (m, 2H), 2.97-2.90 (m, 4H), 2.79-2.73 (m, 2H), 1.51 (t, J=7.0 Hz, 3H). ¹³C NMR (201 MHz, CDCl₃) δ 164.68, 151.13, 144.58, 143.94, 143.65, 140.90, 138.74, 129.86, 127.94, 123.92, 123.30, 120.68, 118.49, 115.91, 111.94, 111.20, 107.18, 64.53, 58.75, 50.57, 50.21, 34.30, 20.95, 15.08. HRMS (ESI) calculated for C₂₄H₂₅N₂O₃⁺ [M+H]⁺: 389.1860, found: 389.1865. HPLC: 95.13% (λ=254 nm, t_{R}=11.68 min).

### Example 28: Preparation of compound 1-((8-ethoxy-3,4-dihydrobenzofuro[2,3-c]pyridin-2(1H)-yl)methyl)cyclohexan-1-ol (I-A28) (IHCH-5232)

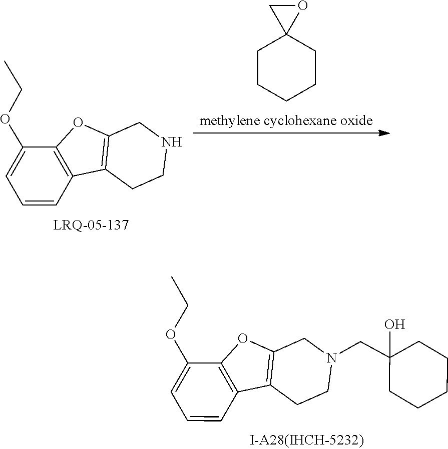 [SMILES: CCOc1cccc2c3c(oc12)CNCC3 ; C1CCC2(CC1)CO2 ; CCOc1cccc2c3c(oc12)CN(CC1(O)CCCCC1)CC3]

LRQ-05-137 (50 mg, 0.23 mmol) was dissolved in anhydrous ethanol (10 mL), then methylene cyclohexane oxide (128 mg, 1.15 mmol) was added thereto, and the reaction mixture was heated to 60° C. and reacted overnight. After the reaction was completed, the solvent was removed by evaporation under reduced pressure, and the residue was purified by silica gel column chromatography (methanol/dichloromethane=1:10) to obtain a white solid (29 mg, yield: 38%). ¹H NMR (800 MHz, CDCl₃) δ 7.12 (t, J=7.8 Hz, 1H), 7.03 (d, J=7.7 Hz, 1H), 6.79-6.75 (m, 1H), 4.25 (q, J=7.0 Hz, 2H), 3.87 (s, 2H), 3.06-2.94 (m, 2H), 2.76-2.68 (m, 2H), 2.56 (s, 2H), 1.71-1.63 (m, 2H), 1.63-1.53 (m, 3H), 1.51 (t, J=7.0 Hz, 3H), 1.49-1.42 (m, 2H), 1.39-1.34 (m, 2H), 1.30-1.23 (m, 1H). ¹³C NMR (201 MHz, CDCl₃) δ 148.08, 144.63, 143.82, 129.85, 123.36, 112.07, 111.15, 107.18, 70.72, 64.53, 55.72, 52.85, 52.82, 36.63 (2C), 26.02, 22.28 (2C), 21.97, 15.07. HRMS (ESI) calculated for C₂₀H₂₈NO₃⁺ [M+H]⁺: 330.2064, found: 330.2069. HPLC: 97.60% (λ=254 nm, t_{R}=11.58 min).

### Example 29: Preparation of compound 7-(4-(8-ethoxy-3,4-dihydrobenzofuro[2,3-c]pyridin-2(1H)-yl)butoxy)-1,8-naphthyridin-2(1H)-one (I-A29) (IHCH-5213)

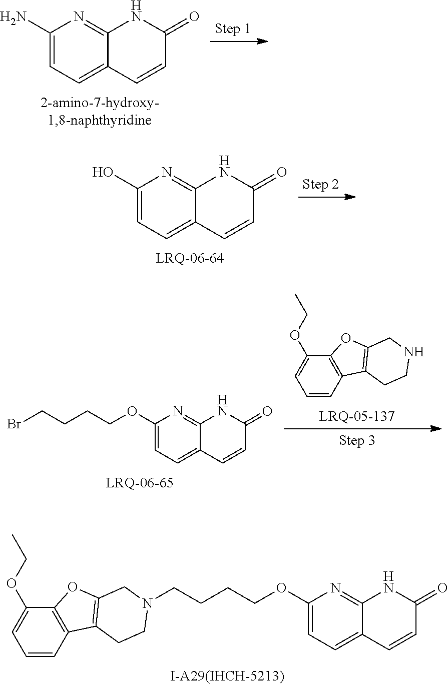 [SMILES: Nc1ccc2ccc(=O)[nH]c2n1 ; O=c1ccc2ccc(O)nc2[nH]1 ; CCOc1cccc2c3c(oc12)CNCC3 ; O=c1ccc2ccc(OCCCCBr)nc2[nH]1 ; CCOc1cccc2c3c(oc12)CN(CCCCOc1ccc2ccc(=O)[nH]c2n1)CC3]

Step 1: Concentrated sulfuric acid (6 mL) was cooled to 0° C., and 2-amino-7-hydroxy-1,8-naphthyridine (1.00 g, 6.21 mmol) and sodium nitrite (684 mg, 9.92 mmol) were sequentially added thereto. The reaction mixture was reacted for 15 minutes, then warmed to room temperature, and reacted for another 15 minutes. After the reaction was completed, the reaction mixture was poured into ice water, added with saturated sodium bicarbonate aqueous solution to adjust the pH to 8, extracted with ethyl acetate (50 mL*3), washed with saturated brine, dried over anhydrous sodium sulfate, and the solvent was removed by evaporation under reduced pressure to obtain LRQ-06-64 as a gray-brown solid, which was directly used in the next reaction step without purification. HRMS (ESI) calculated for C₈H₇N₂O₂⁺ [M+H]⁺: 163.0502, found: 163.0507.

Step 2: LRQ-06-64 was dissolved in DMF (20 mL), then K₂CO₃ (858 mg, 6.21 mmol) and 1,4-dibromobutane (2.68 g, 12.42 mmol) were sequentially added thereto, and the reaction mixture was reacted at room temperature overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:2) to obtain LRQ-06-65 (202 mg, two-step reaction yield: 11%) as a reddish brown solid. ¹H NMR (800 MHz, CDCl₃) δ 10.09 (s, 1H), 7.72 (d, J=8.4 Hz, 1H), 7.64 (d, J=9.4 Hz, 1H), 6.59 (d, J=8.4 Hz, 1H), 6.55 (d, J=9.4 Hz, 1H), 4.41 (t, J=6.3 Hz, 2H), 3.49 (t, J=6.7 Hz, 2H), 2.09-2.01 (m, 2H), 1.99-1.92 (m, 2H). HRMS (ESI) calculated for C₁₂H₁₄BrN₂O₂⁺ [M+H]⁺: 297.0233, found: 297.0237.

Step 3: LRQ-05-137 (50 mg, 0.23 mmol) was dissolved in DMF (8 mL), then DIPEA (178 mg, 1.38 mmol) and LRQ-06-65 (104 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a light yellow solid (10 mg, yield: 10%). ¹H NMR (800 MHz, CDCl₃) δ 9.82 (s, 1H), 7.69 (d, J=8.4 Hz, 1H), 7.61 (d, J=9.4 Hz, 1H), 7.10 (t, J=7.8 Hz, 1H), 7.01 (d, J=7.6 Hz, 1H), 6.77-6.73 (m, 1H), 6.58 (d, J=8.4 Hz, 1H), 6.52 (d, J=9.4 Hz, 1H), 4.43-4.36 (m, 2H), 4.24 (q, J=7.0 Hz, 2H), 3.72 (s, 2H), 2.92-2.84 (m, 2H), 2.75-2.71 (m, 2H), 2.71-2.65 (m, 2H), 1.91-1.82 (m, 2H), 1.82-1.75 (m, 2H), 1.50 (t, J=7.0 Hz, 3H). ¹³C NMR (201 MHz, CDCl₃) δ 164.86, 163.87, 151.28, 148.53, 144.55, 143.89, 139.31, 138.45, 129.87, 123.27, 119.18, 111.85, 111.17, 108.77, 107.52, 107.11, 66.69, 64.51, 57.06, 50.54, 50.19, 26.87, 24.02, 20.90, 15.07. HRMS (ESI) calculated for C₂₅H₂₈N₃O₄⁺ [M+H]⁺: 434.2074, found: 434.2071. HPLC: 95.39% (λ=254 nm, t_{R}=12.05 min).

### Example 30: Preparation of compound 6-(4-(8-ethoxy-3,4-dihydrobenzofuro[2,3-c]pyridin-2(1H)-yl)butoxy)-2H-benzo[b][1,4]oxazin-3(4H)-one (I-A30) (IHCH-5194)

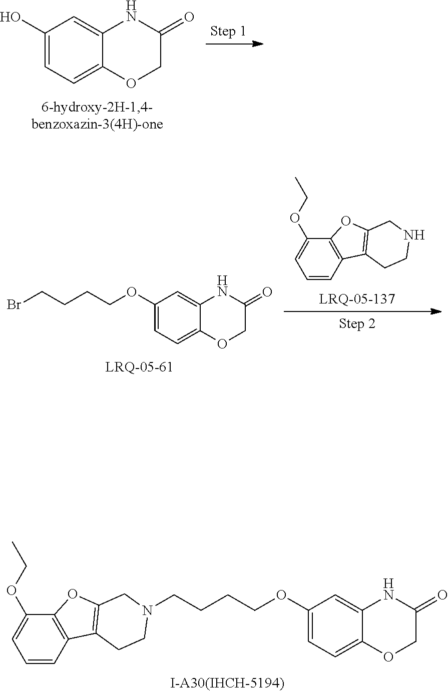 [SMILES: O=C1COc2ccc(O)cc2N1 ; CCOc1cccc2c3c(oc12)CNCC3 ; O=C1COc2ccc(OCCCCBr)cc2N1 ; CCOc1cccc2c3c(oc12)CN(CCCCOc1ccc2c(c1)NC(=O)CO2)CC3]

Step 1: 6-Hydroxy-2H-1,4-benzoxazin-3(4H)-one (0.3 g, 1.77 mmol) was dissolved in DMF (10 mL), then K₂CO₃ (0.25 g, 1.77 mmol) and 1,4-dibromobutane (1.15 g, 5.31 mmol) were sequentially added thereto, and the reaction mixture was reacted at room temperature overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:2) to obtain LRQ-05-61 (0.20 g, yield: 38%) as a yellow solid. ¹H NMR (800 MHz, CDCl₃) δ 7.72 (s, 1H), 6.89 (d, J=8.8 Hz, 1H), 6.53-6.46 (m, 1H), 6.37-6.32 (m, 1H), 4.56 (s, 2H), 3.94 (t, J=6.1 Hz, 2H), 3.48 (t, J=6.6 Hz, 2H), 2.09-2.01 (m, 2H), 1.96-1.88 (m, 2H). HRMS (ESI) calculated for C₁₂H₁₅BrNO₃⁺ [M+H]⁺: 300.0230, found: 300.0233.

Step 2: LRQ-05-137 (50 mg, 0.23 mmol) was dissolved in DMF (8 mL), then DIPEA (178 mg, 1.38 mmol) and LRQ-05-61 (105 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain an orange solid (31 mg, yield: 31%). ¹H NMR (800 MHz, CDCl₃) δ 8.62 (s, 1H), 7.11 (t, J=7.8 Hz, 1H), 7.02 (d, J=7.7 Hz, 1H), 6.86 (d, J=8.8 Hz, 1H), 6.78-6.74 (m, 1H), 6.51-6.47 (m, 1H), 6.37 (d, J=2.6 Hz, 1H), 4.54 (s, 2H), 4.25 (q, J=6.9 Hz, 2H), 3.97-3.89 (m, 2H), 3.73 (s, 2H), 2.92-2.84 (m, 2H), 2.76-2.72 (m, 2H), 2.72-2.65 (m, 2H), 1.87-1.81 (m, 2H), 1.81-1.75 (m, 2H), 1.51 (t, J=7.0 Hz, 3H). ¹³C NMR (201 MHz, CDCl₃) δ 166.46, 154.82, 151.21, 144.59, 143.94, 137.71, 129.85, 126.94, 123.33, 117.32, 111.88, 111.18, 109.53, 107.19, 102.92, 68.41, 67.57, 64.55, 57.00, 50.53, 50.17, 27.22, 23.97, 20.84, 15.07. HRMS (ESI) calculated for C₂₅H₂₉N₂O₅⁺ [M+H]⁺: 437.2071, found: 437.2072. HPLC: 96.67% (λ=254 nm, t_{R}=11.92 min).

### Example 31: Preparation of compound 2-(3-(benzo[d][1,3]dioxol-5-yloxy)propyl)-8-ethoxy-1,2,3,4-tetrahydrobenzofuro[2,3-c]pyridine (I-A31) (IHCH-5193)

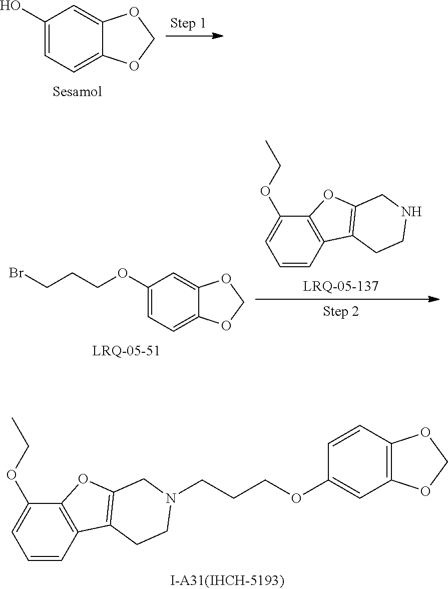 [SMILES: Oc1ccc2c(c1)OCO2 ; CCOc1cccc2c3c(oc12)CNCC3 ; BrCCCOc1ccc2c(c1)OCO2 ; CCOc1cccc2c3c(oc12)CN(CCCOc1ccc2c(c1)OCO2)CC3]

Step 1: Sesamol (1.00 g, 7.23 mmol) was dissolved in DMF (10 mL), then K₂CO₃ (1.00 g, 7.23 mmol) and 1,3-dibromopropane (4.39 g, 21.72 mmol) were sequentially added thereto, and the reaction mixture was reacted at room temperature overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:10) to obtain LRQ-05-51 (0.80 g, yield: 43%) as a yellow solid. ¹H NMR (800 MHz, CDCl₃) δ 6.70 (d, J=8.4 Hz, 1H), 6.51-6.49 (m, 1H), 6.35-6.31 (m, 1H), 5.92 (s, 2H), 4.03 (t, J=5.8 Hz, 2H), 3.62-3.55 (m, 2H), 2.31-2.24 (m, 2H). HRMS (ESI) calculated for C₁₀H₁₂BrO₃⁺ [M+H]⁺: 258.9964, found: 258.9970.

Step 2: LRQ-05-137 (50 mg, 0.23 mmol) was dissolved in DMF (8 mL), then DIPEA (178 mg, 1.38 mmol) and LRQ-05-51 (90 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a white solid (39 mg, yield: 42%). ¹H NMR (800 MHz, CDCl₃) δ 7.15-7.09 (m, 1H), 7.04-7.00 (m, 1H), 6.76 (d, J=7.9 Hz, 1H), 6.72-6.67 (m, 1H), 6.52-6.48 (m, 1H), 6.36-6.30 (m, 1H), 5.90 (s, 2H), 4.25 (q, J=7.0 Hz, 2H), 4.03-3.95 (m, 2H), 3.73 (s, 2H), 2.91-2.83 (m, 2H), 2.83-2.76 (m, 2H), 2.76-2.70 (m, 2H), 2.08-1.99 (m, 2H), 1.51 (t, J=7.0 Hz, 3H). ¹³C NMR (201 MHz, CDCl₃) δ 154.59, 151.31, 148.33, 144.56, 143.91, 141.68, 129.89, 123.25, 111.87, 111.16, 108.03, 107.13, 105.79, 101.19, 98.21, 67.07, 64.51, 54.13, 50.56, 50.34, 27.49, 20.95, 15.06. HRMS (ESI) calculated for C₂₃H₂₆NO₅⁺ [M+H]⁺: 396.1805, found: 396.1800. HPLC: 97.98% (λ=254 nm, t_{R}=12.72 min).

### Example 32: Preparation of compound N-(2-(8-ethoxy-3,4-dihydrobenzofuro[2,3-c]pyridin-2(1H)-yl)ethyl)cycloheptanecarboxamide (I-A32) (IHCH-5215)

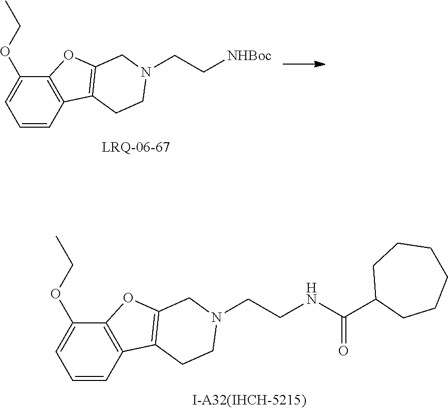 [SMILES: CCOc1cccc2c3c(oc12)CN(CCNC(=O)OC(C)(C)C)CC3 ; CCOc1cccc2c3c(oc12)CN(CCNC(=O)C1CCCCCC1)CC3]

LRQ-06-67 (70 mg, 0.19 mmol) was dissolved in DCM (10 mL), and the reaction mixture was cooled to 0° C. Trifluoroacetic acid (1 mL) was added thereto, and the reaction mixture was reacted for 2 hours. After the reaction was completed, the solvent was removed by evaporation under reduced pressure to obtain a colorless oily liquid, which was then dissolved in dichloromethane (10 mL). DIPEA (150 mg, 1.16 mmol) and cycloheptanecarbonyl chloride (46 mg, 0.28 mmol) were added thereto, and the reaction mixture was reacted at room temperature overnight. After the reaction was completed, the reaction mixture was diluted with saturated sodium bicarbonate aqueous solution (10 mL), extracted with dichloromethane (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain a light yellow solid (35 mg, yield: 47%). ¹H NMR (800 MHz, CDCl₃) δ 7.13 (t, J=7.8 Hz, 1H), 7.05-7.02 (m, 1H), 6.80-6.75 (m, 1H), 6.11 (s, 1H), 4.25 (q, J=7.0 Hz, 2H), 3.74 (s, 2H), 3.46-3.39 (m, 2H), 2.93-2.85 (m, 2H), 2.80-2.76 (m, 2H), 2.76-2.71 (m, 2H), 2.25-2.18 (m, 1H), 1.89-1.83 (m, 2H), 1.78-1.71 (m, 2H), 1.68-1.62 (m, 2H), 1.58-1.49 (m, 7H), 1.46-1.40 (m, 2H). ¹³C NMR (201 MHz, CDCl₃) δ 177.64, 152.38, 144.64, 143.95, 129.69, 123.46, 111.92, 111.17, 107.32, 64.57, 55.61, 50.15, 50.06, 47.56, 36.35, 31.83 (2C), 28.24 (2C), 26.72 (2C), 20.74, 15.07. HRMS (ESI) calculated for C₂₃H₃₃N₂O₃⁺ [M+H]⁺: 385.2486, found: 385.2487. HPLC: 96.48% (Q=254 nm, t_{R}=13.09 min).

### Example 33: Preparation of compound N-(2-(8-ethoxy-3,4-dihydrobenzofuro[2,3-c]pyridin-2(1H)-yl)ethyl)cyclopentanecarboxamide (I-A33) (IHCH-5214)

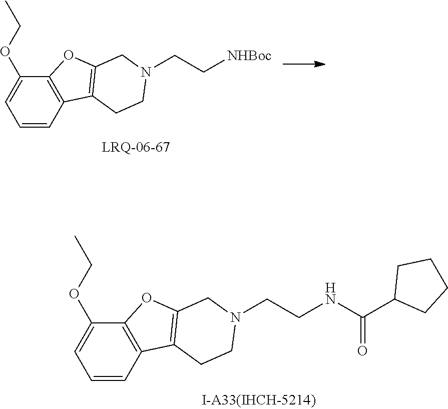 [SMILES: CCOc1cccc2c3c(oc12)CN(CCNC(=O)OC(C)(C)C)CC3 ; CCOc1cccc2c3c(oc12)CN(CCNC(=O)C1CCCC1)CC3]

LRQ-06-67 (70 mg, 0.19 mmol) was dissolved in DCM (10 mL), and the reaction mixture was cooled to 0° C. Trifluoroacetic acid (1 mL) was added thereto, and the reaction mixture was reacted for 2 hours. After the reaction was completed, the solvent was removed by evaporation under reduced pressure to obtain a colorless oily liquid, which was then dissolved in dichloromethane (10 mL). DIPEA (150 mg, 1.16 mmol) and cyclopentanecarbonyl chloride (40 mg, 0.28 mmol) were added thereto, and the reaction mixture was reacted at room temperature overnight. After the reaction was completed, the reaction mixture was diluted with saturated sodium bicarbonate aqueous solution (10 mL), extracted with dichloromethane (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain a light yellow solid (20 mg, yield: 29%). ¹H NMR (800 MHz, CDCl₃) δ 7.12 (t, J=7.8 Hz, 1H), 7.02 (d, J=7.4 Hz, 1H), 6.79-6.74 (m, 1H), 6.18 (s, 1H), 4.24 (q, J=7.0 Hz, 2H), 3.71 (s, 2H), 3.47-3.40 (m, 2H), 2.90-2.84 (m, 2H), 2.78-2.73 (m, 2H), 2.73-2.68 (m, 2H), 2.54-2.46 (m, 1H), 1.87-1.79 (m, 2H), 1.77-1.67 (m, 4H), 1.59-1.52 (m, 2H), 1.50 (t, J=7.0 Hz, 3H). ¹³C NMR (201 MHz, CDCl₃) δ 176.56, 150.83, 144.59, 143.89, 129.70, 123.38, 111.88, 111.13, 107.25, 64.53, 55.56, 50.12, 50.01, 45.94, 36.51, 30.58 (2C), 25.99 (2C), 20.77, 15.03. HRMS (ESI) calculated for C₂₁H₂₉N₂O₃⁺ [M+H]⁺: 357.2173, found: 357.2170. HPLC: 96.44% (λ=254 nm, t_{R}=12.37 min).

### Example 34: Preparation of compound 3-(3-(2-(8-ethoxy-3,4-dihydrobenzofuro[2,3-c]pyridin-2(1H)-yl)ethyl)cyclobutyl)-1,1-dimethylurea (I-A34) (IHCH-5195)

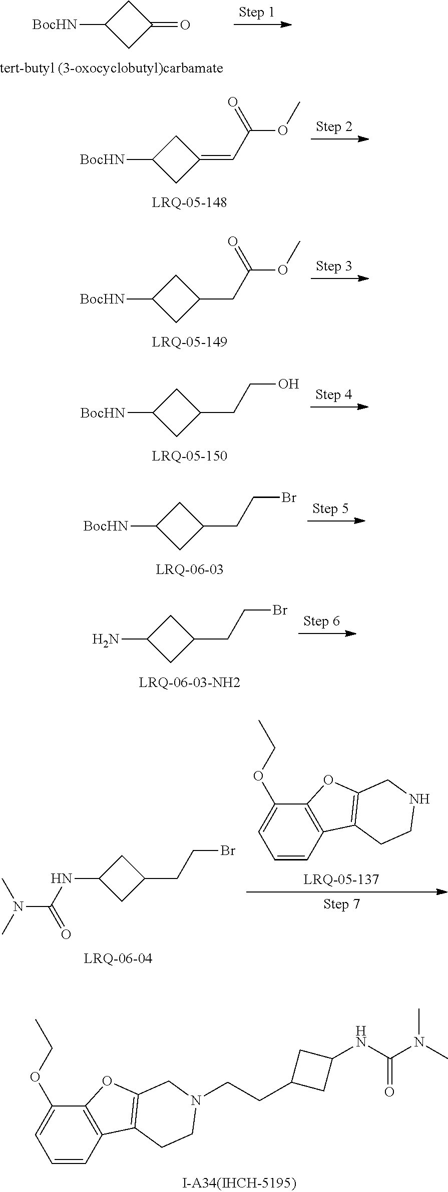

Step 1: tert-Butyl (3-oxocyclobutyl)carbamate (2.00 g, 10.80 mmol) was dissolved in DCM (10 mL), then methyl (triphenylphosphoranylidene)acetate (4.33 g, 12.96 mmol) was added thereto, and the reaction mixture was reacted at room temperature overnight. After the reaction was completed, the solvent was removed by evaporation under reduced pressure, and the residue was purified by silica gel column chromatography (ethyl acetate/petroleum ether=1:4) to obtain LRQ-05-148 (2.22 g, yield: 85%) as a yellow solid. ¹H NMR (600 MHz, CDCl₃) δ 5.70 (s, 1H), 4.81 (s, 1H), 4.31-4.15 (m, 1H), 3.69 (s, 3H), 3.62-3.47 (m, 1H), 3.26-3.11 (m, 1H), 3.00-2.84 (m, 1H), 2.79-2.65 (m, 1H), 1.45 (s, 9H). HRMS (ESI) calculated for C₁₂H₂₀NO₄⁺ [M+H]⁺: 242.1387, found: 242.1384.

Step 2: LRQ-05-148 (2.00 g, 8.30 mmol) was dissolved in anhydrous methanol (20 mL), then 10% Pd/C was added thereto, and the reaction mixture was replaced with hydrogen three times. Hydrogen was introduced thereto, and the reaction mixture was reacted at room temperature overnight. After the reaction was completed, the reaction mixture was filtered, and the solvent was removed by evaporation under reduced pressure to obtain LRQ-05-149 as a white solid, which was directly used in the next reaction step without purification. HRMS (ESI) calculated for C₁₂H₂₂NO₄⁺ [M+H]⁺: 244.1543, found: 244.1546.

Step 3: LRQ-05-149 was dissolved in THE (20 mL). The reaction mixture was replaced with nitrogen three times, then cooled to −10° C., and DIBAL-H (25 mL, 1.0 M in tetrahydrofuran) was added thereto. The reaction mixture was then warmed to 0° C. and reacted for 6 hours. After the reaction was completed, the reaction mixture was added with saturated potassium sodium tartrate aqueous solution (10 mL), then stirred at room temperature for 1 hour, extracted with ethyl acetate (20 mL*3), washed with saturated brine, dried over anhydrous sodium sulfate, and the solvent was removed by evaporation under reduced pressure to obtain LRQ-05-150 as a light yellow oily liquid, which was directly used in the next reaction step without purification. HRMS (ESI) calculated for C₁₁H₁₂NO₃⁺ [M+H]⁺: 216.1594, found: 216.1589.

Step 4: LRQ-05-150 was dissolved in DCM (10 mL), and the reaction mixture was cooled to 0° C. Carbon tetrabromide (2.79 g, 8.41 mmol) and triphenylphosphine (2.94 g, 11.21 mmol) were sequentially added thereto, and the reaction mixture was reacted at room temperature overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate) to obtain LRQ-06-03 (1.38 g, three-step reaction yield: 60%) as a white solid. ¹H NMR (600 MHz, CDCl₃) δ 4.25-4.07 (m, 1H), 3.36-3.27 (m, 2H), 2.56-1.86 (m, 7H), 1.44-1.39 (m, 9H). HRMS (ESI) calculated for C₁₁H₂₀BrNO₂Na⁺ [M+H]⁺: 300.0570, found: 300.0575.

Step 5: LRQ-06-03 (250 mg, 1.41 mmol) was dissolved in DCM (10 mL), and the reaction mixture was cooled to 0° C. Trifluoroacetic acid (1 mL) was added thereto, and the reaction mixture was reacted for 2 hours. After the reaction was completed, the solvent was removed by evaporation under reduced pressure to obtain LRQ-06-03-NH2 as a light yellow oily liquid, which was directly used in the next reaction step without purification. HRMS (ESI) calculated for C₆H₁₃BrN⁺ [M+H]⁺: 178.0226, found: 178.0227.

Step 6: LRQ-06-03-NH2 was dissolved in DCM (10 mL), then triethylamine (428 mg, 4.23 mmol) and dimethylcarbamoyl chloride (182 mg, 1.69 mmol) were sequentially added thereto, and the reaction mixture was reacted at room temperature overnight. After the reaction was completed, the reaction mixture was diluted with saturated sodium bicarbonate aqueous solution (10 mL), extracted with dichloromethane (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-06-04 (132 mg, two-step reaction yield: 59%) as a light yellow oily liquid. ¹H NMR (600 MHz, CDCl₃) δ 4.23-4.07 (m, 1H), 3.39-3.24 (m, 2H), 2.94-2.83 (m, 6H), 2.61-1.90 (m, 7H). HRMS (ESI) calculated for C₉H₁₈BrN₂O⁺ [M+H]⁺: 249.0597, found: 249.0600.

Step 7: LRQ-05-137 (50 mg, 0.23 mmol) was dissolved in DMF (8 mL), then DIPEA (178 mg, 1.38 mmol) and LRQ-06-04 (87 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain an orange solid (38 mg, yield: 42%). ¹H NMR (800 MHz, CDCl₃) δ 7.12-7.08 (m, 1H), 7.01 (d, J=7.7 Hz, 1H), 6.75 (d, J=7.9 Hz, 1H), 4.52-4.40 (m, 1H), 4.26-4.21 (m, 2H), 3.69 (s, 2H), 2.89-2.86 (m, 6H), 2.86-2.82 (m, 2H), 2.74-2.69 (m, 2H), 2.54-2.51 (m, 2H), 2.35-1.60 (m, 7H), 1.50 (t, J=7.0 Hz, 3H). HRMS (ESI) calculated for C₂₂H₃₂N₃O₃⁺[M+H]⁺: 386.2438, found: 386.2431. HPLC: 95.46% (λ=254 nm, t_{R}=11.55 min).

### Example 35: Preparation of compound N-(2-(8-ethoxy-3,4-dihydrobenzofuro[2,3-c]pyridin-2(1H)-yl)ethyl)benzamide (I-A35) (IHCH-5212)

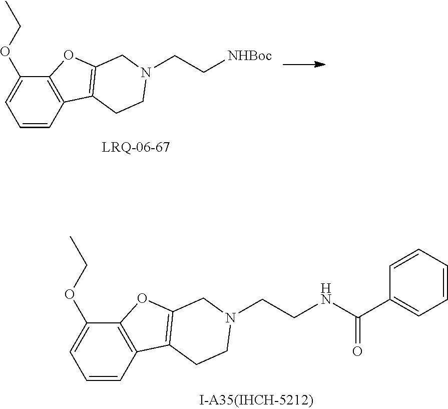 [SMILES: CCOc1cccc2c3c(oc12)CN(CCNC(=O)OC(C)(C)C)CC3 ; CCOc1cccc2c3c(oc12)CN(CCNC(=O)c1ccccc1)CC3]

LRQ-06-67 (70 mg, 0.19 mmol) was dissolved in DCM (10 mL), and the reaction mixture was cooled to 0° C. Trifluoroacetic acid (1 mL) was added thereto, and the reaction mixture was reacted for 2 hours. After the reaction was completed, the solvent was removed by evaporation under reduced pressure to obtain a colorless oily liquid, which was then dissolved in dichloromethane (10 mL). DIPEA (150 mg, 1.16 mmol) and benzoyl chloride (40 mg, 0.28 mmol) were added thereto, and the reaction mixture was reacted at room temperature overnight. After the reaction was completed, the reaction mixture was diluted with saturated sodium bicarbonate aqueous solution (10 mL), extracted with dichloromethane (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain a light yellow solid (10 mg, yield: 14%). ¹H NMR (800 MHz, MeOH-d₄) δ 8.31 (s, 1H), 7.88-7.80 (m, 2H), 7.51 (t, J=7.4 Hz, 1H), 7.47-7.39 (m, 2H), 7.11 (t, J=7.8 Hz, 1H), 7.03 (d, J=7.7 Hz, 1H), 6.85-6.79 (m, 1H), 4.20 (q, J=7.0 Hz, 2H), 3.93 (s, 2H), 3.72-3.63 (m, 2H), 3.12-3.06 (m, 2H), 3.04-2.98 (m, 2H), 2.83-2.75 (m, 2H), 1.43 (t, J=7.0 Hz, 3H). ¹³C NMR (201 MHz, MeOH-d₄) δ 170.41, 150.46, 145.88, 145.45, 135.38, 132.75, 130.57, 129.55 (2C), 128.29 (2C), 124.62, 112.80, 112.15, 109.11, 65.63, 57.18, 51.56, 50.74, 38.01, 21.03, 15.26. HRMS (ESI) calculated for C₂₂H₂₅N₂O₃⁺ [M+H]⁺: 365.1860, found: 365.1863. HPLC: 96.03% (λ=254 nm, t_{R}=12.20 min).

### Example 36: Preparation of compound N-(2-(8-ethoxy-3,4-dihydrobenzofuro[2,3-c]pyridin-2(1H)-yl)ethyl)thiophene-2-carboxamide (I-A36) (IHCH-5211)

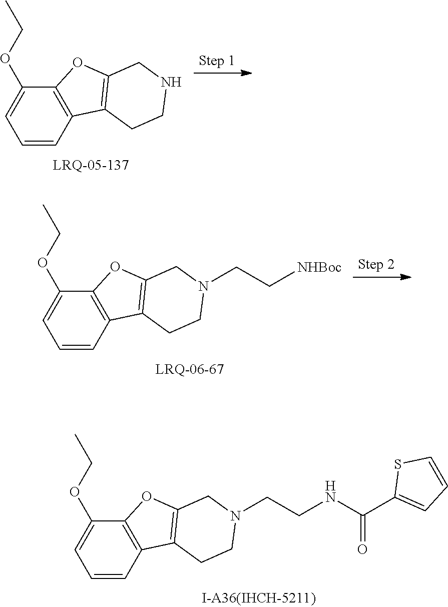 [SMILES: CCOc1cccc2c3c(oc12)CNCC3 ; CCOc1cccc2c3c(oc12)CN(CCNC(=O)OC(C)(C)C)CC3 ; CCOc1cccc2c3c(oc12)CN(CCNC(=O)c1cccs1)CC3]

Step 1: LRQ-05-137 (320 mg, 1.48 mmol) was dissolved in DMF (10 mL), then DIPEA (1.15 g, 8.88 mmol) and N-Boc-2-bromoethylamine (498 mg, 2.22 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-06-67 (293 mg, yield: 55%) as a yellow solid. ¹H NMR (800 MHz, CDCl₃) δ 8.02 (s, 1H), 7.15-7.09 (m, 1H), 7.05-7.00 (m, 1H), 6.81-6.74 (m, 1H), 4.25 (q, J=7.0 Hz, 2H), 3.77 (s, 2H), 3.42-3.27 (m, 2H), 2.99-2.92 (m, 2H), 2.84-2.71 (m, 4H), 1.51 (t, J=7.0 Hz, 3H), 1.44 (s, 9H). HRMS (ESI) calculated for C₂₀H₂₉N₂O₄⁺ [M+H]⁺: 361.2122, found: 361.2120.

Step 2: LRQ-06-67 (70 mg, 0.19 mmol) was dissolved in DCM (10 mL), and the reaction mixture was cooled to 0° C. Trifluoroacetic acid (1 mL) was added thereto, and the reaction mixture was reacted for 2 hours. After the reaction was completed, the solvent was removed by evaporation under reduced pressure to obtain a colorless oily liquid, which was then dissolved in dichloromethane (10 mL). DIPEA (150 mg, 1.16 mmol) and 2-thiophenecarbonyl chloride (42 mg, 0.28 mmol) were added thereto, and the reaction mixture was reacted at room temperature overnight. After the reaction was completed, the reaction mixture was diluted with saturated sodium bicarbonate aqueous solution (10 mL), extracted with dichloromethane (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain a light yellow solid (14 mg, yield: 19%). ¹H NMR (800 MHz, MeOH-d₄) δ 8.32 (s, 1H), 7.69-7.66 (m, 1H), 7.64-7.61 (m, 1H), 7.13-7.08 (m, 2H), 7.03 (d, J=7.7 Hz, 1H), 6.82 (d, J=7.9 Hz, 1H), 4.20 (q, J=7.0 Hz, 2H), 3.92 (s, 2H), 3.68-3.61 (m, 2H), 3.10-3.04 (m, 2H), 3.01-2.96 (m, 2H), 2.81-2.75 (m, 2H), 1.43 (t, J=7.0 Hz, 3H). ¹³C NMR (201 MHz, MeOH-d₄) δ 164.67, 150.40, 145.87, 145.44, 139.94, 131.76, 130.55, 129.76, 128.80, 124.63, 112.80, 112.15, 109.11, 65.63, 57.22, 51.55, 50.71, 37.85, 21.00, 15.27. HRMS (ESI) calculated for C₂₀H₂₃N₂O₃S⁺ [M+H]⁺: 371.1424, found: 371.1430. HPLC: 96.23% (Q=254 nm, t_{R}=12.02 min).

### Example 37: Preparation of compound 2-(2-(8-ethoxy-3,4-dihydrobenzofuro[2,3-c]pyridin-2(1H)-yl)ethyl)-3,4-dihydroisoquinolin-1(2H)-one (I-A37) (IHCH-5198)

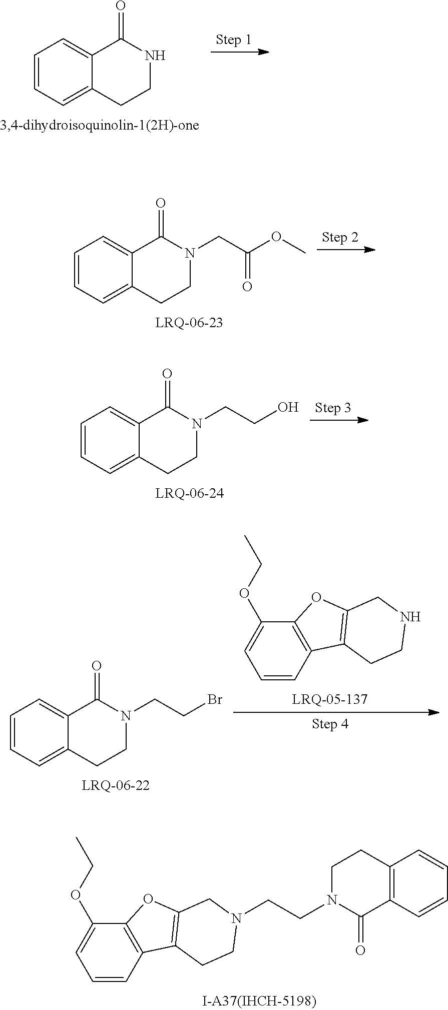 [SMILES: O=C1NCCc2ccccc21 ; COC(=O)CN1CCc2ccccc2C1=O ; O=C1c2ccccc2CCN1CCO ; O=C1c2ccccc2CCN1CCBr ; CCOc1cccc2c3c(oc12)CNCC3 ; CCOc1cccc2c3c(oc12)CN(CCN1CCc2ccccc2C1=O)CC3]

Step 1: 3,4-Dihydroisoquinolin-1(2H)-one (526 mg, 3.57 mmol) was dissolved in toluene (10 mL), and the reaction mixture was cooled to 0° C. NaH (167 mg, 4.18 mmol) was added thereto, and the reaction mixture was refluxed for 1 hour. The reaction mixture was then cooled to room temperature, added with methyl bromoacetate (639 mg, 4.18 mmol), and refluxed overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:4) to obtain LRQ-06-23 (746 mg, yield: 95%) as a light yellow oily liquid. ¹H NMR (800 MHz, CDCl₃) δ 8.01-7.96 (m, 1H), 7.37-7.31 (m, 1H), 7.25 (t, J=7.6 Hz, 1H), 7.10 (d, J=7.6 Hz, 1H), 4.26 (s, 2H), 3.66 (s, 3H), 3.57 (t, J=6.7 Hz, 2H), 2.99-2.93 (m, 2H). HRMS (ESI) calculated for C₁₂H₁₄NO₃⁺ [M+H]⁺: 220.0968, found: 220.0972.

Step 2: LRQ-06-23 (526 mg, 2.40 mmol) was dissolved in THE (10 mL). The reaction mixture was replaced with nitrogen three times, then cooled to −10° C., and DIBAL-H (7 mL, 1.0 M in tetrahydrofuran) was added thereto. The reaction mixture was then warmed to 0° C. and reacted for 6 hours. After the reaction was completed, the reaction mixture was added with saturated potassium sodium tartrate aqueous solution (10 mL), then stirred at room temperature for 1 hour, extracted with ethyl acetate (20 mL*3), washed with saturated brine, dried over anhydrous sodium sulfate, and the solvent was removed by evaporation under reduced pressure to obtain LRQ-06-24 as a light yellow oily liquid, which was directly used in the next reaction step without purification. HRMS (ESI) calculated for C₁₁H₁₄NO₂⁺ [M+H]⁺: 192.1019, found: 192.1015.

Step 3: LRQ-06-24 was dissolved in DCM (10 mL), and the reaction mixture was cooled to 0° C. Carbon tetrabromide (1.19 g, 3.60 mmol) and triphenylphosphine (1.26 g, 4.80 mmol) were sequentially added thereto, and the reaction mixture was reacted at room temperature overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:4) to obtain LRQ-06-22 (255 mg, two-step reaction yield: 42%) as a yellow solid. ¹H NMR (800 MHz, CDCl₃) δ 8.09-8.05 (m, 1H), 7.46-7.41 (m, 1H), 7.38-7.33 (m, 1H), 7.19 (d, J=7.5 Hz, 1H), 3.95 (t, J=6.3 Hz, 2H), 3.72 (t, J=6.5 Hz, 2H), 3.65 (t, J=6.3 Hz, 2H), 3.07-3.03 (m, 2H). HRMS (ESI) calculated for C₁₁H₁₃BrNO⁺ [M+H]⁺: 254.0175, found: 254.0179.

Step 4: LRQ-05-137 (40 mg, 0.19 mmol) was dissolved in DMF (8 mL), then DIPEA (178 mg, 1.38 mmol) and LRQ-06-22 (74 mg, 0.29 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (15 mg, yield: 21%). ¹H NMR (800 MHz, MeOH-d₄) δ 7.94-7.90 (m, 1H), 7.46-7.40 (m, 1H), 7.35-7.29 (m, 1H), 7.23 (d, J=7.4 Hz, 1H), 7.11-7.06 (m, 1H), 7.03-6.99 (m, 1H), 6.81-6.77 (m, 1H), 4.21 (q, J=7.0 Hz, 2H), 3.87-3.78 (m, 4H), 3.70-3.61 (m, 2H), 3.04-2.99 (m, 2H), 2.99-2.96 (m, 2H), 2.96-2.90 (m, 2H), 2.77-2.69 (m, 2H), 1.44 (t, J=7.0 Hz, 3H). ¹³C NMR (201 MHz, MeOH-d₄) δ 166.61, 151.61, 145.73, 145.26, 140.05, 133.10, 130.79, 130.12, 128.61, 128.21, 127.91, 124.36, 112.75, 112.05, 108.87, 65.59, 55.49, 51.46, 51.06, 47.81, 46.02, 28.81, 21.49, 15.28. HRMS (ESI) calculated for C₂₄H₂₇N₂O₃⁺ [M+H]⁺: 391.2016, found: 391.2011. HPLC: 98.96% (λ=254 nm, t_{R}=11.33 min).

### Example 38: Preparation of compound 2-(2-(8-ethoxy-3,4-dihydrobenzofuro[2,3-c]pyridin-2(1H)-yl)ethyl)-6-methyl-3,4-trihydro-2,7-naphthyridin-1(2H)-one (I-A38) (IHCH-5199)

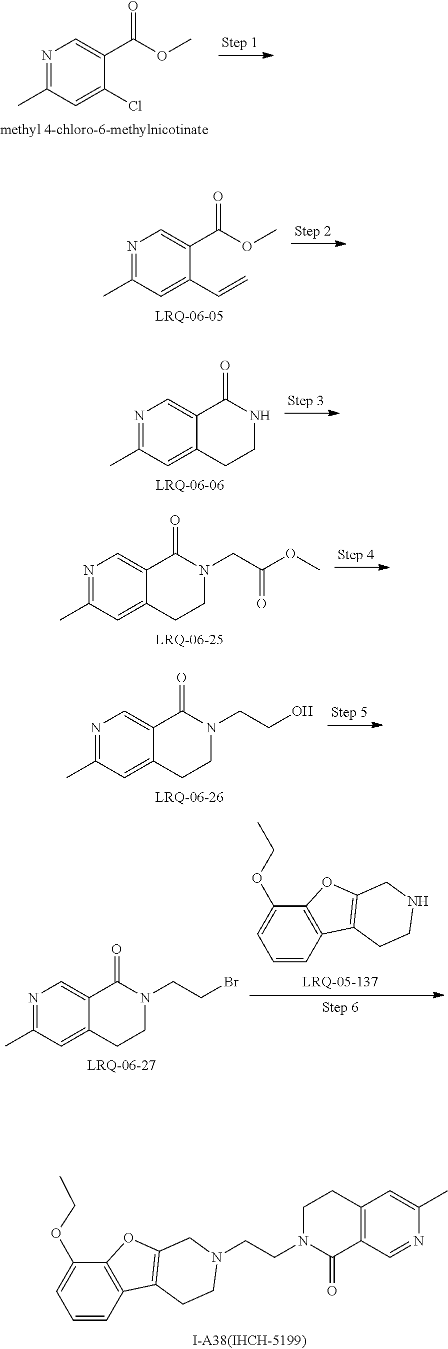

Step 1: Methyl 4-chloro-6-methylnicotinate (4.03 g, 21.78 mmol), tributyl(vinyl)tin (10.36 g, 32.67 mmol), and tetrakis(triphenylphosphine)palladium (1.76 g, 1.52 mmol) were dissolved in toluene (10 mL) and 1,4-dioxane (10 mL). The reaction mixture was replaced with nitrogen three times, heated to 90° C., and reacted overnight. After the reaction was completed, the reaction mixture was diluted with saturated sodium bicarbonate aqueous solution (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:10) to obtain LRQ-06-05 (3.2 g, yield: 83%) as an orange-yellow oily liquid. ¹H NMR (800 MHz, CDCl₃) δ 8.99 (s, 1H), 7.55-7.47 (m, 1H), 7.32 (s, 1H), 5.84 (d, J=17.5 Hz, 1H), 5.53 (d, J=11.0 Hz, 1H), 3.92 (s, 3H), 2.61 (s, 3H). HRMS (ESI) calculated for C₁₀H₁₂NO₂⁺ [M+H]⁺: 178.0863, found: 178.0868.

Step 2: LRQ-06-05 (3.20 g, 18.08 mmol) was mixed with 50 mL of ammonia (50 mL, 7.0 M in methanol) in a sealed tube, and the reaction mixture was heated to 100° C. and stirred overnight. After the reaction was completed, the solvent was removed by evaporation under reduced pressure to obtain LRQ-06-06 as a pink solid, which was directly used in the next reaction step without purification. HRMS (ESI) calculated for C₉H₁₁N₂O⁺ [M+H]⁺: 163.0866, found: 163.0869.

Step 3: LRQ-06-06 was dissolved in toluene (10 mL), and the reaction mixture was cooled to 0° C. NaH (844 mg, 21.17 mmol) was added thereto, and the reaction mixture was refluxed for 1 hour. The reaction mixture was then cooled to room temperature, added with methyl bromoacetate (3.23 g, 21.17 mmol), and refluxed overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:4) to obtain LRQ-06-25 (3.72 g, two-step reaction yield: 88%) as a yellow solid. ¹H NMR (800 MHz, CDCl₃) δ 9.09 (s, 1H), 7.02 (s, 1H), 4.34 (s, 2H), 3.76 (s, 3H), 3.67 (t, J=6.6 Hz, 2H), 3.03 (t, J=6.6 Hz, 2H), 2.60 (s, 3H). HRMS (ESI) calculated for C₁₂H₁₅N₂₀₃₊[M+H]⁺: 235.1077, found: 235.1082.

Step 4: LRQ-06-25 (600 mg, 2.56 mmol) was dissolved in THE (10 mL). The reaction mixture was replaced with nitrogen three times, then cooled to −10° C., and DIBAL-H (8 mL, 1.0 M in tetrahydrofuran) was added thereto. The reaction mixture was then warmed to 0° C. and reacted for 6 hours. After the reaction was completed, the reaction mixture was added with saturated potassium sodium tartrate aqueous solution (10 mL), then stirred at room temperature for 1 hour, extracted with ethyl acetate (20 mL*3), washed with saturated brine, dried over anhydrous sodium sulfate, and the solvent was removed by evaporation under reduced pressure to obtain LRQ-06-26 as a light yellow oily liquid, which was directly used in the next reaction step without purification. HRMS (ESI) calculated for C₁₁H₁₅N₂O₂⁺ [M+H]⁺: 207.1128, found: 207.1130.

Step 5: LRQ-06-26 was dissolved in DCM (10 mL), and the reaction mixture was cooled to 0° C. Carbon tetrabromide (1.27 g, 3.84 mmol) and triphenylphosphine (1.34 g, 5.12 mmol) were sequentially added thereto, and the reaction mixture was reacted at room temperature overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:4) to obtain LRQ-06-27 (206 mg, two-step reaction yield: 30%) as a yellow solid. ¹H NMR (800 MHz, MeOH-d₄) δ 8.95 (s, 1H), 7.76 (s, 1H), 3.83 (t, J=6.6 Hz, 2H), 3.80 (t, J=5.5 Hz, 2H), 3.73-3.69 (m, 2H), 3.28 (t, J=6.6 Hz, 2H), 2.76 (s, 3H). HRMS (ESI) calculated for C₁₁H₁₄BrN₂O⁺ [M+H]⁺: 269.0284, found: 269.0283.

Step 6: LRQ-05-137 (50 mg, 0.23 mmol) was dissolved in DMF (8 mL), then DIPEA (191 mg, 1.48 mmol) and LRQ-06-27 (94 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (27 mg, yield: 29%). ¹H NMR (800 MHz, CDCl₃) δ 9.07 (s, 1H), 7.11 (t, J=7.8 Hz, 1H), 7.02 (d, J=7.7 Hz, 1H), 6.96 (s, 1H), 6.77 (d, J=7.9 Hz, 1H), 4.27-4.21 (m, 2H), 3.88-3.76 (m, 4H), 3.69-3.62 (m, 2H), 3.03-2.96 (m, 2H), 2.95-2.90 (m, 4H), 2.77-2.70 (m, 2H), 2.56 (s, 3H), 1.50 (t, J=7.0 Hz, 3H). ¹³C NMR (201 MHz, CDCl₃) δ 163.36, 161.52, 150.69, 149.22, 147.00, 144.54, 143.86, 129.70, 123.34, 122.65, 121.06, 111.89, 111.14, 107.24, 64.51, 54.78, 50.55, 50.30, 46.56, 45.26, 27.66, 24.62, 20.81, 15.01. HRMS (ESI) calculated for C₂₄H₂₈N₃O₃⁺ [M+H]⁺: 406.2125, found: 406.2121. HPLC: 95.82% (λ=254 nm, t_{R}=11.88 min).

### Example 39: Preparation of compound 5-(2-(8-ethoxy-3,4-dihydrobenzofuro[2,3-c]pyridin-2(1H)-yl)ethyl)-2,3-dimethyl-2,5,6,7-tetrahydro-4H-pyrazolo[4,3-c]pyridin-4-one (I-A39) (IHCH-5216)

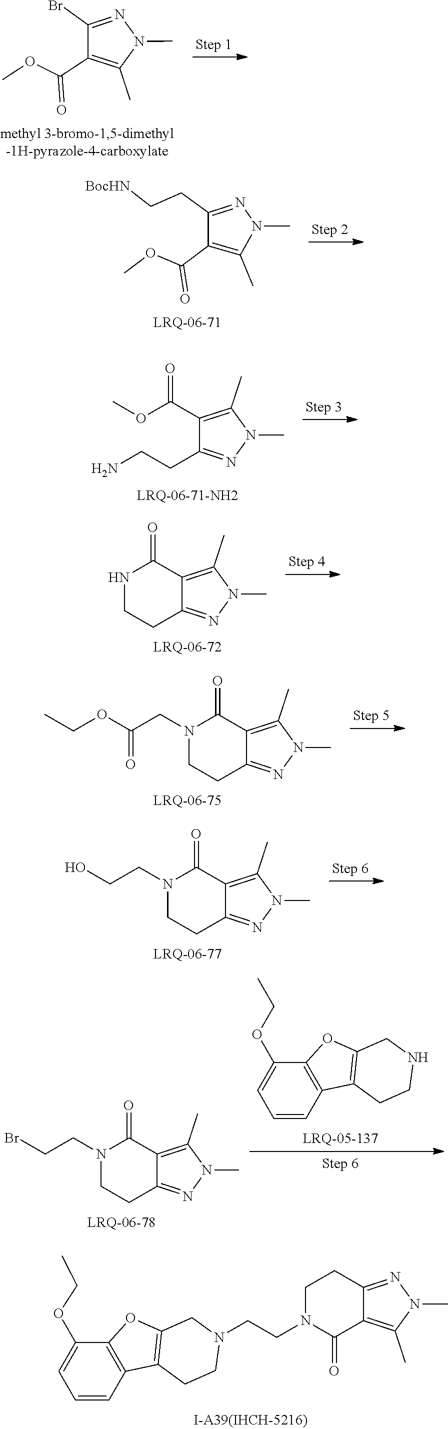

Step 1: Methyl 3-bromo-1,5-dimethyl-1H-pyrazole-4-carboxylate (0.46 g, 2.04 mmol), potassium (2((tert-butoxycarbonyl)amino)ethyl)trifluoroborate (0.76 g, 3.04 mmol), cesium carbonate (1.64 g, 5.06 mmol), and [1,1′-bis(diphenylphosphino)ferrocene]palladium(II)dichloride dichloromethane complex (0.16 g, 0.18 mmol) were dissolved in toluene (16 mL) and water (8 mL). The reaction mixture was replaced with nitrogen three times, heated to 100° C., and reacted for 3 hours. After the reaction was completed, the reaction mixture was diluted with water (10 mL), extracted with ethyl acetate (20 mL*3), washed with saturated brine, dried over anhydrous sodium sulfate, and the solvent was removed by evaporation under reduced pressure to obtain LRQ-06-71 as an orange-yellow oily liquid, which was directly used in the next reaction step without purification. HRMS (ESI) calculated for C₁₄H₂₄N₃O₄⁺[M+H]⁺: 298.1761, found: 298.1760.

Step 2: LRQ-06-71 was dissolved in DCM (10 mL), and the reaction mixture was cooled to 0° C. Trifluoroacetic acid (1 mL) was added thereto, and the reaction mixture was reacted for 2 hours. After the reaction was completed, the solvent was removed by evaporation under reduced pressure to obtain LRQ-06-71-NH2 as a light yellow oily liquid, which was directly used in the next reaction step without purification. HHRMS (ESI) calculated for C₉H₁₆N₃O₂⁺ [M+H]⁺: 198.1237, found: 198.1239.

Step 3: LRQ-06-71-NH2 was dissolved in methanol (10 mL), then sodium methoxide (2 mL, 5.0 M in methanol) was added thereto, and the reaction mixture was reacted at room temperature for 0.5 hours. After the reaction was completed, the solvent was removed by evaporation under reduced pressure. The reaction mixture was then diluted with water (10 mL), extracted with dichloromethane (20 mL*3), washed with water, dried over anhydrous sodium sulfate, and the solvent was removed by evaporation under reduced pressure to obtain LRQ-06-72 as a light yellow solid, which was directly used in the next reaction step without purification. HRMS (ESI) calculated for C₈H₁₂N₃₀⁺ [M+H]⁺: 166.0975, found: 166.0969.

Step 4: LRQ-06-72 was dissolved in toluene (10 mL), and the reaction mixture was cooled to 0° C. NaH (94 mg, 2.36 mmol) was added thereto, and the reaction mixture was refluxed for 1 hour. The reaction mixture was then cooled to room temperature, added with ethyl bromoacetate (394 mg, 2.36 mmol), and refluxed overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-06-75 (220 mg, four-step reaction yield: 43%) as a light yellow solid. ¹H NMR (800 MHz, CDCl₃) δ 4.24 (s, 2H), 4.20 (q, J=7.1 Hz, 2H), 3.79-3.73 (m, 3H), 3.67-3.62 (m, 2H), 3.00-2.91 (m, 2H), 2.56-2.50 (m, 3H), 1.28 (t, J=7.1 Hz, 3H). HHRMS (ESI) calculated for C₁₂H₁₈N₃O₃⁺ [M+H]⁺: 252.1343, found: 252.1341.

Step 5: LRQ-06-75 (220 mg, 0.87 mmol) was dissolved in THE (5 mL). The reaction mixture was replaced with nitrogen three times, then cooled to −10° C., and DIBAL-H (3 mL, 1.0 M in tetrahydrofuran) was added thereto. The reaction mixture was then warmed to 0° C. and reacted for 6 hours. After the reaction was completed, the reaction mixture was added with saturated potassium sodium tartrate aqueous solution (10 mL), then stirred at room temperature for 1 hour, extracted with ethyl acetate (20 mL*3), washed with saturated brine, dried over anhydrous sodium sulfate, and the solvent was removed by evaporation under reduced pressure to obtain LRQ-06-77 as a light yellow oily liquid, which was directly used in the next reaction step without purification. HRMS (ESI) calculated for C₁₀H₁₆N₃O₂⁺ [M+H]⁺: 210.1237, found: 210.1238.

Step 6: LRQ-06-77 was dissolved in DCM (10 mL), and the reaction mixture was cooled to 0° C. Carbon tetrabromide (432 mg, 1.31 mmol) and triphenylphosphine (456 mg, 1.74 mmol) were sequentially added thereto, and the reaction mixture was reacted at room temperature overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-06-78 (40 mg, two-step reaction yield: 17%) as a yellow solid. ¹H NMR (600 MHz, CDCl₃) δ 3.87 (t, J=6.4 Hz, 2H), 3.83 (s, 3H), 3.72 (t, J=6.6 Hz, 2H), 3.57 (t, J=6.4 Hz, 2H), 2.99 (t, J=6.5 Hz, 2H), 2.55 (s, 3H). HRMS (ESI) calculated for C₁₀H₁₅BrN₃O⁺ [M+H]⁺: 272.0393, found: 272.0391.

Step 7: LRQ-05-137 (22 mg, 0.10 mmol) was dissolved in DMF (8 mL), then DIPEA (78 mg, 0.60 mmol) and LRQ-06-78 (40 mg, 0.15 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a reddish brown solid (6 mg, yield: 14%). ¹H NMR (800 MHz, CDCl₃) δ 7.11 (t, J=7.8 Hz, 1H), 7.02 (d, J=7.7 Hz, 1H), 6.78-6.73 (m, 1H), 4.25 (q, J=7.0 Hz, 2H), 3.79 (s, 2H), 3.74 (s, 3H), 3.72-3.69 (m, 2H), 3.65-3.62 (m, 2H), 2.97-2.91 (m, 2H), 2.88-2.83 (m, 4H), 2.73-2.71 (m, 2H), 2.53 (s, 3H), 1.50 (t, J=7.0 Hz, 3H). HRMS (ESI) calculated for C₂₃H₂₉N₄O₃⁺ [M+H]+: 409.2234, found: 409.2237. HPLC: 95.42% (λ=254 nm, t_{R}=12.10 min).

### Example 40: Preparation of compound 2-(2-(8-ethoxy-3,4-dihydrobenzofuro[2,3-c]pyridin-2(1H)-yl)ethyl)-2,3,4,5-tetrahydro-1H-benzo[c]azepin-1-one (I-A40) (IHCH-5209)

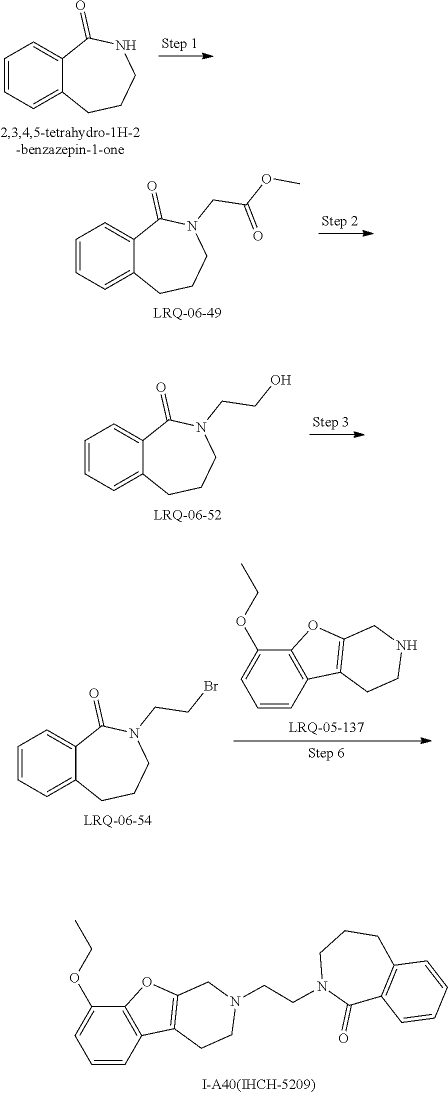

Step 1: 2,3,4,5-Tetrahydro-1H-2-benzazepin-1-one (1.00 g, 6.20 mmol) was dissolved in toluene (10 mL), and the reaction mixture was cooled to 0° C. NaH (290 mg, 7.26 mmol) was added thereto, and the reaction mixture was refluxed for 1 hour. The reaction mixture was then cooled to room temperature, added with methyl bromoacetate (1.11 g, 7.26 mmol), and refluxed overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:2) to obtain LRQ-06-49 (1.20 g, yield: 83%) as a light yellow oily liquid. ¹H NMR (800 MHz, CDCl₃) δ 7.71-7.67 (m, 1H), 7.41-7.37 (m, 1H), 7.35-7.30 (m, 1H), 7.18-7.14 (m, 1H), 4.36 (s, 2H), 3.77 (s, 3H), 3.33-3.25 (m, 2H), 2.94-2.84 (m, 2H), 2.16-2.00 (m, 2H). HRMS (ESI) calculated for C₁₃H₁₆NO₃⁺ [M+H]⁺: 234.1125, found: 234.1127.

Step 2: LRQ-06-49 (1.00 g, 4.30 mmol) was dissolved in THE (20 mL). The reaction mixture was replaced with nitrogen three times, then cooled to −10° C., and DIBAL-H (13 mL, 1.0 M in tetrahydrofuran) was added thereto. The reaction mixture was then warmed to 0° C. and reacted for 6 hours. After the reaction was completed, the reaction mixture was added with saturated potassium sodium tartrate aqueous solution (10 mL), then stirred at room temperature for 1 hour, extracted with ethyl acetate (20 mL*3), washed with saturated brine, dried over anhydrous sodium sulfate, and the solvent was removed by evaporation under reduced pressure to obtain LRQ-06-52 as a light yellow solid, which was directly used in the next reaction step without purification. HRMS (ESI) calculated for C₁₂H₁₆NO₂⁺ [M+H]⁺: 206.1176, found: 206.1180.

Step 3: LRQ-06-52 was dissolved in DCM (10 mL), and the reaction mixture was cooled to 0° C. Carbon tetrabromide (2.14 g, 6.45 mmol) and triphenylphosphine (2.26 g, 8.60 mmol) were sequentially added thereto, and the reaction mixture was reacted at room temperature overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate) to obtain LRQ-06-54 (69 mg, two-step reaction yield: 6%) as a light yellow oily liquid. ¹H NMR (600 MHz, CDCl₃) δ 7.70-7.64 (m, 1H), 7.41-7.36 (m, 1H), 7.36-7.30 (m, 1H), 7.15 (d, J=7.3 Hz, 1H), 4.01-3.92 (m, 2H), 3.69-3.60 (m, 2H), 3.37-3.26 (m, 2H), 2.88-2.79 (m, 2H), 2.15-2.02 (m, 2H). HRMS (ESI) calculated for C₁₂H₁₅BrNO⁺ [M+H]⁺: 268.0332, found: 268.0334.

Step 4: LRQ-05-137 (40 mg, 0.19 mmol) was dissolved in DMF (8 mL), then DIPEA (178 mg, 1.38 mmol) and LRQ-06-54 (69 mg, 0.28 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a reddish brown solid (17 mg, yield: 23%). ¹H NMR (800 MHz, CDCl₃) δ 7.65 (d, J=7.5 Hz, 1H), 7.34 (t, J=7.4 Hz, 1H), 7.30 (t, J=7.5 Hz, 1H), 7.14-7.08 (m, 2H), 7.02 (d, J=7.7 Hz, 1H), 6.76 (d, J=7.9 Hz, 1H), 4.24 (q, J=7.0 Hz, 2H), 3.84-3.80 (m, 2H), 3.79 (s, 2H), 3.24 (t, J=6.4 Hz, 2H), 2.97-2.92 (m, 2H), 2.92-2.87 (m, 2H), 2.79-2.75 (m, 2H), 2.75-2.69 (m, 2H), 2.05-1.98 (m, 2H), 1.50 (t, J=7.0 Hz, 3H). ¹³C NMR (201 MHz, CDCl₃) δ 171.29, 151.23, 144.56, 143.86, 137.68, 136.37, 130.82, 129.87, 128.57, 128.27, 126.96, 123.27, 111.95, 111.16, 107.12, 64.51, 55.78, 50.66, 50.36, 47.37, 45.72, 30.38, 30.32, 21.03, 15.05. HHRMS (ESI) calculated for C₂₅H₂₉N₂O₃⁺ [M+H]⁺: 405.2173, found: 405.2180. HPLC: 96.07% (λ=254 nm, t_{R}=12.48 min).

### Example 41: Preparation of compound 5-(2-(8-ethoxy-3,4-dihydrobenzofuro[2,3-c]pyridin-2(1H)-yl)ethyl)-1-methyl-5,6,7,8-tetrahydropyrrolo[3,2-c]azepin-4(1H)-one (I-A41) (IHCH-5210)

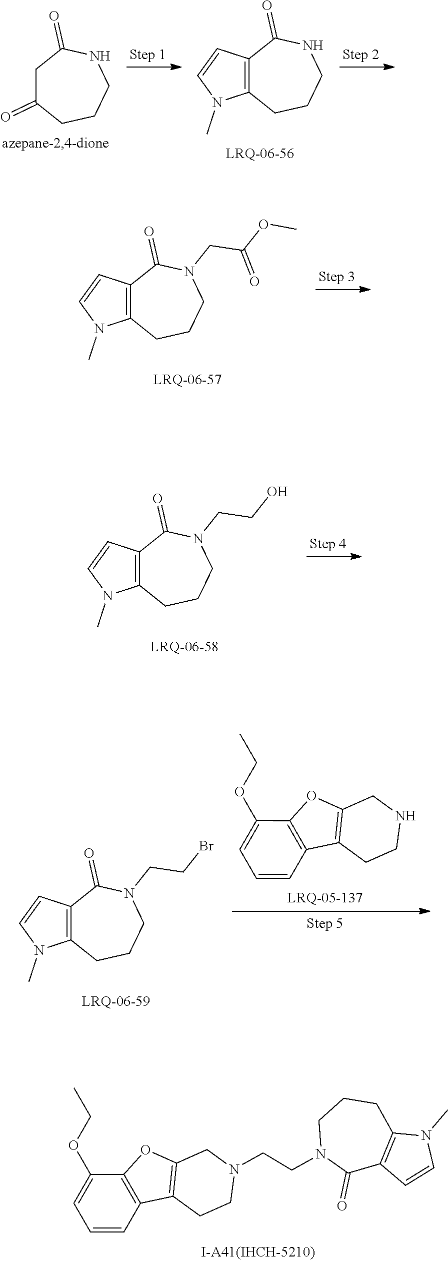

Step 1: Azepane-2,4-dione (1.00 g, 7.86 mmol) was dissolved in N-methylpyrrolidone (20 mL), then methylaminoacetaldehyde dimethyl acetal (9.37 g, 78.65 mmol), methanesulfonic acid (1 mL), and anhydrous magnesium sulfate (5.68 g, 47.16 mmol) were sequentially added thereto, and the reaction mixture was heated to 110° C. and reacted for 1 hour. The reaction mixture was then heated to 150° C. and reacted for 2 hours. After the reaction was completed, the reaction mixture was cooled to room temperature, added with 10% sodium hydroxide aqueous solution to adjust the pH to 9, extracted with dichloromethane (20 mL*3), washed with water, dried over anhydrous sodium sulfate, and the solvent was removed by evaporation under reduced pressure to obtain LRQ-06-56 as a red oily liquid, which was directly used in the next reaction step without purification. HHRMS (ESI) calculated for C₉H₁₃N₂O⁺ [M+H]⁺: 165.1022, found: 165.1026.

Step 2: LRQ-06-56 was dissolved in toluene (10 mL), and the reaction mixture was cooled to 0° C. NaH (368 mg, 9.20 mmol) was added thereto, and the reaction mixture was refluxed for 1 hour. The reaction mixture was then cooled to room temperature, added with methyl bromoacetate (1.41 g, 9.20 mmol), and refluxed overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-06-57 (909 mg, two-step reaction yield: 49%) as a light yellow solid. ¹H NMR (800 MHz, CDCl₃) δ 6.70 (d, J=3.0 Hz, 1H), 6.57-6.54 (m, 1H), 4.33 (s, 2H), 3.73 (s, 3H), 3.54-3.48 (m, 5H), 2.83-2.78 (m, 2H), 2.27-2.18 (m, 2H). HHRMS (ESI) calculated for C₁₂H₁₇N₂O₃⁺ [M+H]⁺: 237.1234, found: 237.1237.

Step 3: LRQ-06-57 (400 mg, 1.69 mmol) was dissolved in THE (20 mL). The reaction mixture was replaced with nitrogen three times, then cooled to −10° C., and DIBAL-H (5 mL, 1.0 M in tetrahydrofuran) was added thereto. The reaction mixture was then warmed to 0° C. and reacted for 6 hours. After the reaction was completed, the reaction mixture was added with saturated potassium sodium tartrate aqueous solution (10 mL), then stirred at room temperature for 1 hour, extracted with ethyl acetate (20 mL*3), washed with saturated brine, dried over anhydrous sodium sulfate, and the solvent was removed by evaporation under reduced pressure to obtain LRQ-06-58 as a light yellow oily liquid, which was directly used in the next reaction step without purification. HRMS (ESI) calculated for C₁₁H₁₇N₂O₂⁺ [M+H]⁺: 209.1285, found: 209.1287.

Step 4: LRQ-06-58 was dissolved in DCM (10 mL), and the reaction mixture was cooled to 0° C. Carbon tetrabromide (841 mg, 2.54 mmol) and triphenylphosphine (887 mg, 3.38 mmol) were sequentially added thereto, and the reaction mixture was reacted at room temperature overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate) to obtain LRQ-06-59 (200 mg, two-step reaction yield: 44%) as a black solid. ¹H NMR (800 MHz, MeOH-d₄) δ 6.88-6.85 (m, 1H), 6.59 (d, J=3.2 Hz, 1H), 4.94-4.89 (m, 2H), 4.26-4.21 (m, 2H), 3.87-3.83 (m, 2H), 3.66 (s, 3H), 3.13-3.05 (m, 2H), 2.33-2.24 (m, 2H). HRMS (ESI) calculated for C₁₁H₁₆BrN₂O⁺ [M+H]⁺: 271.0441, found: 271.0441.

Step 5: LRQ-05-137 (50 mg, 0.23 mmol) was dissolved in DMF (8 mL), then DIPEA (191 mg, 1.48 mmol) and LRQ-06-59 (95 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a light yellow solid (48 mg, yield: 51%). ¹H NMR (800 MHz, CDCl₃) δ 7.10 (t, J=7.8 Hz, 1H), 7.01 (d, J=7.6 Hz, 1H), 6.75 (d, J=7.9 Hz, 1H), 6.67 (d, J=2.9 Hz, 1H), 6.56-6.52 (m, 1H), 4.24 (q, J=7.0 Hz, 2H), 3.80-3.73 (m, 4H), 3.50-3.43 (m, 5H), 2.97-2.90 (m, 2H), 2.86-2.81 (m, 2H), 2.77-2.73 (m, 2H), 2.73-2.66 (m, 2H), 2.14-2.07 (m, 2H), 1.50 (t, J=7.0 Hz, 3H). ¹³C NMR (201 MHz, CDCl₃) δ 166.22, 151.34, 144.54, 143.86, 133.06, 129.93, 123.24, 121.57, 117.23, 111.95, 111.17, 110.79, 107.14, 64.54, 55.46, 50.69, 50.32, 49.26, 47.45, 34.16, 26.60, 26.45, 21.01, 15.06. HRMS (ESI) calculated for C₂₄H₃₀N₃O₃⁺ [M+H]⁺: 408.2282, found: 408.2279. HPLC: 96.12% (λ=254 nm, t_{R}=12.07 min).

### Example 42: Preparation of compound 5-(2-(8-ethoxy-3,4-dihydrobenzofuro[2,3-c]pyridin-2(1H)-yl)ethyl)-1-methyl-5,6,7,8-tetrahydropyrazolo[4,3-c]azepin-4(1H)-one (I-A42) (IHCH-5207)

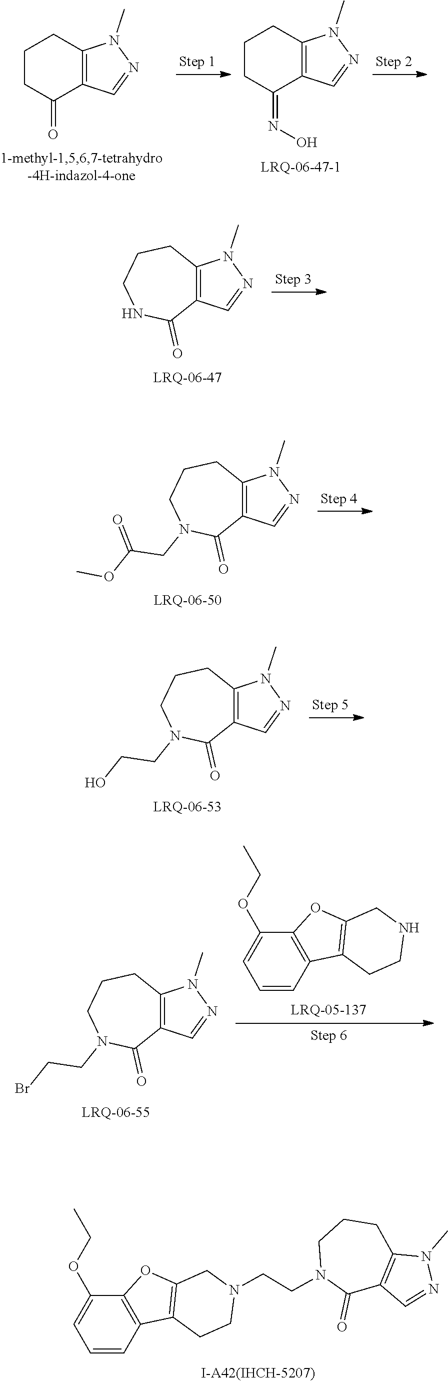

Step 1: 1-Methyl-1,5,6,7-tetrahydro-4H-indazol-4-one (1.00 g, 6.66 mmol), sodium acetate (1.05 g, 4.20 mmol), and hydroxylamine hydrochloride (0.21 g, 0.25 mmol) were dissolved in anhydrous ethanol (16 mL), and the reaction mixture was heated to 60° C. and reacted overnight. After the reaction was completed, the reaction mixture was filtered, and the filtrate was removed by evaporation under reduced pressure to remove the solvent to obtain LRQ-06-47-1 as a white solid, which was directly used in the next reaction step without purification. HRMS (ESI) calculated for C₈H₁₂N₃₀⁺ [M+H]⁺: 166.0975, found: 166.0977.

Step 2: LRQ-06-47-1 and triethylamine (2.02 g, 19.98 mmol) were dissolved in DCM (20 mL), then p-toluenesulfonyl chloride (1.46 g, 7.66 mmol) was added thereto, and the reaction mixture was refluxed for 0.5 hours. After the reaction was completed, the reaction mixture was diluted with saturated sodium bicarbonate aqueous solution (10 mL), extracted with ethyl acetate (50 mL*3), washed with saturated brine, dried over anhydrous sodium sulfate, and the solvent was removed by evaporation under reduced pressure to obtain a yellow solid, which was then dissolved in trifluoroacetic acid (5 mL), and the reaction mixture was refluxed for 0.5 hours. After the reaction was completed, the solvent was removed by evaporation under reduced pressure. The reaction mixture was diluted with saturated sodium bicarbonate aqueous solution (10 mL), extracted with dichloromethane (50 mL*3), washed with water, dried over anhydrous sodium sulfate, and the solvent was removed by evaporation under reduced pressure to obtain LRQ-04-47 as a yellow solid, which was directly used in the next reaction step without purification. HRMS (ESI) calculated for C₈H₁₂N₃₀⁺ [M+H]⁺: 166.0975, found: 166.0975.

Step 3: LRQ-04-47 was dissolved in toluene (10 mL), and the reaction mixture was cooled to 0° C. NaH (311 mg, 7.79 mmol) was added thereto, and the reaction mixture was refluxed for 1 hour. The reaction mixture was then cooled to room temperature, added with methyl bromoacetate (1.19 g, 7.79 mmol), and refluxed overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-06-50 (620 mg, three-step reaction yield: 39%) as a light yellow solid. ¹H NMR (800 MHz, CDCl₃) δ 8.16 (s, 1H), 4.71 (s, 2H), 3.80 (s, 3H), 3.77 (s, 3H), 2.71 (t, J=6.2 Hz, 2H), 2.55-2.45 (m, 2H), 2.08-1.98 (m, 2H) HHRMS (ESI) calculated for C₁₁H₁₆N₃O₃⁺ [M+H]⁺: 238.1186, found: 238.1187.

Step 4: LRQ-06-50 (600 mg, 2.53 mmol) was dissolved in THE (5 mL). The reaction mixture was replaced with nitrogen three times, then cooled to −10° C., and DIBAL-H (8 mL, 1.0 M in tetrahydrofuran) was added thereto. The reaction mixture was then warmed to 0° C. and reacted for 6 hours. After the reaction was completed, the reaction mixture was added with saturated potassium sodium tartrate aqueous solution (10 mL), then stirred at room temperature for 1 hour, extracted with ethyl acetate (20 mL*3), washed with saturated brine, dried over anhydrous sodium sulfate, and the solvent was removed by evaporation under reduced pressure to obtain LRQ-06-53 as a light yellow solid, which was directly used in the next reaction step without purification. HHRMS (ESI) calculated for C₁₀H₁₆N₃O₂⁺ [M+H]⁺: 210.1237, found: 210.1242.

Step 5: LRQ-06-53 was dissolved in DCM (10 mL), and the reaction mixture was cooled to 0° C. Carbon tetrabromide (1.26 g, 3.79 mmol) and triphenylphosphine (1.33 g, 5.06 mmol) were sequentially added thereto, and the reaction mixture was reacted at room temperature overnight. After the reaction was completed, the reaction mixture was diluted with water (10 mL), extracted with ethyl acetate (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain LRQ-06-55 (261 mg, two-step reaction yield: 38%) as a colorless oily liquid. ¹H NMR (800 MHz, MeOH-d₄) δ 8.03 (s, 1H), 5.03-4.97 (m, 2H), 4.31-4.25 (m, 2H), 3.92-3.90 (m, 2H), 3.89 (s, 3H), 3.20-3.15 (m, 2H), 2.34-2.27 (m, 2H). HRMS (ESI) calculated for C₁₀H₁₅BrN₃O⁺ [M+H]⁺: 272.0393, found: 272.0397.

Step 6: LRQ-05-137 (50 mg, 0.23 mmol) was dissolved in DMF (8 mL), then DIPEA (191 mg, 1.48 mmol) and LRQ-06-55 (93 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a light yellow solid (29 mg, yield: 31%). ¹H NMR (800 MHz, CDCl₃) δ 7.97-7.94 (m, 1H), 7.10 (t, J=7.8 Hz, 1H), 7.03-6.99 (m, 1H), 6.76 (d, J=7.9 Hz, 1H), 4.27-4.21 (m, 2H), 3.78-3.73 (m, 7H), 3.52-3.47 (m, 2H), 2.96-2.91 (m, 2H), 2.87-2.82 (m, 2H), 2.82-2.76 (m, 2H), 2.74-2.69 (m, 2H), 2.16-2.12 (m, 2H), 1.50 (t, J=7.0 Hz, 3H). ¹³C NMR (201 MHz, CDCl₃) δ 164.25, 151.44, 144.57, 143.88, 142.11, 140.80, 129.81, 123.34, 116.31, 111.95, 111.17, 107.20, 64.54, 55.23, 50.64, 50.28, 49.29, 47.17, 36.68, 26.00, 25.79, 20.89, 15.05. HRMS (ESI) calculated for C₂₃H₂₉N₄O₃⁺ [M+H]⁺: 409.2234, found: 409.2238. HPLC: 95.13% (Q=254 nm, t_{R}=12.08 min).

### Example 43: Preparation of compound 2-(4-(8-ethoxy-3,4-dihydrobenzofuro[2,3-c]pyridin-2(1H)-yl)butyl)-4-methyl-1,2,4-triazine-3,5(2H,4H)-dione (I-A43) (IHCH-5196)

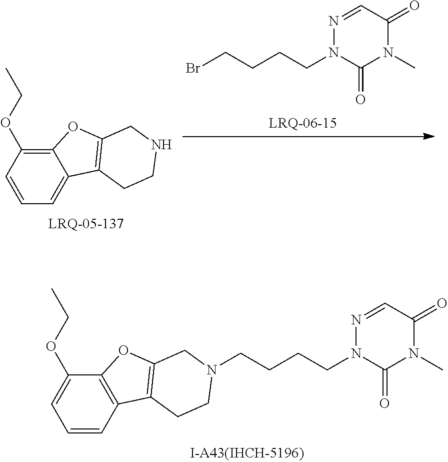 [SMILES: CCOc1cccc2c3c(oc12)CNCC3 ; Cn1c(=O)cnn(CCCCBr)c1=O ; CCOc1cccc2c3c(oc12)CN(CCCCn1ncc(=O)n(C)c1=O)CC3]

LRQ-05-137 (50 mg, 0.23 mmol) was dissolved in DMF (8 mL), then DIPEA (178 mg, 1.38 mmol) and LRQ-06-15 (92 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a light yellow solid (52 mg, yield: 56%). ¹H NMR (800 MHz, CDCl₃) δ 7.38 (s, 1H), 7.10 (t, J=7.8 Hz, 1H), 7.01 (d, J=7.7 Hz, 1H), 6.79-6.71 (m, 1H), 4.24 (q, J=7.0 Hz, 2H), 4.04-4.00 (m, 2H), 3.69 (s, 2H), 3.33 (s, 3H), 2.86-2.81 (m, 2H), 2.74-2.69 (m, 2H), 2.67-2.62 (m, 2H), 1.87-1.78 (m, 2H), 1.67-1.60 (m, 2H), 1.50 (t, J=7.0 Hz, 3H). ¹³C NMR (201 MHz, CDCl₃) δ 156.31, 151.21, 148.94, 144.56, 143.90, 133.88, 129.85, 123.26, 111.84, 111.14, 107.14, 64.51, 56.83, 51.76, 50.54, 50.27, 27.08, 26.18, 24.41, 20.93, 15.06. HRMS (ESI) calculated for C₂₁H₂₇N₄O₄⁺[M+H]⁺: 399.2027, found: 399.2023. HPLC: 98.26% (λ=254 nm, t_{R}=11.67 min).

### Example 44: Preparation of compound 2-(3-(8-ethoxy-3,4-dihydrobenzofuro[2,3-c]pyridin-2(1H)-yl)propyl)-4-methyl-1,2,4-triazine-3,5(2H,4H)-dione (I-A44) (IHCH-5197)

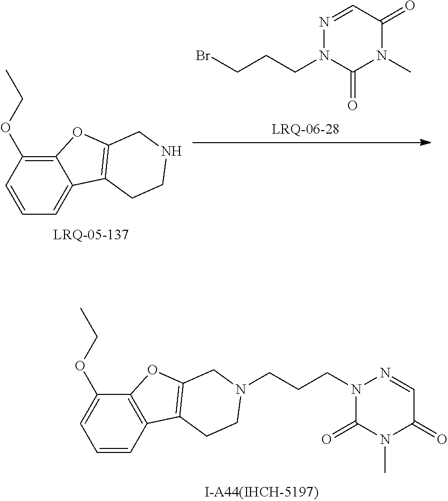 [SMILES: CCOc1cccc2c3c(oc12)CNCC3 ; Cn1c(=O)cnn(CCCBr)c1=O ; CCOc1cccc2c3c(oc12)CN(CCCn1ncc(=O)n(C)c1=O)CC3]

Step 1: LRQ-05-137 (50 mg, 0.23 mmol) was dissolved in DMF (8 mL), then DIPEA (178 mg, 1.38 mmol) and LRQ-06-28 (86 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a light yellow solid (10 mg, yield: 11%). ¹H NMR (800 MHz, MeOH-d₄) δ 7.41 (s, 1H), 7.09 (t, J=7.8 Hz, 1H), 7.02-6.99 (m, 1H), 6.81 (d, J=7.8 Hz, 1H), 4.21 (q, J=7.0 Hz, 2H), 4.12-4.06 (m, 2H), 3.68 (s, 2H), 3.19 (s, 3H), 2.90-2.82 (m, 2H), 2.78-2.72 (m, 2H), 2.72-2.66 (m, 2H), 2.09-2.03 (m, 2H), 1.44 (t, J=7.0 Hz, 3H). ¹³C NMR (201 MHz, MeOH-d₄) δ 158.04, 151.64, 150.58, 145.84, 145.36, 134.97, 130.80, 124.46, 112.76, 112.09, 108.96, 65.64, 55.68, 51.42, 51.31, 50.92, 27.04, 26.36, 21.60, 15.28. HRMS (ESI) calculated for C₂₀H₂₅N₄O₄⁺ [M+H]⁺: 385.1870, found: 385.1864. HPLC: 98.94% (λ=254 nm, t_{R}=11.81 min).

### Example 45: Preparation of compound 1-(4-(8-ethoxy-3,4-dihydrobenzofuro[2,3-c]pyridin-2(1H)-yl)butyl)-4,4-dimethylpiperidine-2,6-dione (I-A45) (IHCH-5191)

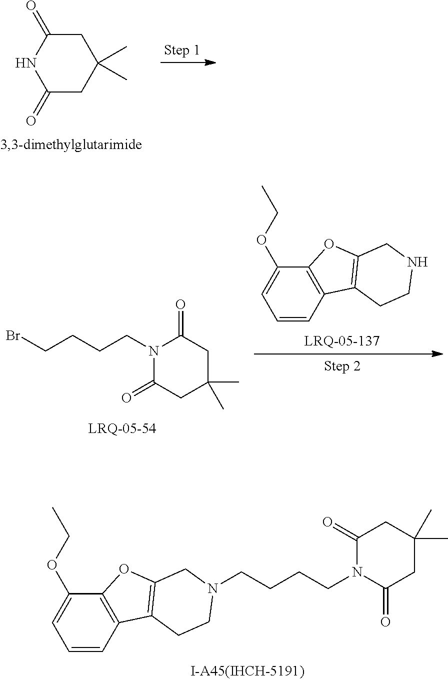

Step 1: 3,3-Dimethylglutarimide (1.00 g, 7.08 mmol) was dissolved in DMF (10 mL), then K₂CO₃ (0.98 g, 7.08 mmol) and 1,4-dibromobutane (4.59 g, 21.25 mmol) were sequentially added thereto, and the reaction mixture was reacted at room temperature overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:4) to obtain LRQ-05-54 (1.60 g, yield: 82%) as a light yellow oily liquid. ¹H NMR (800 MHz, CDCl₃) δ 3.79 (t, J=7.3 Hz, 2H), 3.41 (t, J=6.7 Hz, 2H), 2.50 (s, 4H), 1.88-1.81 (m, 2H), 1.71-1.64 (m, 2H), 1.07 (s, 6H). HHRMS (ESI) calculated for C₁₁H₁₉BrNO₂⁺ [M+H]⁺: 276.0594, found: 276.0590.

Step 2: LRQ-05-137 (50 mg, 0.23 mmol) was dissolved in DMF (8 mL), then DIPEA (178 mg, 1.38 mmol) and LRQ-05-54 (97 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a light yellow solid (60 mg, yield: 63%). ¹H NMR (800 MHz, MeOH-d₄) δ 7.12 (t, J=7.8 Hz, 1H), 7.07-7.01 (m, 1H), 6.84 (d, J=7.9 Hz, 1H), 4.22 (q, J=7.0 Hz, 2H), 3.84 (s, 2H), 3.83-3.79 (m, 2H), 3.07-2.99 (m, 2H), 2.84-2.75 (m, 4H), 2.56 (s, 4H), 1.72-1.62 (m, 2H), 1.62-1.54 (m, 2H), 1.44 (t, J=7.0 Hz, 3H), 1.06 (s, 6H). ¹³C NMR (201 MHz, MeOH-d₄) δ 174.20 (2C), 146.78, 146.02, 145.80, 129.72, 125.16, 112.75, 112.31, 109.67, 65.65, 57.23, 51.60, 49.91, 46.81 (2C), 39.43, 30.00, 27.69 (2C), 26.22, 23.44, 19.65, 15.23. HRMS (ESI) calculated for C₂₄H₃₃N₂O₄⁺ [M+H]⁺: 413.2435, found: 413.2433. HPLC: 98.10% (λ=254 nm, t_{R}=12.32 min).

### Example 46: Preparation of compound 8-(4-(8-ethoxy-3,4-dihydrobenzofuro[2,3-c]pyridin-2(1H)-yl)butyl)-8-azaspiro[4.5]decane-7,9-dione (I-A46) (IHCH-5192)

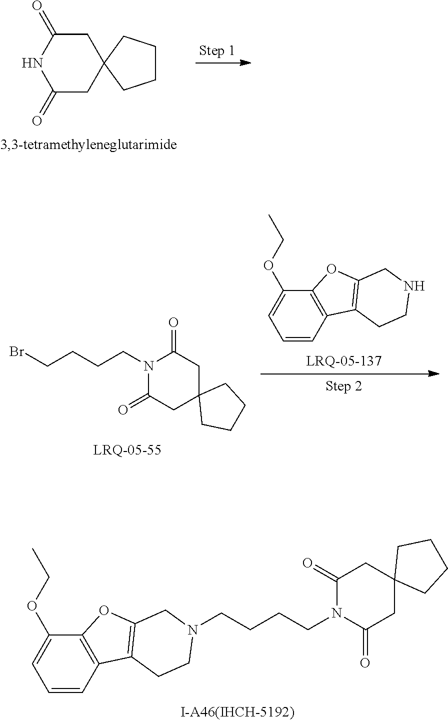 [SMILES: O=C1CC2(CCCC2)CC(=O)N1 ; O=C1CC2(CCCC2)CC(=O)N1CCCCBr ; CCOc1cccc2c3c(oc12)CNCC3 ; CCOc1cccc2c3c(oc12)CN(CCCCN1C(=O)CC2(CCCC2)CC1=O)CC3]

Step 1: 3,3-Tetramethyleneglutarimide (1.00 g, 5.98 mmol) was dissolved in DMF (10 mL), then K₂CO₃ (0.83 g, 5.98 mmol) and 1,4-dibromobutane (3.87 g, 17.94 mmol) were sequentially added thereto, and the reaction mixture was reacted at room temperature overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:4) to obtain LRQ-05-55 (1.60 g, yield: 88%) as a light yellow oily liquid. ¹H NMR (800 MHz, CDCl₃) δ 3.79 (t, J=7.3 Hz, 2H), 3.41 (t, J=6.7 Hz, 2H), 2.59 (s, 4H), 1.87-1.78 (m, 2H), 1.74-1.63 (m, 6H), 1.52-1.43 (m, 4H). HRMS (ESI) calculated for C₁₃H₂₁BrNO₂⁺ [M+H]⁺: 302.0750, found: 302.0752.

Step 2: LRQ-05-137 (50 mg, 0.23 mmol) was dissolved in DMF (8 mL), then DIPEA (178 mg, 1.38 mmol) and LRQ-05-55 (110 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a light yellow solid (42 mg, yield: 41%). ¹H NMR (800 MHz, MeOH-d₄) δ 7.17 (t, J=7.9 Hz, 1H), 7.12-7.06 (m, 1H), 6.90 (d, J=7.8 Hz, 1H), 4.31 (s, 2H), 4.22 (q, J=7.0 Hz, 2H), 3.84-3.78 (m, 2H), 3.50-3.44 (m, 2H), 3.23-3.18 (m, 2H), 3.00-2.95 (m, 2H), 2.64 (s, 4H), 1.81-1.74 (m, 2H), 1.74-1.68 (m, 4H), 1.65-1.59 (m, 2H), 1.53-1.47 (m, 4H), 1.44 (t, J=7.0 Hz, 3H). ¹³C NMR (201 MHz, MeOH-d₄) δ 174.46 (2C), 146.85, 146.04, 145.83, 129.74, 125.15, 112.75, 112.30, 109.67, 65.66, 57.25, 51.61, 49.93, 45.39 (2C), 40.61, 39.48, 38.40 (2C), 26.26, 25.15 (2C), 23.44, 19.68, 15.23. HRMS (ESI) calculated for C₂₆H₃₅N₂O₄⁺ [M+H]⁺: 439.2591, found: 439.2587. HPLC: 97.68% (λ=254 nm, t_{R}=12.64 min).

### Example 47: Preparation of compound 1-(2-(-ethoxy-3,4-dihydrobenzofuro[2,3-c]pyridin-2(1H)-yl)ethyl)-1,3-dihydro-2H-benzo[d]imidazol-2-one (I-A47) (IHCH-5224)

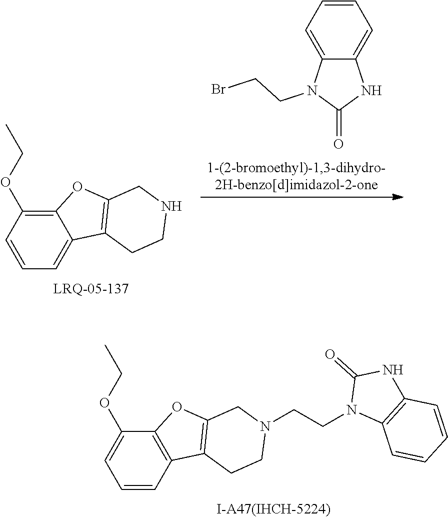 [SMILES: CCOc1cccc2c3c(oc12)CNCC3 ; O=c1[nH]c2ccccc2n1CCBr ; CCOc1cccc2c3c(oc12)CN(CCn1c(=O)[nH]c2ccccc21)CC3]

LRQ-05-137 (50 mg, 0.23 mmol) was dissolved in DMF (8 mL), then DIPEA (178 mg, 1.38 mmol) and 1-(2-bromoethyl)-1,3-dihydro-2H-benzo[d]imidazol-2-one (84 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (27 mg, yield: 31%). ¹H NMR (800 MHz, CDCl₃) δ 9.68 (s, 1H), 7.14-7.03 (m, 5H), 7.00 (d, J=7.7 Hz, 1H), 6.76 (d, J=7.9 Hz, 1H), 4.25 (q, J=7.0 Hz, 2H), 4.15-4.09 (m, 2H), 3.85 (s, 2H), 3.04-2.95 (m, 4H), 2.75-2.69 (m, 2H), 1.50 (t, J=7.0 Hz, 3H). ¹³C NMR (201 MHz, CDCl₃) δ 155.54, 150.23, 144.58, 143.92, 130.40, 129.81, 128.05, 123.32, 121.69, 121.50, 111.92, 111.19, 109.80, 108.00, 107.25, 64.56, 54.61, 50.55, 50.37, 39.14, 20.81, 15.08. HRMS (ESI) calculated for C₂₂H₂₄N₃₀₃₊[M+H]⁺: 378.1812, found: 378.1815. HPLC: 96.61% (λ=254 nm, t_{R}=11.84 min).

### Example 48: Preparation of compound 7-(4-(8-methoxy-1,3,4,9-tetrahydro-2H-pyrido[3,4-b]indol-2-yl)butoxy)quinolin-2(1H)-one (I-B1)

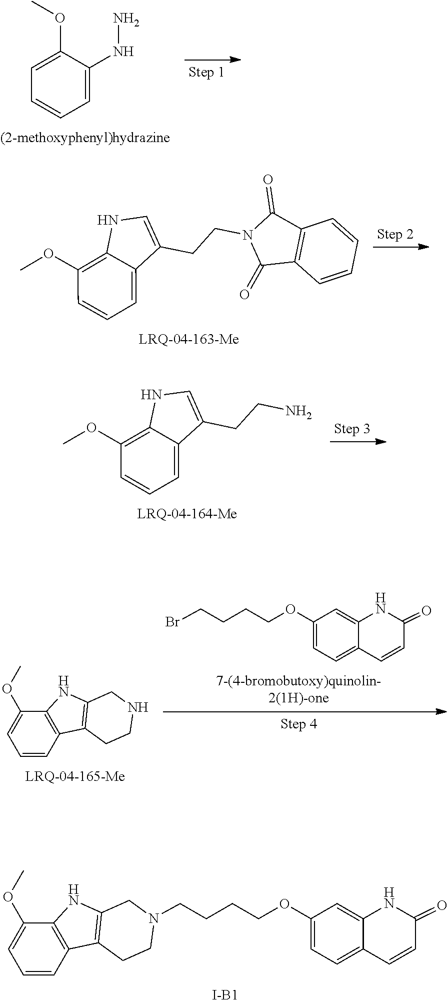

Steps 1, 2, and 3: LRQ-04-165-Me was synthesized in the same way as in U.S. Pat. No. 5,631,265 A 19970520. LRQ-04-165-Me was a yellow solid (three-step reaction yield: 17%). ¹H NMR (800 MHz, MeOH-d₄) δ 7.05 (d, J=7.9 Hz, 1H), 6.97 (t, J=7.8 Hz, 1H), 6.69-6.66 (m, 1H), 4.34 (s, 2H), 3.94 (s, 3H), 3.51-3.47 (m, 2H), 3.02-2.99 (m, 2H). HRMS (ESI) calculated for C₁₂H₁₅N₂O⁺ [M+H]⁺: 203.1179, found: 203.1185.

Step 4: LRQ-04-165-Me (99 mg, 0.49 mmol) was dissolved in DMF (8 mL), then DIPEA (189 mg, 1.47 mmol) and 7-(4-bromobutoxy)quinolin-2(1H)-one (218 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (72 mg, yield: 35%). ¹H NMR (800 MHz, MeOH-d₄) δ 7.88 (d, J=9.4 Hz, 1H), 7.56 (d, J=8.7 Hz, 1H), 7.04 (d, J=7.8 Hz, 1H), 6.95 (t, J=7.8 Hz, 1H), 6.90-6.87 (m, 1H), 6.86 (d, J=2.3 Hz, 1H), 6.66 (d, J=7.6 Hz, 1H), 6.44 (d, J=9.4 Hz, 1H), 4.21 (s, 2H), 4.16 (t, J=5.9 Hz, 2H), 3.94 (s, 3H), 3.42-3.35 (m, 2H), 3.18-3.11 (m, 2H), 3.00 (t, J=5.8 Hz, 2H), 2.03-1.98 (m, 2H), 1.98-1.92 (m, 2H). ¹³C NMR (201 MHz, MeOH-d₄) δ 163.98, 161.27, 147.76, 146.20, 140.61, 132.22, 131.32, 128.54, 125.51, 121.31, 117.88, 117.00, 113.53, 111.82, 107.15, 103.71, 100.27, 69.10, 56.84, 55.84, 52.40, 50.43, 27.21, 22.62, 19.84. HRMS (ESI) calculated for C₂₅H₂₈N₃O₃⁺ [M+H]⁺: 418.2125, found: 418.2126. HPLC: 97.90% (λ254 nm, t_{R}=11.51 min).

### Example 49: Preparation of compound 7-(4-(8-ethoxy-1,3,4,9-tetrahydro-2H-pyrido[3,4-b]indol-2-yl)butoxy)quinolin-2(1H)-one (I-B2)

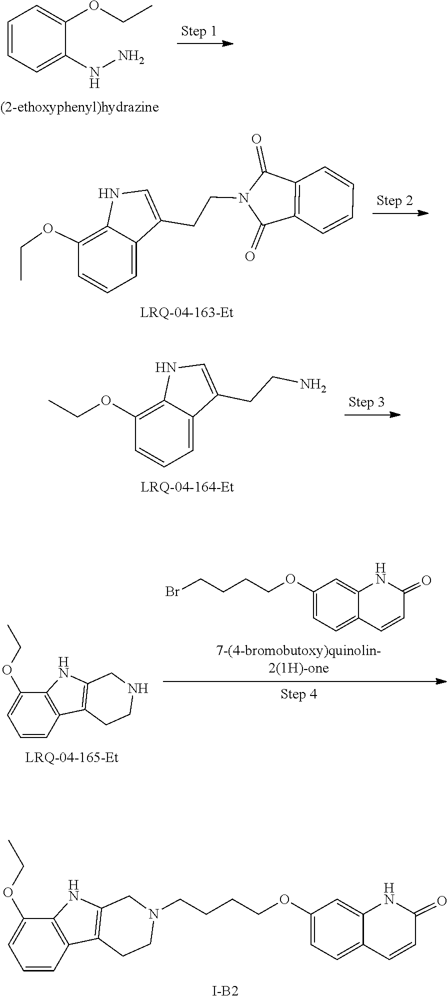 [SMILES: CCOc1ccccc1NN ; CCOc1cccc2c(CCN3C(=O)c4ccccc4C3=O)c[nH]c12 ; CCOc1cccc2c(CCN)c[nH]c12 ; CCOc1cccc2c3c([nH]c12)CNCC3 ; O=c1ccc2ccc(OCCCCBr)cc2[nH]1 ; CCOc1cccc2c3c([nH]c12)CN(CCCCOc1ccc2ccc(=O)[nH]c2c1)CC3]

Steps 1, 2, and 3: LRQ-04-165-Et was synthesized in the same way as in U.S. Pat. No. 5,631,265 A 19970520, using (2-ethoxyphenyl)hydrazine as the starting material. LRQ-04-165-Et was a yellow solid (three-step reaction yield: 16%). ¹H NMR (800 MHz, MeOH-d₄) δ 7.01 (d, J=7.8 Hz, 1H), 6.90 (t, J=7.8 Hz, 1H), 6.62 (d, J=7.6 Hz, 1H), 4.18 (q, J=7.0 Hz, 2H), 4.13 (s, 2H), 3.30-3.24 (m, 2H), 2.89-2.82 (m, 2H), 1.47 (t, J=7.0 Hz, 3H). HRMS (ESI) calculated for C₁₃H₁₇N₂O⁺ [M+H]⁺: 217.1335, found: 217.1337.

Step 4: LRQ-04-165-Et (105 mg, 0.49 mmol) was dissolved in DMF (8 mL), then DIPEA (189 mg, 1.47 mmol) and 7-(4-bromobutoxy)quinolin-2(1H)-one (218 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (36 mg, yield: 17%). ¹H NMR (800 MHz, MeOH-d₄) δ 7.88 (d, J=9.4 Hz, 1H), 7.57 (d, J=8.7 Hz, 1H), 7.04 (d, J=7.8 Hz, 1H), 6.95 (t, J=7.8 Hz, 1H), 6.90-6.87 (m, 1H), 6.86 (d, J=2.3 Hz, 1H), 6.67 (d, J=7.6 Hz, 1H), 6.44 (d, J=9.4 Hz, 1H), 4.42-4.29 (m, 2H), 4.20-4.16 (m, 4H), 3.58-3.44 (m, 2H), 3.35-3.32 (m, 1H), 3.08-3.02 (m, 2H), 2.08-2.02 (m, 2H), 1.99-1.94 (m, 2H), 1.47 (t, J=7.0 Hz, 3H). ¹³C NMR (201 MHz, MeOH-d₄) δ 163.68, 160.60, 146.94, 141.94, 140.79, 131.12, 129.86, 126.99, 125.49, 121.26, 116.30, 115.24, 111.71, 107.11, 104.68, 104.49, 100.22, 68.98, 64.75, 56.89, 52.42, 50.45, 27.20, 22.60, 19.85, 15.31. HRMS (ESI) calculated for C₂₆H₃₀N₃O₃⁺ [M+H]⁺: 432.2282, found: 432.2284. HPLC: 97.97% (λ=254 nm, t_{R}=11.06 min).

### Example 50: Preparation of compound 7-(3-(8-methoxy-1,3,4,9-tetrahydro-2H-pyrido[3,4-b]indol-2-yl)propoxy)quinolin-2(1H)-one (I-B3)

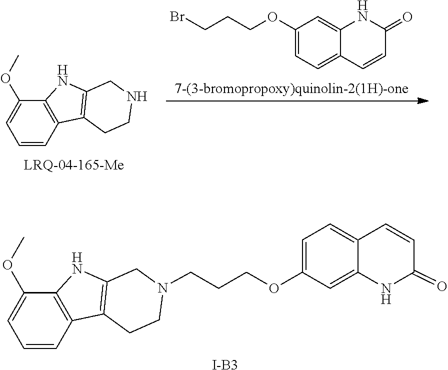

LRQ-04-165-Me (99 mg, 0.49 mmol) was dissolved in DMF (8 mL), then DIPEA (189 mg, 1.47 mmol) and 7-(3-bromopropoxy)quinolin-2(1H)-one (208 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a reddish brown solid (98 mg, yield: 50%). ¹H NMR (800 MHz, MeOH-d₄) δ 7.78 (d, J=9.4 Hz, 1H), 7.48 (d, J=8.7 Hz, 1H), 7.03 (d, J=7.8 Hz, 1H), 6.94 (t, J=7.8 Hz, 1H), 6.85-6.82 (m, 1H), 6.80 (d, J=1.9 Hz, 1H), 6.62-6.58 (m, 1H), 6.43 (d, J=9.3 Hz, 1H), 4.13 (t, J=5.9 Hz, 2H), 3.92 (s, 3H), 3.85 (s, 2H), 3.02 (s, 2H), 2.94-2.90 (m, 2H), 2.89-2.84 (m, 2H), 2.21-2.14 (m, 2H). ¹³C NMR (201 MHz, MeOH-d₄) δ 165.07, 161.63, 145.22, 141.96, 141.35, 134.23, 129.88, 128.79, 127.34, 120.01, 117.95, 116.83, 113.25, 111.31, 107.91, 102.36, 99.37, 66.92, 55.62, 54.80, 51.77, 50.82, 27.04, 21.36. HRMS (ESI) calculated for C₂₄H₂₆N₃O₃⁺ [M+H]⁺: 404.1969, found: 404.1970. HPLC: 99.24% (λ=254 nm, t_{R}=11.88 min).

### Example 51: Preparation of compound 7-(3-(8-ethoxy-1,3,4,9-tetrahydro-2H-pyrido[3,4-b]indol-2-yl)propoxy)quinolin-2(1H)-one (I-B4)

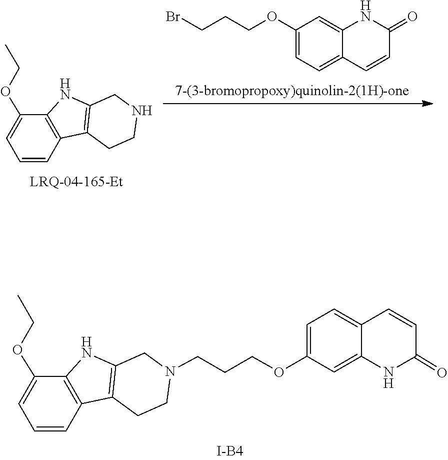 [SMILES: CCOc1cccc2c3c([nH]c12)CNCC3 ; O=c1ccc2ccc(OCCCBr)cc2[nH]1 ; CCOc1cccc2c3c([nH]c12)CN(CCCOc1ccc2ccc(=O)[nH]c2c1)CC3]

LRQ-04-165-Et (105 mg, 0.49 mmol) was dissolved in DMF (8 mL), then DIPEA (189 mg, 1.47 mmol) and 7-(3-bromopropoxy)quinolin-2(1H)-one (208 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (98 mg, yield: 48%). ¹H NMR (800 MHz, MeOH-d₄) δ 7.75 (d, J=9.4 Hz, 1H), 7.46 (d, J=8.7 Hz, 1H), 7.03 (d, J=7.8 Hz, 1H), 6.92 (t, J=7.8 Hz, 1H), 6.83-6.80 (m, 1H), 6.78 (d, J=2.2 Hz, 1H), 6.58 (d, J=7.7 Hz, 1H), 6.43 (d, J=9.4 Hz, 1H), 4.19-4.14 (m, 2H), 4.11 (t, J=6.1 Hz, 2H), 3.83 (s, 2H), 3.00 (t, J=5.5 Hz, 2H), 2.92-2.87 (m, 2H), 2.86 (t, J=5.8 Hz, 2H), 2.21-2.10 (m, 2H), 1.45 (t, J=7.0 Hz, 3H). ¹³C NMR (201 MHz, MeOH-d₄) δ 164.87, 161.75, 145.78, 141.75, 140.52, 133.40, 129.69, 128.67, 127.12, 119.89, 117.84, 114.91, 113.11, 111.11, 107.85, 103.28, 99.21, 66.79, 64.12, 54.53, 51.51, 50.64, 26.91, 21.18, 15.15. HRMS (ESI) calculated for C₂₅H₂₈N₃O₃⁺ [M+H]⁺: 418.2125, found: 418.2124. HPLC: 96.54% (Q=254 nm, t_{R}=12.22 min).

### Example 52: Preparation of compound 7-(3-(8-methoxy-1,3,4,9-tetrahydro-2H-pyrido[3,4-b]indol-2-yl)propoxy)-3,4-dihydroquinolin-2(1H)-one (I-B5)

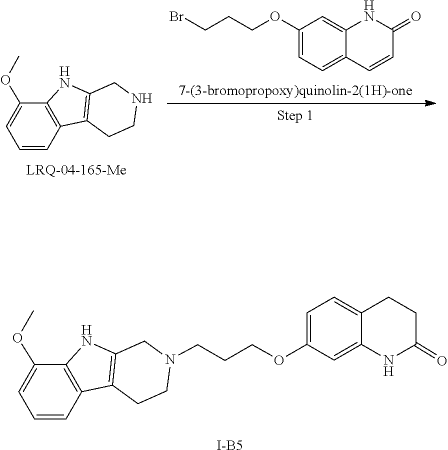

LRQ-04-165-Me (99 mg, 0.49 mmol) was dissolved in DMF (8 mL), then DIPEA (189 mg, 1.47 mmol) and 7-(3-bromopropoxy)-3,4-dihydroquinolin-2(1H)-one (209 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (75 mg, yield: 38%). ¹H NMR (800 MHz, MeOH-d₄) δ 7.04 (d, J=7.8 Hz, 1H), 7.00 (d, J=8.3 Hz, 1H), 6.95 (t, J=7.8 Hz, 1H), 6.59 (d, J=7.7 Hz, 1H), 6.51-6.47 (m, 1H), 6.38 (d, J=2.3 Hz, 1H), 3.99 (t, J=6.0 Hz, 2H), 3.91 (s, 3H), 3.87 (s, 2H), 3.03 (t, J=5.4 Hz, 2H), 2.92-2.88 (m, 2H), 2.87 (t, J=5.6 Hz, 2H), 2.84 (t, J=7.6 Hz, 2H), 2.56-2.49 (m, 2H), 2.14-2.06 (m, 2H). ¹³C NMR (201 MHz, MeOH-d₄) δ 172.96, 158.67, 146.41, 138.49, 136.51, 128.89, 128.38, 126.80, 119.89, 116.31, 111.13, 109.23, 105.43, 102.57, 102.24, 66.38, 55.53, 54.33, 51.30, 50.39, 31.15, 26.74, 24.68, 20.79. HRMS (ESI) calculated for C₂₄H₂₈N₃O₃⁺ [M+H]⁺: 406.2125, found: 406.2123. HPLC: 96.96% (λ=254 nm, t_{R}=12.32 min).

### Example 53: Preparation of compound 7-(3-(8-ethoxy-1,3,4,9-tetrahydro-2H-pyrido[3,4-b]indol-2-yl)propoxy)-3,4-dihydroquinolin-2(1H)-one (I-B6)

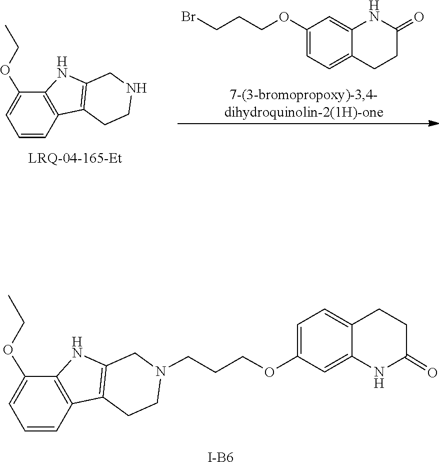

LRQ-04-165-Et (105 mg, 0.49 mmol) was dissolved in DMF (8 mL), then DIPEA (189 mg, 1.47 mmol) and 7-(3-bromopropoxy)-3,4-dihydroquinolin-2(1H)-one (209 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a reddish brown solid (84 mg, yield: 41%). ¹H NMR (800 MHz, CDCl₃) δ 8.18 (s, 1H), 8.09 (s, 1H), 7.07 (d, J=7.9 Hz, 1H), 7.02 (d, J=8.3 Hz, 1H), 6.98 (t, J=7.8 Hz, 1H), 6.61 (d, J=7.7 Hz, 1H), 6.54-6.50 (m, 1H), 6.33-6.29 (m, 1H), 4.19 (q, J=7.0 Hz, 2H), 4.02 (t, J=6.3 Hz, 2H), 3.76 (s, 2H), 2.93 (t, J=5.4 Hz, 2H), 2.87 (t, J=7.5 Hz, 2H), 2.83 (t, J=5.5 Hz, 2H), 2.81 (t, J=7.2 Hz, 2H), 2.62-2.58 (m, 2H), 2.10-2.05 (m, 2H), 1.46 (t, J=7.0 Hz, 3H). ¹³C NMR (201 MHz, CDCl₃) δ 171.78, 158.69, 145.27, 138.24, 136.41, 128.78, 128.56, 126.55, 122.29, 119.92, 115.92, 110.94, 108.90, 102.89, 102.29, 66.44, 63.73, 54.11, 51.18, 50.51, 31.23, 29.84, 27.26, 24.72, 15.19. HRMS (ESI) calculated for C₂₅H₃₀N₃O₃⁺ [M+H]⁺: 420.2282, found: 420.2283. HPLC: 99.77% (Q=254 nm, t_{R}=12.32 min).

### Example 54: Preparation of compound 3-(trans-4-(2-(8-methoxy-1,3,4,9-tetrahydro-2H-pyrido[3,4-b]indol-2-yl)ethyl)cyclohexyl)-1,1-dimethylurea (I-B7)

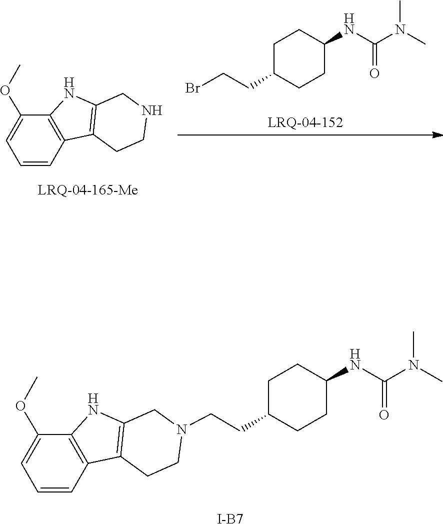

LRQ-04-165-Me (24 mg, 0.12 mmol) was dissolved in DMF (8 mL), then DIPEA (45 mg, 0.35 mmol) and LRQ-04-152 (47 mg, 0.17 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a white solid (13 mg, yield: 27%). ¹H NMR (800 MHz, MeOH-d₄) δ 7.03-6.98 (m, 1H), 6.91 (t, J=7.8 Hz, 1H), 6.62 (d, J=7.7 Hz, 1H), 3.93 (s, 3H), 3.84-3.74 (m, 2H), 3.54-3.46 (m, 1H), 3.02-2.96 (m, 2H), 2.91-2.82 (m, 8H), 2.80-2.70 (m, 2H), 1.94-1.88 (m, 2H), 1.87-1.82 (m, 2H), 1.62-1.55 (m, 2H), 1.35-1.31 (m, 1H), 1.30-1.26 (m, 2H), 1.16-1.07 (m, 2H). ¹³C NMR (201 MHz, MeOH-d₄) δ 153.70, 144.70, 134.33, 129.52, 128.07, 120.52, 115.01, 111.67, 108.14, 56.78, 55.75, 52.52, 51.37, 51.18, 36.72, 36.43 (2C), 35.41, 34.29 (2C), 33.35 (2C), 21.71. HRMS (ESI) calculated for C₂₃H₃₅N₄O₂⁺ [M+H]⁺: 399.2755, found: 399.2752. HPLC: 97.46% (λ254 nm, t_{R}=11.33 min).

### Example 55: Preparation of compound 3-(trans-4-(2-(8-ethoxy-1,3,4,9-tetrahydro-2H-pyrido[3,4-b]indol-2-yl)ethyl)cyclohexyl)-1,1-dimethylurea (I-B8)

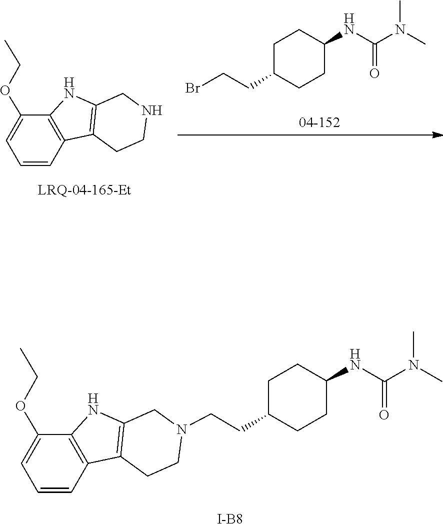

LRQ-04-165-Et (26 mg, 0.12 mmol) was dissolved in DMF (8 mL), then DIPEA (45 mg, 0.35 mmol) and LRQ-04-152 (47 mg, 0.17 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a white solid (5 mg, yield: 10%). ¹H NMR (800 MHz, CDCl₃) δ 7.00 (d, J=7.9 Hz, 1H), 6.94 (t, J=7.8 Hz, 1H), 6.57 (d, J=7.7 Hz, 1H), 4.13 (q, J=7.0 Hz, 2H), 4.01 (s, 2H), 3.56-3.49 (m, 1H), 3.14-3.06 (m, 2H), 2.92-2.87 (m, 2H), 2.85 (s, 6H), 2.82-2.74 (m, 2H), 2.00-1.91 (m, 2H), 1.74-1.66 (m, 2H), 1.63-1.53 (m, 2H), 1.42 (t, J=7.0 Hz, 3H), 1.27-1.24 (m, 1H), 1.08-0.99 (m, 4H). ¹³C NMR (201 MHz, CDCl₃) δ 157.91, 145.41, 133.63, 127.94, 126.88, 120.03, 110.77, 107.55, 103.26, 63.76, 55.16, 54.20, 50.38, 49.75, 36.24 (2C), 35.21, 33.79 (2C), 31.90 (2C), 29.75, 20.98, 15.07. HHRMS (ESI) calculated for C₂₄H₃₇N₄O₂⁺ [M+H]⁺: 413.2911, found: 413.2913. HPLC: 97.72% (λ=254 nm, t_{R}=11.31 min).

### Example 56: Preparation of compound 1-((8-methoxy-1,3,4,9-tetrahydro-2H-pyrido[3,4-b]indol-2-yl)methyl)cyclohexan-1-ol (I-B9) (IHCH-5229)

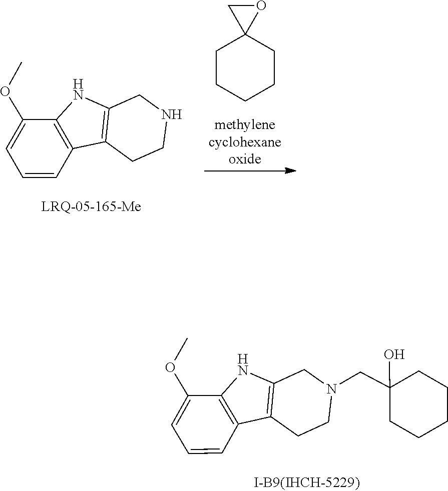 [SMILES: COc1cccc2c3c([nH]c12)CNCC3 ; C1CCC2(CC1)CO2 ; COc1cccc2c3c([nH]c12)CN(CC1(O)CCCCC1)CC3]

LRQ-05-165-Me (50 mg, 0.25 mmol) was dissolved in anhydrous ethanol (10 mL), then methylene cyclohexane oxide (138 mg, 1.25 mmol) was added thereto, and the reaction mixture was heated to 60° C. and reacted overnight. After the reaction was completed, the solvent was removed by evaporation under reduced pressure, and the residue was purified by silica gel column chromatography (methanol/dichloromethane=1:10) to obtain IHCH-5229 (5 mg, yield: 6%) as a light yellow solid. ¹H NMR (800 MHz, CDCl₃) δ 8.02 (s, 1H), 7.10 (d, J=7.9 Hz, 1H), 7.02 (t, J=7.8 Hz, 1H), 6.65-6.61 (m, 1H), 3.94 (s, 3H), 3.91 (s, 2H), 3.08-3.00 (m, 2H), 2.87-2.79 (m, 2H), 2.59 (s, 2H), 1.70-1.65 (m, 2H), 1.63-1.54 (m, 3H), 1.50-1.45 (m, 2H), 1.40-1.37 (m, 2H), 1.31-1.29 (m, 1H). ¹³C NMR (201 MHz, CDCl₃) δ 146.02, 136.02, 128.45, 126.19, 120.04, 114.41, 111.04, 102.07, 70.64, 66.22, 55.48, 53.53, 53.01, 36.73 (2C), 26.02, 22.28 (2C), 21.13. HRMS (ESI) calculated for C₁₉H₂₇N₂O₂⁺ [M+H]⁺: 315.2067, found: 315.2069. HPLC: 95.97% (λ=254 nm, t_{R}=11.90 min).

### Example 57: Preparation of compound 3-(3-(2-(8-methoxy-1,3,4,9-tetrahydro-2H-pyrido[3,4-b]indol-2-yl)ethyl)cyclobutyl)-1,1-dimethylurea (I-B10) (IHCH-5226)

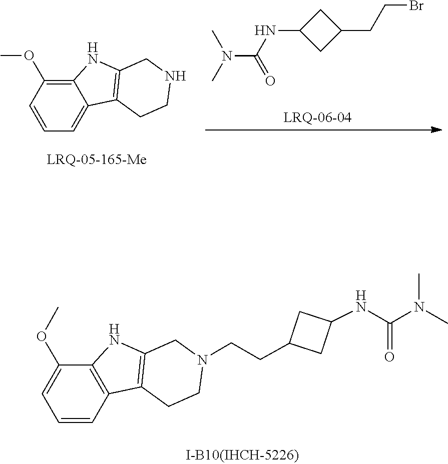

LRQ-05-165-Me (50 mg, 0.23 mmol) was dissolved in DMF (8 mL), then DIPEA (178 mg, 1.38 mmol) and LRQ-06-04 (87 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (13 mg, yield: 14%). ¹H NMR (800 MHz, CDCl₃) δ 8.70 (d, J=67.5 Hz, 1H), 7.09-7.01 (m, 1H), 7.01-6.93 (m, 1H), 6.62-6.57 (m, 1H), 4.64-4.57 (m, 1H), 4.39-4.09 (m, 1H), 3.95-3.86 (m, 3H), 3.82-3.74 (m, 2H), 2.98-2.90 (m, 2H), 2.89-2.84 (m, 6H), 2.84-2.78 (m, 2H), 2.61-2.53 (m, 2H), 2.52-2.43 (m, 1H), 2.22-1.83 (m, 3H), 1.84-1.76 (m, 1H), 1.77-1.69 (m, 1H), 1.49-1.41 (m, 1H). HRMS (ESI) calculated for C₂₁H₃₁N₄O₂⁺ [M+H]⁺: 371.2442, found: 371.2443. HPLC: 95.02% (λ=254 nm, t_{R}=11.25 min).

### Example 58: Preparation of compound 3-(3-(2-(8-ethoxy-1,3,4,9-tetrahydro-2H-pyrido[3,4-b]indol-2-yl)ethyl)cyclobutyl)-1,1-dimethylurea (I-B1l) (IHCH-5227)

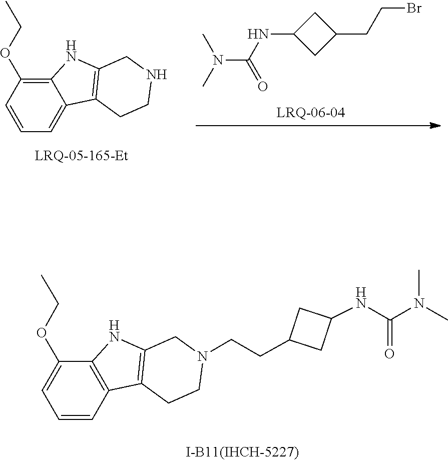 [SMILES: CCOc1cccc2c3c([nH]c12)CNCC3 ; CN(C)C(=O)NC1CC(CCBr)C1 ; CCOc1cccc2c3c([nH]c12)CN(CCC1CC(NC(=O)N(C)C)C1)CC3]

LRQ-05-165-Et (50 mg, 0.23 mmol) was dissolved in DMF (8 mL), then DIPEA (178 mg, 1.38 mmol) and LRQ-06-04 (87 mg, 0.35 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a reddish brown solid (20 mg, yield: 23%). ¹H NMR (800 MHz, CDCl₃) δ 7.01-6.97 (m, 1H), 6.96 (t, J=7.7 Hz, 1H), 6.60-6.56 (m, 1H), 5.14-4.79 (m, 1H), 4.36-4.25 (m, 2H), 4.13-4.09 (m, 2H), 3.34-3.26 (m, 2H), 3.02-2.94 (m, 2H), 2.91-2.81 (m, 8H), 2.46-2.19 (m, 2H), 2.07-2.02 (m, 1H), 2.00-1.94 (m, 2H), 1.85-1.76 (m, 1H), 1.61-1.55 (m, 1H), 1.42 (t, J=3.5 Hz, 3H). HRMS (ESI) calculated for C₂₂H₃₃N₄O₂⁺ [M+H]⁺: 385.2598, found: 385.2595. HPLC: 95.17% (λ=254 nm, t_{R}=11.25 min).

### Example 59: Preparation of compound 7-(4-(9-methoxy-3,4-dihydropyrazino[1,2-a]indol-2(1H)-yl)butoxy)quinolin-2(1H)-one (I-C₁)

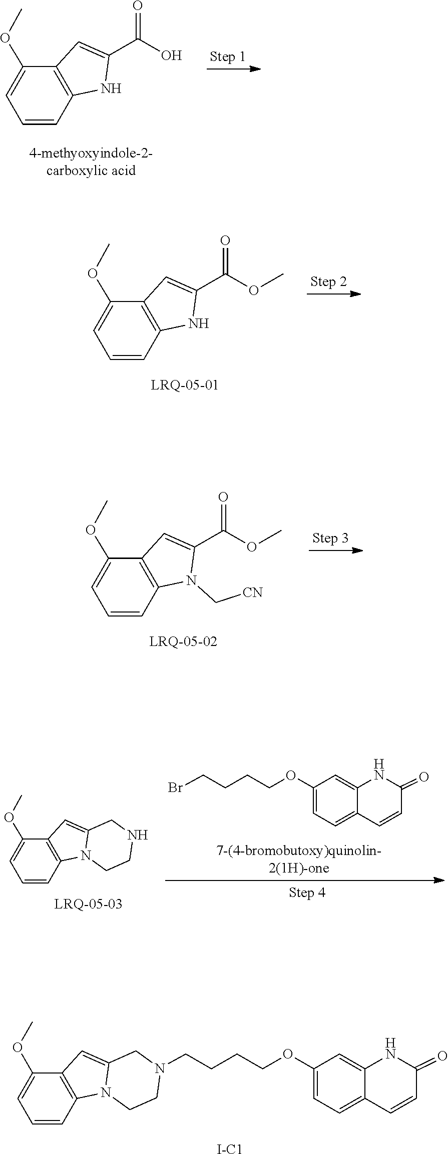

Steps 1 to 3: LRQ-05-03 was synthesized in the same way as in patent WO2004099212A1. LRQ-05-03 was a reddish brown solid (three-step reaction yield: 57%). ¹H NMR (800 MHz, MeOH-d₄) δ 7.03 (t, J=7.9 Hz, 1H), 6.93 (d, J=8.2 Hz, 1H), 6.53 (d, J=7.7 Hz, 1H), 6.26-6.21 (m, 1H), 4.14 (s, 2H), 4.04-4.00 (m, 2H), 3.90 (s, 3H), 3.31-3.30 (m, 2H). HRMS (ESI) calculated for C₁₂H₁₅N₂O⁺ [M+H]⁺: 203.1179, found: 203.1174.

Step 4: LRQ-05-03 (99 mg, 0.49 mmol) was dissolved in DMF (8 mL), then DIPEA (189 mg, 1.47 mmol) and 7-(4-bromobutoxy)quinolin-2(1H)-one (218 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a reddish brown solid (59 mg, yield: 29%). ¹H NMR (800 MHz, CDCl₃) δ 12.06 (s, 1H), 7.71 (d, J=9.4 Hz, 1H), 7.43 (d, J=8.5 Hz, 1H), 7.07 (t, J=7.9 Hz, 1H), 6.90 (d, J=8.1 Hz, 1H), 6.84-6.77 (m, 2H), 6.59-6.46 (m, 2H), 6.30 (s, 1H), 4.11 (t, J=6.1 Hz, 2H), 4.07 (t, J=5.5 Hz, 2H), 3.94 (s, 3H), 3.85 (s, 2H), 3.00 (t, J=5.4 Hz, 2H), 2.65 (t, J=7.2 Hz, 2H), 1.94-1.87 (m, 2H), 1.83-1.79 (m, 2H). ¹³C NMR (201 MHz, MeOH-d₄) δ 164.55, 161.52, 152.95, 141.40, 140.14, 137.62, 132.17, 129.35, 121.75, 118.69, 117.57, 114.48, 112.88, 102.48, 100.31, 98.92, 94.21, 68.04, 57.33, 55.42, 51.48, 50.59, 41.76, 27.06, 23.57. HRMS (ESI) calculated for C₂₅H₂₈N₃O₃⁺ [M+H]⁺: 418.2125, found: 418.2120. HPLC: 97.08% (λ=254 nm, t_{R}=11.84 min).

### Example 60: Preparation of compound 7-(4-(9-methoxy-3,4-dihydropyrazino[1,2-a]indol-2(1H)-yl)butoxy)-3,4-dihydroquinolin-2(1H)-one (I-C₂)

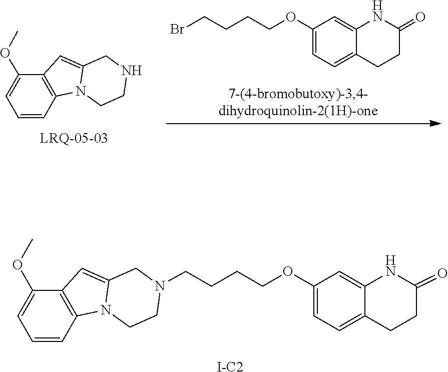 [SMILES: COc1cccc2c1cc1n2CCNC1 ; O=C1CCc2ccc(OCCCCBr)cc2N1 ; COc1cccc2c1cc1n2CCN(CCCCOc2ccc3c(c2)NC(=O)CC3)C1 ; "I-C2"]

LRQ-05-03 (99 mg, 0.49 mmol) was dissolved in DMF (8 mL), then DIPEA (189 mg, 1.47 mmol) and 7-(4-bromobutoxy)-3,4-dihydroquinolin-2(1H)-one (220 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a reddish brown solid (64 mg, yield: 31%). ¹H NMR (800 MHz, CDCl₃) δ 8.04 (s, 1H), 7.07 (t, J=7.9 Hz, 1H), 7.03 (d, J=8.3 Hz, 1H), 6.90 (d, J=8.1 Hz, 1H), 6.55-6.49 (m, 2H), 6.32-6.27 (m, 2H), 4.06 (t, J=5.6 Hz, 2H), 4.00-3.91 (m, 5H), 3.83 (s, 2H), 2.98 (t, J=5.6 Hz, 2H), 2.88 (t, J=7.5 Hz, 2H), 2.66-2.55 (m, 4H), 1.89-1.81 (m, 2H), 1.80-1.74 (m, 2H). ¹³C NMR (201 MHz, CDCl₃) δ 171.76, 158.77, 153.07, 138.26, 137.59, 132.91, 128.81, 121.51, 118.79, 115.91, 108.82, 102.35, 102.34, 100.20, 93.92, 67.97, 57.38, 55.50, 51.57, 50.71, 42.13, 31.24, 27.21, 24.74, 23.83. HRMS (ESI) calculated for C₂₅H₃₀N₃O₃⁺ [M+H]⁺: 420.2282, found: 420.2276. HPLC: 96.64% (Q=254 nm, t_{R}=11.86 min).

### Example 61: Preparation of compound 7-(3-(9-methoxy-3,4-dihydropyrazino[1,2-a]indol-2(1H)-yl)propoxy)quinolin-2(1H)-one (I-C₃)

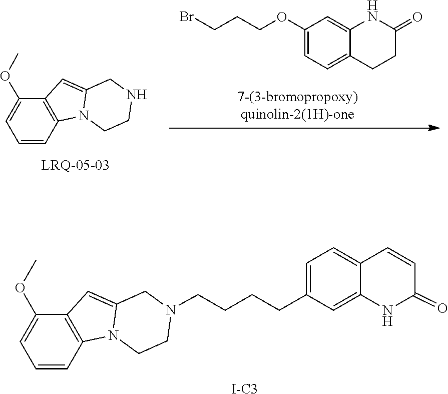 [SMILES: COc1cccc2c1cc1n2CCNC1 ; O=C1CCc2ccc(OCCCBr)cc2N1 ; COc1cccc2c1cc1n2CCN(CCCCc2ccc3ccc(=O)[nH]c3c2)C1 ; "I-C3"]

LRQ-05-03 (99 mg, 0.49 mmol) was dissolved in DMF (8 mL), then DIPEA (189 mg, 1.47 mmol) and 7-(3-bromopropoxy)quinolin-2(1H)-one (208 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (53 mg, yield: 27%). ¹H NMR (800 MHz, MeOH-d₄) δ 7.77 (d, J=9.3 Hz, 1H), 7.48 (d, J=8.7 Hz, 1H), 7.09 (t, J=8.0 Hz, 1H), 6.93 (d, J=8.1 Hz, 1H), 6.89-6.83 (m, 1H), 6.81 (d, J=2.4 Hz, 1H), 6.56 (d, J=7.7 Hz, 1H), 6.47 (d, J=9.4 Hz, 1H), 6.28 (s, 3H), 4.16 (t, J=6.1 Hz, 2H), 4.11 (t, J=5.7 Hz, 2H), 3.95 (s, 3H), 3.90 (s, 2H), 3.07 (t, J=5.6 Hz, 2H), 2.82 (t, J=7.4 Hz, 2H), 2.19-2.10 (m, 2H). ¹³C NMR (201 MHz, MeOH-d₄) δ 164.75, 161.64, 153.12, 141.60, 140.33, 137.82, 132.38, 129.54, 121.94, 118.87, 117.72, 114.74, 112.98, 102.66, 100.50, 99.14, 94.34, 66.51, 55.54, 54.55, 51.75, 50.88, 42.02, 26.95. HHRMS (ESI) calculated for C₂₄H₂₆N₃O₃⁺ [M+H]⁺: 404.1969, found: 404.1972. HPLC: 99.27% (λ=254 nm, t_{R}=11.56 min).

### Example 62: Preparation of compound 7-(3-(9-methoxy-3,4-dihydropyrazino[1,2-a]indol-2(1H)-yl)propoxy)-3,4-dihydroquinolin-2(1H)-one (I-C₄)

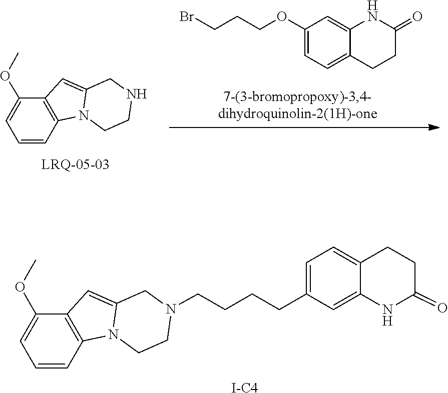 [SMILES: COc1cccc2c1cc1n2CCNC1 ; O=C1CCc2ccc(OCCCBr)cc2N1 ; COc1cccc2c1cc1n2CCN(CCCCc2ccc3c(c2)NC(=O)CC3)C1 ; "I-C4"]

LRQ-05-03 (99 mg, 0.49 mmol) was dissolved in DMF (8 mL), then DIPEA (189 mg, 1.47 mmol) and 7-(3-bromopropoxy)-3,4-dihydroquinolin-2(1H)-one (209 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (65 mg, yield: 33%). ¹H NMR (800 MHz, CDCl₃) δ 8.26 (s, 1H), 7.08 (t, J=7.9 Hz, 1H), 7.03 (d, J=8.3 Hz, 1H), 6.90 (d, J=8.1 Hz, 1H), 6.56-6.49 (m, 2H), 6.36-6.32 (m, 1H), 6.31 (s, 1H), 4.07 (t, J=5.5 Hz, 2H), 4.03 (t, J=6.1 Hz, 2H), 3.94 (s, 3H), 3.86 (s, 2H), 3.01 (t, J=5.3 Hz, 2H), 2.88 (t, J=7.5 Hz, 2H), 2.75 (t, J=7.0 Hz, 2H), 2.60 (t, J=7.5 Hz, 2H), 2.09-2.02 (m, 2H). ¹³C NMR (201 MHz, CDCl₃) δ 171.91, 158.69, 153.06, 138.28, 137.56, 132.66, 128.79, 121.59, 118.75, 115.96, 108.77, 102.36, 102.36, 100.19, 94.01, 66.18, 55.49, 54.27, 51.56, 50.79, 42.08, 31.21, 27.12, 24.71. HRMS (ESI) calculated for C₂₄H₂₈N₃O₃⁺ [M+H]⁺: 406.2125, found: 406.2131. HPLC: 97.30% (λ=254 nm, t_{R}=11.64 min).

### Example 63: Preparation of compound (E)-7-((4-(9-methoxy-3,4-dihydropyrazino[1,2-a]indol-2(1H)-yl)but-2-en-1-yl)oxy)quinolin-2(1H)-one (I-C₅)

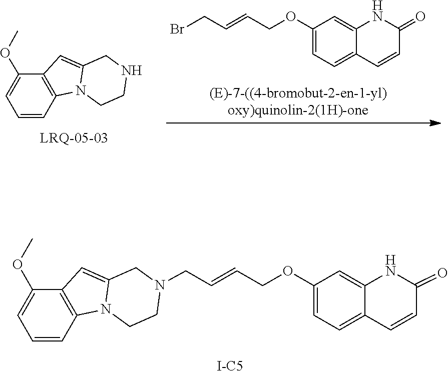 [SMILES: COc1cccc2c1cc1n2CCNC1 ; O=c1ccc2ccc(OC/C=C/CBr)cc2[nH]1 ; COc1cccc2c1cc1n2CCN(C/C=C/COc2ccc3ccc(=O)[nH]c3c2)C1 ; "I-C5"]

LRQ-05-03 (99 mg, 0.49 mmol) was dissolved in DMF (8 mL), then DIPEA (189 mg, 1.47 mmol) and (E)-7-((4-bromobut-2-en-1-yl)oxy)quinolin-2(1H)-one (217 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (47 mg, yield: 23%). ¹H NMR (800 MHz, MeOH-d₄) δ 7.73-7.70 (m, 1H), 7.44 (d, J=8.7 Hz, 1H), 7.03 (t, J=7.9 Hz, 1H), 6.86 (d, J=8.1 Hz, 1H), 6.83-6.81 (m, 1H), 6.78 (d, J=2.3 Hz, 1H), 6.50 (d, J=7.7 Hz, 1H), 6.44 (d, J=9.4 Hz, 1H), 6.25-6.23 (m, 1H), 5.99-5.90 (m, 2H), 4.62 (d, J=4.6 Hz, 2H), 4.03 (t, J=5.7 Hz, 2H), 3.89 (s, 3H), 3.80 (s, 2H), 3.24 (d, J=5.5 Hz, 2H), 2.98-2.94 (m, 2H). ¹³C NMR (201 MHz, MeOH-d₄) δ 164.52, 160.95, 152.97, 141.33, 140.13, 137.65, 132.11, 130.28, 129.41, 129.21, 121.78, 118.70, 117.86, 114.65, 112.98, 102.48, 100.33, 99.38, 94.26, 68.27, 59.34, 55.42, 51.48, 50.22, 41.82. HRMS (ESI) calculated for C₂₅H₂₆N₃O₃⁺ [M+H]⁺: 416.1969, found: 416.1971. HPLC: 98.09% (λ254 nm, t_{R}=11.75 min).

### Example 64: Preparation of compound (E)-7-((4-(9-methoxy-3,4-dihydropyrazino[1,2-a]indol-2(1H)-yl)but-2-en-1-yl)oxy)-3,4-dihydroquinolin-2(1H)-one (I-C6)

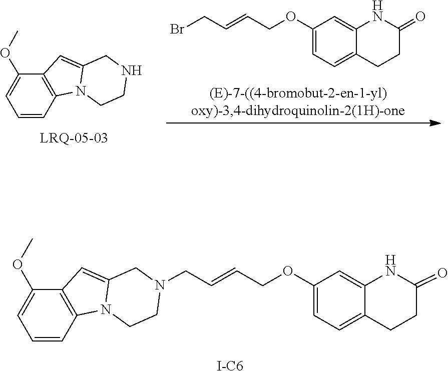 [SMILES: COc1cccc2c1cc1n2CCNC1 ; O=C1CCc2ccc(OC/C=C/CBr)cc2N1 ; COc1cccc2c1cc1n2CCN(C/C=C/COc2ccc3c(c2)NC(=O)CC3)C1 ; "I-C6"]

LRQ-05-03 (99 mg, 0.49 mmol) was dissolved in DMF (8 mL), then DIPEA (189 mg, 1.47 mmol) and (E)-7-((4-bromobut-2-en-1-yl)oxy)-3,4-dihydroquinolin-2(1H)-one (218 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (72 mg, yield: 35%). ¹H NMR (800 MHz, CDCl₃) δ 8.19 (s, 1H), 7.08 (t, J=7.9 Hz, 1H), 7.05 (d, J=8.3 Hz, 1H), 6.90 (d, J=8.1 Hz, 1H), 6.57-6.50 (m, 2H), 6.36 (d, J=2.3 Hz, 1H), 6.28 (s, 1H), 5.98-5.90 (m, 2H), 4.53 (d, J=4.5 Hz, 2H), 4.06 (t, J=5.6 Hz, 2H), 3.94 (s, 3H), 3.81 (s, 2H), 3.25 (d, J=5.3 Hz, 2H), 2.98 (t, J=5.6 Hz, 2H), 2.90 (t, J=7.5 Hz, 2H), 2.65-2.58 (m, 2H). ¹³C NMR (201 MHz, CDCl₃) δ 171.85, 158.22, 153.05, 138.32, 137.58, 132.70, 130.64, 128.96, 128.83, 121.55, 118.75, 116.24, 109.05, 102.65, 102.35, 100.16, 94.02, 68.30, 59.50, 55.48, 51.54, 50.38, 42.12, 31.19, 24.73. HRMS (ESI) calculated for C₂₅H₂₈N₃O₃⁺ [M+H]⁺: 418.2125, found: 418.2122. HPLC: 98.74% (λ=254 nm, t_{R}=11.84 min).

### Example 65: Preparation of compound 3-(trans-4-(2-(9-methoxy-3,4-dihydropyrazino[1,2-a]indol-2(1H)-yl)ethyl)cyclohexyl)-1,1-dimethylurea (I-C₇)

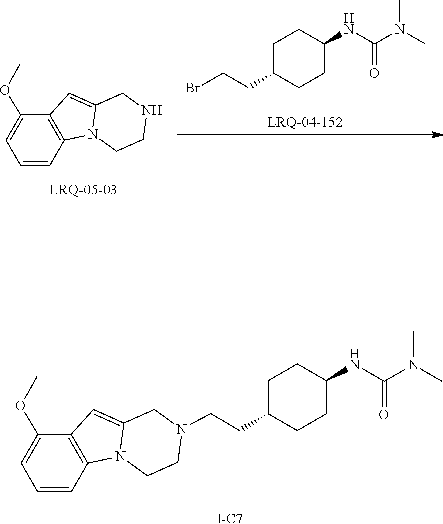 [SMILES: COc1cccc2c1cc1n2CCNC1 ; CN(C)C(=O)N[C@H]1CC[C@H](CCBr)CC1 ; COc1cccc2c1cc1n2CCN(CC[C@H]2CC[C@H](NC(=O)N(C)C)CC2)C1 ; "I-C7"]

LRQ-05-03 (24 mg, 0.12 mmol) was dissolved in DMF (8 mL), then DIPEA (45 mg, 0.35 mmol) and LRQ-04-152 (47 mg, 0.17 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (11 mg, yield: 23%). ¹H NMR (800 MHz, CDCl₃) δ 7.07 (t, J=7.9 Hz, 1H), 6.93-6.85 (m, 1H), 6.55-6.48 (m, 1H), 6.29 (s, 1H), 4.06 (t, J=5.6 Hz, 2H), 3.94 (s, 3H), 3.80 (s, 2H), 3.61-3.54 (m, 1H), 2.95 (t, J=5.6 Hz, 2H), 2.87 (s, 6H), 2.60-2.50 (m, 2H), 2.06-1.97 (m, 2H), 1.81-1.76 (m, 2H), 1.53-1.47 (m, 2H), 1.35-1.27 (m, 1H), 1.14-1.00 (m, 4H). ¹³C NMR (201 MHz, CDCl₃) δ 157.98, 153.06, 137.58, 132.49, 121.46, 118.79, 102.35, 100.18, 93.88, 55.69, 55.50, 51.66, 50.71, 49.98, 42.10, 36.28 (2C), 35.35, 34.22, 34.13 (2C), 32.20 (2C). HRMS (ESI) calculated for C₂₃H₃₅N₄O₂⁺ [M+H]⁺: 399.2755, found: 399.2749. HPLC: 96.99% (λ=254 nm, t_{R}=11.84 min).

### Example 66: Preparation of compound N-(2-(9-methoxy-3,4-dihydropyrazino[1,2-a]indol-2(1H)-yl)ethyl)tetrahydro-2H-pyran-4-carboxamide (I-C₈)

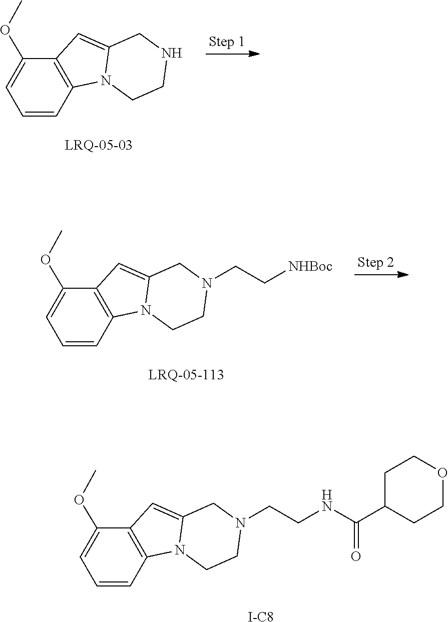 [SMILES: COc1cccc2c1cc1n2CCNC1 ; COc1cccc2c1cc1n2CCN(CCNC(=O)OC(C)(C)C)C1 ; COc1cccc2c1cc1n2CCN(CCNC(=O)C2CCOCC2)C1 ; "I-C8"]

Step 1: LRQ-05-03 (920 mg, 4.55 mmol) and DIPEA (3.53 g, 27.30 mmol) were dissolved in DMF (10 mL), then N-Boc-2-bromoethylamine (561 mg, 2.51 mmol) was added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain a yellow solid (800 mg, yield: 51%). ¹H NMR (800 MHz, CDCl₃) δ 7.09 (t, J=7.9 Hz, 1H), 6.91 (d, J=8.1 Hz, 1H), 6.54 (d, J=7.7 Hz, 1H), 6.32 (s, 1H), 4.14-4.04 (m, 2H), 3.94 (s, 3H), 3.86 (s, 2H), 3.43-3.26 (m, 2H), 3.14-2.91 (m, 2H), 2.80-2.63 (m, 2H), 1.43 (s, 9H). HRMS (ESI) calculated for C₁₉H₂₈N₃O₃⁺ [M+H]⁺: 346.2125, found: 346.2131.

Step 2: LRQ-05-113 (70 mg, 0.20 mmol) was dissolved in DCM (10 mL), and the reaction mixture was cooled to 0° C. Trifluoroacetic acid (1 mL) was added thereto, and the reaction mixture was reacted for 2 hours. After the reaction was completed, the solvent was removed by evaporation under reduced pressure to obtain a colorless oily liquid, which was then dissolved in dichloromethane (10 mL). DIPEA (155 mg, 1.20 mmol) and tetrahydro-2H-pyran-4-carbonyl chloride (44 mg, 0.30 mmol) were added thereto, and the reaction mixture was reacted at room temperature overnight. After the reaction was completed, the reaction mixture was diluted with saturated sodium bicarbonate aqueous solution (10 mL), extracted with dichloromethane (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain a yellow solid (43 mg, yield: 36%). ¹H NMR (800 MHz, CDCl₃) δ 7.10 (t, J=7.9 Hz, 1H), 6.91 (d, J=8.1 Hz, 1H), 6.55 (d, J=7.7 Hz, 1H), 6.31 (s, 1H), 6.18-6.07 (m, 1H), 4.07 (t, J=5.5 Hz, 2H), 4.00-3.95 (m, 2H), 3.94 (s, 3H), 3.83 (s, 2H), 3.49-3.42 (m, 2H), 3.40-3.32 (m, 2H), 3.00 (t, J=5.5 Hz, 2H), 2.72 (t, J=5.8 Hz, 2H), 2.34-2.27 (m, 1H), 1.82-1.74 (m, 2H), 1.72-1.66 (m, 2H). ¹³C NMR (201 MHz, CDCl₃) δ 174.56, 153.12, 137.54, 132.26, 121.87, 118.68, 102.33, 100.32, 94.20, 67.36 (2C), 55.94, 55.50, 51.38, 50.35, 42.24, 42.17, 36.11, 29.38 (2C). HRMS (ESI) calculated for C₂₀H₂₈N₃O₃⁺ [M+H]⁺: 358.2125, found: 358.2123. HPLC: 97.41% (λ=254 nm, t_{R}=11.75 min).

### Example 67: Preparation of compound 4,4-difluoro-N-(2-(9-methoxy-3,4-dihydropyrazino[1,2-a]indol-2(1H)-yl)ethyl)cyclohexane-1-carboxamide (I-C₉)

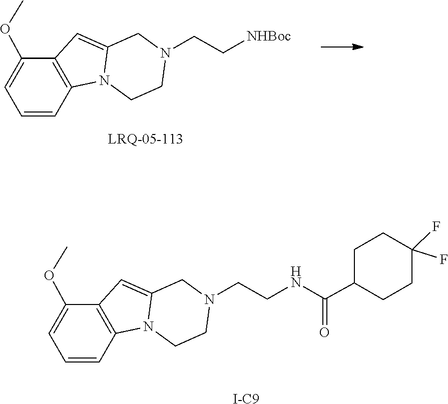 [SMILES: COc1cccc2c1cc1n2CCN(CCNC(=O)OC(C)(C)C)C1 ; COc1cccc2c1cc1n2CCN(CCNC(=O)C2CCC(F)(F)CC2)C1 ; "I-C9"]

LRQ-05-116 (70 mg, 0.20 mmol) was dissolved in DCM (10 mL), and the reaction mixture was cooled to 0° C. Trifluoroacetic acid (1 mL) was added thereto, and the reaction mixture was reacted for 2 hours. After the reaction was completed, the solvent was removed by evaporation under reduced pressure to obtain a colorless oily liquid, which was then dissolved in dichloromethane (10 mL). DIPEA (155 mg, 1.20 mmol) and 4,4-difluoro-cyclohexanecarbonyl chloride (68 mg, 0.30 mmol) were added thereto, and the reaction mixture was reacted at room temperature overnight. After the reaction was completed, the reaction mixture was diluted with saturated sodium bicarbonate aqueous solution (10 mL), extracted with dichloromethane (20 mL*3), washed with saturated brine, and dried over anhydrous sodium sulfate. The solvent was removed by evaporation under reduced pressure, and the residue was purified by silica gel column chromatography (ethyl acetate/petroleum ether=1:1) to obtain a yellow solid (91 mg, yield: 73%). ¹H NMR (800 MHz, CDCl₃) δ 7.12-7.08 (m, 1H), 6.94-6.88 (m, 1H), 6.55 (d, J=7.7 Hz, 1H), 6.32 (s, 1H), 6.13-6.06 (m, 1H), 4.07 (t, J=5.6 Hz, 2H), 3.95 (s, 3H), 3.83 (s, 2H), 3.48-3.42 (m, 2H), 3.00 (t, J=5.5 Hz, 2H), 2.71 (t, J=5.8 Hz, 2H), 2.19-2.08 (m, 3H), 1.92-1.86 (m, 2H), 1.84-1.78 (m, 2H), 1.74-1.65 (m, 2H). ¹³C NMR (201 MHz, CDCl₃) δ 174.30, 153.13, 137.54, 132.28, 121.91, 121.55 (t, J=549.9 Hz), 118.67, 102.34, 100.34, 94.19, 55.93, 55.51, 51.39, 50.38, 42.85, 42.21, 36.13, 32.95 (t, J=25.2 Hz) (2C), 26.05, 26.01. HRMS (ESI) calculated for C₂₁H₂₈F₂N₃O₂⁺ [M+H]⁺: 392.2144, found: 392.2138. HPLC: 96.36% (λ=254 nm, t_{R}=11.90 min).

### Example 68: Preparation of compound 7-(4-(9-ethoxy-3,4-dihydropyrazino[1,2-a]indol-2(1H)-yl)butoxy)quinolin-2(1H)-one (I-C₁₀)

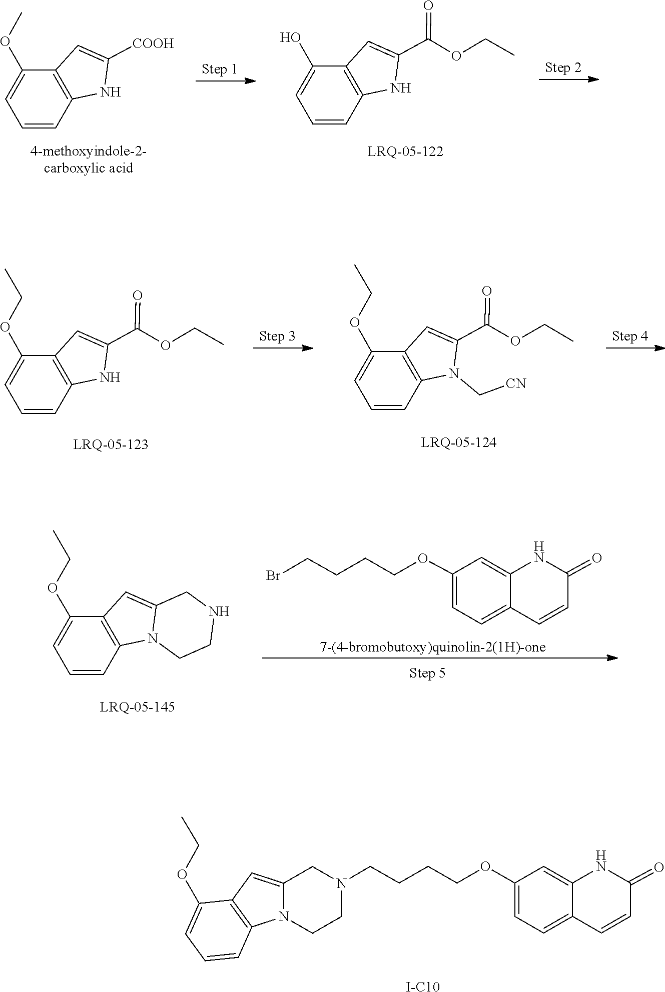

Step 1: 4-Methoxyindole-2-carboxylic acid (5.0 g, 26.15 mmol) was dissolved in dichloromethane (50 mL). The reaction mixture was replaced with nitrogen three times, then cooled to −78° C., and BBr₃ (39.2 mL, 2.0 M in dichloromethane) was added thereto. The reaction mixture was then warmed to room temperature and reacted for 6 hours. After the reaction was completed, the reaction mixture was cooled to −30° C., quenched with methanol (5 mL), then stirred at room temperature for 1 hour, diluted with water (30 mL), extracted with ethyl acetate (50 mL*3), washed with saturated brine, dried over anhydrous sodium sulfate, and the solvent was removed by evaporation under reduced pressure to obtain a reddish brown oily liquid, which was then dissolved in ethanol (50 mL). Sulfuric acid (0.5 mL) was added thereto, and the reaction mixture was refluxed overnight. After the reaction was completed, the solvent was removed by evaporation under reduced pressure. The reaction mixture was diluted with saturated sodium bicarbonate aqueous solution (30 mL), extracted with ethyl acetate (50 mL*3), washed with saturated brine, dried over anhydrous sodium sulfate, and the solvent was removed by evaporation under reduced pressure to obtain LRQ-05-122 as a reddish brown oily liquid, which was directly used in the next reaction step without purification. HRMS (ESI) calculated for C₁₁H₁₂NO₃⁺ [M+H]⁺: 206.0812, found: 206.0814.

Step 2: LRQ-05-122 and K₂CO₃ (3.6 g, 26.15 mmol) were dissolved in DMF (50 mL), then iodoethane (4.49 g, 28.76 mmol) was added thereto, and the reaction mixture was stirred and reacted at room temperature overnight. After the reaction was completed, the reaction mixture was diluted with water (20 mL), extracted with ethyl acetate (50 mL*3), washed with saturated brine, dried over anhydrous sodium sulfate, and the solvent was removed by evaporation under reduced pressure to obtain LRQ-05-123 as an orange-red oily liquid, which was directly used in the next reaction step without purification. HRMS (ESI) calculated for C₁₃H₁₆NO₃⁺ [M+H]⁺: 234.1125, found: 234.1121.

Step 3: LRQ-05-123 and potassium tert-butoxide (6.27 g, 52.30 mmol) were dissolved in DMF (50 mL). The reaction mixture was reacted at room temperature for 40 minutes, added with bromoacetonitrile (4.40 g, 39.23 mmol), then heated to 60° C., and reacted for 30 minutes. The reaction mixture was then cooled to room temperature and reacted overnight. After the reaction was completed, the reaction mixture was diluted with water (30 mL), extracted with ethyl acetate (50 mL*3), washed with saturated brine, and dried over anhydrous sodium sulfate. The solvent was removed by evaporation under reduced pressure, and the residue was purified by silica gel column chromatography (ethyl acetate/petroleum ether=1:10) to obtain LRQ-05-124 (0.96 g, three-step reaction yield: 13%) as a yellow solid. ¹H NMR (800 MHz, CDCl₃) δ 7.53 (s, 1H), 7.35 (t, J=8.1 Hz, 1H), 7.01-6.97 (m, 1H), 6.59 (d, J=7.8 Hz, 1H), 5.58 (s, 2H), 4.40 (q, J=7.1 Hz, 2H), 4.19 (q, J=7.0 Hz, 2H), 1.50 (t, J=7.0 Hz, 3H), 1.42 (t, J=7.1 Hz, 3H). HRMS (ESI) calculated for C₁₅H₁₇N₂O₃⁺ [M+H]⁺: 273.1234, found: 273.1231.

Step 4: LRQ-05-124 (0.96 g, 3.53 mmol) was dissolved in tetrahydrofuran (10 mL), then lithium aluminum hydride (536 mg, 14.1 mmol) was added thereto, and the reaction mixture was refluxed for 4 hours. After the reaction was completed, the reaction mixture was cooled to 0° C., quenched with saturated ammonium chloride aqueous solution (5 mL), then stirred at room temperature for 1 hour, diluted with water (30 mL), extracted with ethyl acetate (50 mL*3), washed with saturated brine, dried over anhydrous sodium sulfate, and the solvent was removed by evaporation under reduced pressure to obtain LRQ-05-145 as an orange-yellow solid, which was directly used in the next reaction step without purification. HRMS (ESI) calculated for C₁₃H₁₇N₂O⁺ [M+H]⁺: 217.1335, found: 217.1329.

Step 5: LRQ-05-145 (15 mg, 0.07 mmol) was dissolved in DMF (8 mL), then DIPEA (54 mg, 0.42 mmol) and 7-(4-bromobutoxy)quinolin-2(1H)-one (31 mg, 0.11 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain an orange-yellow solid (14 mg, yield: 47%). ¹H NMR (800 MHz, CDCl₃) δ 7.73-7.68 (m, 1H), 7.42 (d, J=8.6 Hz, 1H), 7.05 (t, J=7.9 Hz, 1H), 6.88 (d, J=8.1 Hz, 1H), 6.83 (s, 1H), 6.80-6.77 (m, 1H), 6.55-6.48 (m, 2H), 6.34-6.31 (m, 1H), 4.17 (q, J=7.0 Hz, 2H), 4.12-4.04 (m, 4H), 3.88 (s, 2H), 3.08-2.98 (m, 2H), 2.72-2.62 (m, 2H), 1.94-1.87 (m, 2H), 1.85-1.78 (m, 2H), 1.47 (t, J=7.0 Hz, 3H). HRMS (ESI) calculated for C₂₆H₃₀N₃O₃⁺ [M+H]⁺: 432.2282, found: 432.2284. HPLC: 97.54% (λ=254 nm, t_{R}=11.84 min).

### Example 69: Preparation of compound 3-(trans-4-(2-(9-ethoxy-3,4-dihydropyrazino[1,2-a]indol-2(1H)-yl)ethyl)cyclohexyl)-1,1-dimethylurea (I-C₁₁)

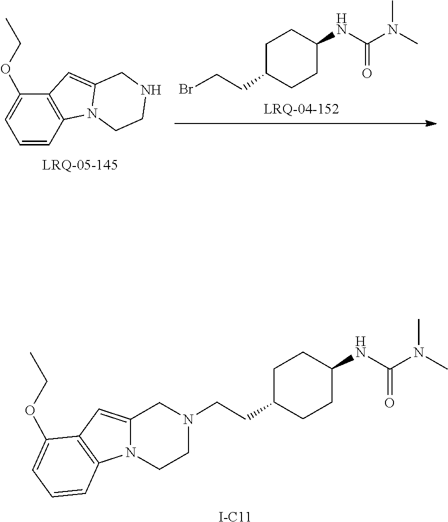 [SMILES: CCOc1cccc2c1cc1n2CCNC1 ; CN(C)C(=O)N[C@H]1CC[C@H](CCBr)CC1 ; CCOc1cccc2c1cc1n2CCN(CC[C@H]2CC[C@H](NC(=O)N(C)C)CC2)C1 ; "I-C11"]

Step 1: LRQ-05-145 (25 mg, 0.11 mmol) was dissolved in DMF (8 mL), then DIPEA (85 mg, 0.66 mmol) and LRQ-04-152 (47 mg, 0.17 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (17 mg, yield: 36%). ¹H NMR (800 MHz, CDCl₃) δ 7.04 (t, J=7.9 Hz, 1H), 6.88 (d, J=8.1 Hz, 1H), 6.52 (d, J=7.7 Hz, 1H), 6.31 (s, 1H), 4.17 (q, J=7.0 Hz, 2H), 4.08-4.01 (m, 2H), 3.80 (s, 2H), 3.62-3.54 (m, 1H), 2.98-2.92 (m, 2H), 2.87 (s, 6H), 2.60-2.54 (m, 2H), 2.03-2.00 (m, 2H), 1.83-1.75 (m, 2H), 1.52-1.48 (m, 2H), 1.46 (t, J=7.0 Hz, 3H), 1.31-1.29 (m, 1H), 1.10-1.06 (m, 4H). HRMS (ESI) calculated for C₂₄H₃₇N₄O₂⁺ [M+H]⁺: 413.2911, found: 413.2907. HPLC: 97.84% (λ=254 nm, t_{R}=11.82 min).

### Example 70: Preparation of compound 1-((9-methoxy-3,4-dihydropyrazino[1,2-a]indol-2(1H)-yl)methyl)cyclohexan-1-ol (I-C₁₂) (IHCH-5231)

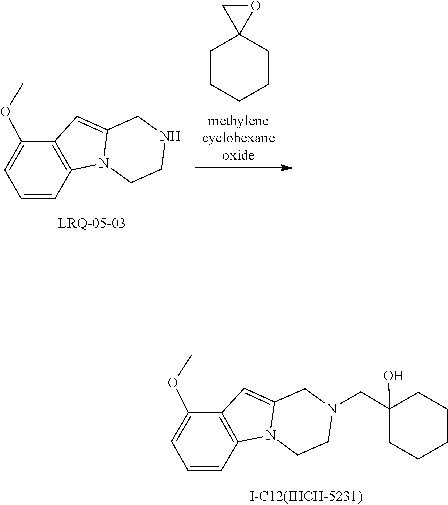 [SMILES: COc1cccc2c1cc1n2CCNC1 ; C1CCC2(CC1)CO2 ; COc1cccc2c1cc1n2CCN(CC2(O)CCCCC2)C1]

LRQ-05-03 (50 mg, 0.25 mmol) was dissolved in anhydrous ethanol (10 mL), then methylene cyclohexane oxide (138 mg, 1.25 mmol) was added thereto, and the reaction mixture was heated to 60° C. and reacted overnight. After the reaction was completed, the solvent was removed by evaporation under reduced pressure, and the residue was purified by silica gel column chromatography (methanol/dichloromethane=1:10) to obtain a yellow solid (29 mg, yield: 37%). ¹H NMR (800 MHz, CDCl₃) δ 7.09 (t, J=7.9 Hz, 1H), 6.91 (d, J=8.1 Hz, 1H), 6.54 (d, J=7.7 Hz, 1H), 6.30 (s, 1H), 4.07 (t, J=5.5 Hz, 2H), 4.01 (s, 2H), 3.94 (s, 3H), 3.23-3.12 (m, 2H), 2.54 (s, 2H), 1.70-1.63 (m, 2H), 1.62-1.53 (m, 3H), 1.50-1.45 (m, 2H), 1.42-1.34 (m, 2H), 1.32-1.24 (m, 1H). ¹³C NMR (201 MHz, CDCl₃) δ 153.09, 137.61, 132.35, 121.74, 118.59, 102.34, 100.26, 98.41, 70.90, 66.39, 55.50, 53.93, 53.08, 41.93, 36.51 (2C), 25.98, 22.20 (2C). HRMS (ESI) calculated for C₁₉H₂₇N₂O₂⁺ [M+H]⁺: 315.2067, found: 315.2068. HPLC: 98.92% (λ=254 nm, t_{R}=11.45 min).

### Example 71: Preparation of compound 7-(4-(9-methoxy-3,4-dihydrobenzo[4,5]imidazo[1,2-a]pyrazin-2(1H)-yl)butoxy)quinolin-2(1H)-one (I-D1)

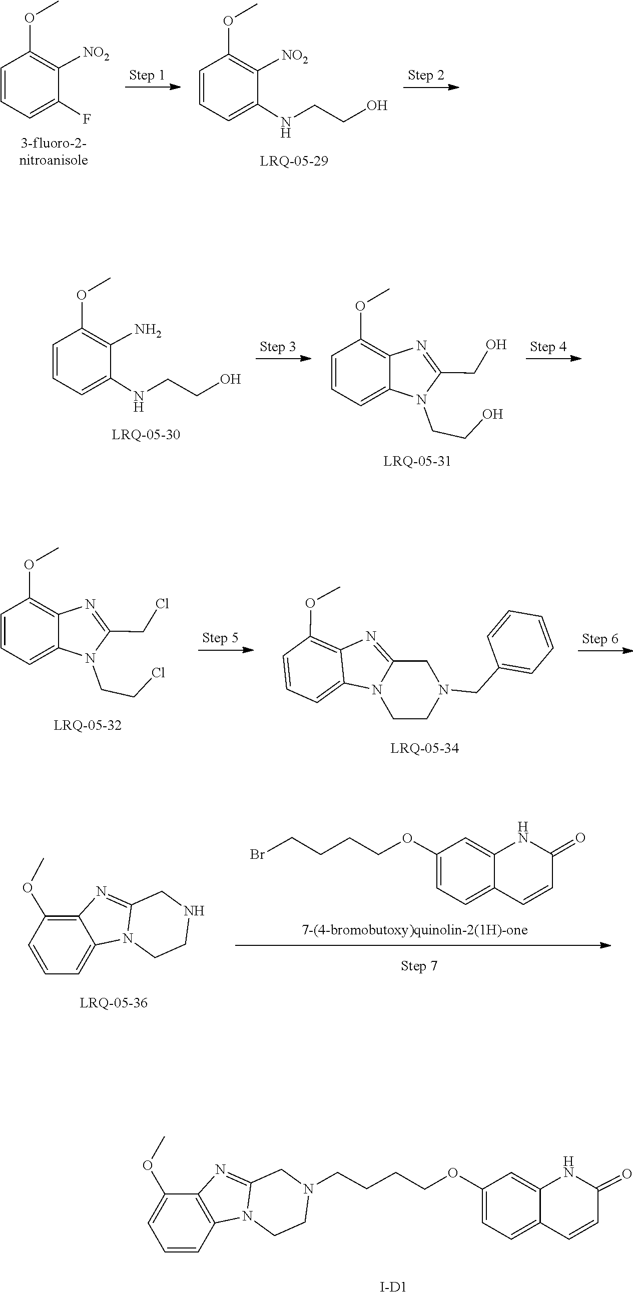

Steps 1, 2, 3, 4, 5, and 6: LRQ-05-36 was synthesized in the same way as in the literature European Journal of Medicinal Chemistry 186 (2020) 111881, using 3-fluoro-2-nitroanisole as the starting material. LRQ-05-03 was a reddish brown solid (six-step reaction yield: 20%). ¹H NMR (800 MHz, CDCl₃) δ 7.17 (t, J=7.9 Hz, 1H), 6.92 (d, J=8.0 Hz, 1H), 6.70 (d, J=7.9 Hz, 1H), 4.38-4.25 (m, 2H), 4.16-4.05 (m, 2H), 3.99 (s, 3H), 3.49-3.33 (m, 2H). ¹³C NMR (201 MHz, CDCl₃) δ 151.28, 147.47, 135.85, 132.46, 123.14, 103.49, 101.93, 55.87, 44.81, 43.04, 42.72. HRMS (ESI) calculated for C₁H₁₄N₃₀⁺ [M+H]⁺: 204.1131, found: 204.1134.

Step 7: LRQ-05-36 (100 mg, 0.49 mmol) was dissolved in DMF (8 mL), then DIPEA (189 mg, 1.47 mmol) and 7-(4-bromobutoxy)quinolin-2(1H)-one (218 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (119 mg, yield: 58%). ¹H NMR (800 MHz, CDCl₃) δ 12.07 (s, 1H), 7.69 (d, J=9.4 Hz, 1H), 7.41 (d, J=8.7 Hz, 1H), 7.15 (t, J=8.0 Hz, 1H), 6.91 (d, J=8.0 Hz, 1H), 6.85 (s, 1H), 6.81-6.77 (m, 1H), 6.69 (d, J=7.9 Hz, 1H), 6.54-6.48 (m, 1H), 4.12-4.07 (m, 4H), 4.00 (s, 3H), 3.93 (s, 2H), 3.03 (t, J=5.5 Hz, 2H), 2.69 (t, J=7.1 Hz, 2H), 1.93-1.86 (m, 2H), 1.83-1.76 (m, 2H). ¹³C NMR (201 MHz, CDCl₃) δ 164.91, 161.43, 151.24, 148.08, 140.96, 140.44, 135.59, 132.83, 129.16, 122.96, 118.01, 114.31, 112.71, 103.26, 101.95, 99.14, 68.05, 57.15, 55.84, 52.07, 49.78, 42.22, 26.95, 23.62. HRMS (ESI) calculated for C₂₄H₂₇N₄O₃⁺ [M+H]⁺: 419.2078, found: 419.2072. HPLC: 95.92% (λ=254 nm, t_{R}=12.47 min).

### Example 72: Preparation of compound 7-(4-(9-methoxy-3,4-dihydrobenzo[4,5]imidazo[1,2-a]pyrazin-2(1H)-yl)butoxy)-3,4-dihydroquinolin-2(1H)-one (I-D2)

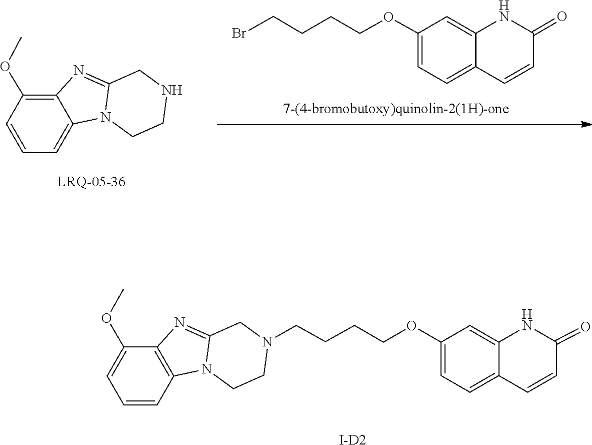 [SMILES: COc1cccc2c1nc1n2CCNC1 ; O=c1ccc2ccc(OCCCCBr)cc2[nH]1 ; COc1cccc2c1nc1n2CCN(CCCCOc2ccc3ccc(=O)[nH]c3c2)C1 ; "I-D2"]

LRQ-05-36 (100 mg, 0.49 mmol) was dissolved in DMF (8 mL), then DIPEA (189 mg, 1.47 mmol) and 7-(4-bromobutoxy)-3,4-dihydroquinolin-2(1H)-one (220 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (58 mg, yield: 28%). ¹H NMR (800 MHz, CDCl₃) δ 8.23 (s, 1H), 7.17 (t, J=8.0 Hz, 1H), 7.01 (d, J=8.3 Hz, 1H), 6.92 (d, J=8.0 Hz, 1H), 6.70 (d, J=7.9 Hz, 1H), 6.53-6.46 (m, 1H), 6.34 (d, J=2.0 Hz, 1H), 4.09 (t, J=5.5 Hz, 2H), 4.00 (s, 3H), 3.98-3.93 (m, 4H), 3.03 (t, J=5.5 Hz, 2H), 2.86 (t, J=7.5 Hz, 2H), 2.68 (t, J=7.1 Hz, 2H), 2.61-2.57 (m, 2H), 1.87-1.82 (m, 2H), 1.80-1.74 (m, 2H). ¹³C NMR (201 MHz, CDCl₃) δ 171.83, 158.68, 151.18, 148.00, 138.29, 135.48, 132.87, 128.74, 123.12, 115.91, 108.67, 103.41, 102.41, 101.94, 67.79, 57.13, 55.87, 51.88, 49.76, 42.19, 31.21, 27.02, 24.70, 23.60. HRMS (ESI) calculated for C₂₄H₂₉N₄O₃⁺ [M+H]⁺: 421.2234, found: 421.2239. HPLC: 96.98% (λ=254 nm, t_{R}=12.33 min).

### Example 73: Preparation of compound 7-(3-(9-methoxy-3,4-dihydrobenzo[4,5]imidazo[1,2-a]pyrazin-2(1H)-yl)propoxy)quinolin-2(1H)-one (I-D3)

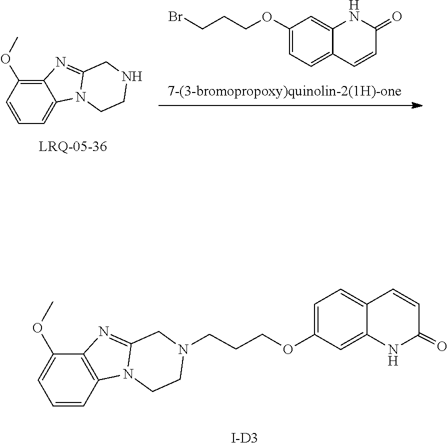 [SMILES: COc1cccc2c1nc1n2CCNC1 ; O=c1ccc2ccc(OCCCBr)cc2[nH]1 ; COc1cccc2c1nc1n2CCN(CCCOc2ccc3ccc(=O)[nH]c3c2)C1 ; "I-D3"]

LRQ-05-36 (100 mg, 0.49 mmol) was dissolved in DMF (8 mL), then DIPEA (189 mg, 1.47 mmol) and 7-(3-bromopropoxy)quinolin-2(1H)-one (208 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (61 mg, yield: 31%). ¹H NMR (800 MHz, CDCl₃) δ 12.19 (s, 1H), 7.71-7.67 (m, 1H), 7.42 (d, J=8.7 Hz, 1H), 7.15 (t, J=8.0 Hz, 1H), 6.92 (d, J=8.0 Hz, 1H), 6.86 (d, J=1.8 Hz, 1H), 6.82-6.78 (m, 1H), 6.69 (d, J=7.9 Hz, 1H), 6.51 (d, J=9.3 Hz, 1H), 4.15 (t, J=6.0 Hz, 2H), 4.11 (t, J=5.5 Hz, 2H), 4.00 (s, 3H), 3.96 (s, 2H), 3.06 (t, J=5.5 Hz, 2H), 2.82 (t, J=7.0 Hz, 2H), 2.12-2.07 (m, 2H). ¹³C NMR (201 MHz, CDCl₃) δ 164.95, 161.31, 151.25, 147.94, 140.92, 140.45, 135.58, 132.39, 129.17, 123.02, 118.12, 114.39, 112.54, 103.30, 101.97, 99.30, 66.14, 55.85, 54.09, 52.06, 49.96, 42.21, 26.94. HRMS (ESI) calculated for C₂₃H₂₅N₄O₃⁺ [M+H]⁺: 405.1921, found: 405.1918. HPLC: 96.89% (λ254 nm, t_{R}=11.48 min).

### Example 74: Preparation of compound 7-(3-(9-methoxy-3,4-dihydrobenzo[4,5]imidazo[1,2-a]pyrazin-2(1H)-yl)propoxy)-3,4-dihydroquinolin-2(1H)-one (I-D4)

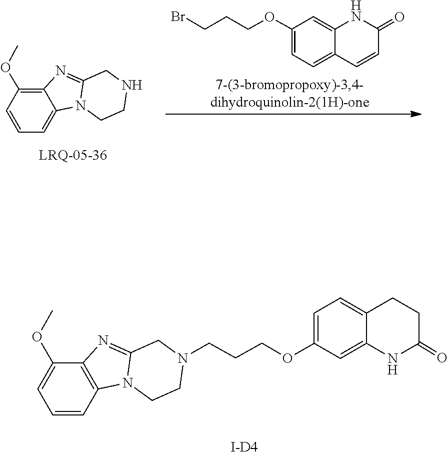

LRQ-05-36 (100 mg, 0.49 mmol) was dissolved in DMF (8 mL), then DIPEA (189 mg, 1.47 mmol) and 7-(3-bromopropoxy)-3,4-dihydroquinolin-2(1H)-one (209 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (74 mg, yield: 37%). ¹H NMR (800 MHz, CDCl₃) δ 7.16 (t, J=7.9 Hz, 1H), 7.02 (d, J=8.2 Hz, 1H), 6.95-6.88 (m, 1H), 6.69 (d, J=7.9 Hz, 1H), 6.55-6.48 (m, 1H), 6.35 (s, 1H), 4.10 (t, J=5.4 Hz, 2H), 4.02 (t, J=6.0 Hz, 2H), 4.00 (s, 3H), 3.93 (s, 2H), 3.04 (t, J=5.4 Hz, 2H), 2.87 (t, J=7.4 Hz, 2H), 2.79 (t, J=7.0 Hz, 2H), 2.60 (t, J=7.5 Hz, 2H), 2.08-2.00 (m, 2H). ¹³C NMR (201 MHz, CDCl₃) δ 171.84, 158.64, 151.34, 147.96, 138.21, 135.65, 133.02, 128.78, 122.92, 115.97, 108.71, 103.19, 102.33, 101.90, 65.93, 55.82, 54.14, 52.12, 50.02, 42.19, 31.17, 27.07, 24.70. HRMS (ESI) calculated for C₂₃H₂₇N₄O₃⁺ [M+H]⁺: 407.2078, found: 407.2073. HPLC: 98.49% (Q=254 nm, t_{R}=11.55 min).

### Example 75: Preparation of compound (E)-7-((4-(9-methoxy-3,4-dihydrobenzo[4,5]imidazo[1,2-a]pyrazin-2(1H)-yl)but-2-en-1-yl)oxy)-3,4-dihydroquinolin-2(1H)-one (I-D5)

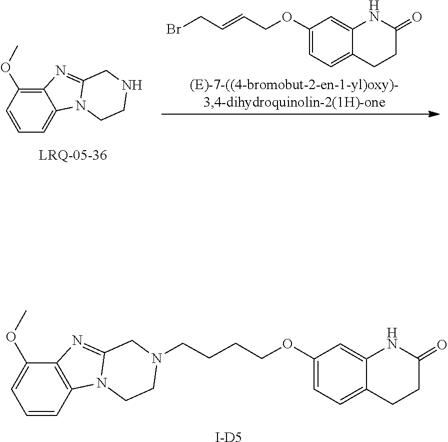 [SMILES: COc1cccc2c1nc1n2CCNC1 ; O=C1CCc2ccc(OC/C=C/CBr)cc2N1 ; COc1cccc2c1nc1n2CCN(CCCCOc2ccc3c(c2)NC(=O)CC3)C1 ; "I-D5"]

LRQ-05-36 (100 mg, 0.49 mmol) was dissolved in DMF (8 mL), then DIPEA (189 mg, 1.47 mmol) and (E)-7-((4-bromobut-2-en-1-yl)oxy)-3,4-dihydroquinolin-2(1H)-one (218 mg, 0.74 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (109 mg, yield: 53%). ¹H NMR (800 MHz, CDCl₃) δ 8.27 (s, 1H), 7.16 (t, J=8.0 Hz, 1H), 7.04 (d, J=8.3 Hz, 1H), 6.92 (d, J=8.0 Hz, 1H), 6.69 (d, J=7.9 Hz, 1H), 6.56-6.51 (m, 1H), 6.36 (d, J=2.4 Hz, 1H), 5.99-5.87 (m, 2H), 4.55-4.48 (m, 2H), 4.09 (t, J=5.5 Hz, 2H), 4.00 (s, 3H), 3.92 (s, 2H), 3.29 (d, J=5.3 Hz, 2H), 3.02 (t, J=5.5 Hz, 2H), 2.89 (t, J=7.5 Hz, 2H), 2.64-2.56 (m, 2H). ¹³C NMR (201 MHz, CDCl₃) δ 171.87, 158.20, 151.36, 147.87, 138.32, 135.68, 133.12, 129.81, 129.40, 128.84, 122.90, 116.25, 109.01, 103.20, 102.63, 101.90, 68.17, 59.10, 55.84, 52.14, 49.31, 42.13, 31.18, 24.72. HRMS (ESI) calculated for C₂₄H₂₇N₄O₃⁺ [M+H]⁺: 419.2078, found: 419.2081. HPLC: 98.33% (λ=254 nm, t_{R}=11.81 min).

### Example 76: Preparation of compound 3-(trans-4-(2-(9-methoxy-3,4-dihydrobenzo[4,5]imidazo[1,2-a]pyrazin-2(1H)-yl)ethyl)cyclohexyl)-1,1-dimethylurea (I-D6)

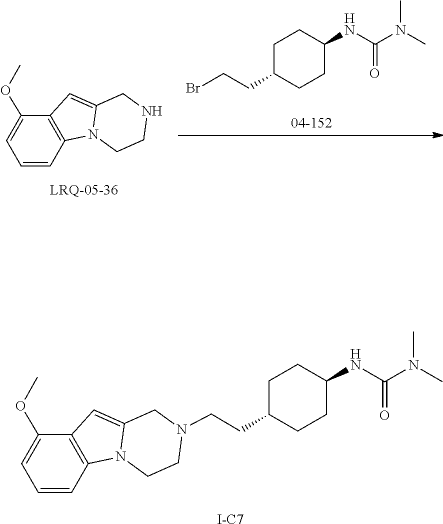 [SMILES: COc1cccc2c1cc1n2CCNC1 ; CN(C)C(=O)N[C@H]1CC[C@H](CCBr)CC1 ; COc1cccc2c1cc1n2CCN(CC[C@H]2CC[C@H](NC(=O)N(C)C)CC2)C1 ; "I-C7"]

LRQ-05-36 (24 mg, 0.12 mmol) was dissolved in DMF (8 mL), then DIPEA (45 mg, 0.35 mmol) and LRQ-04-152 (47 mg, 0.17 mmol) were sequentially added thereto, and the reaction mixture was reacted at 100° C. overnight. After the reaction was completed, the reaction mixture was diluted with water (10 mL), extracted with ethyl acetate (50 mL*3), washed with saturated brine, and dried over anhydrous sodium sulfate. The solvent was removed by evaporation under reduced pressure, and the residue was purified by silica gel column chromatography (methanol/dichloromethane=1:10) to obtain a yellow solid (12 mg, yield: 25%). ¹H NMR (800 MHz, CDCl₃) δ 7.16 (t, J=8.0 Hz, 1H), 6.93 (d, J=8.0 Hz, 1H), 6.70 (d, J=7.9 Hz, 1H), 4.12 (d, J=7.5 Hz, 1H), 4.10 (t, J=5.5 Hz, 2H), 4.01 (s, 3H), 3.89 (s, 2H), 3.61-3.54 (m, 1H), 3.00 (t, J=5.5 Hz, 2H), 2.87 (s, 6H), 2.65-2.59 (m, 2H), 2.05-1.99 (m, 2H), 1.81-1.77 (m, 2H), 1.53-1.47 (m, 2H), 1.35-1.28 (m, 1H), 1.14-1.03 (m, 4H). ¹³C NMR (201 MHz, CDCl₃) δ 157.97, 151.28, 148.17, 135.65, 132.94, 122.89, 103.21, 101.93, 55.84, 55.44, 52.21, 49.94, 49.79, 42.20, 36.27 (2C), 35.10, 34.07 (2C), 34.03, 32.11 (2C). HHRMS (ESI) calculated for C₂₂H₃₄N₅₀₂⁺ [M+H]⁺: 400.2707, found: 400.2699. HPLC: 97.29% (Q=254 nm, t_{R}=11.14 min).

### Example 77: Preparation of compound 1-((9-methoxy-3,4-dihydrobenzo[4,5]imidazo[1,2-a]pyrazin-2(1H)-yl)methyl)cyclohexan-1-ol (I-D7) (IHCH-5230)

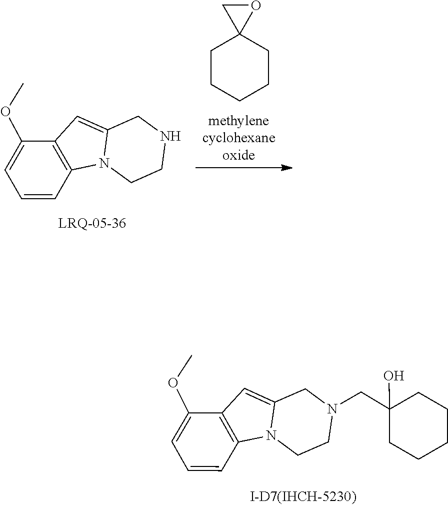 [SMILES: COc1cccc2c1cc1n2CCNC1 ; C1CCC2(CC1)CO2 ; COc1cccc2c1cc1n2CCN(CC2(O)CCCCC2)C1]

LRQ-05-36 (50 mg, 0.25 mmol) was dissolved in anhydrous ethanol (10 mL), then methylene cyclohexane oxide (138 mg, 1.25 mmol) was added thereto, and the reaction mixture was heated to 60° C. and reacted overnight. After the reaction was completed, the solvent was removed by evaporation under reduced pressure, and the residue was purified by silica gel column chromatography (methanol/dichloromethane=1:10) to obtain a white solid (10 mg, yield: 13%). ¹H NMR (800 MHz, CDCl₃) δ 7.16 (t, J=8.0 Hz, 1H), 6.95-6.89 (m, 1H), 6.69 (d, J=7.9 Hz, 1H), 4.14-4.05 (m, 4H), 3.99 (s, 3H), 3.25-3.16 (m, 2H), 2.58 (s, 2H), 1.67-1.60 (m, 2H), 1.60-1.51 (m, 3H), 1.49-1.43 (m, 2H), 1.42-1.36 (m, 2H), 1.30-1.26 (m, 1H). ¹³C NMR (201 MHz, CDCl₃) δ 151.31, 148.03, 135.64, 132.85, 123.01, 103.19, 101.90, 71.27, 66.48, 55.79, 54.53, 52.18, 42.09, 36.25 (2C), 25.90, 22.12 (2C). HHRMS (ESI) calculated for C₁₈H₂₆N₃O₂⁺ [M+H]⁺: 316.2020, found: 316.2025. HPLC: 99.78% (λ=254 nm, t_{R}=11.67 min).

### Bioassay Example 1: Affinity Testing of Compounds of General Formula (I) for Dopamine D₂ Receptors

The affinity of the compounds of the present disclosure for dopamine D₂ receptors was determined by a radioligand competition binding assay.

### Experimental Steps:

In the first step, a cell membrane component containing specific dopamine D₂ receptors was prepared. A 10 cm culture dish was used for transfection with 10 ng of dopamine D₂ receptors and 40 μL of polyethylenimine (PEI hereafter). After 48 hours, the 10 cm culture dish was taken out from the cell incubator and the cultured cells had expressed the dopamine D₂ receptors. A vacuum pump was used to suck off the culture medium, 3 mL of lysis buffer was added to each culture dish, and the cells were placed in a 4° C. cold room for 10 minutes. After the cells were detached, the cells were transferred to a 15 mL centrifuge tube and centrifuged at 1500 rpm for 5 minutes at 4° C., and the supernatant was discarded. The cell pellet was transferred to a tissue homogenizer, and 3 mL of lysis buffer was added and fully ground until the cells were broken. Then, cell suspension was equally aliquoted into several EP tubes, centrifuged at 12000 rpm for 5 minutes at 4° C., and the supernatant was discarded. The precipitate was the cell membrane component containing the dopamine D₂ receptors.

In the second step, a ligand-receptor binding assay was performed on 293T membrane component transiently expressing the dopamine D₂ receptors. First, a standard binding buffer was added to the cell membrane component containing the dopamine D₂ receptors, and the cell membrane was disrupted and resuspended with an electric tissue homogenizer. 30 μL of membrane protein suspension was added to each well of a 96-well plate. Then, 30 μL of different drugs were added to the 96-well plate sequentially from bottom to top to ensure that the final drug concentrations were 10⁻⁵ M, 10⁻⁶ M, 10⁻⁷ M, 10⁻⁸ M, 10⁻⁹ M, and 0 M, with two replicates per treatment. Next, 30 μL of [3H]-Methylspiperone was added to each well of the 96-well plate. The plate was incubated at room temperature in the dark for 2 hours. Detection was conducted. The machine readout value reflected the amount of [3H]-Methylspiperone bound on the membrane, and after further data processing, the affinity Kᵢ values of different compounds for the dopamine D₂ receptors were obtained.

The results are shown in Table 1. The results show that compound I-A1 to compound I-D7 all have some affinity activity for dopamine D₂ receptors, indicating that the compounds of the present disclosure all have some affinity activity for dopamine D₂ receptors.

TABLE 1

| Compound | Affinity Kᵢ (pKᵢ ± SEM) (nM) | |
|---|---|---|
| I-A1 | 84.42 | (7.07 ± 0.02) |
| I-A2 | 113.41 | (7.41 ± 0.45) |
| I-A5 | 290.35 | (6.54 ± 0.01) |
| I-A8 | 149.05 | (6.82 ± 0.16) |
| I-A9 | 203.86 | (6.69 ± 0.17) |
| I-A10 | 138.99 | (6.85 ± 0.19) |
| I-A12 | 436.43 | (6.43 ± 0.19) |
| I-A13 | 1721 | (5.80 ± 0.12) |
| I-A14 | 751.9 | |
| I-A15 | 272.2 | |
| I-A16 | 16.54 | (7.78 ± 0.41) |
| I-A18 | 88.78 | (7.05 ± 0.09) |
| I-A19 | 929.68 | (6.03 ± 0.07) |
| I-A20 | 89.53 | (7.05 ± 0.07) |
| I-A21 | 523.6 | (6.28 ± 0.28) |
| I-A25 | 37.97 | (7.42 ± 0.05) |
| I-A28 | 102.56 | (6.99 ± 0.21) |
| I-A29 | 27.67 | (7.56 ± 0.16) |
| I-A30 | 151.18 | (6.82 ± 0.12) |
| I-A34 | 604.64 | (6.21 ± 0.06) |
| I-A45 | 714.49 | (6.14 ± 0.15) |
| I-B1 | 361.63 | (7.08 ± 0.81) |
| I-B2 | 78.91 | (7.11 ± 0.04) |
| I-B3 | 282.16 | (6.54 ± 0.33) |
| I-B4 | 727.78 | (6.13 ± 0.26) |
| I-B6 | 266.07 | (6.58 ± 0.41) |
| I-B7 | 2.71 | (8.74 ± 0.33) |
| I-B8 | 381.07 | (6.41 ± 0.16) |
| I-B10 | 23.25 | (7.63 ± 0.57) |
| I-C1 | 400.83 | (6.57 ± 0.26) |
| I-C2 | 220.95 | (7.21 ± 0.50) |
| I-C5 | 437.5 | (6.36 ± 0.28) |
| I-C8 | 239.2 | |
| I-D6 | 16.83 | (7.89 ± 0.25) |

Note:
[³H]-Methylspiperone (0.3 to 0.5 nM) is used as the mean Kᵢ (pKᵢ ± SEM) of the radioligand competition binding assay.

### Bioassay Example 2: Functional Activity Testing of Compounds for Dopamine D₂ Receptors

To examine the downstream G-protein signaling pathway mediated by dopamine D₂ receptors, on the first day, a 6 cm culture dish was used for transfection with 1 μg of dopamine D₂ receptors, 1 μg of Gαᵢ₁ containing C-terminal seaweed luciferase (Gαᵢ₁-Rluc), 1 μg of G_{β3}, 1 μg of Gγ₉ containing C-terminal green fluorescent protein (G_{γ9}-GFP) mixed with 16 μL of PEI. At the same time, in order to examine the downstream β-arrestin2 signaling pathway mediated by dopamine D₂ receptors, on the first day, a 6 cm culture dish was used for transfection with 500 kg of dopamine D₂ receptors containing C-terminal seaweed luciferase (D₂-Rluc), 500 kg of G protein-coupled receptor kinase 2 (GRK2), 2500 kg of β-arrestin2 containing N-terminal green fluorescent protein (GFP2-ARRB2) mixed with 14 μL of PEI. The next day, the confluent cells of a 6 cm culture dish were digested and reseeded into a 96-well plate with 100 μL of culture medium added to each well. On the third day, the drugs were added for test. The 96-well plate was taken out from the cell incubator to decant the culture medium, 40 μL of the substrate coelenterazine 400a (final concentration of 5 μM) was added to each well, and then 20 μL of different drugs were added sequentially from left to right to ensure that the final concentration of the drug decreased gradually from bottom to top, with two replicates per treatment. Finally, the prepared samples were loaded on machine for test. The machine readout value reflected the recruitment of β-arrestin2 to the membrane or the dissociation of G protein trimer. The former indicated the degree of activation of the β-arrestin2 signaling pathway downstream of the dopamine D₂ receptors, and the latter indicated the degree of activation of the G protein signaling pathway downstream of the dopamine D₂ receptors. Thus, the agonistic effects of different compounds on dopamine D₂ receptors can be revealed. The results are shown in Table 2.

The results show that compound I-A1 to compound I-D7 all have some agonistic activity for dopamine D₂ receptors.

TABLE 2

| | Gαᵢ₁ BRET | β-arrestin2 BRET |
|---|---|---|
| | EC¹₅₀ (Eₘₐₓ %² ± SEM) | EC₅₀ (Eₘₐₓ % ± SEM) |
| Compound | (pEC₅₀ ± SEM) | (pEC₅₀ ± SEM) |
| I-A1 | 48.82 (55 ± 4%) | 27.67 (81 ± 6%) |
| | (7.63 ± 0.59) | (7.62 ± 0.17) |
| I-A2 | 33.96 (49 ± 6%) | 8.88 (94 ± 11%) |
| | (8.31 ± 0.79) | (8.72 ± 0.84) |
| I-A4 | 708.7 (55 ± 3%) | 81.18 (65 ± 7%) |
| | (6.16 ± 0.06) | (7.17 ± 0.18) |
| I-A5 | 311.5 (50 ± 4%) | 325.8 (82 ± 15%) |
| | (6.52 ± 0.07) | (6.51 ± 0.10) |
| I-A7 | 467.8 (41 ± 4%) | 239.2 (75 ± 7%) |
| | (6.43 ± 0.20) | (6.63 ± 0.05) |
| I-A8 | 0.15 (68%) | 52.57 (103%) |
| I-A9 | 0.48 (73%) | 617 (147%) |
| I-A10 | 61.32 (27 ± 4%) | 4.61 (107%) |
| | (7.21 ± 0.12) | |
| I-A11 | 9.34 (48 ± 4%) | 3.80 (94 ± 11%) |
| | (8.03 ± 0.04) | (8.45 ± 0.11) |
| I-A12 | 1.38 (70 ± 1%) | 10.72 (108 ± 12%) |
| | (8.86 ± 0.01) | (8.09 ± 0.26) |
| I-A13 | 46.61 (53 ± 1%) | 4.61 (81 ± 13%) |
| | (7.41 ± 0.20) | (9.08 ± 0.78) |
| I-A16 | | 11.34 (31 ± 6%) |
| | | (7.94 ± 0.11) |
| I-A17 | 4.37 (49 ± 18%) | 6.70 (29 ± 5%) |
| | (8.435 ± 0.20) | (8.17 ± 0.03) |
| I-A18 | | 2.93 (20 ± 6%) |
| | | (8.53 ± 0.88) |
| I-A19 | 10.55 (40 ± 19%) | 8.36 (21 ± 2%) |
| | (7.97 ± 0.25) | (8.08 ± 0.06) |
| I-A20 | 1.98 (91%) | 4.95 (150%) |
| I-A21 | 4.61 (68 ± 11%) | 23.56 (59%) |
| | (8.53 ± 0.32) | |
| I-A22 | 55.96 (79 ± 18%) | 86.39 (89%) |
| | (7.25 ± 0.12) | |
| I-A23 | 412.02 (97%) | 351.4 (92%) |
| I-A24 | 176.7 (61%) | |
| I-A25 | 1.32 (40 ± 19%) | 54.57 (78%) |
| | (8.69 ± 0.15) | |
| I-A26 | | 53.34 (35%) |
| I-A27 | 45.19 (57%) | 55.02 (101%) |
| I-A28 | 32.45 (27 ± 3%) | 238.5 (82%) |
| | (7.35 ± 0.02) | |
| I-A29 | 33.11 (40 ± 19%) | 10.12 (47%) |
| | (7.48 ± 0.25) | |
| I-A30 | | 23.66 (52%) |
| I-A32 | 12.91 (21%) | 232.7 (12%) |
| I-A33 | | 56.7 (18%) |
| I-A34 | | 2.81 (33%) |
| I-A35 | | 3.17 (17%) |
| I-A36 | | 0.75 (12%) |
| I-A43 | | 48.78 (21%) |
| I-A44 | | 206.1 (21%) |
| I-A45 | | 25.39 (30%) |
| I-A46 | | 212.9 (31%) |
| I-B1 | 232.95 (22 ± 3%) | 13.08 (50 ± 2%) |
| | (6.99 ± 0.40) | (7.92 ± 0.13) |
| I-B3 | 12.98 (41 ± 1%) | 0.47 (90%) |
| | (7.88 ± 0.45) | |
| I-B5 | 43.59 (35%) | 6.53 (76%) |
| I-B7 | 17.02 (21 ± 3%) | 0.24 (44 ± 1%) |
| | (7.95 ± 0.32) | (9.97 ± 0.48) |
| I-B9 | 43.32 (14 ± 4%) | 132.75 (51%) |
| | (7.21 ± 0.12) | |
| I-C1 | 7.94 (72 ± 4%) | 219.7 (117 ± 17%) |
| | (8.20 ± 0.23) | (6.66 ± 0.01) |
| I-C2 | 46.25 (81 ± 4%) | 122.16 (115 ± 16%) |
| | (7.35 ± 0.08) | (7.18 ± 0.41) |
| I-C3 | 129.7 (71%) | 18.29 (97%) |
| I-C4 | 9.04 (56%) | 2.94 (100%) |
| I-C5 | 96.49 (64 ± 19%) | 20.85 (127%) |
| | (7.01 ± 0.03) | |
| I-C6 | 276.06 (49 ± 19%) | 2.45 (92%) |
| | (6.56 ± 0.47) | |
| I-C7 | 123.47 (97 ± 2%) | 290.78 (151 ± 19%) |
| | (6.91 ± 0.04) | (6.71 ± 0.27) |
| I-C10 | 117.7 (20 ± 6%) | 827.9 (49 ± 5%) |
| | (6.92 ± 0.38) | (6.08 ± 0.27) |
| I-C11 | 39.77 (23 ± 7%) | 127.6 (45 ± 2%) |
| | (7.4 ± 0.25) | (6.89 ± 0.14) |
| I-C12 | 23.43 (66 ± 12%) | 54.43 (52%) |
| | (7.41 ± 0.13) | |
| I-D1 | 266.7 (77 ± 8%) | 56.02 (100 ± 5%) |
| | (6.58 ± 0.04) | (7.25 ± 0.04) |
| I-D2 | 355.07 (71 ± 3%) | 70.08 (106 ± 6%) |
| | (6.49 ± 0.12) | (7.16 ± 0.04) |
| I-D3 | 129.7 (71%) | 18.29 (97%) |
| I-D4 | 159.3 (81%) | 33.56 (89%) |
| I-D5 | 159.77 (46 ± 11%) | 38.84 (66%) |
| | (6.79 ± 0.29) | |
| I-D7 | 79.03 (67 ± 8%) | 180.18 (41%) |
| | (7.02 ± 0.21) | |
| Aripiprazole | 426.58 (60 ± 1%) | 309.03 (58 ± 3%) |
| | (6.37 ± 0.05) | (6.51 ± 0.10) |
| Cariprazine | 10 nM (58%) | 12 nM (61%) |

Note:
All data are mean ± SEM of three independent measurements (n = 3 independent experiments).
¹EC₅₀ is the compound concentration giving half the maximum response in the experiment.
²Eₘₐₓ % in parentheses indicates the percentage of the maximum reaction intensity (Eₘₐₓ) produced by the compound in the experiment relative to the endogenous ligand dopamine.

### Bioassay Example 3: Affinity Testing of Compounds of General Formula (I) for 5-Hydroxytryptamine 1A (5-HT_{1A}) Receptors

The affinity of the compounds of the present disclosure for 5-hydroxytryptamine 1A (5-HT_{1A}) receptors was determined by a radioligand competition binding assay.

### Experimental Steps:

In the first step, a cell membrane component containing specific 5-HT_{1A} receptors was prepared. A 10 cm culture dish with HEK-293T cells was used for transfection with 10 μg of 5-HT_{1A} receptors and 40 μL of PEI. After 48 hours, the 10 cm culture dish was taken out from the incubator and the cultured cells had expressed the 5-HT_{1A} receptors. A vacuum pump was used to suck off the culture medium, 3 mL of lysis buffer was added to each culture dish, and the cells were placed in a 4° C. cold room for 10 minutes. After the cells were detached, the cells were transferred to a 15 mL centrifuge tube and centrifuged at 1500 rpm for 5 minutes at 4° C., and the supernatant was discarded. The cell pellet was transferred to a tissue homogenizer, and 3 mL of lysis buffer was added and fully ground until the cells were broken. Then, cell suspension was equally aliquoted into several EP tubes, centrifuged at 12000 rpm for 5 minutes at 4° C., and the supernatant was discarded. The precipitate was the cell membrane component containing the 5-HT_{1A} receptors.

In the second step, a ligand-receptor binding assay was performed on 293T membrane component transiently expressing the 5-HT_{1A} receptors. First, a standard binding buffer was added to the cell membrane component containing the 5-HT_{1A} receptors, and the cell membrane was disrupted and resuspended with an electric tissue homogenizer. 30 μL of membrane protein suspension was added to each well of a 96-well plate. Then, 30 μL of different drugs were added to the 96-well plate sequentially from bottom to top to ensure that the final drug concentrations were 10⁻⁵ M, 10⁻⁶ M, 10⁻⁷ M, 10⁻⁸ M, 10⁻⁹ M, and 0 M, with two replicates per treatment. Next, 30 μL of [³H]-LSD was added to each well of the 96-well plate. The plate was incubated at room temperature in the dark for 2 hours. Detection was conducted. The machine readout value reflected the amount of [³H]-LSD bound on the membrane, and after further data processing, the binding affinity Kᵢ values of different compounds for the 5-HT_{1A} receptors were obtained.

### Experimental Steps:

The results are shown in Table 3. The results show that the compounds of the present disclosure have strong affinity activity for 5-hydroxytryptamine 1A (5-HT_{1A}) receptors.

TABLE 3

| Compound | Affinity Kᵢ |
|---|---|
| I-A15 | 5.43 nM |
| I-A22 | 0.073 nM |
| I-A27 | 0.64 nM |
| I-A32 | 2.55 nM |
| I-A41 | 0.78 nM |
| I-A42 | 0.42 nM |
| I-A43 | 0.015 nM |
| Aripiprazole | 53.79 nM |
| Cariprazine | 9.94 nM |

### Bioassay Example 4: Functional Activity Testing of Compounds for 5-Hydroxytryptamine 1A (5-HT_{1A}) Receptors

To examine the downstream G-protein signaling pathway mediated by 5-HT_{1A} receptors, on the first day, a 6 cm culture dish with HEK-293T cells was used for transfection with 1 μg of 5-HT_{1A} receptors, 1 μg of Gαᵢ₁ containing C-terminal seaweed luciferase (Gαᵢ₁-Rluc), 1 μg of G₃, 1 μg of Gγ₉ containing C-terminal green fluorescent protein (G_{γ9}-GFP), and 16 μL of PEI. At the same time, in order to examine the downstream β-arrestin2 signaling pathway mediated by 5-HT_{1A} receptors, on the first day, a 6 cm culture dish was used for transfection with 500 ng of 5-HT_{1A} receptors containing C-terminal seaweed luciferase (5HT_{1A}R-Rluc), 500 ng of G protein-coupled receptor kinase 2 (GRK₂), 2500 ng of β-arrestin2 containing N-terminal green fluorescent protein (GFP2-ARRB2), and 14 μL of PEI. The next day, the confluent cells of a 6 cm culture dish were digested and reseeded into a 96-well plate with 100 μL of culture medium added to each well. On the third day, the drugs were added for test. The 96-well plate was taken out from the cell incubator to decant the culture medium, 40 L of the substrate coelenterazine 400a (final concentration of 5 μM) was added to each well, and then 20 μL of different drugs were added sequentially from left to right to ensure that the final concentration of the drug decreased gradually from bottom to top, with two replicates per treatment. Finally, the prepared samples were loaded on machine for test. The machine readout value reflected the recruitment of β-arrestin2 to the membrane or the dissociation of G protein trimer. The former indicated the degree of activation of the β-arrestin2 signaling pathway downstream of the 5-HT₁ serotonin 1A receptors, and the latter indicated the degree of activation of the G protein signaling pathway downstream of the serotonin 1A receptors. Thus, the agonistic effects of different compounds on serotonin 1A receptors can be revealed.

The results are shown in Table 4.

The results show that the compounds of the present disclosure have moderate to strong agonistic activity for 5-hydroxytryptamine 1A (5-HT_{1A}) receptors.

TABLE 4

| | Gαᵢ₁ BRET | β-arrestin2 BRET |
|---|---|---|
| Compound | EC¹₅₀ (Eₘₐₓ %²) | EC₅₀ (Eₘₐₓ %) |
| I-A1 | 153.5 | |
| I-A12 | 29.36 | |
| I-A14 | 88.21 (108%) | 72.11 (101%) |
| I-A15 | 8.08 (123%) | 2.54 (92%) |
| I-A16 | 138.7 (85.94%) | |
| I-A18 | 359.2 (81.91%) | |
| I-A20 | 29.25 (66.13%) | |
| I-A22 | 11.09 (59.5%) | 0.78 (49.6%) |
| I-A23 | 489.1 (75.2%) | 29.43 (41.8%) |
| I-A24 | 149.3 (107.1%) | 6854 |
| I-A25 | 38.33 (78.05%) | |
| I-A26 | 92.98 (85.69%) | 114.8 (9.8%) |
| I-A27 | 9.68 (64.06%) | |
| I-A29 | 20.85 (90.33%) | 43.52 (8.46%) |
| I-A30 | 182.4 (74.52%) | |
| I-A31 | 188.4 (103.8%) | |
| I-A32 | 7.23 (102.3%) | 137 (31.61%) |
| I-A33 | 22.7 (96.67%) | 656.5 (25.24%) |
| I-A34 | 188 (38.42%) | |
| I-A35 | 15.13 (102.4%) | 459.6 (39.75%) |
| I-A36 | 27.67 (100.5%) | 1179 (38.7%) |
| I-A37 | 391.7 (113.4%) | |
| I-A38 | 37.03 (116.2%) | |
| I-A39 | 25.62 (82.23%) | 5.26 (20.64%) |
| I-A40 | 135.4 (101%) | 3042 (28.92%) |
| I-A41 | 6.44 (98.78%) | 99.68 (14.29%) |
| I-A42 | 7.54 (101.3%) | 382 (21.02%) |
| I-A43 | 13.08 (84.32%) | |
| I-A44 | 387.7 (75.91%) | |
| I-A45 | 70.81 (74.27%) | |
| I-A46 | 115.2 (98.48%) | |
| I-B7 | 301 | |
| I-B8 | 318.2 (37.85%) | |
| I-B11 | 51.46 (22.34%) | |
| I-C8 | 1721 (81%) | 493.8 (79%) |
| Aripiprazole | 436 (75%) | 558 (28%) |
| Cariprazine | 78.25 (80%) | Inactive |

Note:
¹EC₅₀ is the compound concentration giving half the maximum response in the experiment.
²Eₘₐₓ % in parentheses indicates the percentage of the maximum reaction intensity (Eₘₐₓ) produced by the compound in the experiment relative to the endogenous ligand 5-hydroxytryptamine.

### Bioassay Example 5: Affinity Testing of Compounds of the Present Disclosure for 5-HT_{2A} Receptors

The affinity of the compounds of the present disclosure for 5-HT₂A receptors was determined by a radioligand competition binding assay. In the first step, a cell membrane component containing specific 5-HT_{2A} receptor was prepared. A 10 cm dish was used for transfection with 10 ng of 5-HT_{2A} receptors and 40 μL of PEI. After 48 hours, the 10 cm dish was taken out from the cell incubator and the cultured cells had expressed the 5-HT_{2A} receptors. A vacuum pump was used to suck off the culture medium, 3 mL of lysis buffer was added to each culture dish, and the cells were placed in a 4° C. cold room for 10 minutes. After the cells were detached, the cells were transferred to a 15 mL centrifuge tube and centrifuged at 1500 rpm for 5 minutes at 4° C., and the supernatant was discarded. The cell pellet was transferred to a tissue homogenizer, and 3 mL of lysis buffer was added and fully ground until the cells were broken. Then, cell suspension was equally aliquoted into several EP tubes, centrifuged at 12000 rpm for 5 minutes at 4° C., and the supernatant was discarded. The precipitate was the cell membrane component containing the 5-HT_{2A} receptors. In the second step, a ligand-receptor binding assay was performed on 293T membrane component transiently expressing the 5-HT_{2A} receptors. First, a standard binding buffer was added to the cell membrane component containing the 5-HT_{2A} receptors, and the cell membrane was disrupted and resuspended with an electric tissue homogenizer. 30 μL of membrane protein suspension was added to each well of a 96-well plate. Then, 30 μL of different drugs were added to the 96-well plate sequentially from bottom to top to ensure that the final drug concentrations were 10⁻⁵ M, 10⁻⁶ M, 10⁻⁷ M, 10⁻⁸ M, 10⁻⁹ M, and 0 M, with two replicates per treatment. Next, 30 μL of [³H]-ketanserin was added to each well of the 96-well plate. The plate was incubated at room temperature in the dark for 2 hours. Detection was conducted. The machine readout value reflected the amount of [³H]-ketanserin bound on the membrane, and after further data processing, the affinity Kᵢ values of different compounds for the 5-HT_{2A} receptors were obtained. The results are shown in Table 3.

TABLE 5

| Compound | Kᵢ, nM (pKᵢ ± SEM) | |
|---|---|---|
| I-A1 | 1793 nM | |
| | (5.74 ± 0.03) | |
| I-A2 | 1107 nM | |
| | (5.95 ± 0.05) | |
| I-A16 | 1320 | nM |
| I-A20 | 1307 | nM |
| I-A21 | >10000 | nM |
| I-A28 | >10000 | nM |
| I-A29 | 5524 | nM |
| I-B7 | 3514 | nM |
| Aripiprazole | 42.2 | nM |
| Cariprazine | 62.9 | nM |

The compounds of the present disclosure exhibit very weak affinity for 5-HT₂A receptors and have selectivity for D₂ receptors and 5-HT_{1A} receptors relative to 5-HT₂A receptors. The results in Table 5 show that compounds I-A1, I-A2, I-A16, I-A20, I-A21, I-A28, I-A29, and I-B7 exhibit very weak affinity for 5-HT₂A receptors. Comparing the data in Table 1, it can be seen that the compounds of the present disclosure have good selectivity for D₂ receptors relative to 5-HT₂A receptors.

### Bioassay Example 6: Testing of Pharmacokinetic Properties of Compounds of the Present Disclosure in Rats

Testing of pharmacokinetic properties of compounds administered in a single dose to SD rats by intragastric administration and intravenous injection

### (1) Experimental Purpose

After the compound was administered in a single dose to male SD rats, blood samples were collected at different time points. The concentration of the compound in the plasma of the rats was determined by LC-MS/MS and the relevant pharmacokinetic parameters were calculated to investigate the pharmacokinetics of the compound in rats.

### (2) Experimental Methods

Male SD rats were provided by Suzhou JOINN Laboratories Co., Ltd. SD rats were fasted with free access to water for 12 to 14 hours one day before administration, and were fed 4 hours after administration.

For each compound, the mice were divided into two groups: intravenous injection and intragastric administration, with 3 rats in each group. For both intravenous injection (administered at a dose of 3 mg/kg and a concentration of 0.6 mL/kg) and intragastric administration (administered at a dose of 10 mg/kg and a concentration of 1 mL/kg), 5% DMSO+5% Solutol+90% saline was used as the vehicle.

Sample collection: 0.10 mL of blood was collected from the orbit of each animal in IV/PO groups each time at 5 minutes, 15 minutes, 30 minutes, 1 hour, 2 hours, 4 hours, 6 hours, 8 hours, and 24 hours after administration of the test compound, and anticoagulated with EDTA-K₂. Blood samples were placed on ice after collection, and plasma was separated by centrifugation within 30 minutes (centrifugation conditions: 5000 rpm, 10 minutes, 4° C.). The collected plasma was stored at −80° C. before analysis.

Data processing: The data acquisition and control system software was Analyst 1.5.1 software (Applied Biosystem). The peak integration method of chromatographic samples was automatic integration; the ratio of the peak area of the sample to the peak area of the internal standard was used as an index for regression with the concentration of the sample. Regression method: linear regression, with a weight coefficient of 1/X². Pharmacokinetic parameters were analyzed with a non-compartmental model using WinNonlin Professional v6.3 (Pharsight, USA). Cₘₐₓ was the measured maximum plasma concentration, the area under the plasma concentration-time curve AUC_{(0→t)} was calculated by the trapezoidal method, and tₘₐₓ was the time to peak plasma concentration after administration. Experimental data were presented as “Mean±SD” (n≥3) or “Mean” (n=2).

### (3) Experimental Results

The metabolic properties of the compounds of the present disclosure in SD rats are shown in Table 6 to 8. As can be seen from the data presented in Tables 6 to 8, the compounds of the present disclosure have good pharmacokinetic properties, reasonable half-life, and good oral bioavailability in SD rats.

TABLE 6
Metabolic properties of compounds I-A1 and I-A12 in rats

| | | I-A1 | | I-A12 | |
|---|---|---|---|---|---|
| | | Intravenous | Oral | Intravenous | Oral |
| Pharmacokinetic parameters | | injection | administration | injection | administration |
| Dose | mg/kg | 3 | 10 | 3 | 10 |
| Elimination half-life t_{1/2} | h | 3.48 ± 0.1 | 3.91 ± 0.3 | 4.14 ± 0.7 | 3.58 ± 0.5 |
| Time to peak | h | — | 0.50 ± 0 | — | 0.833 ± 1.0 |
| concentration tₘₐₓ | | | | | |
| Peak drug concentration | ng/mL | 1327 ± 117 | 1555 ± 362 | 824 ± 299 | 963 ± 322 |
| C₀ or Cₘₐₓ | | | | | |
| Area under the drug- | h*ng/mL | 2991 ± 212 | 8916 ± 3642 | 1099 ± 215 | 5662 ± 2659 |
| time curve AUC₀₋ₜ | | | | | |
| Clearance CL | mL/min/kg | 16.7 ± 1.12 | — | 45.4 ± 7.9 | — |
| Mean residence time | h | 3.96 ± 0.1 | 4.60 ± 0.40 | 3.36 ± 0.2 | 4.42 ± 0.66 |
| MRT | | | | | |
| Apparent volume of | L/kg | 3.96 ± 0.4 | — | 9.14 ± 1.5 | — |
| distribution Vdss | | | | | |
| Bioavailability F | % | — | 88.8 ± 36% | — | 151 ± 71% |

TABLE 7
Metabolic properties of compounds I-B7 and I-C1 in rats

| | | I-B7 | | I-C1 | |
|---|---|---|---|---|---|
| | | Intravenous | Oral | Intravenous | Oral |
| Pharmacokinetic parameters | | injection | administration | injection | administration |
| Dose | mg/kg | 3 | 10 | 3 | 10 |
| Elimination half-life t_{1/2} | h | 3.91 ± 0.3 | 5.12 ± 0.7 | 3.63 ± 0.7 | 7.36 ± 4.2 |
| Time to peak | h | — | 3.17 ± 4 | — | 0.25 ± 0 |
| concentration tₘₐₓ | | | | | |
| Peak drug concentration | ng/mL | 330 ± 61 | 734 ± 34 | 3308 ± 349 | 1154 ± 198 |
| Cₘₐₓ | | | | | |
| Area under the drug- | h*ng/mL | 2352 ± 127 | 10192 ± 945 | 4970 ± 751 | 8138 ± 2054 |
| time curve AUC₀₋ₜ | | | | | |
| Clearance CL | mL/min/kg | 21.0 ± 1.2 | — | 10.1 ± 1.4 | — |
| Mean residence time | h | 6.01 ± 0.2 | 8.08 ± 0.23 | 3.58 ± 0.3 | 10.0 ± 6.5 |
| MRT | | | | | |
| Apparent volume of | L/kg | 7.56 ± 0.4 | — | 2.16 ± 0.1 | — |
| distribution Vdss | | | | | |
| Bioavailability F | % | — | 128 ± 12% | — | 48.8 ± 12.0% |

TABLE 8
Metabolic properties of compounds I-C2 and I-C7 in rats

| | | I-C2 | | I-C7 | |
|---|---|---|---|---|---|
| | | Intravenous | Oral | Intravenous | Oral |
| Pharmacokinetic parameters | | injection | administration | injection | administration |
| Dose | mg/kg | 3 | 10 | 10 | 10 |
| Elimination half-life t_{1/2} | h | 1.79 ± 1.1 | 4.09 ± 1.6 | 3.67 ± 0.1 | 8.54 ± 2.30 |
| Time to peak | h | — | 0.33 ± 0.14 | — | 2.00 ± 0 |
| concentration tₘₐₓ | | | | | |
| Peak drug concentration | ng/mL | 3340 ± 629 | 1310 ± 701 | 765 ± 74 | 1004 ± 195 |
| Cₘₐₓ | | | | | |
| Area under the drug- | h*ng/mL | 2790 ± 842 | 7727 ± 467 | 2521 ± 446 | 10374 ± 1178 |
| time curve AUC₀₋ₜ | | | | | |
| Clearance CL | mL/min/kg | 19.1 ± 6.8 | — | 20.1 ± 3.6 | — |
| Mean residence time | h | 1.38 ± 0.3 | 5.46 ± 0.09 | 3.98 ± 0.4 | 11.5 ± 4.1 |
| MRT | | | | | |
| Apparent volume of | L/kg | 1.51 ± 0.3 | — | 4.75 ± 0.4 | — |
| distribution Vdss | | | | | |
| Bioavailability F | % | — | 82.4 ± 5.0% | — | 123 ± 14% |

Note:
“—” indicates not applicable.

### Bioassay Example 7: Brain Permeability Test of Compounds Administered Intraperitoneally in a Single Dose to C57 Male Mice

Using the same method as that in the pharmacokinetic experiment, 0.030 mL of blood was collected from the orbit of each animal at 0.5 hours, 2.0 hours, and 4.0 hours, and anticoagulated with EDTA-K₂. Blood samples were placed on ice after collection, and plasma was separated by centrifugation within 30 minutes (centrifugation conditions: 5000 rpm, 10 minutes, 4° C.). The collected plasma was stored at −80° C. before analysis. After the animals were bled and euthanized, brain tissue samples were taken and homogenized at a ratio of 1:3 in 50% methanol (m/v=1:3) based on body weight. The homogenate was stored at −80° C. before analysis. Drug concentrations in plasma and brain tissues were analyzed by LC/MS/MS for comparison.

The compound concentrations in plasma and brain tissues as well as the ratios are shown in Table 9. As can be seen from Table 9, the compounds of the present disclosure have good brain permeability properties.

TABLE 9
Brain permeability properties of compounds of the present disclosure in
mice

| | I-A1 | I-A12 | I-B7 | I-C1 | I-C2 | I-C7 |
|---|---|---|---|---|---|---|
| 0.5 h brain | 1106 ± 43 | 1399 ± 220 | 392 ± 15 | 1943 ± 63 | 2805 ± 208 | 1026 ± 96 |
| 0.5 h plasma | 2215 ± 447 | 830 ± 119 | 598 ± 19 | 4062 ± 367 | 3282 ± 456 | 336 ± 28 |
| 0.5 h ratio | 0.499 | 1.69 | 0.656 | 0.478 | 0.855 | 3.05 |
| 2.0 h brain | 236 ± 25 | 455 ± 61 | 73.0 ± 129 | 732 ± 56 | 1256 ± 279 | 714 ± 143 |
| 2.0 h plasma | 397 ± 26.3 | 202 ± 56.2 | 114 ± 67.9 | 1103 ± 89 | 1506 ± 545 | 267 ± 47 |
| 2.0 h ratio | 0.594 | 2.26 | 0.643 | 0.663 | 0.834 | 2.67 |
| 4.0 h brain | — | — | — | 161 ± 55 | 599 ± 54 | 491 ± 80 |
| 4.0 h plasma | — | — | — | 297 ± 106 | 558 ± 100 | 219 ± 15 |
| 4.0 h ratio | — | — | — | 0.541 | 0.931 | 2.24 |
| AUC brain | — | — | — | 3385 | 5561 | 2767 |
| AUC plasma | — | — | — | 6289 | 6517 | 1022 |
| AUC ratio | — | — | — | 0.538 | 0.853 | 2.71 |

Note: Intraperitoneal administration, 5 mg/kg, vehicle: 5% DMSO+95% saline; “--”: not tested; compound concentration in plasma and brain in ng/mL; AUC in hr ng/mL.

### Bioassay Example 8: Pharmacodynamic Test of Compounds on Schizophrenia-Like Animal Behavioral Model

### Open Field Test

Experimental method: The experimental animals were C57B6 male mice, n=8 in each group. In this model, C57B6 mice were used as experimental animals. Hyperlocomotion of the mice in open field was induced by acute injection of a NMDA antagonist MK801, and modeled to test the inhibitory effect of different compounds on hyperlocomotion phenotype induced by MK801 All mouse behavior experiments were carried out during the light period. The whole process was recorded by the camera, and locomotive data were tracked and analyzed by behavioral tracking software. The compounds were administered intraperitoneally. Immediately after the injection, the mice entered the open field and their movement trajectories were recorded by the camera. After 30 minutes, the mice received 0.2 mg/kg of MK801 by intraperitoneal injection and returned to the open field immediately after administration, and the movement trajectories were further recorded for 120 minutes. The accumulative travelled distance of the mice was counted in a 5-minute bin. Data statistics were performed using Student-t-test analysis, *p<0.05, **p<0.01, ***p<0.001, ****p<0.0001. The total travelled distance of the mice in the 0-45 minute interval after administration of the compounds of the present disclosure at different doses in combination with MK801 (0.2 mg/kg) is shown in Table 10.

TABLE 10
Inhibitory activity of compounds of the present disclosure
on MK801-induced hyperlocomotion in mice

| | | | | Distance (cm) travelled |
|---|---|---|---|---|
| Administration conditions | | | | in 45 minutes |
| Saline | Saline | | | 3551 |
| MK801 | Saline | | | 12958 |
| (0.2 mg/kg) | I-A1 | 0.0156 | mg/kg | 9023 |
| | | 0.0625 | mg/kg | 10706 |
| | | 0.25 | mg/kg | 3630 |
| | I-A2 | 1 | mg/kg | 8795.03 |
| | | 4 | mg/kg | 6152.62 |
| | | 16 | mg/kg | 3028.99 |
| | I-A12 | 0.0156 | mg/kg | 12729 |
| | | 0.0625 | mg/kg | 5648 |
| | | 0.25 | mg/kg | 2267 |
| | I-B7 | 0.25 | mg/kg | 13668 |
| | | 1 | mg/kg | 10199 |
| | | 4 | mg/kg | 4360 |

The results of the open field test shown in Table 10 indicate that the compounds of the present disclosure, such as I-A1, I-A12, and I-B7, can significantly inhibit MK801-induced hyperlocomotion in mice at different doses.

1. A compound of formula I, or a pharmaceutically acceptable salt, isotope derivative, enantiomer, diastereomer, tautomer, or solvate thereof, 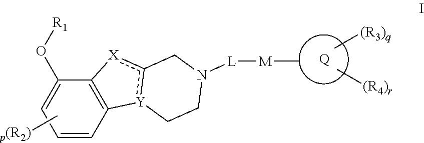
wherein
X is N, O, NRₐ, or CR_{b};
Y is C or N; [symbol]
is a double bond or a single bond;
Rₐ and R_{b} are each independently H or C₁-C₆ alkyl;
L is —(CR_{c}R_{d})ₘ— or —(CR_{c}R_{d})ₙ₁—CH═CH—(CR_{c}R_{d})ₙ₂—;
R_{c} and R_{d} are each independently H or C₁-C₆ alkyl;
m, n1, and n2 are each independently 1, 2, 3, 4, 5, or 6;
M is absent, —O—, or —NH—C(O)—;
ring Q is a saturated or partially unsaturated 4- to 8-membered carbocyclic ring, a saturated or partially unsaturated 3- to 8-membered heterocyclic ring, a 6- to 10-membered aromatic ring, a 5- to 10-membered heteroaromatic ring, or an 8- to 11-membered fused bicyclic ring; one of the rings in the 8- to 11-membered fused bicyclic ring is a saturated or partially unsaturated 5- to 7-membered carbocyclic ring or a saturated or partially unsaturated 5- to 7-membered heterocyclic ring, and the other ring is benzene ring or a 5- to 6-membered heteroaromatic ring;
R₁ is C₁-C₆ alkyl or halo-C₁-C₆ alkyl;
each R₂ is independently F, Cl, Br, I, hydroxyl, C₁-C₆ alkyl, C₁-C₆ alkoxy, halo-C₁-C₆ alkyl, or halo-C₁-C₆ alkoxy;
each R₃ is independently F, Cl, Br, I, hydroxyl, C₁-C₆ alkyl, C₁-C₆ alkoxy, halo-C₁-C₆ alkyl, halo-C₁-C₆ alkoxy, —NH—C(O)Rₑ, or —NH—S(O)₂Rₑ;
or, two R₃ together with the atom to which they are attached form a 3- to 8-membered cycloalkyl ring;
Rₑ is C₁-C₆ alkyl, —NH₂, —NHR_{g}, —NR_{f}R_{g}, or 5- to 6-membered heteroaryl;
R_{f} and R_{g} are each independently C₁-C₆ alkyl;
R₄ is oxo (═O);
p, q, and r are each independently 0, 1, 2, 3, or 4;
the number of heteroatoms in the heterocyclic ring, heteroaromatic ring, and heteroaryl is each independently 1, 2, or 3, and the heteroatoms are each independently N, O, or S.

2. The compound, or the pharmaceutically acceptable salt, isotope derivative, enantiomer, diastereomer, tautomer, or solvate thereof according to claim 1, wherein the compound satisfies one or more of the following conditions:
(1) Rₐ and R_{b} are H;
(2) R_{c} and R_{d} are H;
(3) m is 1, 2, 3, or 4;
(4) n1 and n2 are 1;
(5) in M, nitrogen atom of —NH—C(O)— is attached to L;
(6) each R₁ is independently methyl, ethyl, or isopropyl;
(7) each R₃ is independently F, Cl, Br, I, hydroxyl, C₁-C₆ alkyl, —NH—C(O)Rₑ, or —NH—S(O)₂Rₑ;
(8) p is 0;
(9) ring Q is attached to M through a C atom;
(10) in ring Q, the 4- to 8-membered carbocyclic ring is a 4-membered, 5-membered, 6-membered, or 7-membered carbocyclic ring;
(11) in ring Q, the 3- to 8-membered heterocyclic ring is a 6-membered heterocyclic ring;
(12) in ring Q, the heteroatom in the 3- to 8-membered heterocyclic ring is N or O;
(13) in ring Q, the 6- to 10-membered aromatic ring is benzene ring;
(14) in ring Q, the number of heteroatoms in the 5- to 10-membered heteroaromatic ring is 1 or 2; and
(15) in ring Q, one of the rings in the 8- to 11-membered fused bicyclic ring is a saturated or partially unsaturated 5- to 7-membered heterocyclic ring, and the other ring is benzene ring or a 5- to 6-membered heteroaromatic ring, wherein the number of heteroatoms in the 5- to 7-membered heterocyclic ring and the 5- to 6-membered heteroaromatic ring is independently 1 or 2, and the heteroatom is N.

3. The compound, or the pharmaceutically acceptable salt, isotope derivative, enantiomer, diastereomer, tautomer, or solvate thereof according to claim 14, wherein the compound satisfies one or more of the following conditions:
(1) L is —(CH₂)—, —(CH₂)₂—, —(CH₂)₃—, —(CH₂)₄—, or —(CH₂)—CH═CH—(CH₂)—; and
(1) ring Q is

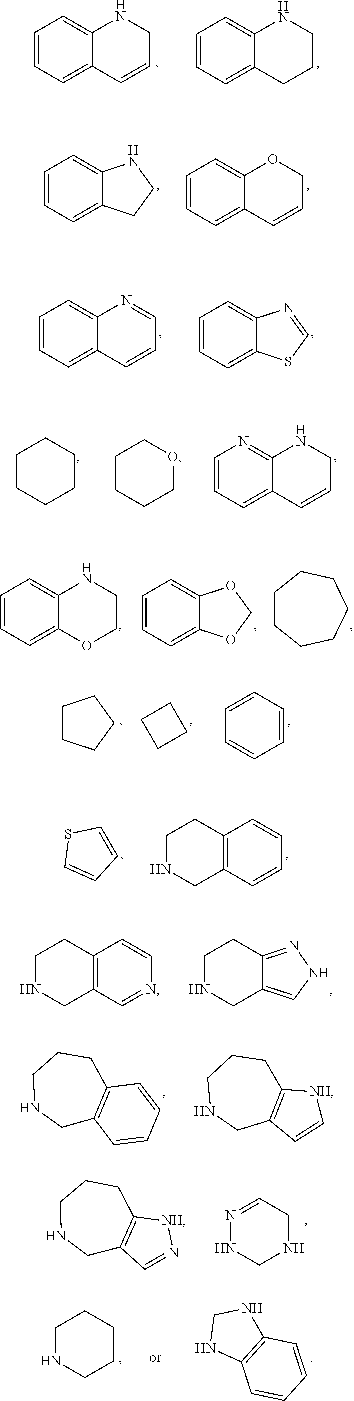

4. The compound, or the pharmaceutically acceptable salt, isotope derivative, enantiomer, diastereomer, tautomer, or solvate thereof according to claim 1, wherein the compound satisfies one or more of the following conditions: 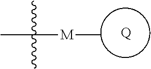
is 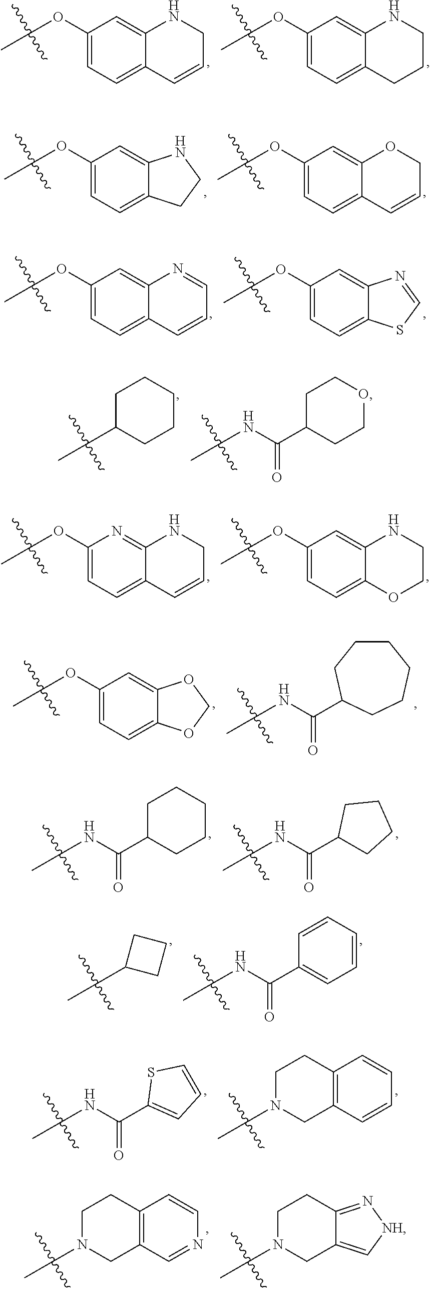 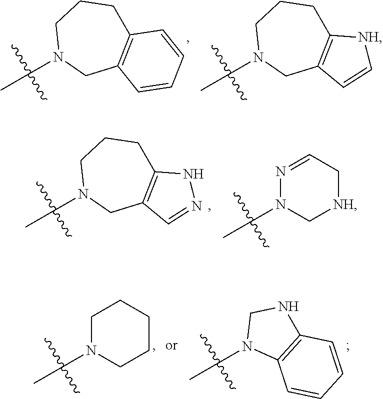 [SMILES: *N1CCCc2ccccc2C1 ; *N1CCCc2[nH]ccc2C1 ; *N1CCCc2[nH]ncc2C1 ; *N1CNCC=N1 ; *N1CCCCC1 ; *N1CNc2ccccc21]
(2) q is 0, 1, or 2; and
(2) r is 0, 1, or 2.

5. The compound, or the pharmaceutically acceptable salt, isotope derivative, enantiomer, diastereomer, tautomer, or solvate thereof according to claim 1, wherein 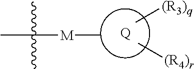
is 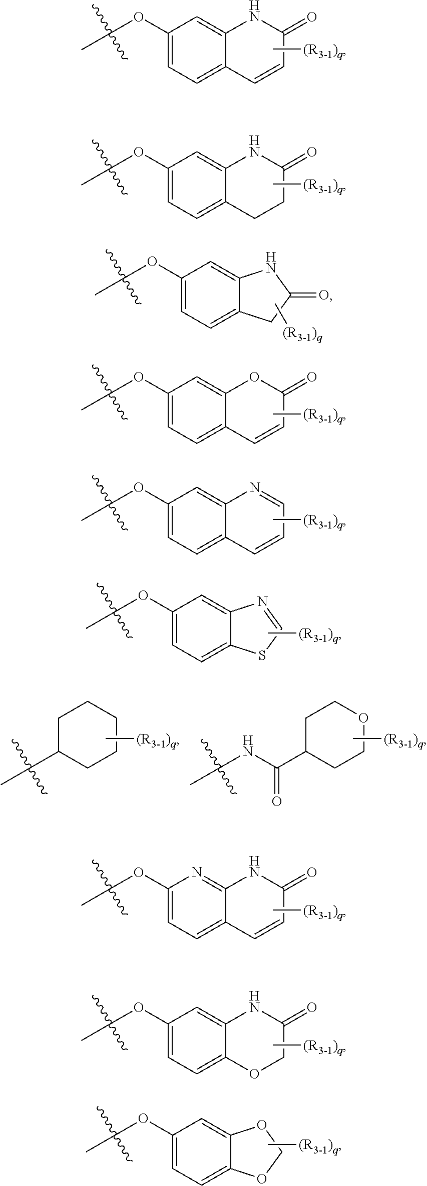 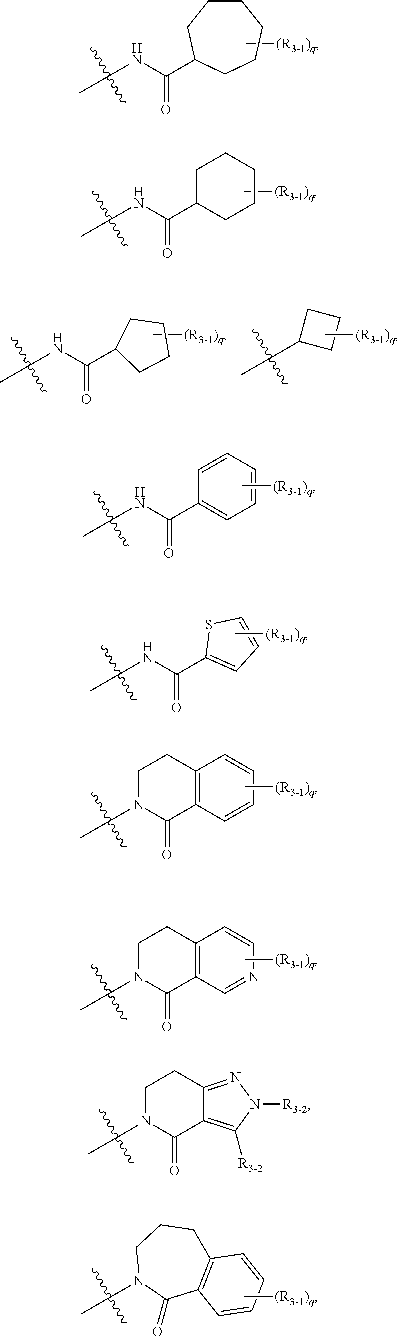 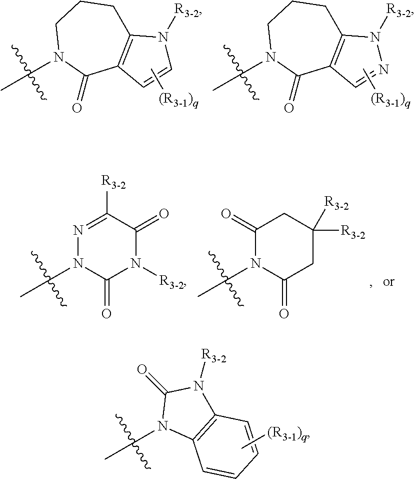
wherein R₃₋₁ is R₃, and R₃₋₂ is H or R₃.

6. The compound, or the pharmaceutically acceptable salt, isotope derivative, enantiomer, diastereomer, tautomer, or solvate thereof according to claim 1, wherein 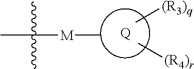
is any one of the following schemes:
scheme (1): 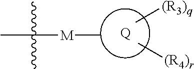
is 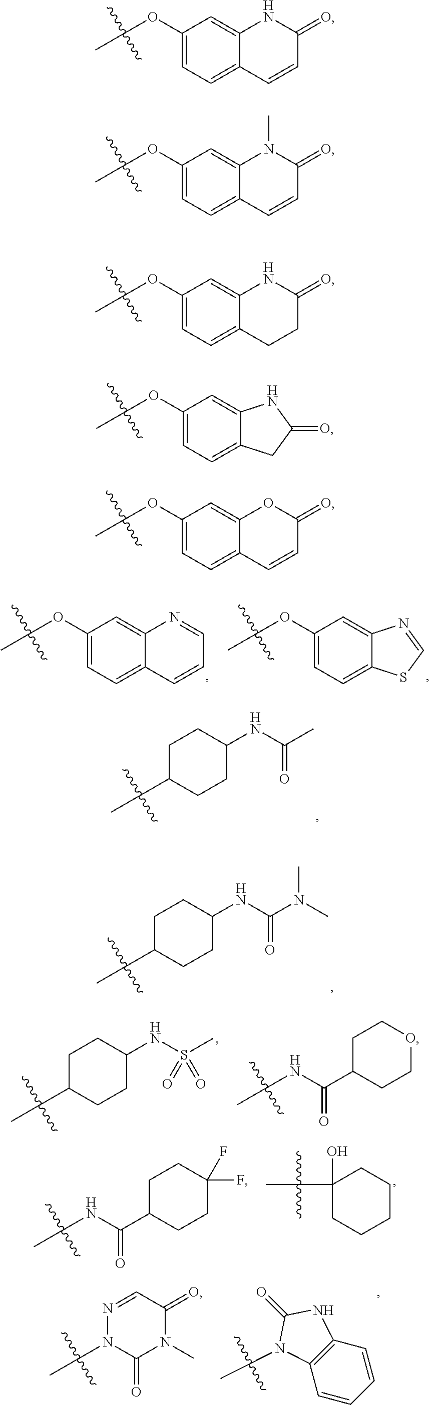 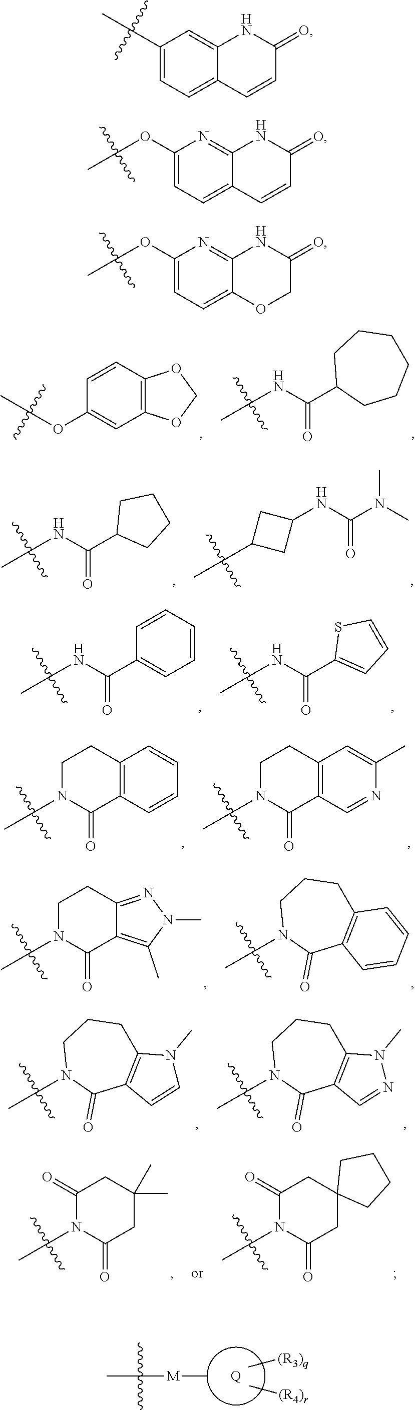
scheme (2): 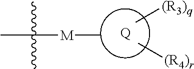
is 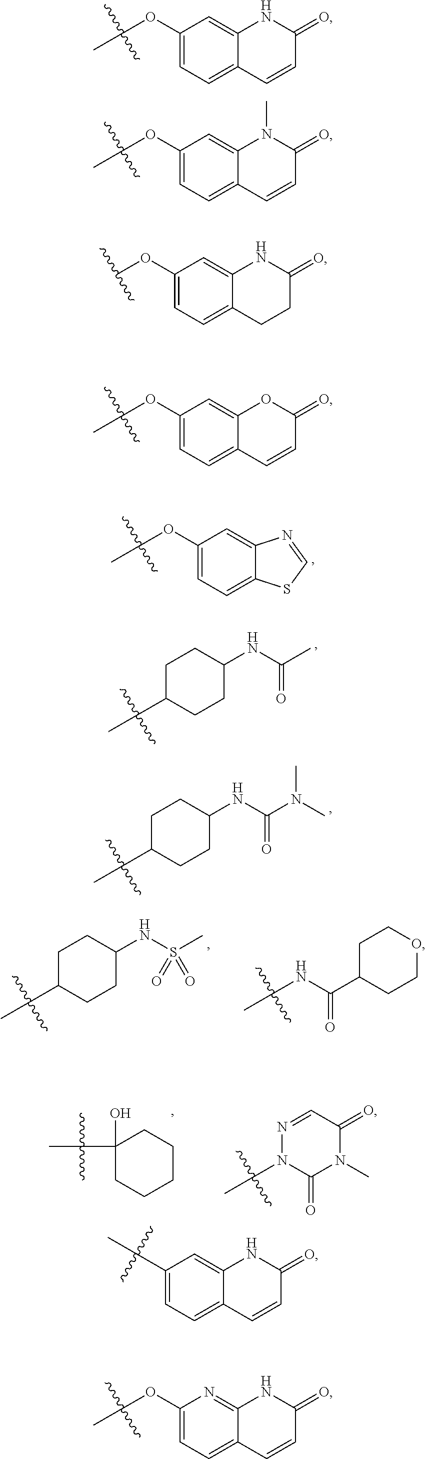 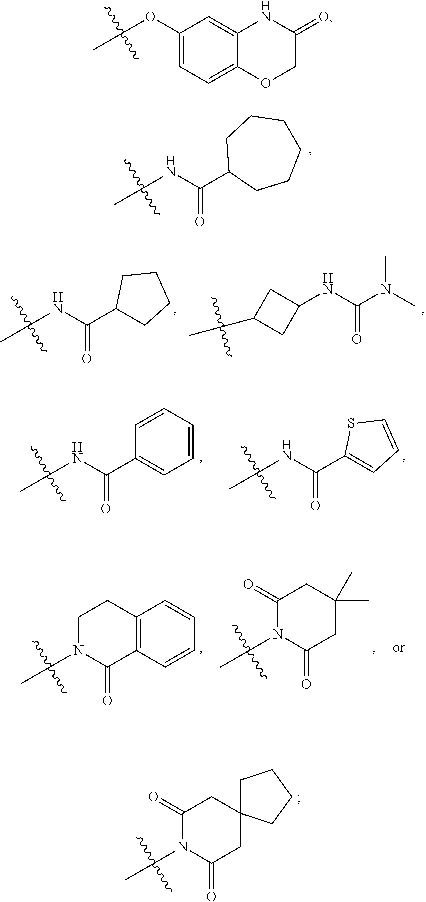
scheme (3): 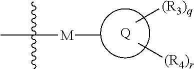
is 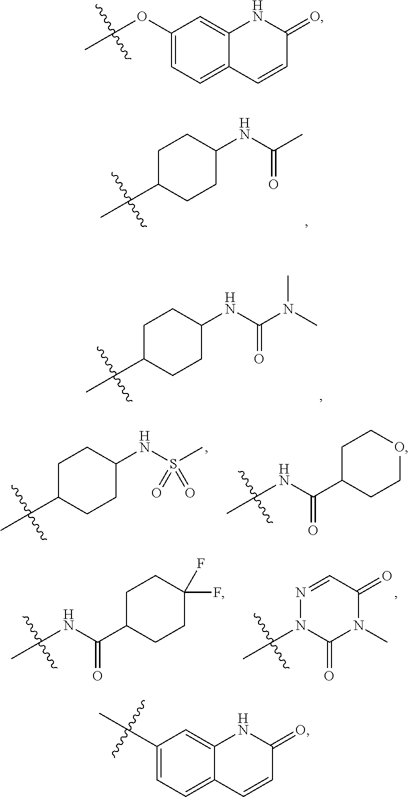 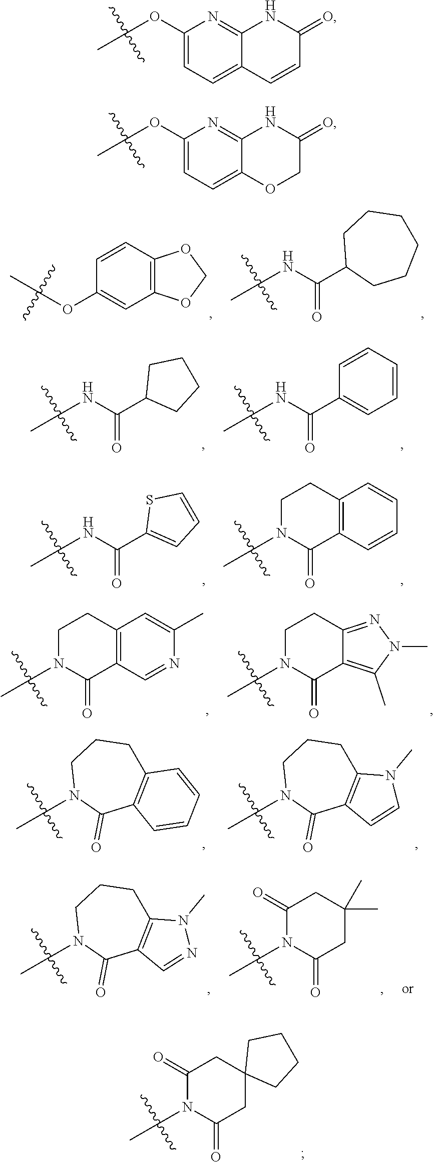
scheme 4: 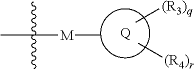
is 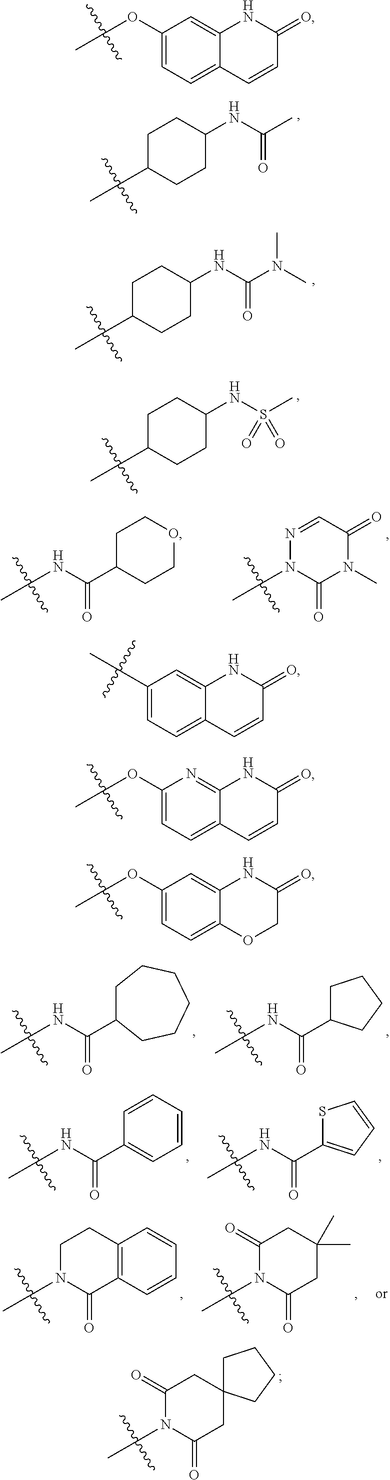
scheme (5): 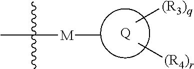
is 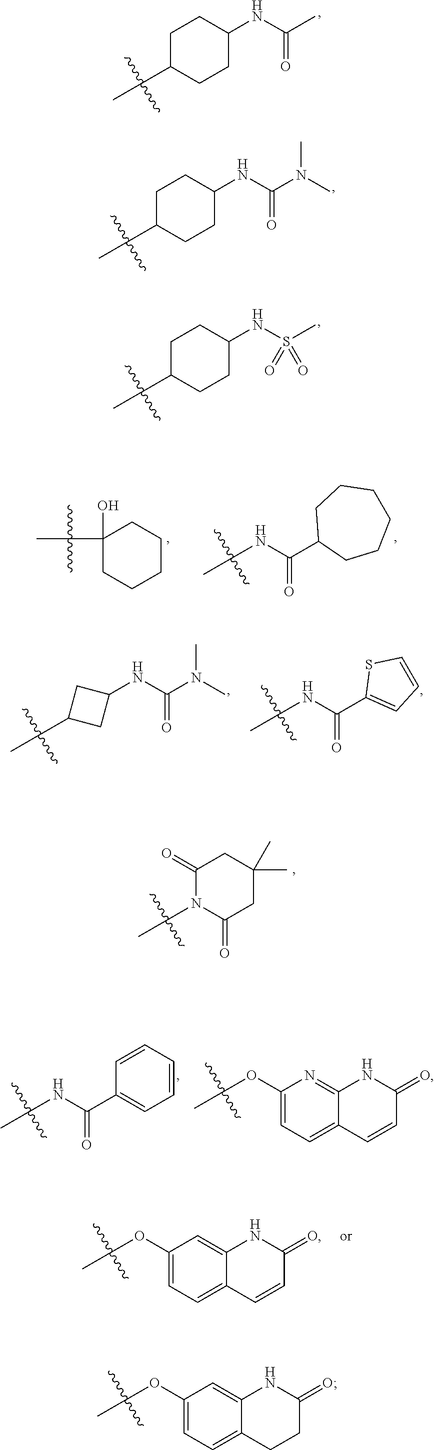
scheme (6): 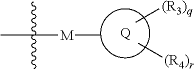
is 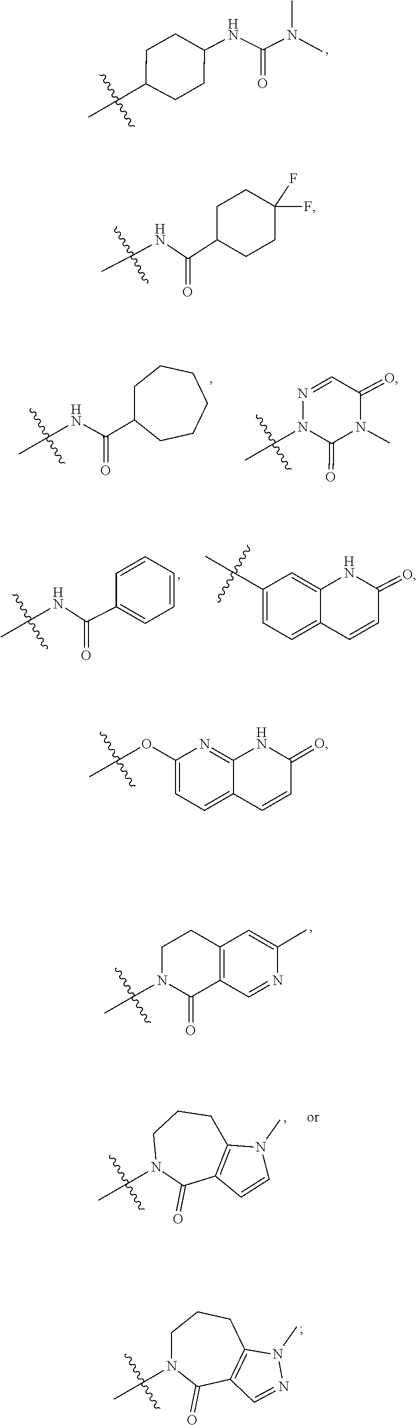
and
scheme (7): 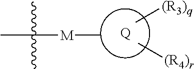
is

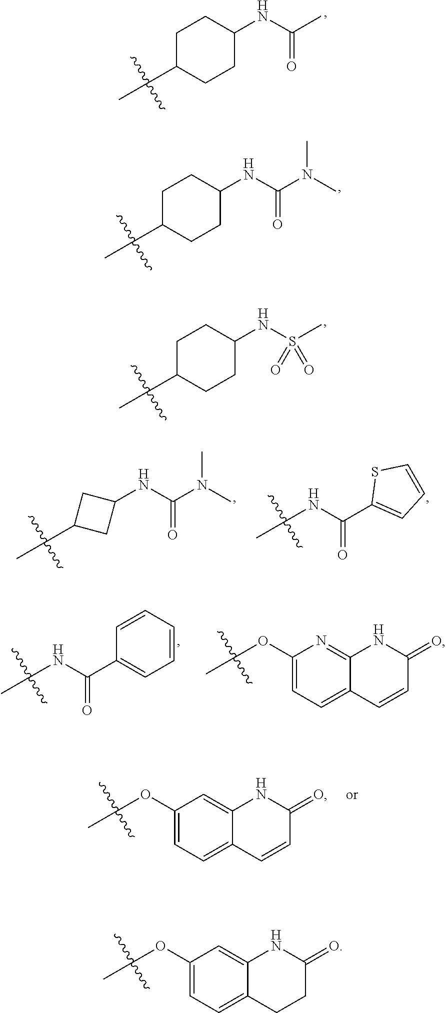

7. The compound, or the pharmaceutically acceptable salt, isotope derivative, enantiomer, diastereomer, tautomer, or solvate thereof according to claim 1, wherein L and M are as defined in any one of the following cases:
(1) L is —(CR_{c}R_{d})₂—, and M is absent or —NH—C(O)—;
(2) L is —(CR_{c}R_{d})₃—, and M is —O— or absent;
(3) L is —(CR_{c}R_{d})₄—, and M is —O— or absent;
(4) L is —(CR_{c}R_{d})—CH═CH—(CR_{c}R_{d})—, and M is —O—; and
(5) L is —(CR_{c}R_{d})—, and M is absent.

8. The compound, or the pharmaceutically acceptable salt, isotope derivative, enantiomer, diastereomer, tautomer, or solvate thereof according to claim 1, wherein the compound has any one of the following structures:

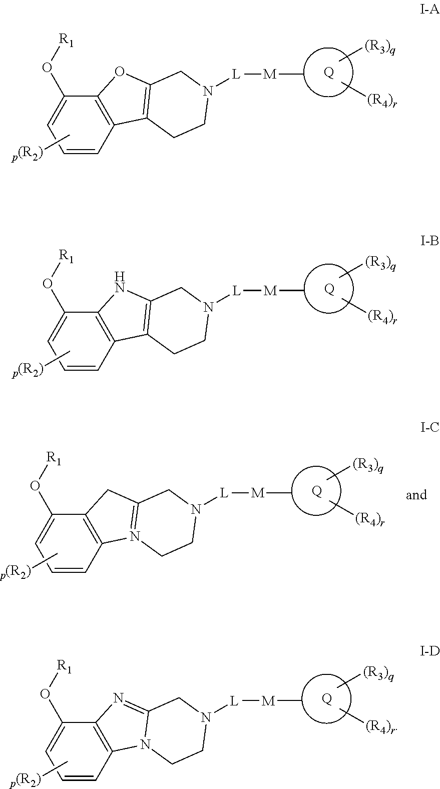

9. The compound, or the pharmaceutically acceptable salt, isotope derivative, enantiomer, diastereomer, tautomer, or solvate thereof according to claim 1, wherein the compound is any one of the following cases:
(1) in ring Q, the 4- to 8-membered carbocyclic ring is a 5- to 8-membered carbocyclic ring or a 4- to 6-membered carbocyclic ring;
(2) in ring Q, the 3- to 8-membered heterocyclic ring is a nitrogen-containing 6-membered heterocyclic ring;
(3) in ring Q, the 5- to 10-membered heteroaromatic ring is 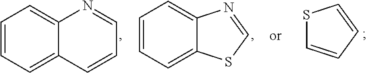
(4) in ring Q, the 8- to 11-membered fused bicyclic ring is a 8- to 10-membered fused bicyclic ring;
(5) in ring Q, one of the rings in the 8- to 11-membered fused bicyclic ring is a saturated or partially unsaturated 5- to 7-membered heterocyclic ring, wherein the 5- to 7-membered heterocyclic ring is a 5- to 6-membered heterocyclic ring;
(6) in ring Q, one of the rings in the 8- to 11-membered fused bicyclic ring is a saturated or partially unsaturated 5- to 7-membered heterocyclic ring, wherein the 5- to 7-membered heterocyclic ring contains at most one nitrogen atom or at most one oxygen atom;
(7) L is —(CR_{c}R_{d})₂—, and in ring Q, the saturated or partially unsaturated 5- to 7-membered heterocyclic ring in the 8- to 11-membered fused bicyclic ring contains at most one N;
(8) L is —(CR_{c}R_{d})₄—, M is —O—, and the benzene ring or the 5- to 6-membered heteroaromatic ring in the 8- to 11-membered fused bicyclic ring is a 5- to 6-membered aromatic heterocyclic ring;
(9) ring Q is a saturated 4- to 8-membered carbocyclic ring, a saturated or partially unsaturated 3- to 8-membered heterocyclic ring, a 6- to 10-membered aromatic ring, a 5- to 10-membered heteroaromatic ring, or an 8- to 11-membered fused bicyclic ring; one of the rings in the 8- to 11-membered fused bicyclic ring is a saturated or partially unsaturated 5- to 7-membered carbocyclic ring or a saturated or partially unsaturated 5- to 7-membered heterocyclic ring, and the other ring is benzene ring or a 5- to 6-membered heteroaromatic ring; and
(10) ring Q is a saturated 4- to 8-membered carbocyclic ring, a saturated or partially unsaturated 3- to 8-membered heterocyclic ring, phenyl, a 5- to 10-membered heteroaromatic ring, or an 8- to 10-membered fused bicyclic ring; one of the rings in the 8- to 10-membered fused bicyclic ring is a saturated or partially unsaturated 5- to 7-membered carbocyclic ring or a saturated or partially unsaturated 5- to 6-membered heterocyclic ring, and the other ring is benzene ring or a 5- to 6-membered heteroaromatic ring.

10. The compound, or the pharmaceutically acceptable salt, isotope derivative, enantiomer, diastereomer, tautomer, or solvate thereof according to claim 1, wherein
the compound is any one of the following schemes:
scheme (1):
X is N, O, NRₐ, or CR_{b};
Y is C or N;
L is —(CR_{c}R_{d})ₘ— or —(CR_{c}R_{d})ₙ₁—CH═CH—(CR_{c}R_{d})ₙ₂—;
R_{c} and R_{d} are each independently H;
m is 1, 2, 3, or 4;
n1 and n2 are 1;
P is 0;
r is 0, 1, or 2;
q is 0, 1, or 2;
M is absent, —O—, or —NH—C(O)—;
ring Q is a saturated 4- to 8-membered carbocyclic ring, a saturated or partially unsaturated 3- to 8-membered heterocyclic ring, a 6- to 10-membered aromatic ring, a 5- to 10-membered heteroaromatic ring, or an 8- to 11-membered fused bicyclic ring; one of the rings in the 8- to 11-membered fused bicyclic ring is a saturated or partially unsaturated 5- to 7-membered carbocyclic ring or a saturated or partially unsaturated 5- to 7-membered heterocyclic ring, and the other ring is benzene ring or a 5- to 6-membered heteroaromatic ring;
R₁ is C₁-C₆ alkyl;
each R₃ is independently F, Cl, Br, I, hydroxyl, C₁-C₆ alkyl, —NH—C(O)Rₑ, or —NH—S(O)₂Rₑ; or, two R₃ together with the atom to which they are attached form a 3- to 8-membered cycloalkyl ring;
Rₑ is C₁-C₆ alkyl, —NH₂, —NHR_{g}, —NR_{f}R_{g}, or 5- to 6-membered heteroaryl;
R_{f} and R_{g} are each independently C₁-C₆ alkyl;
R₄ is oxo (═O);
the number of heteroatoms in the heterocyclic ring, heteroaromatic ring, and heteroaryl is each independently 1, 2, or 3, and the heteroatoms are each independently N, O, or S;
scheme (2):
L is —(CR_{c}R_{d})ₘ— or —(CR_{c}R_{d})ₙ₁—CH═CH—(CR_{c}R_{d})ₙ₂—;
R_{c} and R_{d} are each independently H;
m is 1, 2, 3, or 4;
n1 and n2 are 1;
P is 0;
r is 0, 1, or 2;
q is 0, 1, or 2;
M is absent, —O—, or —NH—C(O)—;
ring Q is a saturated 4- to 8-membered carbocyclic ring, a saturated or partially unsaturated 3- to 8-membered heterocyclic ring, phenyl, a 5- to 10-membered heteroaromatic ring, or an 8- to 10-membered fused bicyclic ring; one of the rings in the 8- to 10-membered fused bicyclic ring is a saturated or partially unsaturated 5- to 7-membered carbocyclic ring or a saturated or partially unsaturated 5- to 6-membered heterocyclic ring, and the other ring is benzene ring or a 5- to 6-membered heteroaromatic ring; in the saturated or partially unsaturated 5- to 6-membered heterocyclic ring in the 8- to 10-membered fused bicyclic ring, the 5- to 6-membered heterocyclic ring contains one oxygen atom, one nitrogen atom, two nitrogen atoms, or one oxygen atom and one nitrogen atom;
R₁ is C₁-C₆ alkyl;
R₃ is hydroxyl, C₁-C₆ alkyl, —NH—C(O)Rₑ, or —NH—S(O)₂Rₑ; or, two R₃ together with the atom to which they are attached form a 3- to 8-membered cycloalkyl ring;
Rₑ is C₁-C₆ alkyl, —NH₂, —NHR_{g}, —NR_{f}R_{g}, or 5- to 6-membered heteroaryl;
R_{f} and R_{g} are each independently C₁-C₆ alkyl;
R₄ is oxo (═O);
the number of heteroatoms in the heterocyclic ring, heteroaromatic ring, and heteroaryl is each independently 1, 2, or 3, and the heteroatoms are each independently N, O, or S;
scheme (3):
X is N or O;
Y is C;
P is 0;
r is 0, 1, or 2;
q is 0, 1, or 2;
L is —(CR_{c}R_{d})ₘ—; R_{c} and R_{d} are each independently H; m is 2, 3, or 4;
when m is 2, M is absent or —NH—C(O)—, and ring Q is a saturated 5- to 8-membered carbocyclic ring, a saturated or partially unsaturated 3- to 8-membered heterocyclic ring, phenyl, a 5- to 10-membered heteroaromatic ring, or an 8- to 11-membered fused bicyclic ring; one of the rings in the 8- to 11-membered fused bicyclic ring is a saturated or partially unsaturated 5- to 7-membered carbocyclic ring or a saturated or partially unsaturated 5- to 7-membered heterocyclic ring, and the other ring is benzene ring or a 5- to 6-membered heteroaromatic ring;
when m is 3, M is absent; ring Q is a partially unsaturated 3- to 8-membered heterocyclic ring or an 8- to 11-membered fused bicyclic ring; one of the rings in the 8- to 11-membered fused bicyclic ring is a saturated or partially unsaturated 5- to 7-membered heterocyclic ring, and the other ring is benzene ring;
when m is 4, M is —O—, and ring Q is an 8- to 11-membered fused bicyclic ring; one of the rings in the 8- to 11-membered fused bicyclic ring is a saturated or partially unsaturated 5- to 7-membered heterocyclic ring, and the other ring is a 5- to 6-membered heteroaromatic ring;
or, when m is 4, M is absent;
R₁ is C₁-C₆ alkyl;
each R₃ is independently F, Cl, Br, I, C₁-C₆ alkyl, or —NH—C(O)Rₑ; or, two R₃ together with the atom to which they are attached form a 3- to 8-membered cycloalkyl ring;
Rₑ is C₁-C₆ alkyl, —NH₂, —NHR_{g}, —NR_{f}R_{g}, or 5- to 6-membered heteroaryl;
R_{f} and R_{g} are each independently C₁-C₆ alkyl;
R₄ is oxo (═O);
the number of heteroatoms in the heterocyclic ring, heteroaromatic ring, and heteroaryl is each independently 1, 2, or 3, and the heteroatoms are each independently N, O, or S;
in the saturated or partially unsaturated 5- to 7-membered heterocyclic ring in the 8- to 11-membered fused bicyclic ring, the 5- to 7-membered heterocyclic ring contains one oxygen atom, one nitrogen atom, two oxygen atoms, or one oxygen atom and one nitrogen atom;
scheme (4):
X is N or O;
Y is C;
P is 0;
r is 0, 1, or 2;
q is 0, 1, or 2;
L is —(CR_{c}R_{d})ₘ—; R_{c} and R_{d} are each independently H; m is 2, 3, or 4;
when m is 2, M is absent or —NH—C(O)—, and ring Q is a saturated 5- to 8-membered carbocyclic ring, a saturated or partially unsaturated 3- to 8-membered heterocyclic ring, phenyl, a 5- to 10-membered heteroaromatic ring, or an 8- to 10-membered fused bicyclic ring; one of the rings in the 8- to 10-membered fused bicyclic ring is a saturated or partially unsaturated 5- to 7-membered carbocyclic ring or a saturated or partially unsaturated 5- to 6-membered heterocyclic ring, and the other ring is benzene ring or a 5- to 6-membered heteroaromatic ring;
when m is 3, M is absent, and ring Q is a partially unsaturated 3- to 8-membered heterocyclic ring or an 8- to 10-membered fused bicyclic ring; one of the rings in the 8 to 10-membered fused bicyclic ring is a saturated or partially unsaturated 5- to 6-membered heterocyclic ring, and the other ring is benzene ring;
when m is 4, M is —O—, and ring Q is an 8- to 10-membered fused bicyclic ring; one of the rings in the 8- to 10-membered fused bicyclic ring is a saturated or partially unsaturated 5- to 6-membered heterocyclic ring, and the other ring is a 5- to 6-membered heteroaromatic ring;
or, when m is 4, M is absent; ring Q is a saturated or partially unsaturated 3- to 8-membered heterocyclic ring or an 8- to 10-membered fused bicyclic ring; one of the rings in the 8- to 10-membered fused bicyclic ring is a saturated or partially unsaturated 5- to 6-membered heterocyclic ring, and the other ring is benzene ring;
R₁ is C₁-C₆ alkyl;
each R₃ is independently C₁-C₆ alkyl or —NH—C(O)Rₑ; or, two R₃ together with the atom to which they are attached form a 3- to 8-membered cycloalkyl ring;
Rₑ is C₁-C₆ alkyl, —NH₂, —NHR_{g}, —NR_{f}R_{g}, or 5- to 6-membered heteroaryl;
R_{f} and R_{g} are each independently C₁-C₆ alkyl;
R₄ is oxo (═O);
the number of heteroatoms in the heterocyclic ring, heteroaromatic ring, and heteroaryl is each independently 1, 2, or 3, and the heteroatoms are each independently N, O, or S;
in the saturated or partially unsaturated 5- to 6-membered heterocyclic ring in the 8- to 10-membered fused bicyclic ring, the 5- to 6-membered heterocyclic ring contains one oxygen atom, one nitrogen atom, or one oxygen atom and one nitrogen atom;
scheme (5):
X is O; Y is C; P is 0;
L is —(CR_{c}R_{d})ₘ— or —(CR_{c}R_{d})ₙ₁—CH═CH—(CR_{c}R_{d})ₙ₂—; R_{c} and R_{d} are each independently H; m is 1, 2, 3, or 4;
when L is —(CR_{c}R_{d})ₙ₁—CH═CH—(CR_{c}R_{d})ₙ₂—, n1 and n2 are 1, M is —O—, and ring Q is 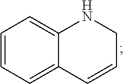
when L is —(CR_{c}R_{d})ₘ— and m is 1, then M is absent, ring Q is a saturated 6-membered carbocyclic ring, and R₃ is hydroxyl;
when L is —(CR_{c}R_{d})ₘ— and m is 2, then M is absent or —NH—C(O)—, ring Q is a saturated 4- to 8-membered carbocyclic ring, piperidine, thiophene, or phenyl, each R₃ is independently C₁-C₆ alkyl or —NH—C(O)Rₑ, and Rₑ is C₁-C₆ alkyl;
when L is —(CR_{c}R_{d})ₘ— and m is 3, then M is —O—, and ring Q is 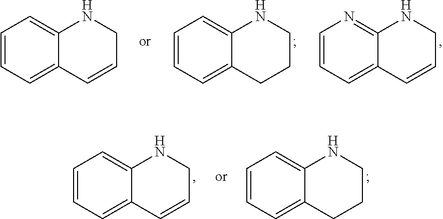
when L is —(CR_{c}R_{d})ₘ— and m is 4, then M is —O— or absent, and ring Q is piperidine,
when ring Q is piperidine, each R₃ is independently C₁-C₆ alkyl;
when ring Q is q is 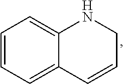
R₁ is C₁-C₆ alkyl;
scheme (6):
X is N; Y is C; P is 0;
L is —(CR_{c}R_{d})ₘ—; R_{c} and R_{d} are each independently H; m is 2 or 3;
when L is —(CR_{c}R_{d})ₘ— and m is 2, then M is absent, ring Q is a saturated 5- to 7-membered carbocyclic ring, each R₃ is independently —NH—C(O)Rₑ, and Rₑ is C₁-C₆ alkyl;
when L is —(CR_{c}R_{d})ₘ— and m is 3, then M is —O—, ring Q is 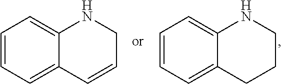
and q is 0;
R₁ is methyl;
scheme (7):
X is C; Y is N; P is 0;
L is —(CR_{c}R_{d})ₘ— or —(CR_{c}R_{d})ₙ₁—CH═CH—(CR_{c}R_{d})ₙ₂—; R_{c} and R_{d} are each independently H; m is 3 or 4;
when L is —(CR_{c}R_{d})ₙ₁—CH═CH—(CR_{c}R_{d})ₙ₂—, n1 and n2 are 1, M is —O—, and ring Q is 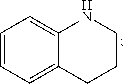
when L is —(CR_{c}R_{d})ₘ— and m is 3, then M is —O—, and ring Q is 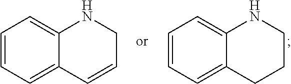 [SMILES: C1=Cc2ccccc2NC1 ; c1ccc2c(c1)CCCN2]
when L is —(CR_{c}R_{d})ₘ— and m is 4, then M is —O—, and ring Q is 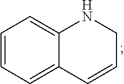
scheme (8):
X is O; Y is C; P is 0;
L is —(CR_{c}R_{d})ₘ—; R_{c} and R_{d} are each independently H; m is 2 or 4;
when L is —(CR_{c}R_{d})ₘ— and m is 2, then M is absent or —NH—C(O)—, and ring Q is a saturated 6- to 7-membered carbocyclic ring, phenyl, or an 8- to 11-membered fused bicyclic ring; one of the rings in the 8- to 11-membered fused bicyclic ring is a saturated 5- to 7-membered heterocyclic ring, and the other ring is a 5- to 6-membered heteroaromatic ring; the 5- to 6-membered heteroaromatic ring contains one or two nitrogen atoms;
when L is —(CR_{c}R_{d})ₘ— and m is 4, then M is absent or —O—;
when L is —(CR_{c}R_{d})ₘ—, m is 4, and M is absent, then ring Q is a partially unsaturated 6-membered heterocyclic ring or 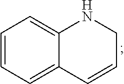
when ring Q is a partially unsaturated 6-membered heterocyclic ring, the heteroatom in the partially unsaturated 6-membered heterocyclic ring is a nitrogen atom, and the number of heteroatoms is each independently 1, 2, or 3;
when L is —(CR_{c}R_{d})ₘ—, m is 4, and M is —O—, then ring Q is a partially unsaturated 6-membered heterocyclic ring or 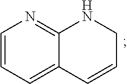
each R₃ is independently F, C₁-C₆ alkyl, or —NH—C(O)Rₑ, and Rₑ is C₁-C₆ alkyl;
scheme (9):
X is O; Y is C; P is 0;
L is —(CR_{c}R_{d})ₘ— or —(CR_{c}R_{d})ₙ₁—CH═CH—(CR_{c}R_{d})ₙ₂—; R_{c} and R_{d} are each independently H; m is 2 or 4;
when L is —(CR_{c}R_{d})ₙ₁—CH═CH—(CR_{c}R_{d})ₙ₂—, n1 and n2 are 1, M is —O—, and ring Q is 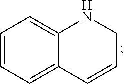
when L is —(CR_{c}R_{d})ₘ— and m is 2, then M is absent or —NH—C(O)—, ring Q is a saturated 4- to 6-membered carbocyclic ring, thiophene, or phenyl, each R₃ is independently C₁-C₆ alkyl or —NH—C(O)Rₑ, and Rₑ is C₁-C₆ alkyl;
when L is —(CR_{c}R_{d})ₘ— and m is 4, then M is —O— or absent, and ring Q is piperidine, 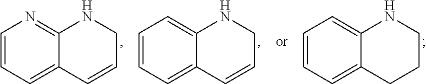
q is 0;
R₁ is C₁-C₆ alkyl;
scheme (10):
X is N; Y is C; P is 0;
L is —(CR_{c}R_{d})ₘ—; R_{c} and R_{d} are each independently H; m is 2 or 3;
when L is —(CR_{c}R_{d})ₘ— and m is 2, then M is absent, ring Q is a saturated 6-membered carbocyclic ring, each R₃ is independently —NH—C(O)Rₑ, and Rₑ is C₁-C₆ alkyl;
when L is —(CR_{c}R_{d})ₘ— and m is 3, then M is —O—, ring Q is 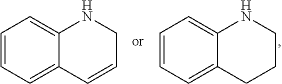
and q is 0;
R₁ is methyl;
scheme (11):
X is C; Y is N; P is 0;
L is —(CR_{c}R_{d})ₘ— or —(CR_{c}R_{d})ₙ₁—CH═CH—(CR_{c}R_{d})ₙ₂—; R_{c} and R_{d} are each independently H; m is 3 or 4;
when L is —(CR_{c}R_{d})ₙ₁—CH═CH—(CR_{c}R_{d})ₙ₂—, n1 and n2 are 1, M is —O—, and ring Q is 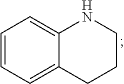
when L is —(CR_{c}R_{d})ₘ— and m is 3, then M is —O—, and ring Q is 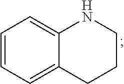
when L is —(CR_{c}R_{d})ₘ— and m is 4, then M is —O—, and ring Q is

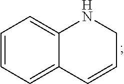

11. The compound, or the pharmaceutically acceptable salt, isotope derivative, enantiomer, diastereomer, tautomer, or solvate thereof according to claim 1, wherein the compound has any one of the following structures:

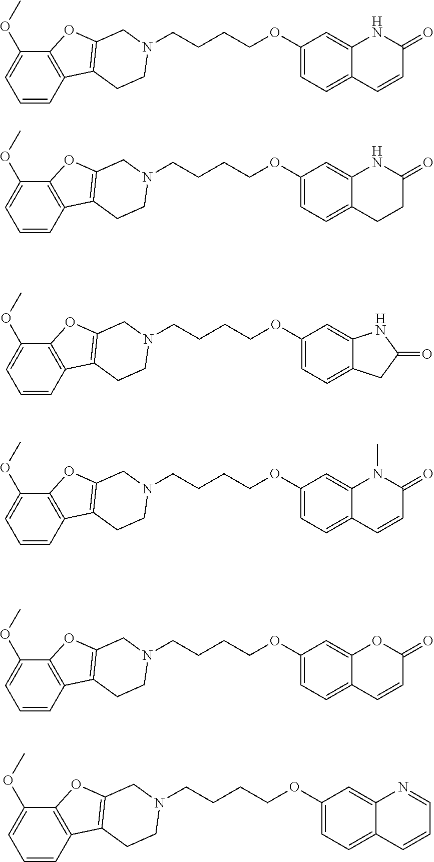

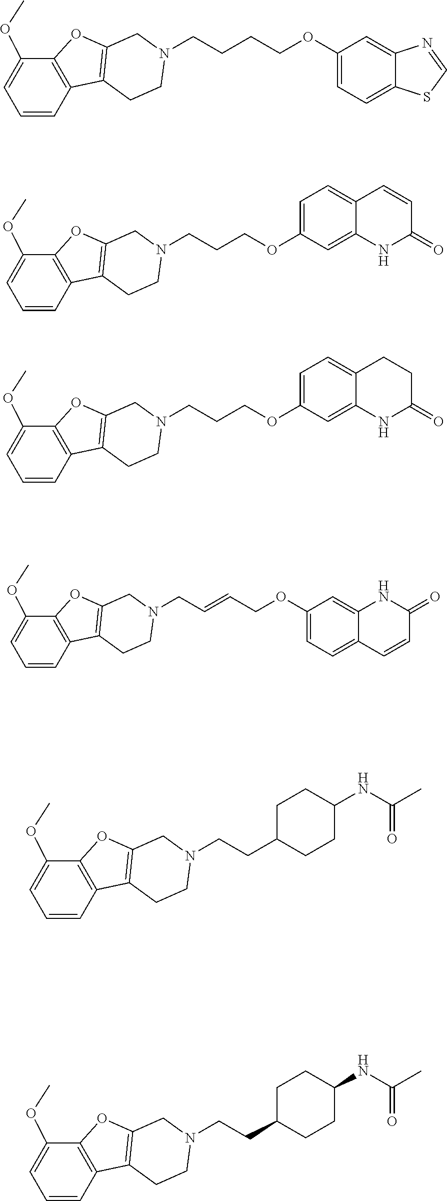 [SMILES: COc1cccc2c3c(oc12)CN(CCCCOc1ccc2scnc2c1)CC3 ; COc1cccc2c3c(oc12)CN(CCCOc1ccc2ccc(=O)[nH]c2c1)CC3 ; COc1cccc2c3c(oc12)CN(CCCOc1ccc2c(c1)NC(=O)CC2)CC3 ; COc1cccc2c3c(oc12)CN(C/C=C/COc1ccc2ccc(=O)[nH]c2c1)CC3 ; COc1cccc2c3c(oc12)CN(CCC1CCC(NC(C)=O)CC1)CC3 ; COc1cccc2c3c(oc12)CN(CC[C@H]1CC[C@@H](NC(C)=O)CC1)CC3]

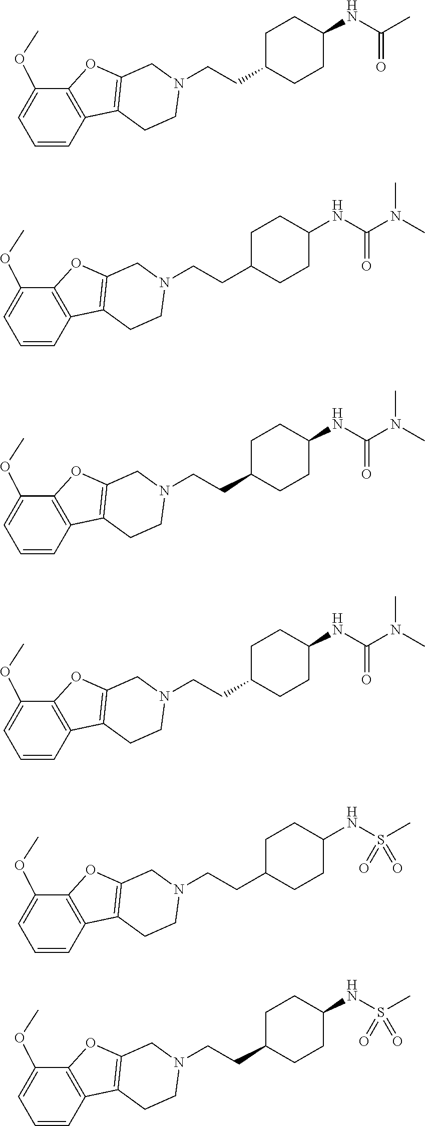 [SMILES: COc1cccc2c3c(oc12)CN(CC[C@H]1CC[C@H](NC(C)=O)CC1)CC3 ; COc1cccc2c3c(oc12)CN(CCC1CCC(NC(=O)N(C)C)CC1)CC3 ; COc1cccc2c3c(oc12)CN(CC[C@H]1CC[C@@H](NC(=O)N(C)C)CC1)CC3 ; COc1cccc2c3c(oc12)CN(CC[C@H]1CC[C@H](NC(=O)N(C)C)CC1)CC3 ; COc1cccc2c3c(oc12)CN(CCC1CCC(NS(C)(=O)=O)CC1)CC3 ; COc1cccc2c3c(oc12)CN(CC[C@H]1CC[C@@H](NS(C)(=O)=O)CC1)CC3]

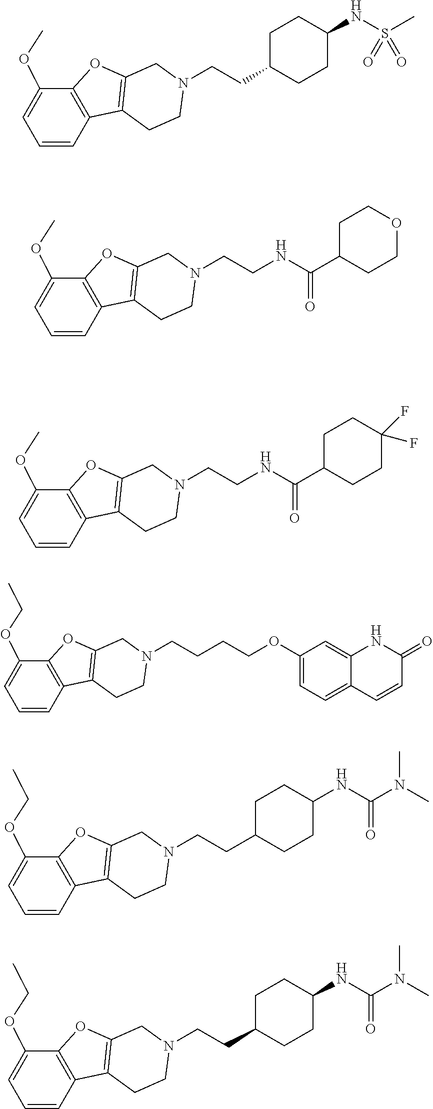

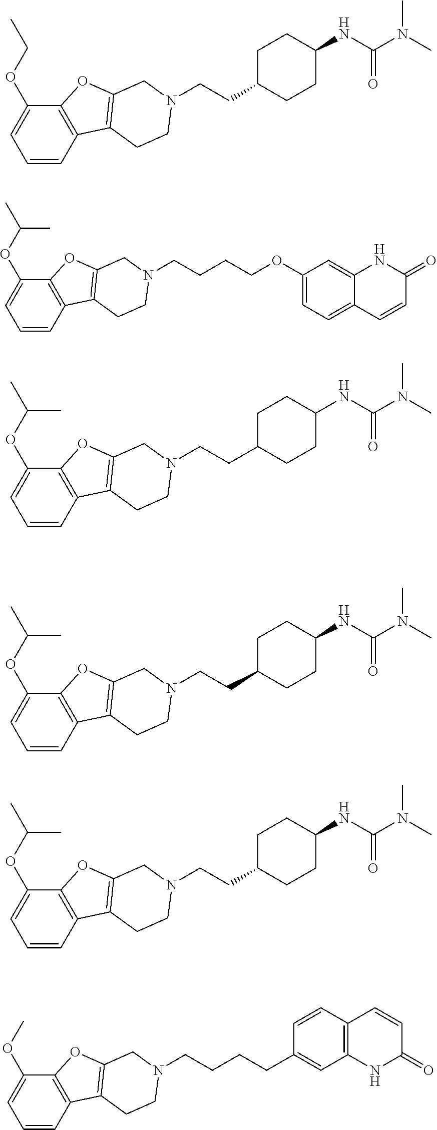

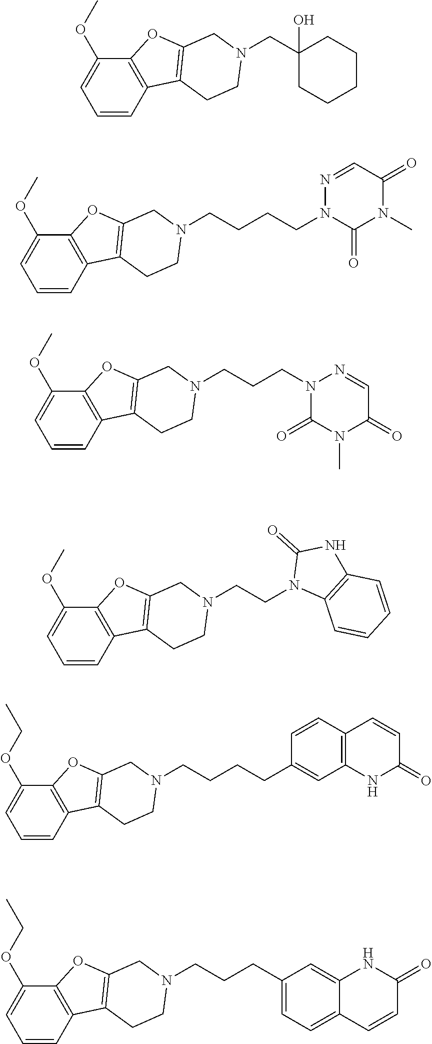

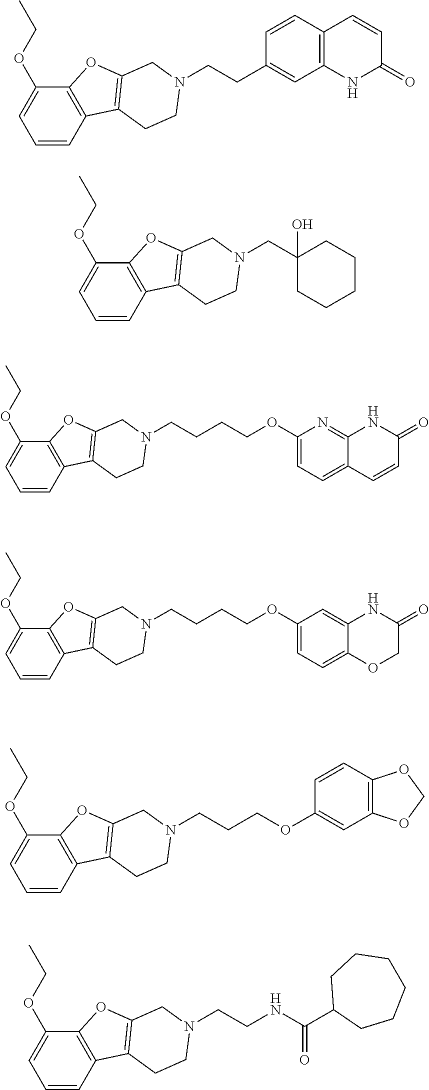

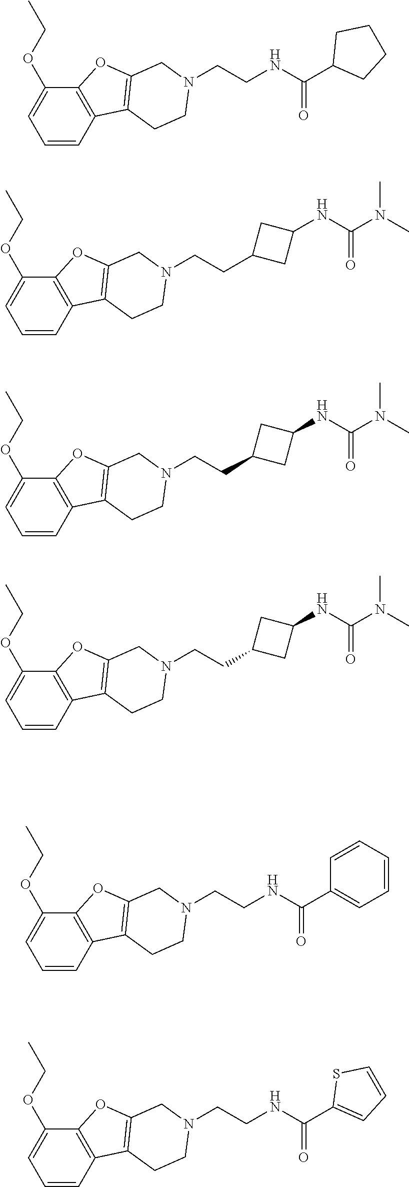 [SMILES: CCOc1cccc2c3c(oc12)CN(CCNC(=O)C1CCCC1)CC3 ; CCOc1cccc2c3c(oc12)CN(CCC1CC(NC(=O)N(C)C)C1)CC3 ; CCOc1cccc2c3c(oc12)CN(CC[C@H]1C[C@@H](NC(=O)N(C)C)C1)CC3 ; CCOc1cccc2c3c(oc12)CN(CC[C@H]1C[C@H](NC(=O)N(C)C)C1)CC3 ; CCOc1cccc2c3c(oc12)CN(CCNC(=O)c1ccccc1)CC3 ; CCOc1cccc2c3c(oc12)CN(CCNC(=O)c1cccs1)CC3]

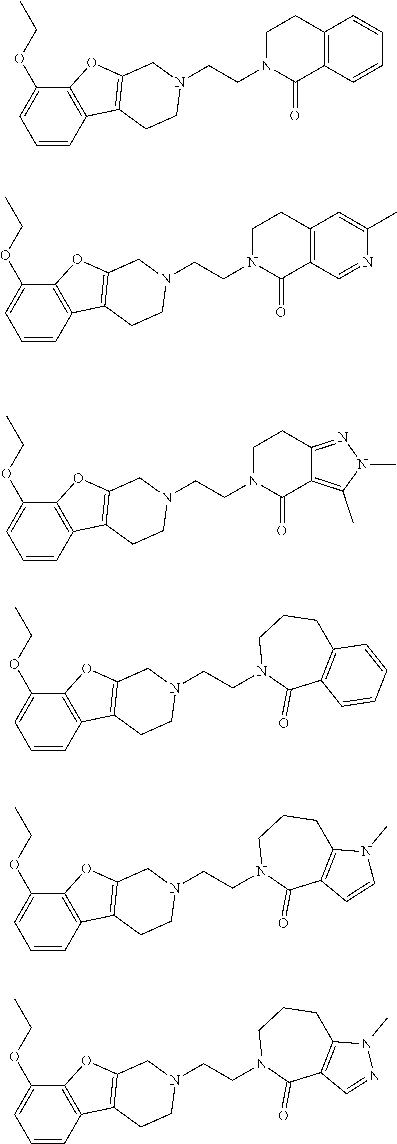 [SMILES: CCOc1cccc2c3c(oc12)CN(CCN1CCc2ccccc2C1=O)CC3 ; CCOc1cccc2c3c(oc12)CN(CCN1CCc2cc(C)ncc2C1=O)CC3 ; CCOc1cccc2c3c(oc12)CN(CCN1CCc2nn(C)c(C)c2C1=O)CC3 ; CCOc1cccc2c3c(oc12)CN(CCN1CCCc2ccccc2C1=O)CC3 ; CCOc1cccc2c3c(oc12)CN(CCN1CCCc2c(ccn2C)C1=O)CC3 ; CCOc1cccc2c3c(oc12)CN(CCN1CCCc2c(cnn2C)C1=O)CC3]

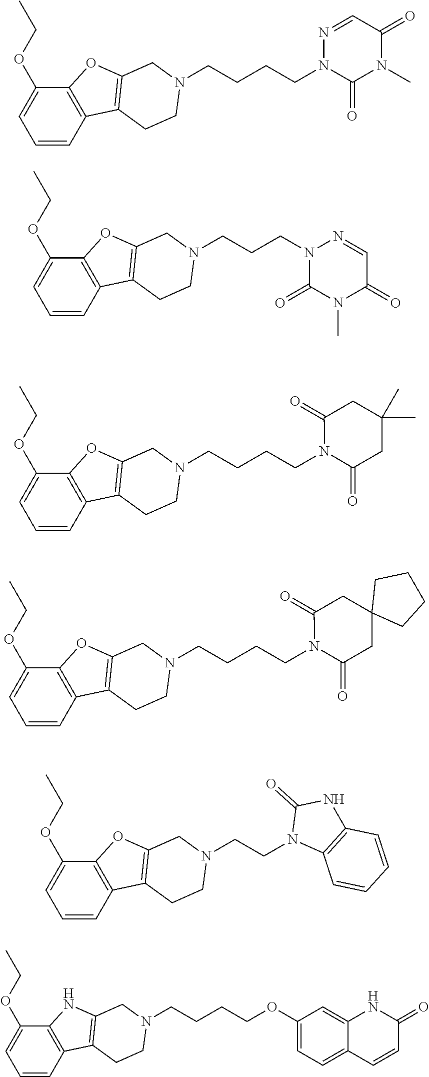

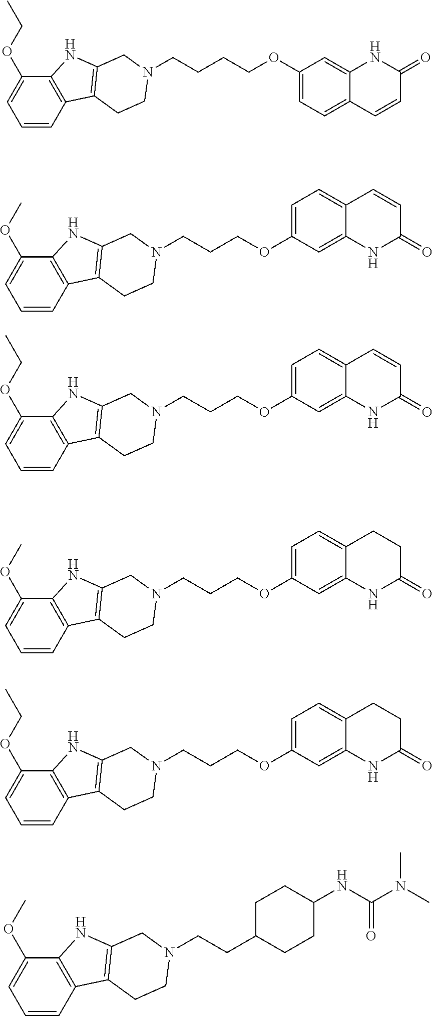

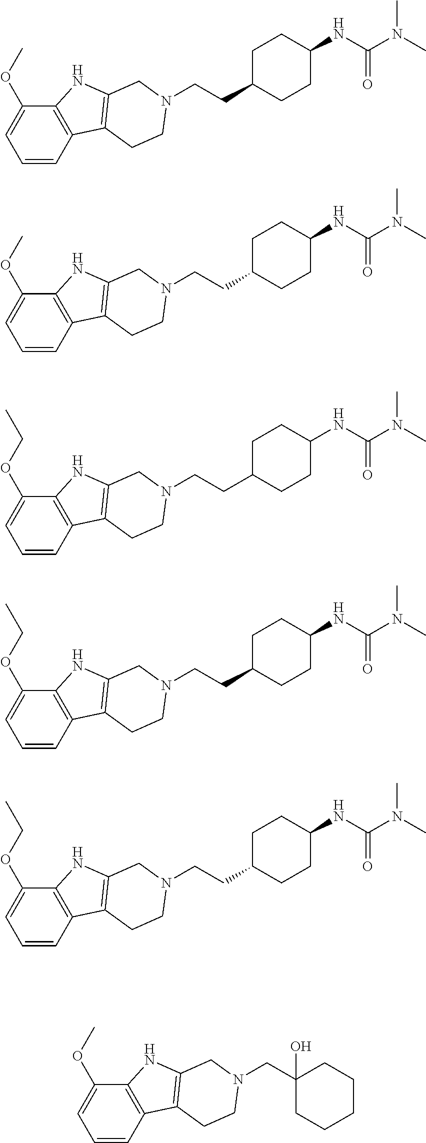 [SMILES: COc1cccc2c3c([nH]c12)CN(CC[C@H]1CC[C@@H](NC(=O)N(C)C)CC1)CC3 ; COc1cccc2c3c([nH]c12)CN(CC[C@H]1CC[C@H](NC(=O)N(C)C)CC1)CC3 ; CCOc1cccc2c3c([nH]c12)CN(CCC1CCC(NC(=O)N(C)C)CC1)CC3 ; CCOc1cccc2c3c([nH]c12)CN(CC[C@H]1CC[C@@H](NC(=O)N(C)C)CC1)CC3 ; CCOc1cccc2c3c([nH]c12)CN(CC[C@H]1CC[C@H](NC(=O)N(C)C)CC1)CC3 ; COc1cccc2c3c([nH]c12)CN(CC1(O)CCCCC1)CC3]

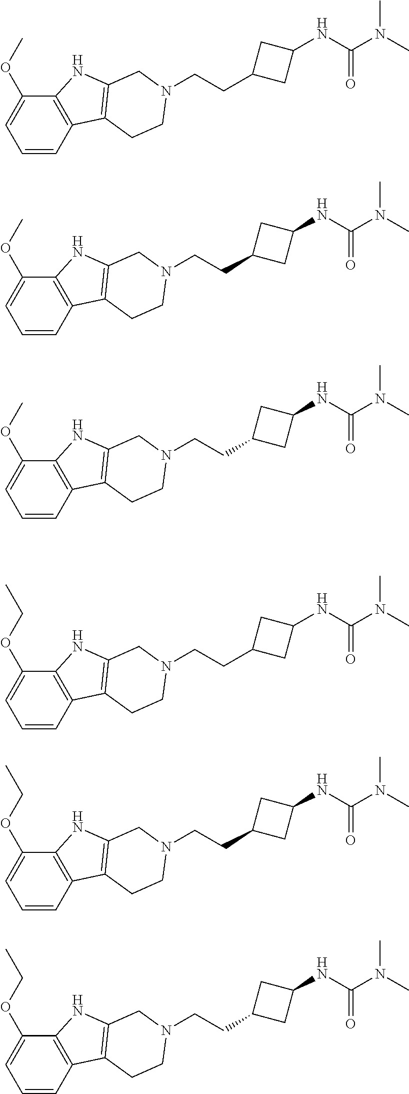

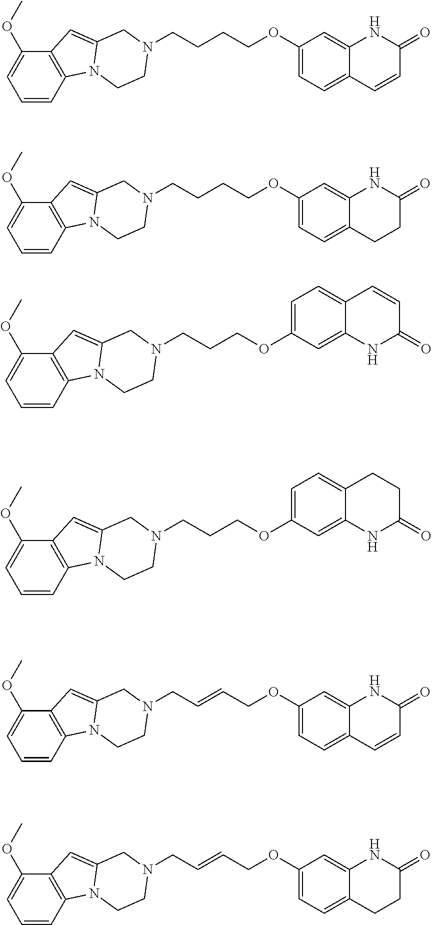 [SMILES: COc1cccc2c1cc1n2CCN(CCCCOc2ccc3ccc(=O)[nH]c3c2)C1 ; COc1cccc2c1cc1n2CCN(CCCCOc2ccc3c(c2)NC(=O)CC3)C1 ; COc1cccc2c1cc1n2CCN(CCCOc2ccc3ccc(=O)[nH]c3c2)C1 ; COc1cccc2c1cc1n2CCN(CCCOc2ccc3c(c2)NC(=O)CC3)C1 ; COc1cccc2c1cc1n2CCN(C/C=C/COc2ccc3ccc(=O)[nH]c3c2)C1 ; COc1cccc2c1cc1n2CCN(C/C=C/COc2ccc3c(c2)NC(=O)CC3)C1]

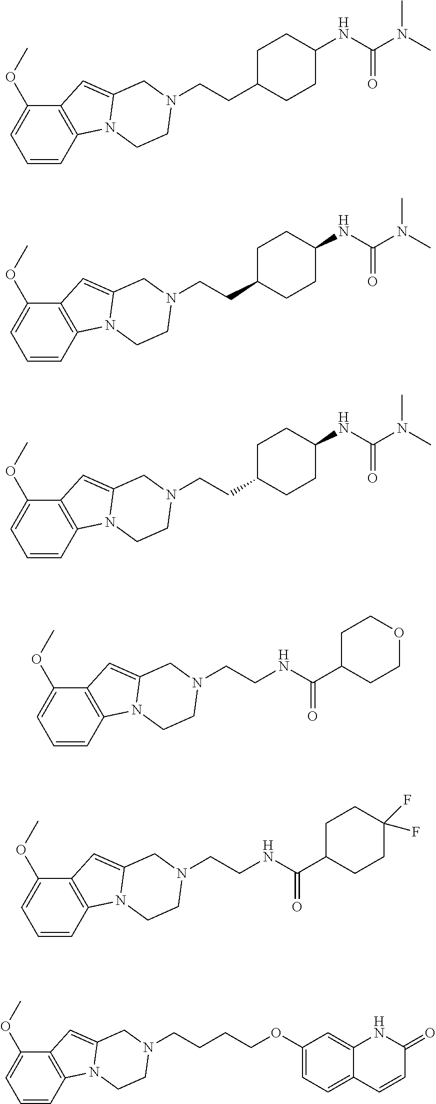 [SMILES: COc1cccc2c1cc1n2CCN(CCC2CCC(NC(=O)N(C)C)CC2)C1 ; COc1cccc2c1cc1n2CCN(CC[C@H]2CC[C@@H](NC(=O)N(C)C)CC2)C1 ; COc1cccc2c1cc1n2CCN(CC[C@H]2CC[C@H](NC(=O)N(C)C)CC2)C1 ; COc1cccc2c1cc1n2CCN(CCNC(=O)C2CCOCC2)C1 ; COc1cccc2c1cc1n2CCN(CCNC(=O)C2CCC(F)(F)CC2)C1 ; COc1cccc2c1cc1n2CCN(CCCCOc2ccc3ccc(=O)[nH]c3c2)C1]

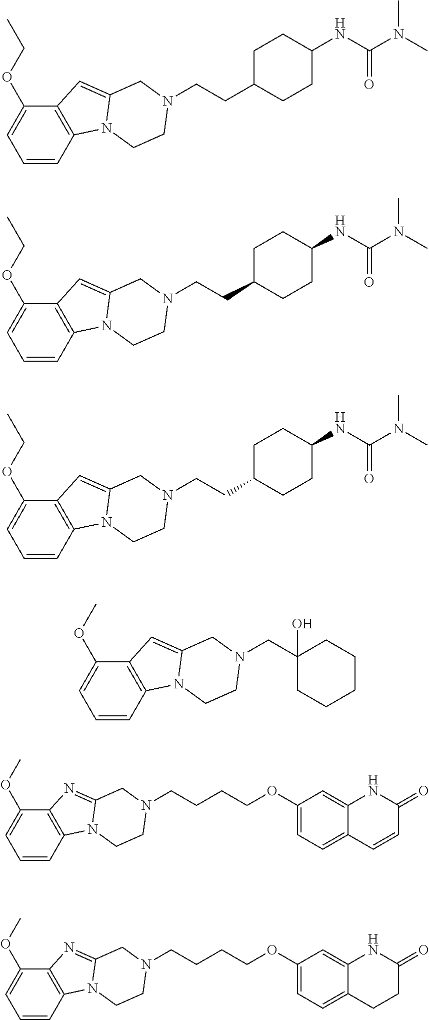 [SMILES: CCOc1cccc2c1cc1n2CCN(CCC2CCC(NC(=O)N(C)C)CC2)C1 ; CCOc1cccc2c1cc1n2CCN(CC[C@H]2CC[C@@H](NC(=O)N(C)C)CC2)C1 ; CCOc1cccc2c1cc1n2CCN(CC[C@H]2CC[C@H](NC(=O)N(C)C)CC2)C1 ; COc1cccc2c1cc1n2CCN(CC2(O)CCCCC2)C1 ; COc1cccc2c1nc1n2CCN(CCCCOc2ccc3ccc(=O)[nH]c3c2)C1 ; COc1cccc2c1nc1n2CCN(CCCCOc2ccc3c(c2)NC(=O)CC3)C1]

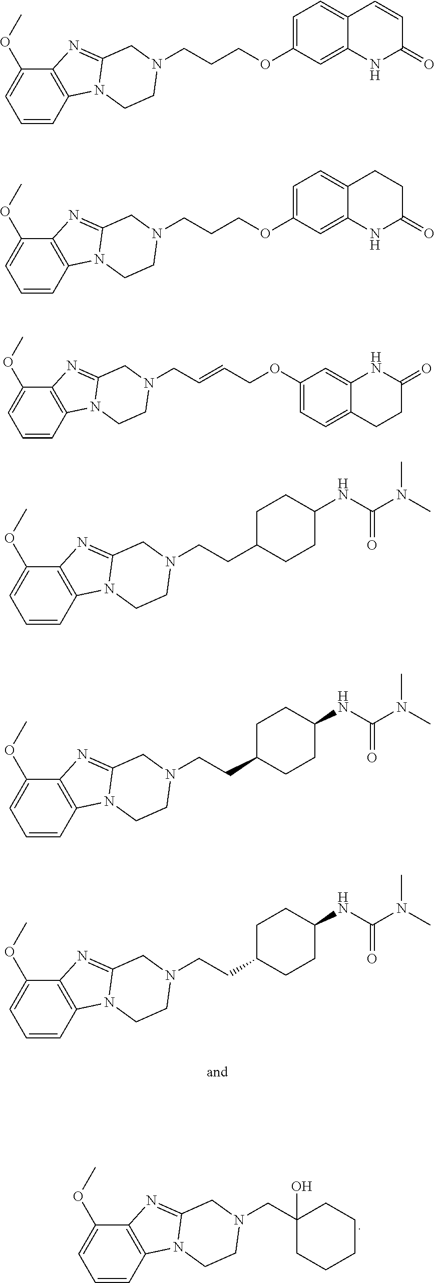

12. A preparation method for a compound of formula I, comprising the step of: conducting a coupling reaction as follows between a compound of formula II and a compound of formula III in a solvent to obtain the compound of formula I; 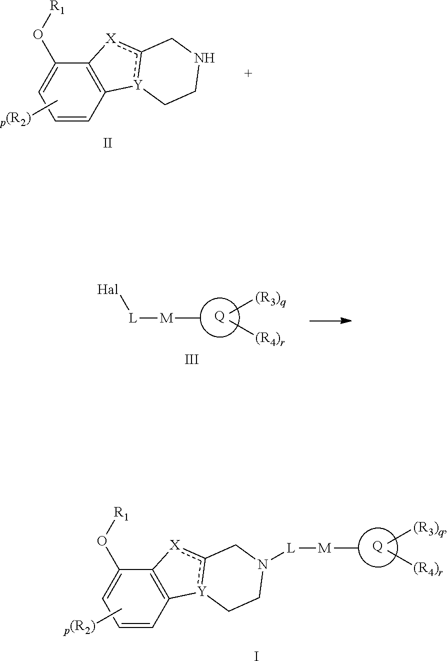
wherein Hal is halogen; X, Y, [symbol] , L, M, ring Q, R₁, R₂, R₃, R₄, p, q, and r are as defined claim 1.

13. A pharmaceutical composition comprising the compound, or the pharmaceutically acceptable salt, isotope derivative, enantiomer, diastereomer, tautomer, or solvate thereof according to claim 1, and a pharmaceutically acceptable excipient.

14. A method for the treatment of a mental disease or neurodegenerative disease in a subject in need thereof, comprising administering a therapeutically effective amount of the compound, or the pharmaceutically acceptable salt, isotope derivative, enantiomer, diastereomer, tautomer, or solvate thereof according to claim 1 to the subject.

15. The method according to claim 14, wherein the mental disease or neurodegenerative disease is schizophrenia, depression, or Parkinson's disease.

16. The compound, or the pharmaceutically acceptable salt, isotope derivative, enantiomer, diastereomer, tautomer, or solvate thereof according to claim 2, wherein each R₃ is independently C₁-C₆ alkyl or —NH—C(O)Rₑ.

17. The compound, or the pharmaceutically acceptable salt, isotope derivative, enantiomer, diastereomer, tautomer, or solvate thereof according to claim 9, wherein the compound is any one of the following cases:
(1) in ring Q, the 5- to 8-membered carbocyclic ring is a 5- to 7-membered carbocyclic ring;
(2) in ring Q, the 3- to 8-membered heterocyclic ring is piperidine; and
(3) in ring Q, one of the rings in the 8- to 11-membered fused bicyclic ring is a saturated or partially unsaturated 5- to 7-membered heterocyclic ring, wherein the 5- to 7-membered heterocyclic ring is a 6-membered heterocyclic ring.

18. The compound, or the pharmaceutically acceptable salt, isotope derivative, enantiomer, diastereomer, tautomer, or solvate thereof according to claim 17, wherein the 5- to 7-membered carbocyclic ring is a 6- to 7-membered carbocyclic ring.

19. The compound, or the pharmaceutically acceptable salt, isotope derivative, enantiomer, diastereomer, tautomer, or solvate thereof according to claim 10, wherein,
in scheme (1), each R₃ is independently hydroxyl, C₁-C₆ alkyl, —NH—C(O)Rₑ, or —NH—S(O)₂Rₑ;
or, in scheme (3), each R₃ is independently F, Cl, Br, I, C₁-C₆ alkyl, or —NH—C(O)Rₑ.
